(19) United States
(12) Patent Application Publication
(10) Pub. No.: US 2004/0086913 A1
(43) Pub. Date: May 6, 2004

(54) Human genes and gene expression products XVI

(76) Inventors: Lewis T. Williams, Mill Valley, CA (US); Jaime Escobedo, Alamo, CA (US); Michael A. Innis, Moraga, CA (US); Pablo Dominguez Garcia, San Francisco, CA (US); Julie Sudduth Klinger, Kensington, CA (US); Christoph Reinhard, Alameda, CA (US); Zhijun He, Ithaca, NY (US); Filippo Randazzo, Oakland, CA (US); Giulia C. Kennedy, San Francisco, CA (US); David Pot, Arlington, VA (US); Altaf Kassam, Oakland, CA (US); George Lamson, Moraga, CA (US); Radjoe Drmanac, Palo Alto, CA (US); Mark Dickson, Hollister, CA (US); Ivan Labat, Mountain View, CA (US); Lee William Jones, Sunnyvale, CA (US); Birgit Stache-Crain, Sunnyvale, CA (US)

Correspondence Address: Chiron Corporation Intellectual, Property-R440, P.O. Box 8097, Emeryville, CA, 94662-8097, US

(21) Appl. No.: 10/609,021

(22) Filed: Jun. 26, 2003

Related U.S. Application Data
(63) Continuation of application No. 09/819,150, filed on Mar. 27, 2001, now abandoned.
(60) Provisional application No. 60/192,583, filed on Mar. 28, 2000.

(51) Int. Cl. C12Q 1/68; C07H 21/04; C12N 9/00; C07K 14/705; C07K 14/47; C07K 16/18

(52) U.S. Cl. 435/6; 435/69.1; 435/183; 435/320.1; 435/325; 530/350; 530/388.26; 536/23.2

(57) ABSTRACT

This invention relates to novel human polynucleotides and variants thereof, their encoded polypeptides and variants thereof, to genes corresponding to these polynucleotides and to proteins expressed by the genes. The invention also relates to diagnostic and therapeutic agents employing such novel human polynucleotides, their corresponding genes or gene products, e.g., these genes and proteins, including probes, antisense constructs, and antibodies.

### FIELD OF THE INVENTION

The present invention relates to polynucleotides of human origin and the encoded gene products.

### BACKGROUND OF THE INVENTION

Identification of novel polynucleotides, particularly those that encode an expressed gene product, is important in the advancement of drug discovery, diagnostic technologies, and the understanding of the progression and nature of complex diseases such as cancer. Identification of genes expressed in different cell types isolated from sources that differ in disease state or stage, developmental stage, exposure to various environmental factors, the tissue of origin, the species from which the tissue was isolated, and the like is key to identifying the genetic factors that are responsible for the phenotypes associated with these various differences.

This invention provides novel human polynucleotides, the polypeptides encoded by these polynucleotides, and the genes and proteins corresponding to these novel polynucleotides.

### SUMMARY OF THE INVENTION

This invention relates to novel human polynucleotides and variants thereof, their encoded polypeptides and variants thereof, to genes corresponding to these polynucleotides and to proteins expressed by the genes. The invention also relates to diagnostics and therapeutics comprising such novel human polynucleotides, their corresponding genes or gene products, including probes, antisense nucleotides, and antibodies. The polynucleotides of the invention correspond to a polynucleotide comprising the sequence information of at least one of SEQ ID NOS:1-316.

Various aspects and embodiments of the invention will be readily apparent to the ordinarily skilled artisan upon reading the description provided herein.

### BRIEF DESCRIPTION OF THE FIGURES

FIGS. 1A-1B is a comparison of SEQ ID NO:315 and clone H72034 (SEQ ID NO:317).

FIG. 2 is a comparison of SEQ ID NO:316 and clone AA707002 (SEQ ID NO:318).

### DETAILED DESCRIPTION OF THE INVENTION

The invention relates to polynucleotides comprising the disclosed nucleotide sequences, to full length cDNA, mRNA genomic sequences, and genes corresponding to these sequences and degenerate variants thereof, and to polypeptides encoded by the polynucleotides of the invention and polypeptide variants. The following detailed description describes the polynucleotide compositions encompassed by the invention, methods for obtaining cDNA or genomic DNA encoding a full-length gene product, expression of these polynucleotides and genes, identification of structural motifs of the polynucleotides and genes, identification of the function of a gene product encoded by a gene corresponding to a polynucleotide of the invention, use of the provided polynucleotides as probes and in mapping and in tissue profiling, use of the corresponding polypeptides and other gene products to raise antibodies, and use of the polynucleotides and their encoded gene products for therapeutic and diagnostic purposes.

Polynucleotide Compositions

The scope of the invention with respect to polynucleotide compositions includes, but is not necessarily limited to, polynucleotides having a sequence set forth in any one of SEQ ID NOS:1-316; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicted.

The invention features polynucleotides that are expressed in human tissue, specifically human colon, breast, and/or lung tissue. Novel nucleic acid compositions of the invention of particular interest comprise a sequence set forth in any one of SEQ ID NOS:1-316 or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from any one of SEQ ID NOS:1-316.

The polynucleotides of the invention also include polynucleotides having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well Known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences (SEQ ID NOS:1-316) under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

Preferably, hybridization is performed using at least 15 contiguous nucleotides (nt) of at least one of SEQ ID NOS:1-316. That is, when at least 15 contiguous nt of one of the disclosed SEQ ID NOS. is used as a probe, the probe will preferentially hybridize with a nucleic acid comprising the complementary sequence, allowing the identification and retrieval of the nucleic acids that uniquely hybridize to the selected probe. Probes from more than one SEQ ID NO. can hybridize with the same nucleic acid if the cDNA from which they were derived corresponds to one mRNA. Probes of more than 15 nt can be used, e.g., probes of from about 18 nt to about 100 nt, but 15 nt represents sufficient sequence for unique identification.

The polynucleotides of the invention also include naturally occurring variants of the nucleotide sequences (e.g., degenerate variants, allelic variants, etc.). Variants of the polynucleotides of the invention are identified by hybridization of putative variants with nucleotide sequences disclosed herein, preferably by hybridization under stringent conditions. For example, by using appropriate wash conditions, variants of the polynucleotides of the invention can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as single bp mismatch.

The invention also encompasses homologs corresponding to the polynucleotides of SEQ ID NOS:1-316, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST, described in Altschul, et al. Nucleic Acids Res. (1997) 25:3389-3402.

In general, variants of the invention have a sequence identity greater than at least about 65%, preferably at least about 75%, more preferably at least about 85%, and can be greater than at least about 90% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject polypeptides. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments of the invention comprise at least about 10, about 15, about 20, about 35, about 50, about 100, about 150 to about 200, about 250 to about 300, or about 350 contiguous nt selected from the polynucleotide sequences as shown in SEQ ID NOS:1-316. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from the group consisting of the polynucleotides shown in SEQ ID NOS:1-316.

Probes specific to the polynucleotides of the invention can be generated using the polynucleotide sequences disclosed in SEQ ID NOS:1-316. The probes are preferably at least about a 12, 15, 16, 18, 20, 22, 24, or 25 nt fragment of a corresponding contiguous sequence of SEQ ID NOS:1-316, and can be less than 2, 1, 0.5, 0. 1, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of a polynucleotide of one of SEQ ID NOS:1-316. More preferably, probes are designed based on a contiguous sequence of one of the subject polynucleotides that remain unmasked following application of a masking program for masking low complexity (e.g., XBLAST) to the sequence., i.e., one would select an unmasked region, as indicated by the polynucleotides outside the poly-n stretches of the masked sequence produced by the masking program.

The polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The polynucleotides of the invention can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used to, for example, produce polypeptides, as probes for the detection of mRNA of the invention in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of the polynucleotide sequences as shown in SEQ ID NOS:1-316 or variants thereof in a sample. These and other uses are described in more detail below.

Use of Polynucleotides to Obtain Full-Length cDNA, Gene, and Promoter Region

Full-length cDNA molecules comprising the disclosed polynucleotides are obtained as follows. A polynucleotide having a sequence of one of SEQ ID NOS:1-316, or a portion thereof comprising at least 12, 15, 18, or 20 nt, is used as a hybridization probe to detect hybridizing members of a cDNA library using probe design methods, cloning methods, and clone selection techniques such as those described in U.S. Pat. No. 5,654,173. Libraries of cDNA are made from selected tissues, such as normal or tumor tissue, or from tissues of a mammal treated with, for example, a pharmaceutical agent. Preferably, the tissue is the same as the tissue from which the polynucleotides of the invention were isolated, as both the polynucleotides described herein and the cDNA represent expressed genes. Most preferably, the cDNA library is made from the biological material described herein in the Examples. The choice of cell type for library construction can be made after the identity of the protein encoded by the gene corresponding to the polynucleotide of the invention is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human colon cells, more preferably, human colon cancer cells, even more preferably, from a highly metastatic colon cell, Km12L4-A.

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA can be prepared by using primers based on sequence from SEQ ID NOS:1-316. In one embodiment, the cDNA library can be made from only poly-adenylated mRNA. Thus, poly-T primers can be used to prepare cDNA from the mRNA.

Members of the library that are larger than the provided polynucleotides, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2nd Ed., (1989) Cold Spring Harbor Press, Cold Spring Harbor, N.Y. In order to obtain additional sequences 5′ to the end of a partial cDNA, 5′ RACE (PCR Protocols. A Guide to Methods and Applications, (1990) Academic Press, Inc.) can be performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the invention, but this is not essential. Most preferably, the genomic DNA is obtained from the biological material described herein in the Examples. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntsville, Ala., USA, for example. In order to obtain additional 5′ or 3′ sequences, chromosome walking is performed, as described in Sambrook et al., such that adjacent and overlapping fragments of genomic DNA are isolated. These are mapped and pieced together, as is known in the art, using restriction digestion enzymes and DNA ligase.

Using the polynucleotide sequences of the invention, corresponding full-length genes can be isolated using both classical and PCR methods to construct and probe cDNA libraries. Using either method, Northern blots, preferably, are performed on a number of cell types to determine which cell lines express the gene of interest at the highest level. Classical methods of constructing cDNA libraries are taught in Sambrook et a., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods. Gene trapping entails inserting a member of a cDNA library into a vector. The vector then is denatured to produce single stranded molecules. Next, a substrate-bound probe, such a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the polynucleotide sequences of the invention. Random primers or primers specific to the library vector can be used to amplify the trapped cDNA. Such gene trapping techniques are described in Gruber et al., WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs are ligated to an oligonucleotide linker, and amplified by PCR using two primers. One primer is based on sequence from the instant polynucleotides, for which full length sequence is desired, and a second primer comprises sequence that hybridizes to the oligonucleotide linker to amplify the cDNA. A description of this methods is reported in WO 97/19110. In preferred embodiments of RACE, a common primer is designed to anneal to an arbitrary adaptor sequence ligated to cDNA ends (Apte and Siebert, Biotechniques (1993) 15:890-893; Edwards et al., Nuc. Acids Res. (1991) 19:5227-5232). When a single gene-specific RACE primer is paired with the common primer, preferential amplification of sequences between the single gene specific primer and the common primer occurs. Commercial cDNA pools modified for use in RACE are available.

Another PCR-based method generates full-length cDNA library with anchored ends without needing specific knowledge of the cDNA sequence. The method uses lock-docking primers (I-VI), where one primer, poly TV (I-III) locks over the polyA tail of eukaryotic mRNA producing first strand synthesis and a second primer, polyGH (IV-VI) locks onto the polyC tail added by terminal deoxynucleotidyl transferase (TdT)(see, e.g., WO 96/40998).

The promoter region of a gene generally is located 5′ to the initiation site for RNA polymerase II. Hundreds of promoter regions contain the “TATA” box, a sequence such as TATTA or TATAA, which is sensitive to mutations. The promoter region can be obtained by performing 5′ RACE using a primer from the coding region of the gene. Alternatively, the cDNA can be used as a probe for the genomic sequence, and the region 5′ to the coding region is identified by “walking up.” If the gene is highly expressed or differentially expressed, the promoter from the gene can be of use in a regulatory construct for a heterologous gene.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the invention can be synthesized. Thus, the invention encompasses nucleic acid molecules ranging in length from 15 nt (corresponding to at least 15 contiguous nt of one of SEQ ID NOS:1-316) up to a maximum length suitable for one or more biological manipulations, including replication and 30 expression, of the nucleic acid molecule. The invention includes but is not limited to (a) nucleic acid having the size of a full gene, and comprising at least one of SEQ ID NOS:1-316; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b). Once provided with the polynucleotides disclosed herein, construction or preparation of (a)-(e) are well within the skill in the art.

The sequence of a nucleic acid comprising at least 15 contiguous nt of at least any one of SEQ ID NOS:1-316, preferably the entire sequence of at least any one of SEQ ID NOS:1-316, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of any one of SEQ ID NOS:1-316 is within the nucleic acid, the nucleic acid obtained is referred to herein as a polynucleotide comprising the sequence of any one of SEQ ID NOS:1-3 16.

Expression of Polypeptide Encoded by Full-Length cDNA or Full-Length Gene

The provided polynucleotides (e.g., a polynucleotide having a sequence of one of SEQ ID NOS:1-316), the corresponding cDNA, or the full-length gene is used to express a partial or complete gene product. Constructs of polynucleotides having sequences of SEQ ID NOS:1-316 can also be generated synthetically. Alternatively, single-step assembly of a gene and entire plasmid from large numbers of oligodeoxyribonucleotides is described by, e.g. Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process.

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research. The gene product encoded by a polynucleotide of the invention is expressed in any expression system, including, for example, bacterial, yeast, insect, amphibian and mammalian systems. Vectors, host cells and methods for obtaining expression in same are well known in the art. Suitable vectors and host cells are described in U.S. Pat. No. 5,654,173.

Polynucleotide molecules comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in SEQ ID NOS:1-3 16 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670.

Identification of Functional and Structural Motifs of Novel Genes Screening Against Publicly Available Databases

Translations of the nucleotide sequence of the provided polynucleotides, cDNAs or full genes can be aligned with individual known sequences. Similarity with individual sequences can be used to determine the activity of the polypeptides encoded by the polynucleotides of the invention. Also, sequences exhibiting similarity with more than one individual sequence can exhibit activities that are characteristic of either or both individual sequences.

The full length sequences and fragments of the polynucleotide sequences of the nearest neighbors can be used as probes and primers to identify and isolate the full length sequence corresponding to provided polynucleotides. The nearest neighbors can indicate a tissue or cell type to be used to construct a library for the full-length sequences corresponding to the provided polynucleotides.

Typically, a selected polynucleotide is translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences. Databases with individual sequences are described in “Computer Methods for Macromolecular Sequence Analysis” Methods in Enzymology (1996) 266, Doolittle, Academic Press, Inc., a division of Harcourt Brace & Co., San Diego, Calif., USA. Databases include GenBank, EMBL, and DNA Database of Japan (DDBJ).

Query and individual sequences can be aligned using the methods and computer programs described above, and include BLAST 2.0, available over the world wide web at a site supported by the National Center for Biotechnology Information, which is supported by the National Library of Medicine and the National Institutes of Health. See also Altschul, et al. Nucleic Acids Res. (1997) 25:3389-3402. Another alignment algorithm is Fasta, available in the Genetics Computing Group (GCG) package, Madison, Wis., USA, a wholly owned subsidiary of Oxford Molecular Group, Inc. Other techniques for alignment are described in Doolittle, supra. Preferably, an alignment program that permits gaps in the sequence is utilized to align the sequences. The Smith-Waterman is one type of algorithm, that permits gaps in sequence alignments. See Meth. Mol. Biol. (1997) 70: 173-187. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to identify sequences that are distantly related matches, and is especially tolerant of small gaps and nucleotide sequence errors. Amino acid sequences encoded by the provided polynucleotides can be used to search both protein and DNA databases. Incorporated herein by reference are all sequences that have been made public as of the filing date of this application by any of the DNA or protein sequence databases, including the patent databases (e.g., GeneSeq). Also incorporated by reference are those sequences that have been submitted to these databases as of the filing date of the present application but not made public until after the filing date of the present application.

Results of individual and query sequence alignments can be divided into three categories: high similarity, weak similarity, and no similarity. Individual alignment results ranging from high similarity to weak similarity provide a basis for determining polypeptide activity and/or structure. Parameters for categorizing individual results include: percentage of the alignment region length where the strongest alignment is found, percent sequence identity, and p value. The percentage of the alignment region length is calculated by counting the number of residues of the individual sequence found in the region of strongest alignment, e.g., contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is: 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%

P value is the probability that the alignment was produced by chance. For a single alignment, the p value can be calculated according to Karlin et al., Proc. Natl. Acad. Sci. (1990) 87:2264 and Karlin et al., Proc. Natl. Acad. Sci. (1993) 90. The p value of multiple alignments using the same query sequence can be calculated using an heuristic approach described in Altschul et. al., Nat. Genet. (1994) 6:119. Alignment programs such as BLAST program can calculate the p value. See also Altschul et al., Nucleic Acids Res. (1997) 25:3389-3402.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences. The boundaries of the region where the sequences align can be determined according to Doolittle, supra; BLAST 2.0 (see, e.g., Altschul, et al. Nucleic Acids Res. (1997) 25-3389-3402) or FAST programs; or by determining the area where sequence identity is highest.

High Similarity. In general, in alignment results considered to be of high similarity, the percent of the alignment region length is typically at least about 55% of total length query sequence; more typically, at least about 58%; even more typically; at least about 60% of the total residue length of the query sequence. Usually, percent length of the alignment region can be as much as. about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. If high similarity is found, the query sequence is considered to have high similarity with a profile sequence when the p value is less than or equal to about 10⁻²; more usually; less than or equal to about 10⁻³; even more usually; less than or equal to about 10⁻⁴. More typically, the p value is no more than about 10⁻⁵; more typically; no more than or equal to about 10⁻¹⁰; even more typically; no more than or equal to about 10⁻¹⁵ for the query sequence to be considered high similarity.

Weak Similarity In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically; at least about 25 amino acid residues in length. Usually, length of the alignment region can be as much as about 30 amino acid residues; more usually, as much as about 40; even more usually, as much as about 60 amino acid residues. Further, for weak similarity, the region of alignment, typically, exhibits at least about 35% of sequence identity; more typically, at least about 40%; even more typically; at least about 45% sequence identity. Usually, percent sequence identity can be as much as about 50%; more usually, as much as about 55%; even more usually, as much as about 60%.

If low similarity is found, the query sequence is considered to have weak similarity with a profile sequence when the p value is usually less than or equal to about 10⁻²; more usually; less than or equal to about 10⁻³; even more usually; less than or equal to about 10⁻⁴. More typically, the p value is no more than about 10⁻⁵; more usually; no more than or equal to about 10⁻¹⁰; even more usually; no more than or equal to about 10⁻¹⁵ for the query sequence to be considered weak similarity.

Similarity Determined by Sequence Identity Alone. Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, 90 residues; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

Alignments with Profile and Multiple Aligned Sequences. Translations of the provided polynucleotides can be aligned with amino acid profiles that define either protein families or common motifs. Also, translations of the provided polynucleotides can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided polynucleotides or corresponding cDNA or genes. For example, sequences that show an identity or similarity with a chemokine profile or MSA can exhibit chemokine activities.

Profiles can designed manually by (1) creating an NISA, which is an alignment of the amino acid sequence of members that belong to the family and (2) constructing a statistical representation of the alignment. Such methods are described, for example, in Bimey et al., Nucl. Acid Res. (1996) 24(14): 2730-2739. MSAs of some protein families and motifs are publicly available. For example, the Genome Sequencing Center at thw Washington University School of Medicine provides a web set (Pfam) which includes MSAs of 547 different families and motifs. These MSAs are described also in Sonnhammer et al., Proteins (1997) 28: 405-420. Other sources over the world wide web include the site supported by the European Molecular Biology Laboratories in Heidelberg, Germany. A brief description of these MSAs is reported in Pascarella et al., Prot. Eng. (1996) 9(3):249-251. Techniques for building profiles from MSAs are described in Sonnhammer et al., supra; Birney et al., supra; and “Computer Methods for Macromolecular Sequence Analysis,” Methods in Enzymology (1996) 266, Doolittle, Academic Press, Inc., San Diego, Calif., USA.

Similarity between a query sequence and a protein family or motif can be determined by (a) comparing the query sequence against the profile and/or (b) aligning the query sequence with the members of the family or motif. Typically, a program such as Searchwise is used to compare the query sequence to the statistical representation of the multiple alignment, also known as a profile (see Bimey et al., supra). Other techniques to compare the sequence and profile are described in Sonnhammer et al., supra and Doolittle, supra.

Next, methods described by Feng et al., J. Mol. Evol. (1987) 25:351 and Higgins et al., CABIOS (1989) 5:151 can be used align the query sequence with the members of a family or motif, also known as a MSA. Sequence alignments can be generated using any of a variety of software tools. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:443. GAP is best suited for global alignment of sequences. A third method, BestFit, functions by inserting gaps to maximize the number of matches using the local homology algorithm of Smith et al., Adv. Appl. Math. (1981) 2:482. In general, the following factors are used to determine if a similarity between a query sequence and a profile or MSA exists: (1) number of conserved residues found in the query sequence, (2) percentage of conserved residues found in the query sequence, (3) number of frameshifts, and (4) spacing between conserved residues.

Some alignment programs that both translate and align sequences can make any number of frameshifts when translating the nucleotide sequence to produce the best alignment. The fewer frameshifts needed to produce an alignment, the stronger the similarity or identity between the query and profile or MSAs. For example, a weak similarity resulting from no frameshifts can be a better indication of activity or structure of a query sequence, than a strong similarity resulting from two frameshifts. Preferably, three or fewer frameshifts are found in an alignment; more preferably two or fewer frameshifts; even more preferably, one or fewer frameshifts; even more preferably, no frameshifts are found in an alignment of query and profile or MSAs.

Conserved residues are those amino acids found at a particular position in all or some of the family or motif members. Alternatively, a position is considered conserved if only a certain class of amino acids is found in a particular position in all or some of the family members. For example, the N-terminal position can contain a positively charged amino acid, such as lysine, arginine, or histidine.

Typically, a residue of a polypeptide is conserved when a class of amino acids or a single amino acid is found at a particular position in at least about 40% of all class members; more typically, at least about 50%; even more typically, at least about 60% of the members. Usually, a residue is conserved when a class or single amino acid is found in at least about 70% of the members of a family or motif; more usually, at least about 80%; even more usually, at least about 90%; even more usually, at least about 95%.

A residue is considered conserved when three unrelated amino acids are found at a particular position in the some or all of the members; more usually, two unrelated amino acids.

These residues are conserved when the unrelated amino acids are found at particular positions in at least about 40% of all class member; more typically, at least about 50%; even more typically, at least about 60% of the members. Usually, a residue is conserved when a class or single amino acid is found in at least about 70% of the members of a family or motif; more usually, at least about 80%; even more usually, at least about 90%; even more usually, at least about 95%.

A query sequence has similarity to a profile or MSA when the query sequence comprises at least about 25% of the conserved residues of the profile or MSA; more usually, at least about 30%; even more usually; at least about 40%. Typically, the query sequence has a stronger similarity to a profile sequence or MSA when the query sequence comprises at least about 45% of the conserved residues of the profile or MSA; more typically, at least about 50%; even more typically; at least about 55%.

Identification of Secreted & Membrane-Bound Polypeptides

Both secreted and membrane-bound polypeptides of the present invention are of particular interest. For example, levels of secreted polypeptides can be assayed in body fluids that are convenient, such as blood, plasma, serum, and other body fluids such as urine, prostatic fluid and semen. Membrane-bound polypeptides are useful for constructing vaccine antigens or inducing an immune response. Such antigens would comprise all or part of the extracellular region of the membrane-bound polypeptides. Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 190: 207-219.

Another method of identifying secreted and membrane-bound polypeptides is to translate the polynucleotides of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine.

Identification of the Function of an Expression Product of a Full-Length Gene

Ribozymes, antisense constructs, and dominant negative mutants can be used to determine function of the expression product of a gene corresponding to a polynucleotide provided herein. These methods and compositions are particularly useful where the provided novel polynucleotide exhibits no significant or substantial homology to a sequence encoding a gene of kmown function. Antisense molecules and ribozymes can be constructed from synthetic polynucleotides. Typically, the phosphoramidite method of oligonucleotide synthesis is used. See Beaucage et al., Tet. Lett. (1981) 22:1859 and U.S. Pat. No. 4,668,777. Automated devices for synthesis are available to create oligonucleotides using this chemistry. Examples of such devices include Biosearch 8600, Models 392 and 394 by Applied Biosystems, a division of Perkin-Elmer Corp., Foster City, Calif., USA; and Expedite by Perceptive Biosystems, Framingham, Mass., USA. Synthetic RNA, phosphate analog oligonucleotides, and chemically derivatized oligonucleotides can also be produced, and can be covalently attached to other molecules. RNA oligonucleotides can be synthesized, for example, using RNA phosphoramidites. This method can be performed on an automated synthesizer, such as Applied Biosystems, Models 392 and 394, Foster City, Calif., USA.

Phosphorothioate oligonucleotides can also be synthesized for antisense construction. A sulfurizing reagent, such as tetraethylthiruam disulfide (TETD) in acetonitrile can be used to convert the internucleotide cyanoethyl phosphite to the phosphorothioate triester within 15 minutes at room temperature. TETD replaces the iodine reagent, while all other reagents used for standard phosphoramidite chemistry remain the same. Such a synthesis method can be automated using Models 392 and 394 by Applied Biosystems, for example.

Oligonucleotides of up to 200 nt can be synthesized, more typically, 100 nt, more typically 50 nt; even more typically 30 to 40 nt. These synthetic fragments can be annealed and ligated together to construct larger fragments. See, for example, Sambrook et al., supra. Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect. One commonly used ribozyme motif is the hammerhead, for which the substrate sequence requirements are minimal. Design of the hammerhead ribozyme, as well as therapeutic uses of ribozymes, are disclosed in Usman et al., Current Opin. Struct. Biol. (1996) 6:527. Methods for production of ribozymes, including hairpin structure ribozyme fragments, methods of increasing ribozyme specificity, and the like are known in the art.

The hybridizing region of the ribozyme can be modified or can be prepared as a branched structure as described in Horn and Urdea, Nucleic Acids Res. (1989) 17:6959. The basic structure of the ribozymes can also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1.

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence can interfere with expression of the corresponding gene. Antisense polynucleotides are typically generated within the cell by expression from antisense constructs that contain the antisense strand as the transcribed strand. Antisense polynucleotides based on the disclosed polynucleotides will bind and/or interfere with the translation of mRNA comprising a sequence complementary to the antisense polynucleotide. The expression products of control cells and cells treated with the antisense construct are compared to detect the protein product of the gene corresponding to the polynucleotide upon which the antisense construct is based. The protein is isolated and identified using routine biochemical methods.

Given the extensive background literature and clinical experience in antisense therapy, one skilled in the art can use selected polynucleotides of the invention as additional potential therapeutics. The choice of polynucleotide can be narrowed by first testing them for binding to “hot spot” regions of the genome of cancerous cells. If a polynucleotide is identified as binding to a “hot spot”, testing the polynucleotide as an antisense compound in the corresponding cancer cells is warranted.

As an alternative method for identifying function of the gene corresponding to a polynucleotide disclosed herein, dominant negative mutations are readily generated for corresponding proteins that are active as homomultimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants (see, e.g., Herskowitz, Nature (1987) 329:219). Such techniques can be used to create loss of function mutations, which are useful for determining protein function.

Polypeptides and Variants Thereof

The polypeptides of the invention include those encoded by the disclosed polynucleotides, as well as nucleic acids that, by virtue of the degeneracy of the genetic code, are not identical in sequence to the disclosed polynucleotides. Thus, the invention includes within its scope a polypeptide encoded by a polynucleotide having the sequence of any one of SEQ ID NOS:1-316 or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the invention, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any SEQ ID NOS:1-316, or a homolog thereof. The protein variants described herein are encoded by polynucleotides that are within the scope of the invention. The genetic code can be used to select the appropriate codons to construct the corresponding variants.

Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a breast ductal cell affected by cancer relative to a normal (i.e., substantially disease-free) breast cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of SEQ ID NOS:1-316. By plurality is meant at least 2, usually at least 3 and can include up to all of SEQ ID NOS:1-316. The length and number of polynucleotides in the library will vary with the nature of the library, e.g. if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of SEQ ID NOS:1-316, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, et. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information is publicly available. For example, the gapped BLAST (Altschul et al. Nucleic Acids Res. (1997) 25:3389-3402) and BLAZE (Brutlag et al. Comp. Chem. (1993) 17:203) search algorithms on a Sybase system can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information. Search means can be used to identify fragments or regions of the genome that match a particular target sequence or target motif. A variety of known algorithms are publicly known and commercially available, e.g. MacPattern (EMBL), BLASTN and BLASTX (NCBI). A “target sequence” can be any polynucleotide or amino acid sequence of six or more contiguous nucleotides or two or more amino acids, preferably from about 10 to 100 amino acids or from about 30 to 300 nt A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but arc not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile.

As discussed above, the “library” of the invention also encompasses biochemical libraries of the polynucleotides of SEQ ID NOS:1-316, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of SEQ ID NOS:1-316 is represented on the array. By array is meant a an article of manufacture that has at least a substrate with at least two distinct nucleic acid targets on one of its surfaces, where the number of distinct nucleic acids can be considerably higher, typically being at least 10 nt, usually at least 20 nt and often at least 25 nt. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the where the polypeptides of the library will represent at least a portion of the polypeptides encoded by SEQ ID NOS:1-316.

Utilities

Use of Polynucleotide Probes in Mapping, and in Tissue Profiling

Polynucleotide probes, generally comprising at least 12 contiguous nt of a polynucleotide as shown in the Sequence Listing, are used for a variety of purposes, such as chromosome mapping of the polynucleotide and detection of transcription levels. Additional disclosure about preferred regions of the disclosed polynucleotide sequences is found in the Examples. A probe that hybridizes specifically to a polynucleotide disclosed herein should provide a detection signal at least 5-, 10-, or 20-fold higher than the background hybridization provided with other unrelated sequences.

Detection of Expression Levels. Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are used for in situ hybridization to cells to detect expression Probes can also be used in vivo for diagnostic detection of hybridizing sequences. Probes are typically labeled with a radioactive isotope. Other types of detectable labels can be used such as chromophores, fluors, and enzymes. Other examples of nucleotide hybridization assays are described in WO92/02526 and U.S. Pat. No. 5,124,246.

Alternatively, the Polymerase Chain Reaction (PCR) is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer polynucleotides nucleotides that hybridize with the target nucleic acids are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Mapping. Polynucleotides of the present invention can be used to identify a chromosome on which the corresponding gene resides. Such mapping can be useful in identifying the function of the polynucleotide-related gene by its proximity to other genes with known function. Function can also be assigned to the polynucleotide-related gene when particular syndromes or diseases map to the same chromosome. For example, use of polynucleotide probes in identification and quantification of nucleic acid sequence aberrations is described in U.S. Pat. No. 5,783,387. An exemplary mapping method is fluorescence in situ hybridization (FISH), which facilitates comparative genomic hybridization to allow total genome assessment of changes in relative copy number of DNA sequences (see, e.g., Valdes et al., Methods in Molecular Biology (1997) 68:1). Polynucleotides can also be mapped to particular chromosomes using, for example, radiation hybrids or chromosome-specific hybrid panels. See Leach et al., Advances in Genetics, (1995) 33:63-99; Walter et al., Nature Genetics (1994) 7:22; Walter and Goodfellow, Trends in Genetics (1992) 9:352. Panels for radiation hybrid mapping are available from Research Genetics, Inc., Huntsville, Ala., USA. Databases for markers using various panels are available via the world wide web at sites supported by the Stanford Human Genome Center (Stanford University) and the Whitehead Institute for Biomedical Research/MIT Center for Genome Research. The statistical program RHMAP can be used to construct a map based on the data from radiation hybridization with a measure of the relative likelihood of one order versus another. RHMAP is available via the world wide web at a site supported by the Center for Statistical Genetics at the University of Michigan School of Public Health. In addition, commercial programs are available for identifying regions of chromosomes commonly associated with diseases such as cancer.

Tissue Typing or Profiling. Expression of specific mRNA corresponding to the provided polynucleotides can vary in different cell types and can be tissue-specific. This variation of mRNA levels in different cell types can be exploited with nucleic acid probe assays to determine tissue types. For example, PCR, branched DNA probe assays, or blotting techniques utilizing nucleic acid probes substantially identical or complementary to polynucleotides listed in the Sequence Listing can determine the presence or absence of the corresponding cDNA or mRNA.

Tissue typing can be used to identify the developmental organ or tissue source of a metastatic lesion by identifying the expression of a particular marker of that organ or tissue. If a polynucleotide is expressed only in a specific tissue type, and a metastatic lesion is found to express that polynucleotide, then the developmental source of the lesion has been identified. Expression of a particular polynucleotide can be assayed by detection of either the corresponding mRNA or the protein product. As would be readily apparent to any forensic scientist, the sequences disclosed herein are useful in differentiating human tissue from non-human tissue. In particular, these sequences are useful to differentiate human tissue from bird, reptile, and amphibian tissue, for example.

Use of Polymorphisms. A polynucleotide of the invention can be used in forensics, genetic analysis, mapping, and diagnostic applications where the corresponding region of a gene is polymorphic in the human population. Any means for detecting a polymorphism in a gene can be used, including, but not limited to electrophoresis of protein polymorphic variants, differential sensitivity to restriction enzyme cleavage, and hybridization to allele-specific probes.

Antibody Production

Expression products of a polynucleotide of the invention, as well as the corresponding mRNA, cDNA, or complete gene, can be prepared and used for raising antibodies for experimental, diagnostic, and therapeutic purposes. For polynucleotides to which a corresponding gene has not been assigned, this provides an additional method of identifying the corresponding gene. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Methods for production of antibodies that specifically bind a selected antigen are well known in the art. Immunogens for raising antibodies can be prepared by mixing a polypeptide encoded by a polynucleotide of the invention with an adjuvant, and/or by making fusion proteins with larger immunogenic proteins. Polypeptides can also be covalently linked to other larger immunogenic proteins, such as keyhole limpet hemocyanin. Immunogens are typically administered intradermally, subcutaneously, or intramuscularly to experimental animals such as rabbits, sheep, and mice, to generate antibodies. Monoclonal antibodies can be Monoclonal antibodies can be generated by isolating spleen cells and fusing myeloma cells to form hybridomas. Alternatively, the selected polynucleotide is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for polypeptides encoded by a selected polynucleotide are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. Epitopes that involve non-contiguous amino acids may require a longer polypeptide, e.g., at least 15, 25, or 50 amino acids. Antibodies that specifically bind to human polypeptides encoded by the provided polypeptides should provide a detection signal at least 5-, 10-, or 20-fold higher than a detection signal provided with other proteins when used in Western blots or other immunochemical assays. Preferably, antibodies that specifically polypeptides of the invention do not bind to other proteins in immunochemical assays at detectable levels and can immunoprecipitate the specific polypeptide from solution.

The invention also contemplates naturally occurring antibodies specific for a polypeptide of the invention. For example, serum antibodies to a polypeptide of the invention in a human population can be purified by methods well known in the art, e.g., by passing antiserum over a column to which the corresponding selected polypeptide or fusion protein is bound. The bound antibodies can then be eluted from the column, for example using a buffer with a high salt concentration.

In addition to the antibodies discussed above, the invention also contemplates genetically engineered antibodies, antibody derivatives (e.g., single chain antibodies, antibody fragments (e.g., Fab, etc.)), according to methods well known in the art.

Polynucleotides or Arrays for Diagnostics

Polynucleotide arrays provide a high throughput technique that can assay a large number of polynucleotide sequences in a sample. This technology can be used as a diagnostic and as a tool to test for differential expression, e.g., to determine function of an encoded protein. Arrays can be created by spotting polynucleotide probes onto a substrate (e.g., glass, nitrocelllose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. Arrays can be used to, for example, examine differential expression of genes and can be used to determine gene function. For example, arrays can be used to detect differential expression of a polynucleotide between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 16:40.

Differential Expression in Diagnosis

The polynucleotides of the invention can also be used to detect differences in expression levels between two cells, e.g., as a method to identify abnormal or diseased tissue in a human. For polynucleotides corresponding to profiles of protein families, the choice of tissue can be selected according to the putative biological function. In general, the expression of a gene corresponding to a specific polynucleotide is compared between a first tissue that is suspected of being diseased and a second, normal tissue of the human. The tissue suspected of being abnormal or diseased can be derived from a different tissue type of the human, but preferably it is derived from the same tissue type; for example an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon). A difference between the polynucleotide-related gene, mRNA, or protein in the two tissues which are compared, for example in molecular weight, amino acid or nucleotide sequence, or relative abundance, indicates a change in the gene, or a gene which regulates it, in the tissue of the human that was suspected of being diseased. Examples of detection of differential expression and its use in diagnosis of cancer are described in U.S. Pat. Nos. 5,688,641 and 5,677,125.

A genetic predisposition to disease in a human can also be detected by comparing expression levels of an mRNA or protein corresponding to a polynucleotide of the invention in a fetal tissue with levels associated in normal fetal tissue. Fetal tissues that are used for this purpose include, but are not limited to, amniotic fluid, chorionic villi, blood, and the blastomere of an in vitro-fertilized embryo. The comparable normal polynucleotide-related gene is obtained from any tissue. The mRNA or protein is obtained from a normal tissue of a human in which the polynucleotide-related gene is expressed. Differences such as alterations in the nucleotide sequence or size of the same product of the fetal polynucleotide-related gene or mRNA, or alterations in the molecular weight, amino acid sequence, or relative abundance of fetal protein, can indicate a germline mutation in the polynucleotide-related gene of the fetus, which indicates a genetic predisposition to disease. In general, diagnostic, prognostic, and other methods of the invention based on differential expression involve detection of a level or amount of a gene product, particularly a differentially expressed gene product, in a test sample obtained from a patient suspected of having or being susceptible to a disease (e.g., breast cancer, lung cancer, colon cancer and/or metastatic forms thereof), and comparing the detected levels to those levels found in normal cells (e.g., cells substantially unaffected by cancer) and/or other control cells (e.g., to differentiate a cancerous cell from a cell affected by dysplasia). Furthermore, the severity of the disease can be assessed by comparing the detected levels of a differentially expressed gene product with those levels detected in samples representing the levels of differentially gene product associated with varying degrees of severity of disease. It should be noted that use of the term “diagnostic” herein is not necessarily meant to exclude “prognostic” or “prognosis,” but rather is used as a matter of convenience.

The term “differentially expressed gene” is generally intended to encompass a polynucleotide that can, for example, include an open reading frame encoding a gene product (e.g. a polypeptide), and/or introns of such genes and adjacent 5′ and 3′ non-coding nucleotide sequences involved in the regulation of expression, up to about 20 kb beyond the coding region, but possibly further in either direction. The gene can be introduced into an appropriate vector for extrachromosomal maintenance or for integration into a host genome. In general, a difference in expression level associated with a decrease in expression level of at least about 25%, usually at least about 50% to 75%, more usually at least about 90% or more is indicative of a differentially expressed gene of interest, i.e., a gene that is underexpressed or down-regulated in the test sample relative to a control sample. Furthermore, a difference in expression level associated with an increase in expression of at least about 25%, usually at least about 50% to 75%, more usually at least about 90% and can be at least about 1½-fold, usually at least about 2-fold to about 10-fold, and can be about 100-fold to about 1,000-fold increase relative to a control sample is indicative of a differentially expressed gene of interest, i.e., an overexpressed or up-regulated gene.

“Differentially expressed polynucleotide” as used herein means a nucleic acid molecule (RNA or DNA) comprising a sequence that represents a differentially expressed gene, e.g., the differentially expressed polynucleotide comprises a sequence (e.g., an open reading frame encoding a gene product) that uniquely identifies a differentially expressed gene so that detection of the differentially expressed polynucleotide in a sample is correlated with the presence of a differentially expressed gene in a sample. “Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

“Diagnosis” as used herein generally includes determination of a subject's susceptibility to a disease or disorder, determination as to whether a subject is presently affected by a disease or disorder, as well as to the prognosis of a subject affected by a disease or disorder (e.g., identification of pre-metastatic or metastatic cancerous states, stages of cancer, or responsiveness of cancer to therapy). The present invention particularly encompasses diagnosis of subjects in the context of breast cancer (e.g., carcinoma in situ (e.g., ductal carcinoma in situ), estrogen receptor (ER)-positive breast cancer, ER-negative breast cancer, or other forms and/or stages of breast cancer), lung cancer (e.g., small cell carcinoma, non-small cell carcinoma, mesothelioma, and other forms and/or stages of lung cancer), and colon cancer (e.g., adenomatous polyp, colorectal carcinoma, and other forms and/or stages of colon cancer).

“Sample” or “biological sample” as used throughout here are genetally meant to refer to samples of biological fluids or tissues, particularly samples obtained from tissues, especially from cells of the type associated with the disease for which the diagnostic application is designed (e.g., ductal adenocarcinoma), and the like. “Samples” is also meant to encompass derivatives and fractions of such samples (e.g., cell lysates). Where the sample is solid tissue, the cells of the tissue can be dissociated or tissue sections can be analyzed.

Methods of the subject invention useful in diagnosis or prognosis typically involve comparison of the abundance of a selected differentially expressed gene product in a sample of interest with that of a control to determine any relative differences in the expression of the gene product, where the difference can be measured qualitatively and/or quantitatively. Quantitation can be accomplished, for example, by comparing the level of expression product detected in the sample with the amounts of product present in a standard curve. A comparison can be made visually; by using a technique such as densitometry, with or without computerized assistance; by preparing a representative library of cDNA clones of mRNA isolated from a test sample, sequencing the clones in the library to determine that number of cDNA clones corresponding to the same gene product, and analyzing the number of clones corresponding to that same gene product relative to the number of clones of the same gene product in a control sample; or by using an array to detect relative levels of hybridization to a selected sequence or set of sequences, and comparing the hybridization pattern to that of a control. The differences in expression are then correlated with the presence or absence of an abnormal expression pattern. A variety of different methods for determining the nucleic acid abundance in a sample are known to those of skill in the art (see, e.g., WO 97/27317).

In general, diagnostic assays of the invention involve detection of a gene product of a the polynucleotide sequence (e.g., mRNA or polypeptide) that corresponds to a sequence of SEQ ID NOS:1-316 The patient from whom the sample is obtained can be apparently healthy, susceptible to disease (e.g., as determined by family history or exposure to certain environmental factors), or can already be identified as having a condition in which altered expression of a gene product of the invention is implicated.

Diagnosis can be determined based on detected gene product expression levels of a gene product encoded by at least one, preferably at least two or more, at least 3 or more, or at least 4 or more of the polynucleotides having a sequence set forth in SEQ ID NOS:1-316, and can involve detection of expression of genes corresponding to all of SEQ ID NOS:1-3 16 and/or additional sequences that can serve as additional diagnostic markers and/or reference sequences. Where the diagnostic method is designed to detect the presence or susceptibility of a patient to cancer, the assay preferably involves detection of a gene product encoded by a gene corresponding to a polynucleotide that is differentially expressed in cancer. Examples of such differentially expressed polynucleotides are described in the Examples below. Given the provided polynucleotides and information regarding their relative expression levels provided herein, assays using such polynucleotides and detection of their expression levels in diagnosis and prognosis will be readily apparent to the ordinarily skilled artisan.

Any of a variety of detectable labels can be used in connection with the various embodiments of the diagnostic methods of the invention. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The detectable label can involve a two stage systems (e.g., biotin-avidin, hapten-anti-hapten antibody, etc.)

Reagents specific for the polynucleotides and polypeptides of the invention, such as antibodies and nucleotide probes, can be supplied in a kit for detecting the presence of an expression product in a biological sample. The kit can also contain buffers or labeling components, as well as instructions for using the reagents to detect and quantify expression products in the biological sample. Exemplary embodiments of the diagnostic methods of the invention are described below in more detail.

Polypeptide detection in diagnosis. In one embodiment, the test sample is assayed for the level of a differentially expressed polypeptide. Diagnosis can be accomplished using any of a number of methods to determine the absence or presence or altered amounts of the differentially expressed polypeptide in the test sample. For example, detection can utilize staining of cells or histological sections with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

mRNA detection. The diagnostic methods of the invention can also or alternatively involve detection of mRNA encoded by a gene corresponding to a differentially.expressed polynucleotides of the invention. Any suitable qualitative or quantitative methods known in the art for detecting specific mRNAs can be used. mRNA can be detected by, for example, in situ hybridization in tissue sections, by reverse transcriptase-PCR, or in Northern blots containing poly A+ mRNA. One of skill in the art can readily use these methods to determine differences in the size or amount of mRNA transcripts between two samples. mRNA expression levels in a sample can also be determined by generation of a library of expressed sequence tags (ESTs) from the sample, where the EST library is representative of sequences present in the sample (Adams, et al., (1991) Science 252:1651). Enumeration of the relative representation of ESTs within the library can be used to approximate the relative representation of the gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein. Alternatively, gene expression in a test sample can be performed using serial analysis of gene expression (SAGE) methodology (e.g., Velculescu et al., Science (1995) 270:484) or differential display (DD) methodology (see, e.g., U.S. Pat. No. 5,776,683; and U.S. Pat. No. 5,807,680).

Alternatively, gene expression can be analyzed using hybridization analysis. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Use of a single gene in diagnostic applications. The diagnostic methods of the invention can focus on the expression of a single differentially expressed gene. For example, the diagnostic method can involve detecting a differentially expressed gene, or a polymorphism of such a gene (e.g., a polymorphism in an coding region or control region), that is associated with disease. Disease-associated polymorphisms can include deletion or truncation of the gene, mutations that alter expression level and/or affect activity of the encoded protein, etc.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express a differentially expressed gene can be used as a source of mRNA, which can be assayed directly or reverse transcribed into cDNA for analysis. The nucleic acid can be amplified by conventional techniques, such as the polymerase chain reaction (PCR), to provide sufficient amounts for analysis, and a detectable label can be included in the. amplification reaction (e.g., using a detectably labeled primer or detectably labeled oligonucleotides) to facilitate detection. Alternatively, various methods are also known in the art that utilize oligonucleotide ligation as a means of detecting polymorphisms, see e.g., Riley et al., Nucl. Acids Res. (1990) 18:2887; and Delahunty et al., Am. J. Hum. Genet. (1996) 58:1239.

The amplified or cloned sample nucleic acid can be analyzed by one of a number of methods known in the art. The nucleic acid can be sequenced by dideoxy or other methods, and the sequence of bases compared to a selected sequence, e.g., to a wild-type sequence. Hybridization with the polymorphic or variant sequence can also be used to determine its presence in a sample (e.g., by Southern blot, dot blot, etc.). The hybridization pattern of a polymorphic or variant sequence and a control sequence to an array of oligonucleotide probes immobilized on a solid support, as described in U.S. Pat. No. 5,445,934, or in WO 95/35505, can also be used as a means of identifying polymorphic or variant sequences associated with disease. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in a gene can be based on the functional or antigenic characteristics of the protein. Protein truncation assays are useful in detecting deletions that can affect the biological activity of the protein. Various imrnunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

Pattern matching in diagnosis using arrays. In another embodiment, the diagnostic and/or prognostic methods of the invention involve detection of expression of a selected set of genes in a test sample to produce a test expression pattern (TEP). The TEP is compared to a reference expression pattern (REP), which is generated by detection of expression of the selected set of genes in a reference sample (e.g., a positive or negative control sample). The selected set of genes includes at least one of the genes of the invention, which genes correspond to the polynucleotide sequences of SEQ ID NOS:1-316. Of particular interest is a selected set of genes that includes gene differentially expressed in the disease for which the test sample is to be screened.

“Reference sequences” or “reference polynucleotides” as used herein in the context of differential gene expression analysis and diagnosis/prognosis refers to a selected set of polynucleotides, which selected set includes at least one or more of the differentially expressed polynucleotides described herein. A plurality of reference sequences, preferably comprising positive and negative control sequences, can be included as reference sequences. Additional suitable reference sequences are found in GenBank, Unigene, and other nucleotide sequence databases (including, e.g., expressed sequence tag (EST), partial, and full-length sequences).

“Reference array” means an array having reference sequences for use in hybridization with a sample, where the reference sequences include all, at least one of, or any subset of the differentially expressed polynucleotides described herein. Usually such an array will include at least 3 different reference sequences, and can include any one or all of the provided differentially expressed sequences. Arrays of interest can further comprise sequences, including polymorphisms, of other genetic sequences, particularly other sequences of interest for screening for a disease or disorder (e.g., cancer, dysplasia, or other related or unrelated diseases, disorders, or conditions). The oligonucleotide sequence on the array will usually be at least about 12 nt in length, and can be of about the length of the provided sequences, or can extend into the flanking regions to generate fragments of 100 nt to 200 nt in length or more. Reference arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

A “reference expression pattern” or “REP” as used herein refers to the relative levels of expression of a selected set of genes, particularly of differentially expressed genes, that is associated with a selected cell type, e.g., a normal cell, a cancerous cell, a cell exposed to an environmental stimulus, and the like. A “test expression pattern” or “TEP” refers to relative levels of expression of a selected set of genes, particularly of differentially expressed genes, in a test sample (e.g., a cell of unknown or suspected disease state, from which mRNA is isolated).

REPs can be generated in a variety of ways according to methods well known in the art. For example, REPs can be generated by hybridizing a control sample to an array having a selected set of polynucleotides (particularly a selected set of differentially expressed polynucleotides), acquiring the hybridization data from the array, and storing the data in a format that allows for ready comparison of the REP with a TEP. Alternatively, all expressed sequences in a control sample can be isolated and sequenced, e.g., by isolating mRNA from a control sample, converting the mRNA into cDNA, and sequencing the cDNA. The resulting sequence information roughly or precisely reflects the identity and relative number of expressed sequences in the sample. The sequence information can then be stored in a format (e.g., a computer-readable format) that allows for ready comparison of the REP with a TEP. The REP can be normalized prior to or after data storage, and/or can be processed to selectively remove sequences of expressed genes that are of less interest or that might complicate analysis (e.g., some or all of the sequences associated with housekeeping genes can be eliminated from REP data).

TEPs can be generated in a manner similar to REPs, e.g., by hybridizing a test sample to an array having a selected set of polynucleotides, particularly a selected set of differentially expressed polynucleotides, acquiring the hybridization data from the array, and storing the data in a format that allows for ready comparison of the TEP with a REP. The REP and TEP to be used in a comparison can be generated simultaneously, or the TEP can be compared to previously generated and stored REPs.

In one embodiment of the invention, comparison of a TEP with a REP involves hybridizing a test sample with a reference array, where the reference array has one or more reference sequences for use in hybridization with a sample. The reference sequences include all, at least one of, or any subset of the differentially expressed polynucleotides described herein. Hybridization data for the test sample is acquired, the data normalized, and the produced TEP compared with a REP generated using an array having the same or similar selected set of differentially expressed polynucleotides. Probes that correspond to sequences differentially expressed between the two samples will show decreased or increased hybridization efficiency for one of the samples relative to the other.

Methods for collection of data from hybridization of samples with a reference arrays are well known in the art. For example, the polynucleotides of the reference and test samples can be generated using a detectable fluorescent label, and hybridization of the polynucleotides in the samples detected by scanning the microarrays for the presence of the detectable label using, for example, a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample (e.g., a test sample) is compared to the fluorescent signal from another sample (e.g., a reference sample), and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

In general, the test sample is classified as having a gene expression profile corresponding to that associated with a disease or non-disease state by comparing the TEP generated from the test sample to one or more REPs generated from reference samples (e.g., from samples associated with cancer or specific stages of cancer, dysplasia, samples affected by a disease other than cancer, normal samples, etc.). The criteria for a match or a substantial match between a TEP and a REP include expression of the same or substantially the same set of reference genes, as well as expression of these reference genes at substantially the same levels (e.g., no significant difference between the samples for a signal associated with a selected reference sequence after normalization of the samples, or at least no greater than about 25% to about 40% difference in signal strength for a given reference sequence. In general, a pattern match between a TEP and a REP includes a match in expression, preferably a match in qualitative or quantitative expression level, of at least one of, all or any subset of the differentially expressed genes of the invention.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

Diagnosis, Prognosis and Management of Cancer

The polynucleotides of the invention and their gene products are of particular interest as genetic or biochemical markers (e.g., in blood or tissues) that will detect the earliest changes along the carcinogenesis pathway and/or to monitor the efficacy of various therapies and preventive interventions. For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting and gene therapy. Determining expression of certain polynucleotides and comparison of a patients profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the polynucleotides of the invention are staging of the cancerous disorder, and grading the nature of the cancerous tissue.

The polynucleotides of the invention can be useful to monitor patients having or susceptible to cancer to detect potentially malignant events at a molecular level before they are detectable at a gross morphological level. Furthermore, a polynucleotide of the invention identified as important for one type of cancer can also have implications for development or risk of development of other types of cancer, e.g., where a polynucleotide is differentially expressed across various cancer types. Thus, for example, expression of a polynucleotide that has clinical implications for metastatic colon cancer can also have clinical implications for stomach cancer or endometrial cancer.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides of the invention can facilitate fine-tuning of the staging process by identifying markers for the aggresivity of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors being more aggressive than well differentiated or low-grade tumors. The following guidelines are generally used for grading tumors: 1) GX Grade cannot be assessed; 2) G1 Well differentiated; G2 Moderately well differentiated; 3) G3 Poorly differentiated; 4) G4 Undifferentiated. The polynucleotides of the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of lung cancer. The polynucleotides of the invention can be used to detect lung cancer in a subject. Although there are more than a dozen different kinds of lung cancer, the two main types of lung cancer are small cell and nonsmall cell, which encompass about 90% of all lung cancer cases. Small cell carcinoma (also called oat cell carcinoma) usually starts in one of the larger bronchial tubes, grows fairly rapidly, and is likely to be large by the time of diagnosis. Nonsmall cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and can vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

The polynucleotides of the invention, e.g., polynucleotides differentially expressed in normal cells versus cancerous lung cells (e.g., tumor cells of high or low metastatic potential) or between types of cancerous lung cells (e.g., high metastatic versus low metastatic), can be used to distinguish types of lung cancer as well as identifying traits specific to a certain patient's cancer and selecting an appropriate therapy. For example, if the patient's biopsy expresses a polynucleotide that is associated with a low metastatic potential, it may justify leaving a larger portion of the patient's lung in surgery to remove the lesion. Alternatively, a smaller lesion with expression of a polynucleotide that is associated with high metastatic potential may justify a more radical removal of lung tissue and/or the surrounding lymph nodes, even if no metastasis can be identified through pathological examination.

Detection of breast cancer. The majority of breast cancers are adenocarcinomas subtypes, which can be summarized as follows: 1) ductal carcinoma in situ (DCIS), including comedocarcinoma; 2) infiltrating (or invasive) ductal carcinoma (IDC); 3) lobular carcinoma in situ (LCIS); 4) infiltrating (or invasive) lobular carcinoma (ILC); 5) inflammatory breast cancer; 6) medullary carcinoma; 7) mucinous carcinoma; 8) Paget's disease of the nipple; 9) Phyllodes tumor; and 10) tubular carcinoma;

The expression of polynucleotides of the invention can be used in the diagnosis and management of breast cancer, as well as to distinguish between types of breast cancer. Detection of breast cancer can be determined using expression levels of any of the appropriate polynucleotides of the invention, either alone or in combination. Determination of the aggressive nature and/or the metastatic potential of a breast cancer can also be determined by comparing levels of one or more polynucleotides of the invention and comparing levels of another sequence known to vary in cancerous tissue, e.g. ER expression. In addition, development of breast cancer can be detected by examining the ratio of expression of a differentially expressed polynucleotide to the levels of steroid hormones (e.g., testosterone or estrogen) or to other hormones (e.g., growth hormone, insulin). Thus expression of specific marker polynucleotides can be used to discriminate between normal and cancerous breast tissue, to discriminate between breast cancers with different cells of origin, to discriminate between breast cancers with different potential metastatic rates, etc.

Detection of colon cancer. The polynucleotides of the invention exhibiting the appropriate expression pattern can be used to detect colon cancer in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews. The expression of appropriate polynucleotides of the invention can be used in the diagnosis, prognosis and management of colorectal cancer. Detection of colon cancer can be determined using expression levels of any of these sequences alone or in combination with the levels of expression. Determination of the aggressive nature and/or the metastatic potential of a colon cancer can be determined by comparing levels of one or more polynucleotides of the invention and comparing total levels of another sequence known to vary in cancerous tissue, e.g., expression of p53, DCC ras, 1or FAP (see, e.g., Fearon E R, et al., Cell (1990) 61(5):759; Hamilton S R et al., Cancer (1993) 72:957; Bodmer W, et al., Nat Genet. (1994) 4(3):217; Fearon E R, Ann N Y Acad Sci. (1995) 768:101). For example, development of colon cancer can be detected by examining the ratio of any of the polynucleotides of the invention to the levels of oncogenes (e.g. ras) or tumor suppressor genes (e.g. FAP or p53). Thus expression of specific marker polynucleotides can be used to discriminate between normal and cancerous colon tissue, to discriminate between colon cancers with different cells of origin, to discriminate between colon cancers with different potential metastatic rates, etc.

Detection of prostate cancer. The polynucleotides and their corresponding genes and gene products exhibiting the appropriate differential expression pattern can be used to detect prostate cancer in a subject. Over 95% of primary prostate cancers are adenocarcinomas. Signs and symptoms may include: frequent urination, especially at night, inability to urinate, trouble starting or holding back urination, a weak or interrupted urine flow and frequent pain or stiffness in the lower back, hips or upper thighs.

Many of the signs and symptoms of prostate cancer can be caused by a variety of other non-cancerous conditions. For example, one common cause of many of these signs and symptoms is a condition called benign prostatic hypertrophy, or BPH. In BPH, the prostate gets bigger and may block the flow or urine or interfere with sexual function. The methods and compositions of the invention can be used to distinguish between prostate cancer and such non-cancerous conditions. The methods of the invention can be used in conjunction with conventional methods of diagnosis, e.g., digital rectal exam and/or detection of the level of prostate specific antigen (PSA), a substance produced and secreted by the prostate.

Use of Polynucleotides to Screen for Peptide Analogs and Antagonists

Polypeptides encoded by the instant polynucleotides and corresponding full length genes can be used to screen peptide libraries to identify binding partners, such as receptors, from among the encoded polypeptides. Peptide libraries can be synthesized according to methods known in the art (see, e.g., U.S. Pat. No. 5,010,175, and WO 91/17823). Agonists or antagonists of the polypeptides if the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a novel polypeptide binding partner, such as a receptor, encoded by a gene or a cDNA corresponding to a polynucleotide of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding can facilitate development of improved agonists/antagonists of the known receptor.

Pharmaceutical Compositions and Therapeutic Uses

Pharmaceutical compositions of the invention can comprise polypeptides, antibodies, or polynucleotides (including antisense nucleotides and ribozymes) of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Delivery Methods. Once formulated, the compositions of the invention can be (1) administered directly to the subject (e.g., as polynucleotide or polypeptides); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once a gene corresponding to a polynucleotide of the invention has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. Preferably, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeuttics. Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the antisense subgenomic polynucleotides. Where greater expression is desired over a larger area of tissue, larger amounts of antisense subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. For polynucleotide related genes encoding polypeptides or proteins with anti-inflammatory activity, suitable use, doses, and administration are described in U.S. Pat. No. 5,654,173.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5, 219,740; WO 93/11230; WO 93/10218; U.S. Pat. No.4,777,127; GB Patent No. 2,200,65 1; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3: 147); ligand-linked DNA(see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581

Further non-viral delivery suitable for use includes mechanical delivery systems such as the approach described in Woffendin et al., Proc. Natl. Acad. Sci. USA (1994) 91(24): 11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous embodiments. However, it should be noted that these embodiments are illustrative and are not to be construed as restricting the invention in any way.

### EXAMPLES

The following examples are offered primarily for purposes of illustration. It will be readily apparent to those skilled in the art that the formulations, dosages, methods of administration, and other parameters of this invention may be further modified or substituted in various ways without departing from the spirit and scope of the invention.

### Example 1

### Source of Biological Materials and Overview of Novel Polynucleotides Expressed by the Biological Materials

cDNA libraries were constructed from mRNA isolated from the GRRpz or and WOca cells, which were provided by Dr. Donna M. Peehl, Department of Medicine, Stanford University School of Medicine. GRRpz cells were primary cells derived from normal prostate epithelium. The WOca cells were prostate epithelial cells derived from prostate cancer Gleason Grade 4+4. Polynucleotides expressed by these cells were isolated and analyzed; the sequences of these polynucleotides were about 275-300 nucleotides in length.

The sequences of the isolated polynucleotides were first masked to eliminate low complexity sequences using the XBLAST masking program (Claverie “Effective Large-Scale Sequence Similarity Searches,” In: Computer Methods for Macromolecular Sequence Analysis, Doolittle, ed., Meth. Enzymol. 266:212-227 Academic Press, NY, N.Y. (1996); see particularly Claverie, in “Automated DNA Sequencing and Analysis Techniques” Adams et al., eds., Chap. 36, p. 267 Academic Press, San Diego, 1994 and Claverie et al. Comput. Chem. (1993) 17:191). Generally, masking does not influence the final search results, except to eliminate sequences of relative little interest due to their low complexity, and to eliminate multiple “hits” based on similarity to repetitive regions common to multiple sequences, e.g., Alu repeats. The remaining sequences were then used in a BLASTN vs. GenBank search; sequences that exhibited greater than 70% overlap, 99% identity, and a p value of less than 1×10⁻⁴⁰ were discarded. Sequences from this search also were discarded if the inclusive parameters were met, but the sequence was ribosomal or vector-derived.

The resulting sequences from the previous search were classified into three groups (1, 2 and 3 below) and searched in a BLASTX vs. NRP (non-redundant proteins) database search: (1) unknown (no hits in the GenBank search), (2) weak similarity (greater than 45% identity and p value of less than 1×10⁻⁵), and (3) high similarity (greater than 60% overlap, greater than 80% identity, and p value less than 1×10⁻⁵). Sequences having greater than 70% overlap, greater than 99% identity, and p value of less than 1×10⁻⁴⁰ were discarded.

The remaining sequences were classified as unknown (no hits), weak similarity, and high similarity (parameters as above). Two searches were performed on these sequences. First, a BLAST vs. EST database search was performed and sequences with greater than 99% overlap, greater than 99% similarity and a p value of less than 1×10⁻⁴⁰ were discarded. Sequences with a p value of less than 1×10⁻⁶⁵ when compared to a database sequence of human origin were also excluded. Second, a BLASTN vs. Patent GeneSeq database was performed and sequences having greater than 99% identity, p value less than 1×10⁻⁴⁰, and greater than 99% overlap were discarded.

The remaining sequences were subjected to screening using other rules and redundancies in the dataset. Sequences with a p value of less than 1×10⁻¹¹¹ in relation to a database sequence of human origin were specifically excluded. The final result provided the 316 sequences listed as SEQ ID NOS:1-316 in the accompanying Sequence Listing and summarized in Table 1 (inserted prior to claims). Each identified polynucleotide represents sequence from at least a partial mRNA transcript. Many of the sequences include the sequence ggcacgag at the 5′ end; this sequence is a sequencing artifact and not part of the sequence of the polynucleotides of the invention.

Table 1 provides: 1) the SEQ ID NO (“SEQ ID”) assigned to each sequence for use in the present specification; 2) the Cluster Identification No. (“CLUSTER”); 3) the sequence name (“SEQ NAME”) used as an internal identifier of the sequence; 4) the orientation of the sequence (“ORIENT”); 5) the name assigned to the clone from which the sequence was isolated (“CLONE ID”); and the name of the library from which the sequence was isolated (“LIBRARY”). CH22PRC indicates the sequence was isolated from Library 22; CH21PRN indicates the sequence was isolated from Library 21. A description of the libraries is provided in Table 3 below. Because the provided polynucleotides represent partial mRNA transcripts, two or more polynucleotides of the invention may represent different regions of the same mRNA transcript and the same gene. Thus, if two or more SEQ ID NOS: are identified as belonging to the same clone, then either sequence can be used to obtain the full-length mRNA or gene.

### Example 2

### Results of Public Database Search to Identify Function of Gene Products

SEQ ID NOS:1-316 were translated in all three reading frames, and the nucleotide sequences and translated amino acid sequences used as query sequences to search for homologous sequences in either the GenBank (nucleotide sequences) or Non-Redundant Protein (amino acid sequences) databases. Query and individual sequences were aligned using the BLAST 2.0 programs, available over the world wide web at a saite sponsored by the National Center for Biotechnology Information, which is supported by the National Library of Medicine and the National Institutes of Health (see also Altschul, et al. Nucleic Acids Res. (1997) 25:3389-3402). The sequences were masked to various extents to prevent searching of repetitive sequences or poly-A sequences, using the XBLAST program for masking low complexity as described above in Example 1.

Table 2 (inserted before the claims) provide the alignment summaries having a p value of 1×10⁻² or less indicating substantial homology between the sequences of the present invention and those of the indicated public databases. Specifically, Table 2 provides the SEQ ID NO of the query sequence, the accession number of the GenBank database entry of the homologous sequence, and the p value of the alignment. Table 2 also provides the SEQ ID NO of the query sequence, the accession number of the Non-Redundant Protein database entry of the homologous sequence, and the p value of the alignment. The alignments provided in Table 2 are the best available alignment to a DNA or amino acid sequence at a time just prior to filing of the present specification. The activity of the polypeptide encoded by the SEQ ID NOS listed in Table 2 can be extrapolated to be substantially the same or substantially similar to the activity of the reported nearest neighbor or closely related sequence. The accession number of the nearest neighbor is reported, providing a publicly available reference to the activities and functions exhibited by the nearest neighbor. The public information regarding the activities and functions of each of the nearest neighbor sequences is incorporated by reference in this application. Also incorporated by reference is all publicly available information regarding the sequence, as well as the putative and actual activities and functions of the nearest neighbor sequences listed in Table 2 and their related sequences. The search program and database used for the alignment, as well as the calculation of the p value are also indicated.

Full length sequences or fragments of the polynucleotide sequences of the nearest neighbors can be used as probes and primers to identify and isolate the full length sequence of the corresponding polynucleotide. The nearest neighbors can indicate a tissue or cell type to be used to construct a library for the full-length sequences of the corresponding polynucleotides.

### Example 3

### Differential Expression of Polynucleotides of the Invention: Description of Libraries and Detection of Differential Expression

The relative expression levels of the polynucleotides of the invention was assessed in several libraries prepared from various sources, including primary cells, cell lines and patient tissue samples. Table 3 provides a summary of these libraries, including the shortened library name (used hereafter), the mRNA source used to prepared the cDNA library, the “nickname” of the library that is used in the tables below (in quotes), and the approximate number of clones in the library.

TABLE 3
Description of cDNA Libraries

| | | Number |
|---|---|---|
| | | of |
| | | Clones |
| Library | | in |
| (Lib#) | Description | Library |
| 1 | Human Colon Cell Line Km12 L4: High Metastatic | 308731 |
| | Potential (derived from Km12C) | |
| 2 | Human Colon Cell Line Km12C: Low Metastatic | 284771 |
| | Potential | |
| 3 | Human Breast Cancer Cell Line MDA-MB-231: High | 326937 |
| | Metastatic Potential; micro-mets in lung | |
| 4 | Human Breast Cancer Cell Line MCF7: Non | 318979 |
| | Metastatic | |
| 8 | Human Lung Cancer Cell Line MV-522: High | 223620 |
| | Metastatic Potential | |
| 9 | Human Lung Cancer Cell Line UCP-3: Low Metastatic | 312503 |
| | Potential | |
| 12 | Human microvascular endothelial cells (HMVEC) - | 41938 |
| | UNTREATED (PCR (OligodT) cDNA library) | |
| 13 | Human microvascular endothelial cells (HMVEC) - | 42100 |
| | bFGF TREATED (PCR (OligodT) cDNA library) | |
| 14 | Human microvascular endothelial cells (HMVEC) - | 42825 |
| | VEGF TREATED (PCR (OligodT) cDNA library) | |
| 15 | Normal Colon - UC#2 Patient (MICRODISSECTED | 282722 |
| | PCR (OligodT) cDNA library) | |
| 16 | Colon Tumor - UC#2 Patient (MICRODISSECTED | 298831 |
| | PCR (OligodT) cDNA library) | |
| 17 | Liver Metastasis from Colon Tumor of UC#2 Patient | 303467 |
| | (MICRODISSECTED PCR (OligodT) cDNA library) | |
| 18 | Normal Colon - UC#3 Patient (MICRODISSECTED | 36216 |
| | PCR (OligodT) cDNA library) | |
| 19 | Colon Tumor - UC#3 Patient (MICRODISSECTED | 41388 |
| | PCR (OligodT) cDNA library) | |
| 20 | Liver Metastasis from Colon Tumor of UC#3 Patient | 30956 |
| | (MICRODISSECTED PCR (OligodT) cDNA library) | |
| 21 | GRRpz Cells derived from normal prostate epithelium | 164801 |
| 22 | WOca Cells derived from Gleason Grade 4 prostate | 162088 |
| | cancer epithelium | |
| 23 | Normal Lung Epithelium of Patient #1006 | 306198 |
| | (MICRODISSECTED PCR (OligodT) cDNA library) | |
| 24 | Primary tumor, Large Cell Carcinoma of Patient #1006 | 309349 |
| | (MICRODISSECTED PCR (OligodT) cDNA library) | |

The KM12L4 cell line is derived from the KM12C cell line (Morikawa, et al., Cancer Research (1988) 48:6863). The KM12C cell line, which is poorly metastatic (low metastatic) was established in culture from a Dukes' stage B₂ surgical specimen (Morikawa et al. Cancer Res. (1988) 48:6863). The KML4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246). The MDA-MB-231 cell line (Brinkley et al. Cancer Res. (1980) 40:3118-3129) was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma.

The MCF7 cell line was derived from a pleural effusion of a breast adenocarcinoma and is non-metastatic. The MV-522 cell line is derived from a human lung carcinoma and is of high metastatic potential. The UCP-3 cell line is a low metastatic human lung carcinoma cell line; the MV-522 is a high metastatic variant of UCP-3. These cell lines are well-recognized in the art as models for the study of human breast and lung cancer (see, e.g., Chandrasekaran et al., Cancer Res. (1979) 39:870 (MDA-MB-231 and MCF-7); Gastpar et al., J Med Chem (1 998) 41:4965 (MDA-MB-231 and MCF-7); Ranson et al., Br J Cancer (1998) 77:1586 (MDA-MB-231 and MCF-7); Kuang et al., Nucleic Acids Res (1998) 26:1116 (MDA-MB-231 and MCF-7); Varki et al., Int J Cancer (1987) 40:46 (UCP-3); Varki et al., Tumour Biol. (1990) 11:327; (MV-522 and UCP-3); Varki et al., Anticancer Res. (1990) 10:637; (MV-522); Kelner et al., Anticancer Res (1 995) 15:867 (MV-522); and Zhang et al., Anticancer Drugs (1997) 8:696 (MV522)). The samples of libraries 15-20 are derived from two different patients (UC#2, and UC#3). The bFGF-treated HMVEC were prepared by incubation with bFGF at 10 ng/ml for 2 hrs; the VEGF-treated HMVEC were prepared by incubation with 20 ng/ml VEGF for 2 hrs. Following incubation with the respective growth factor, the cells were washed and lysis buffer added for RNA preparation. The GRRpz and WOca cells were provided by Dr. Donna M. Peehl, Department of Medicine, Stanford University School of Medicine. GRRpz cells were derived from normal prostate epithelium. The WOca cells are Gleason Grade 4 cell line.

Each of the libraries is composed of a collection of cDNA clones that in turn are representative of the mRNAs expressed in the indicated mRNA source. In order to facilitate the analysis of the millions of sequences in each library, the sequences were assigned to clusters. The concept of “cluster of clones” is derived from a sorting/grouping of cDNA clones based on their hybridization pattern to a panel of roughly 300 7 bp oligonucleotide probes (see Drmanac et al., Genomics (1996) 37(1):29). Random cDNA clones from a tissue library are hybridized at moderate stringency to 300 7 bp oligonucleotides. Each oligonucleotide has some measure of specific hybridization to that specific clone. The combination of 300 of these measures of hybridization for 300 probes equals the “hybridization signature” for a specific clone. Clones with similar sequence will have similar hybridization signatures. By developing a sorting/grouping algorithm to analyze these signatures, groups of clones in a library can be identified and brought together computationally. These groups of clones are termed “clusters”. Depending on the stringency of the selection in the algorithm (similar to the stringency of hybridization in a classic library cDNA screening protocol), the “purity” of each cluster can be controlled. For example, artifacts of clustering may occur in computational clustering just as artifacts can occur in “wet-lab” screening of a cDNA library with 400 bp cDNA fragments, at even the highest stringency. The stringency used in the implementation of cluster herein provides groups of clones that are in general from the same cDNA or closely related cDNAs. Closely related clones can be a result of different length clones of the same cDNA, closely related clones from highly related gene families, or splice variants of the same cDNA.

Differential expression for a selected cluster was assessed by first determining the number of cDNA clones corresponding to the selected cluster in the first library (Clones in 1^{st}), and the determining the number of cDNA clones corresponding to the selected cluster in the second library (Clones in 2^{nd}). Differential expression of the selected cluster in the first library relative to the second library is expressed as a “ratio” of percent expression between the two libraries. In general, the “ratio” is calculated by: 1) calculating the percent expression of the selected cluster in the first library by dividing the number of clones corresponding to a selected cluster in the first library by the total number of clones analyzed from the first library; 2) calculating the percent expression of the selected cluster in the second library by dividing the number of clones corresponding to a selected cluster in a second library by the total number of clones analyzed from the second library; 3) dividing the calculated percent expression from the first library by the calculated percent expression from the second library. If the “number of clones” corresponding to a selected cluster in a library is zero, the value is set at I to aid in calculation. The formula used in calculating the ratio takes into account the “depth” of each of the libraries being compared, i.e., the total number of clones analyzed in each library.

In general, a polynucleotide is said to be significantly differentially expressed between two samples when the ratio value is greater than at least about 2, preferably greater than at least about 3, more preferably greater than at least about 5, where the ratio value is calculated using the method described above. The significance of differential expression is determined using a z score test (Zar, Biostatistical Analysis, Prentice Hall, Inc., USA, “Differences between Proportions,” pp 296-298 (1974).

Using this approach, a number of polynucleotide sequences were identified as being differentially expressed between, for example, cells derived from high metastatic potential cancer tissue and low metastatic cancer cells, and between cells derived from metastatic cancer tissue and normal tissue. Evaluation of the levels of expression of the genes corresponding to these sequences can be valuable in diagnosis, prognosis, and/or treatment (e.g., to facilitate rationale design of therapy, monitoring during and after therapy, etc.). Moreover, the genes corresponding to differentially expressed sequences described herein can be therapeutic targets due to their involvement in regulation (e.g., inhibition or promotion) of development of, for example, the metastatic phenotype. For example, sequences that correspond to genes that are increased in expression in high metastatic potential cells relative to normal or non-metastatic tumor cells may encode genes or regulatory sequences involved in processes such as angiogenesis, differentiation, cell replication, and metastasis.

Detection of the relative expression levels of differentially expressed polynucleotides described herein can provide valuable information to guide the clinician in the choice of therapy. For example, a patient sample exhibiting an expression level of one or more of these polynucleotides that corresponds to a gene that is increased in expression in metastatic or high metastatic potential cells may warrant more aggressive treatment for the patient. In contrast, detection of expression levels of a polynucleotide sequence that corresponds to expression levels associated with that of low metastatic potential cells may warrant a more positive prognosis than the gross pathology would suggest.

The differential expression of the polynucleotides described herein can thus be used as, for example, diagnostic markers, prognostic markers, for risk assessment, patient treatment and the like. These polynucleotide sequences can also be used in combination with other known molecular and/or biochemical markers.

The differential expression data for polynucleotides of the invention that have been identified as being differentially expressed across various combinations of the libraries described above is summarized in Table 4 (inserted prior to the claims). Table 4 provides: 1) the Sequence Identification Number (“SEQ ID”) assigned to the polynucleotide; 2) the cluster (“CLUST”) to which the polynucleotide has been assigned as described above; 3) the library comparisons that resulted in identifcation of the polynucleotide as being differentially expressed (“PairAB-text”), with shorthand names of the compared libraries provided in parentheses following the library numbers; 4) the number of clones corresponding to the polynucleotide in the first library listed (“A”); 5) the number of clones corresponding to the polynucleotide in the second library listed (“B”); 6) the “RATIO PLUS” where the comparison resulted in a finding that the number of clones in library A is greater than the number of clones in library B; and 7) the “RATIO MINUS” where the comparison resulted in a finding that the number of clones in library B is greater than the number of clones in library A.

### Example 4

### Differential Expression of a Polynucleotides Associated with Metastatic Potential in Breast Cancer

Differential expression was examined in breast cancer cells having either high metastatic potential or low metastatic potential. A single cluster, Cluster Identification No. 10154, was identified as displaying low expression in the high metastatic potential breast cancer cells (Library 3), and significantly increased expression—approximately 100-fold higher—in the low metastatic potential cells (Library 4). Specifically, three clones were identified that were expressed in Library 3, the high metastatic potential breast cancer library, while 317 clones were expressed in Library 4, the low metastatic potential breast cancer library. The two sequences assigned to this particular cluster, SEQ ID NO:315 and SEQ ID NO:316, both displayed this differential expression, suggesting that the two sequences are likely associated with a single transcript.

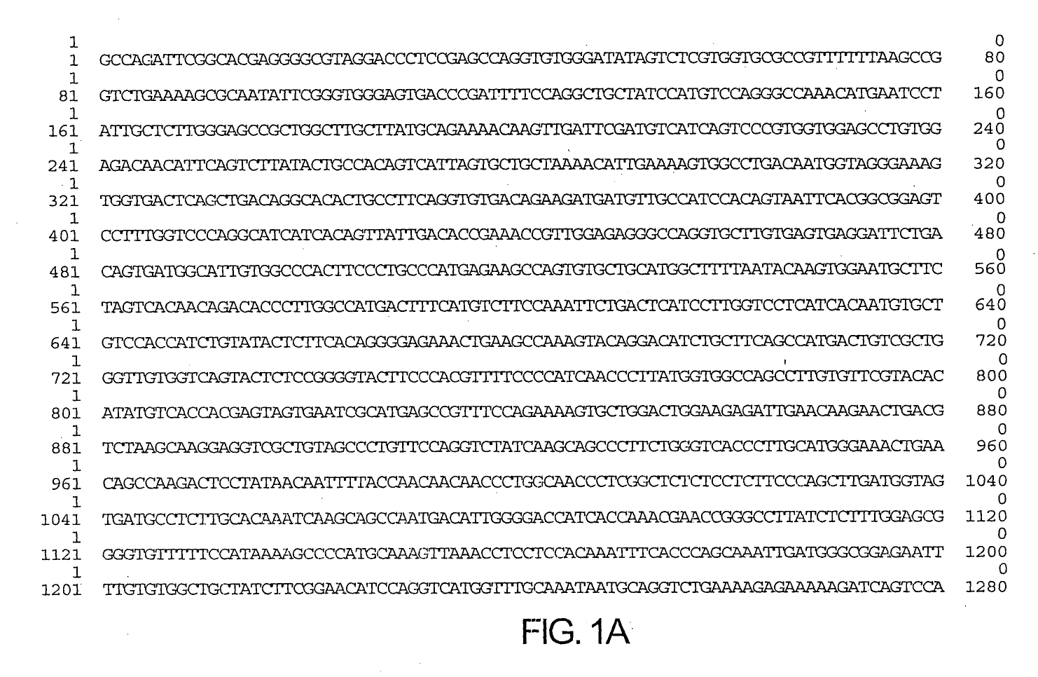

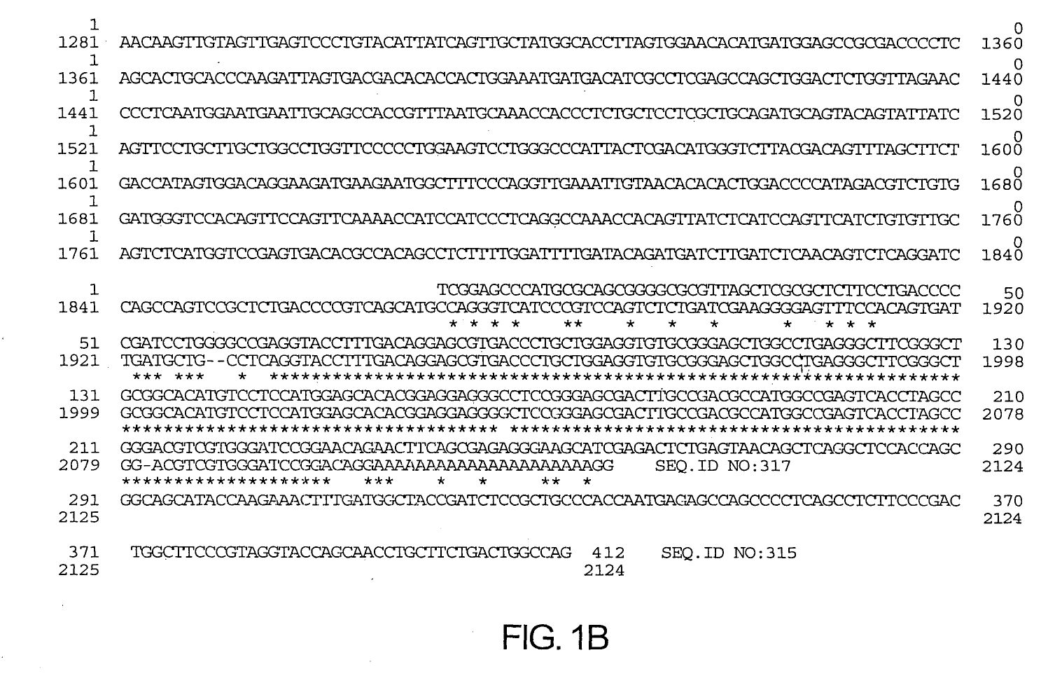

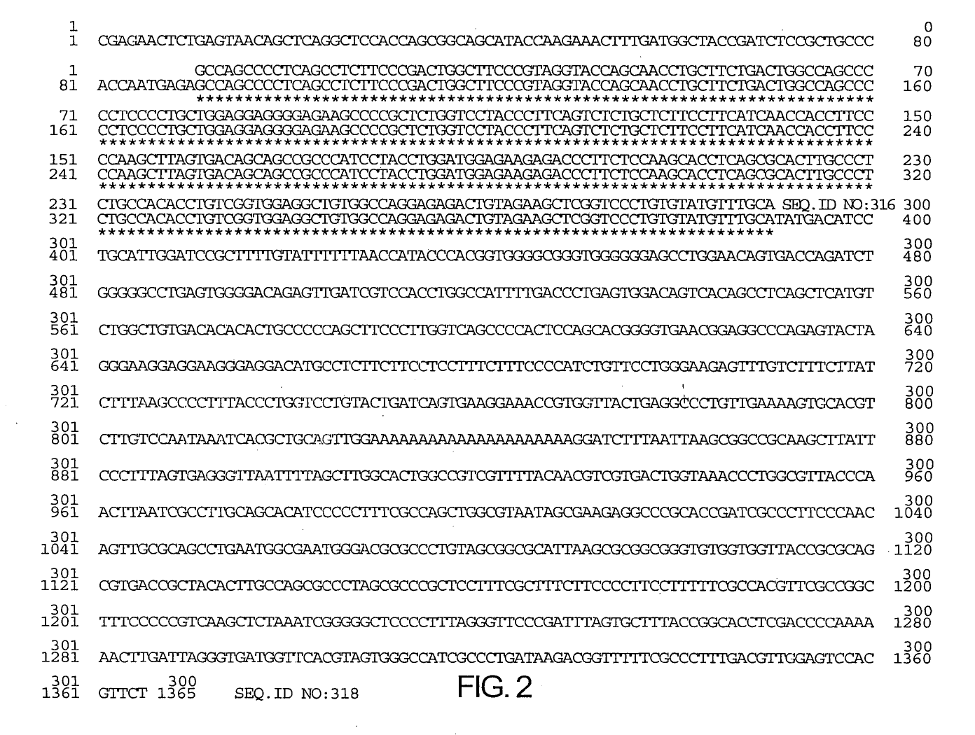

SEQ ID NO:315 and SEQ ID NO:316 were then used as query sequences to search for homologous sequences in GenBank as described in Examples 1 and 2. SEQ ID NO: 315 displayed identity to the GenBank entry H72034 (SEQ ID NO:317) and SEQ ID NO:316 displayed identity to GenBank entry AA707002 (SEQ ID NO:318). SEQ ID NO:315 displays striking identity to the 3′ end of SEQ ID NO:317 (See FIGS. 1A and 1B), while SEQ ID NO:316 displays striking identity to the 5′ end of SEQ ID NO:318 (See FIG. 2). Clones of H72034 and AA707002 were ordered from the I.M.A.G.E. Consortium at the Lawrence Livermore National Laboratories (Livermore, Calif.) for further studies.

Restriction Mapping of Clones H72034 and AA707002

The newly identified sequences were digested with a number of different restriction endonucleases to construct a restriction map of each of the clones. An appropriate amount of each clone, SEQ ID NO:317 or SEQ ID NO:318, was digested with various enzymes, and the restriction fragments identified as follows:

| | | |
|---|---|---|
| Enzyme | #Cuts | Positions |

SEQ ID NO: 317

| | | | | | | | | | |
|---|---|---|---|---|---|---|---|---|---|
| AluI | 5 | 331 | 1029 | 1422 | 1595 | 1977 | | | |
| BamHI | 2 | 1836 | 2089 | | | | | | |
| BstEII | 1 | 936 | | | | | | | |
| BstXI | 1 | 1033 | | | | | | | |
| HaeIII | 12 | 145 | 300 | 453 | 497 | 582 | 780 | | |
| | | 1102 | 1536 | 1561 | 1722 | 1981 | 2062 | | |
| HinfI | 12 | 5 | 154 | 205 | 325 | 397 | 473 | 610 | 820 |
| | | 968 | 1295 | 1426 | 2066 | | | | |
| KpnI | 1 | 1938 | | | | | | | |
| MspI | 6 | 78 | 739 | 1098 | 2038 | 2077 | 2093 | | |
| NcoI | 2 | 2013 | 2058 | | | | | | |
| PstI | 1 | 1501 | | | | | | | |
| PvuII | 2 | 331 | 1422 | | | | | | |
| Sau3AI | 6 | 1270 | 1813 | 1819 | 1836 | 1894 | 2089 | | |
| SphI | 1 | 1870 | | | | | | | |
| XhoI | 1 | 1413 | | | | | | | |

SEQ ID NO: 318

| | | | | | | | | | |
|---|---|---|---|---|---|---|---|---|---|
| AluI | 9 | 19 | 245 | 367 | 553 | 586 | 874 | 904 | 996 |
| | | 1214 | | | | | | | |
| BamHI | 1 | 407 | | | | | | | |
| BglI | 1 | 1056 | | | | | | | |
| BglII | 1 | 475 | | | | | | | |
| BstEI | 1 | 1108 | | | | | | | |
| HaeIII | 10 | 153 | 348 | 485 | 867 | 518 | 628 | 780 | 867 |
| | | 915 | 1016 | 1312 | | | | | |
| HindIII | 2 | 243 | 872 | | | | | | |
| HinfI | 1 | 1353 | | | | | | | |
| KpnI | 1 | 132 | | | | | | | |
| MspI | 2 | 1196 | 1261 | | | | | | |
| PstI | 1 | 823 | | | | | | | |
| PvuII | 1 | 996 | | | | | | | |
| Sau3AI | 7 | 66 | 407 | 475 | 504 | 750 | 850 | 1024 | |

The restriction maps based on the identified sites can be used to determine the position of each clone relative to the genomic sequences, and to confirm the 5′-3′ orientation of the clones.

Amplification and Purification of Transcript

A transcript in this region upregulated in low metastatic cancers which contain sequences from SEQ ID NOS:315-318 is identified using a technique such as polymerase chain reaction (PCR) amplification. Based on the sequences identified and the original sequences of the cluster, primers can be designed to isolate the full length cDNA from a library constructed from the breast cancer cell line with low metastatic potential.

A cDNA template for use in the amplification reaction is generated from total RNA isolated from the high metastatic breast cell line. RNA is reverse transcribed using oligo-dT primer to generate first strand cDNA. cDNA is synthesized by denaturing 3 μl of total RNA, 2 μl oligo-dT primer at 20 μM, and 5 μl DEPC water for 8 minutes at 65° C. followed by reverse transcription at 52° C. for 1 hour in a reaction containing the denatured RNA/primer plus 4 μl 15×cDNA buffer (GibcoBRL), 1 μl 0.1 M dithiothreitol, 1 μl 40 U/1 RNAseOUT (GibcoBRL), 1 μl DEPC water, 2 μl 10 mM dNTP (GibdoBRL), and 1 μl 15 U/1 Thermoscript reverse transcriptase (GibcoBRL). The reaction was terminated by a 5-min incubation at 85° C., and the RNA was removed by 1 μL 2 U/1 RNAse H at 37° C. for thirty minutes.

Based on the determined orientation of the clones, primers are designed to amplify a full-length clone corresponding to the differentially expressed transcript in this region. Forward primers that are used to amplify the full-length clone are taken from the 5′ end of SEQ ID NO: 17 as follows:

| | | |
|---|---|---|
| F1 | 5′-TGGGATATAGTCTCGTGGTGCG-3′ | (SEQ ID NO:319) |
| F2 | 5′-TGATTCGATGTCATCAGTCCCG-3′ | (SEQ ID NO:320) |

Primer F1 is taken from residues 51-62 of SEQ ID NO: 317, and primer F2 is taken from residues 212-233 Of SEQ ID NO:17. Both forward primers are near the 5′ end of this sequence.

Reverse Primers are designed using sequences complementary to the 3′ end of clone 10154-3 as follows:

| | | |
|---|---|---|
| R1 | 5′-TGTGTCACAGCCAGACATGAGC | (SEQ ID NO:321) |
| P2 | 5′-TGCAAACATACACAGGGACCG | (SEQ ID NO:322) |

Primer R1 is based on residues 573-552 of SEQ ID NO:318, and R2 is based on residues 399-379 of SEQ ID NO:318.

PCR is performed using a 5 μl aliquot of the first strand cDNA synthesis reaction, and a primer pair, e.g., F1 and R1, F1 and R2, F2 and R1, or F2 and R2. An open reading frame is amplified using 2 μl of the reverse transcription product as template in a PCR reaction containing 5 μl of 10×PCR buffer (GibcoBRL), 1 μl 50 mM Mg₂SO₄, 1 μl 10 mM dNTP, 1 μl F1 or F2 primer, 1 μl R1 primer, 2.5 U High Fidelity Platinum Taq DNA polymerase (GibcoBRL), and water to 50 μl. The molecule is amplified using 30 rounds of amplification in a thermal cycler at the following temperatures: 1 minute at 95° C.; 1 minute at 55° C. and 2 minutes at 72° C. The 30 cycles was followed by a 10 minute extension at 72° C.

Following amplification of the sequences, the PCR products are loaded on a 1% TEA gel and subjected to gel purification. One or more bands can be isolated from the gel and the DNA was purified using a QIAquick® Gel Extraction Kit (Qiagen, Valencia, Calif.). The purified fragment was cloned into a bacterial vector and transformed into the bacterial strain DH5α. Following cloning of the purified fragment(s), the DNA can be isolated and sequenced to confimn that a band corresponds to a transcript from this genetic region.

The reactions are carried out with two different 5′ and 3′ primers to increase the likelihood that the reaction will yield an amplification product. Other primers may also be designed from the predicted 5′ and/or 3′ end of the sequence, as will be apparent to one skilled in the art upon reading this disclosure, and thus other primers may be designed from the general region of SEQ ID NOS:317 and 318 that may yield better results than the disclosed primers.

In order to obtain additional sequences 5′ to the end of a partial cDNA, 5′ rapid amplification of cDNA ends (RACE) can be performed to ensure that the entire transcript has been identified. See PCR Protocols: A Guide to Methods and Applications, (1990) Academic Press, Inc. Following isolation of a cDNA using the F1-R1 or F2-R1 primer pairs, additional primers can be designed to perform RACE. The primers can be designed from the sequence of 10154-1 as follows:

| | |
|---|---|
| 5′-TTTAGCAGCACTAATGACTGTGGC-3′ | (SEQ ID NO:323) |
| 5′-CGCCGTGAATTACTGTGGATGG-3′ | (SEQ ID NO:324) |

The two RACE primers are designed based residues 286-263 and 396-375 of SEQ ID NO:317, respectively.

These sequences can be used to obtain any transcript sequences 5′ to the amplification products obtained using the PCR protocol described above.

Northem Analysis

Other techniques can be used for confirming differential expression of the full-length transcript. For example, a Northern Blot can be used to verify differential expression of SEQ ID NOS:317 and 318 in a breast cancer cells with low metastatic potential compared to breast cancer cells with high metastatic potential. Northern analysis can be accomplished by methods well-known in the art. Briefly, RNA is individually isolated from breast cancer cells having high metastatic potential and breast cancer cells having low metastatic potential, e.g. a product such as RNeasy Mini Kits (Qiagen, Calif.) or NucleoSpin® RNA II Kit (Clontech, Palo Alto, Calif.). The isolated RNA samples are For Northern analysis, RNA isolated from the cells was electrophoresed on a denaturing formaldehyde agarose gel and transferred onto a membrane such as a supported nitrocellulose membrane (Schleicher & Schuell).

Rapid-Hyb buffer (Amersham Life Science, Little Chalfont, England) with 5 mg/ml denatured single stranded sperm DNA is pre-warmed to 65° C. and the RNA blots are pre-hybridized in the buffer with shaking at 65° C. for 30 minutes. Gene-specific DNA probes (50 ng per reaction) labeled with [α-³²P]dCTP (3000 Ci/mmol, Amersham Pharmacia Biotech Inc., Piscataway, N.J.) (Prime-It RmT Kit, Stratagene, La Jolla, Calif.) and purified with ProbeQuant™ G-50 Micro Columns (Amersham Pharmacia Biotech Inc.) are added and hybridized to the blots with shaking at 65° C. for overnight. The blots are washed in 2×SSC, 0.1%(w/v) SDS at room temperature for 20 minutes, twice in 1×SSC, 0.1%(w/v) SDS at 65° C. for 15 minutes, then exposed to Hyperfilms (Amersham Life Science).

### Example 6

### Identification of Differentially Expressed Genes by Array Analysis with Patient Tissue Samples

Differentially expressed genes corresponding to the polynucleotides described herein were also identified by microarray hybridization analysis using materials obtained from patient tissue samples. The biological materials used in these experiments are described below.

Source of Patient Tissue Samples

Normal and cancerous tissues were collected from patients using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001). Table 8 (inserted following the last page of the Examples ) provides information about each patient from which the samples were isolated, including: the Patient ID and Path ReportID, numbers assigned to the patient and the pathology reports for identification purposes; the anatomical location of the tumor (AnatomicalLoc); The Primary Tumor Size; the Primary Tumor Grade; the Histopathologic Grade; a description of local sites to which the tumor had invaded (Local Invasion); the presence of lymph node metastases (Lymph Node Metastasis); incidence of lymph node metastases (provided as number of lymph nodes positive for metastasis over the number of lymph nodes examined) (Incidence Lymphnode Metastasis); the Regional Lymphnode Grade; the identification or detection of metastases to sites distant to the tumor and their location (Distant Met & Loc);a description of the distant metastases (Description Distant Met); the grade of distant metastasis (Distant let Grade); and general comments about the patient or the tumor (Comments). Adenoma was not described in any of the patients. ; adenoma dysplasia (described as hyperplasia by the pathologist) was described in Patient ID No. 695. Extranodal extensions were described in two patients, Patient ID Nos. 784 and 791. Lymphovascular invasion was described in seven patients, Patient ID Nos. 128, 278, 517, 534, 784, 786, and 791. Crohn's-like infiltrates were described in seven patients, Patient ID Nos. 52, 264, 268, 392, 393, 784, and 791.

Source of Polynucleotides on Arrays

Polynucleotides on Arrays

Polynucleotides spotted on the arrays were generated by PCR amplification of clones derived from cDNA libraries. The clones used for amplification were either the clones from which the sequences described herein (SEQ ID NOS:1-316) were derived, or are clones having inserts with significant polynucleotide sequence overlap with the sequences described herein (SEQ ID NO:1-316) as determined by BLAST2 homology searching.

Microarray Design

Each array used in the examples below had an identical spatial layout and control spot set. Each microarray was divided into two areas, each area having an array with, on each half, twelve groupings of 32×12 spots for a total of about 9,216 spots on each array. The two areas are spotted identically which provide for at least two duplicates of each clone per array. Spotting was accomplished using PCR amplified products from 0.5 kb to 2.0 kb and spotted using a Molecular Dynamics Gen III spotter according to the manufacturer's recommendations. The first row of each of the 24 regions on the array had about 32 control spots, including 4 negative control spots and 8 test polynucleotides.

The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about 4 duplicate measurements for each clone, two of one color and two of the other, for each sample.

Microarray Analysis

cDNA probes were prepared from total RNA isolated from the patient cells described in above (Table 8). Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling. Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red).

The differential expression assay was performed by mixing equal amounts of probes from tumor cells and normal cells of the same patient. The arrays were prebybridized by incubation for about 2 hrs at 60° C in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. For initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

Table 9 below summarize the results of the differential expression analysis. Each table provides: the SEQ ID NO of the polynucleotide corresponding to the polynucleotide on the spot on the array; the Spot ID (an identifier assigned to the spot so as to distinguish it from spots on the same and different arrays), the number of patients for whom there was information obtained from the array (Num Ratios), and the percentage of patients in which expression was detected at greater than or equal to a two-fold increase (>=2×), greater than or equal to a five-fold increase (>=5×), or less than or equal to a ½-fold decrease (<=halfx) relative to matched normal control tissue.

In general, a polynucleotide is said to represent a significantly differentially expressed gene between two samples when there is detectable levels of expression in at least one sample and the ratio value is greater than at least about 1.2 fold, preferably greater than at least about 1.5 fold, more preferably greater than at least about 2 fold, where the ratio value is calculated using the method described above.

A differential expression ratio of 1 indicates that the expression level of the gene in the tumor cell was not statistically different from expression of that gene in normal colon cells of the same patient. A differential expression ratio significantly greater than 1 in cancerous colon cells relative to normal colon cells indicates that the gene is increased in expression in cancerous cells relative to normal cells, indicating that the gene plays a role in the development of the cancerous phenotype, and may be involved in promoting metastasis of the cell. Detection of gene products from such genes can provide an indicator that the cell is cancerous, and may provide a therapeutic and/or diagnostic target.

Likewise, a differential expression ratio significantly less than 1 in cancerous colon cells relative to normal colon cells indicates that, for example, the gene is involved in suppression of the cancerous phenotype. Increasing activity of the gene product encoded by such a gene, or replacing such activity, can provide the basis for chemotherapy. Such gene can also serve as markers of cancerous cells, e.g., the absence or decreased presence of the gene product in a colon cell relative to a normal colon cell indicates that the cell may be cancerous.

TABLE 9

| SEQ ID | | Num | | | |
|---|---|---|---|---|---|
| NO: | SpotID | Ratios | >=2x | >=5x | <=halfx |
| 8 | 579 | 33 | 87.88 | 39.39 | 3.03 |
| 12 | 22300 | 33 | 33.33 | 18.18 | 6.06 |
| 26 | 21886 | 33 | 33.33 | 0.00 | 3.03 |
| 64 | 9487 | 33 | 33.33 | 12.12 | 3.03 |
| 248 | 28179 | 28 | 32.14 | 0.00 | 0.00 |
| 253 | 28179 | 28 | 32.14 | 0.00 | 0.00 |
| 272 | 28179 | 28 | 32.14 | 0.00 | 0.00 |
| 292 | 9111 | 33 | 33.33 | 18.18 | 3.03 |
| 295 | 19980 | 33 | 33.33 | 6.06 | 0.00 |
| 309 | 23993 | 33 | 42.42 | 3.03 | 3.03 |

Deposit Information. The following materials were deposited with the American Type Culture Collection (CMCC=Chiron Master Culture Collection).

TABLE 5
Cell Lines Deposited with ATCC

| | | ATCC | CMCC Accession |
|---|---|---|---|
| Cell Line | Deposit Date | Accession No. | No. |
| KM12L4-A | Mar. 19, 1998 | CRL-12496 | 11606 |
| Km12C | May 15, 1998 | CRL-12533 | 11611 |
| MDA-MB-231 | May 15, 1998 | CRL-12532 | 10583 |
| MCF-7 | Oct. 9, 1998 | CRL-12584 | 10377 |

In addition, pools of selected clones, as well as libraries containing specific clones, were assigned an “ES” number (internal reference) and deposited with the ATCC. Table 6 below provides the ATCC Accession Nos. of the ES deposits, all of which were deposited on or before May 13, 1999. The names of the clones contained within each of these deposits are provided in the Table 7 (inserted before the claims).

TABLE 6
Pools of Clones and Libraries Deposited with
ATCC on or before Mar. 28, 2000

| Cell Line | CMCC | ATCC |
|---|---|---|
| ES75 | 5140 | PTA-1102 |
| ES76 | 5141 | PTA-1103 |
| ES77 | 5142 | PTA-1104 |
| ES78 | 5143 | PTA-1105 |
| ES79 | 5144 | PTA-1106 |
| ES80 | 5145 | PTA-1107 |
| ES81 | 5146 | PTA-1108 |
| ES82 | 5147 | PTA-1109 |
| ES83 | 5148 | PTA-1110 |
| ES84 | 5149 | PTA-1111 |

The deposits described herein are provided merely as convenience to those of skill in the art, and is not an admission that a deposit is required under 35 U.S.C. §112. The sequence of the polynucleotides contained within the deposited material, as well as the amino acid sequence of the polypeptides encoded thereby, are incorporated herein by reference and are controlling in the event of any conflict with the written description of sequences herein. A license may be required to make, use, or sell the deposited material, and no such license is granted hereby.

Retrieval of Individual Clones from Deposit of Pooled Clones. Where the ATCC deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

Those skilled in the art will recognize, or be able to ascertain, using not more than routine experimentation, many equivalents to the specific embodiments of the invention described herein. Such specific embodiments and equivalents are intended to be encompassed by the following claims.

All publications and patent applications cited in this specification are herein incorporated by reference as if each individual publication or patent application were specifically and individually indicated to be incorporated by reference. The entire contents of the priority documents, as recited in the Application Data Sheet accompanying this application, are also incorporated by reference herein. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

TABLE 1

| SEQ | | | | | |
|---|---|---|---|---|---|
| ID | CLUSTER | SEQ NAME | ORIENT | CLONE ID | LIBRARY |
| 1 | 819545 | RTA22200265F.k.06.1.P.Seq | F | M00064554D:A03 | CH22PRC |
| 2 | 377944 | RTA22200251F.j.02.1.P.Seq | F | M00063482A:A08 | CH21PRN |
| 3 | 818497 | RTA22200252F.a.13.1.P.Seq | F | M00063514C:D03 | CH21PRN |
| 4 | 819498 | RTA22200252F.n.05.1.P.Seq | F | M00063638C:G12 | CH21PRN |
| 5 | 455465 | RTA22200264F.e.16.1.P.Seq | F | M00064454A:H10 | CH22PRC |
| 6 | 819069 | RTA22200255F.f.01.1.P.Seq | F | M00063940D:F09 | CH21PRN |
| 7 | 672003 | RTA22200265F.b.09.1.P.Seq | F | M00064517C:F11 | CH22PRC |
| 8 | 728115 | RTA22200253F.o.24.1.P.Seq | F | M00063838B:G08 | CH21PRN |
| 9 | 372700 | RTA22200260F.b.20.1.P.Seq | F | M00063580C:A06 | CH22PRC |
| 10 | 818056 | RTA22200266F.c.13.1.P.Seq | F | M00064593D:C01 | CH22PRC |
| 11 | 818497 | RTA22200255F.a.17.1.P.Seq | F | M00063920D:H02 | CH21PRN |
| 12 | 729832 | RTA22200267F.1.21.1.P.Seq | F | M00064714A:G03 | CH22PRC |
| 13 | 505514 | RTA22200251F.b.21.1.P.Seq | F | M00063158A:A01 | CH21PRN |
| 14 | 376488 | RTA22200254F.c.05.1.P.Seq | F | M00063852B:D08 | CH21PRN |
| 15 | 376488 | RTA22200260F.b.09.1.P.Seq | F | M00063578C:A06 | CH22PRC |
| 16 | 748572 | RTA22200254F.c.07.1.P.Seq | F | M00063852D:F07 | CH21PRN |
| 17 | 549934 | RTA22200253F.k.18.1.P.Seq | F | M00063801B:D04 | CH21PRN |
| 18 | 819069 | RTA22200255F.e.24.1.P.Seq | F | M00063940D:F09 | CH21PRN |
| 19 | 817618 | RTA22200253F.n.16.1.P.Seq | F | M00063828D:E05 | CH21PRN |
| 20 | 124396 | RTA22200263F.a.11.2.P.Seq | F | M00064375B:G07 | CH22PRC |
| 21 | 404375 | RTA22200260F.m.08.1.P.Seq | F | M00063967D:G02 | CH22PRC |
| 22 | 391820 | RTA22200261F.f.02.1.P.Seq | F | M00064000B:C03 | CH22PRC |
| 23 | 672003 | RTA22200267F.i.06.1.P.Seq | F | M00064693D:F08 | CH22PRC |
| 24 | 830620 | RTA22200263F.n.09.1.P.Seq | F | M00064424B:C12 | CH22PRC |
| 25 | 450399 | RTA22200251F.f.23.1.P.Seq | F | M00063467D:H07 | CH21PRN |
| 26 | 450982 | RTA22200261F.n.18.1.P.Seq | F | M00064307B:G02 | CH22PRC |
| 27 | 819894 | RTA22200264F.h.18.1.P.Seq | F | M00064467B:D06 | CH22PRC |
| 28 | 379302 | RTA22200257F.j.02.3.P.Seq | F | M00064178C:C04 | CH21PRN |
| 29 | 379746 | RTA22200256F.e.16.1.P.Seq | F | M00064086C:E01 | CH21PRN |
| 30 | 124863 | RTA22200265F.m.06.1.P.Seq | F | M00064564A:C02 | CH22PRC |
| 31 | 379154 | RTA22200257F.c.11.1.P.Seq | F | M00064151B:C07 | CH21PRN |
| 32 | 830620 | RTA22200262F.l.23.1.P.Seq | F | M00064358C:D09 | CH22PRC |
| 33 | 389409 | RTA22200266F.l.24.1.P.Seq | F | M00064631A:C07 | CH22PRC |
| 34 | 397284 | RTA22200262F.i.22.1.P.Seq | F | M00064346C:B09 | CH22PRC |
| 35 | 819440 | RTA22200264F.e.19.1.P.Seq | F | M00064454C:B06 | CH22PRC |
| 36 | 389409 | RTA22200266F.m.01.1.P.Seq | F | M00064631A:C07 | CH22PRC |
| 37 | 518848 | RTA22200265F.n.15.1.P.Seq | F | M00064571C:C04 | CH22PRC |
| 38 | 830620 | RTA22200263F.a.21.1.P.Seq | F | M00064376A:A05 | CH22PRC |
| 39 | 379154 | RTA22200256F.f.20.1.P.Seq | F | M00064090D:D09 | CH21PRN |
| 40 | 818544 | RTA22200256F.h.04.1.P.Seq | F | M00064105B:A03 | CH21PRN |
| 41 | 817375 | RTA22200251F.a.15.1.P.Seq | F | M00063152C:B07 | CH21PRN |
| 42 | 455264 | RTA22200259F.e.23.1.P.Seq | F | M00063539C:C11 | CH22PRC |
| 43 | 817503 | RTA22200266F.k.11.1.P.Seq | F | M00064624D:C09 | CH22PRC |
| 44 | 377696 | RTA22200256F.d.21.1.P.Seq | F | M00064082D:D10 | CH21PRN |
| 45 | 375596 | RTA22200261F.h.10.1.P.Seq | F | M00064009A:C01 | CH22PRC |
| 46 | 817689 | RTA22200263F.h.05.1.P.Seq | F | M00064399A:E01 | CH22PRC |
| 47 | 831867 | RTA22200262F.i.15.2.P.Seq | F | M00064345A:A03 | CH22PRC |
| 48 | 830085 | RTA22200261F.k.14.1.P.Seq | F | M00064293D:B12 | CH22PRC |
| 49 | 389627 | RTA22200264F.c.10.1.P.Seq | F | M00064447B:C06 | CH22PRC |
| 50 | 397284 | RTA22200259F.k.09.1.P.Seq | F | M00063555B:D01 | CH22PRC |
| 51 | 380063 | RTA22200261F.j.02.1.P.Seq | F | M00064014D:H05 | CH22PRC |
| 52 | 830931 | RTA22200266F.m.23.1.P.Seq | F | M00064633C:A03 | CH22PRC |
| 53 | 819321 | RTA22200257F.l.03.3.P.Seq | F | M00064194C:D02 | CH21PRN |
| 54 | 475587 | RTA22200261F.c.01.1.P.Seq | F | M00063990A:D05 | CH22PRC |
| 55 | 819046 | RTA22200255F.a.18.1.P.Seq | F | M00063920D:H05 | CH21PRN |
| 56 | 817477 | RTA22200253F.g.21.1.P.Seq | F | M00063784A:H12 | CH21PRN |
| 57 | 475587 | RTA22200261F.b.24.1.P.Seq | F | M00063990A:D05 | CH22PRC |
| 58 | 728115 | RTA22200253F.p.01.1.P.Seq | F | M00063838B:G08 | CH21PRN |
| 59 | 389627 | RTA22200260F.i.24.1.P.Seq | F | M00063957A:E02 | CH22PRC |
| 60 | 403453 | RTA22200256F.i.24.1.P.Seq | F | M00064113B:C04 | CH21PRN |
| 61 | 508525 | RTA22200255F.d.10.1.P.Seq | F | M00063931B:F07 | CH21PRN |
| 62 | 819525 | RTA22200261F.n.20.1.P.Seq | F | M00064307C:G03 | CH22PRC |
| 63 | 817618 | RTA22200255F.i.03.1.P.Seq | F | M00064025D:H12 | CH21PRN |
| 64 | 819403 | RTA22200254F.h.14.1.P.Seq | F | M00063888D:D05 | CH21PRN |
| 65 | 553242 | RTA22200254F.g.20.1.P.Seq | F | M00063886A:B06 | CH21PRN |
| 66 | 817417 | RTA22200255F.a.10.1.P.Seq | F | M00063919C:E07 | CH21PRN |
| 67 | 817618 | RTA22200252F.f.13.1.P.Seq | F | M00063604A:B11 | CH21PRN |
| 68 | 611440 | RTA22200262F.e.04.2.P.Seq | F | M00064328B:H09 | CH22PRC |
| 69 | 817375 | RTA22200260F.m.06.1.P.Seq | F | M00063967C:A12 | CH22PRC |
| 70 | 213577 | RTA22200255F.i.23.1.P.Seq | F | M00064033C:C11 | CH21PRN |
| 71 | 820061 | RTA22200265F.p.10.1.P.Seq | F | M00064579D:E11 | CH22PRC |
| 72 | 455264 | RTA22200259F.m.06.1.P.Seq | F | M00063559D:G03 | CH22PRC |
| 73 | 455264 | RTA22200255F.o.23.1.P.Seq | F | M00064059A:C11 | CH21PRN |
| 74 | 380331 | RTA22200255F.b.19.1.P.Seq | F | M00063926A:H04 | CH21PRN |
| 75 | 380331 | RTA22200252F.b.19.1.P.Seq | F | M00063518D:A01 | CH21PRN |
| 76 | 817455 | RTA22200267F.o.01.1.P.Seq | F | M00064723D:H03 | CH22PRC |
| 77 | 423967 | RTA22200252F.a.20.1.P.Seq | F | M00063515B:H02 | CR21PRN |
| 78 | 220584 | RTA22200261F.m.14.1.P.Seq | F | M00064302A:D10 | CH22PRC |
| 79 | 817688 | RTA22200251F.e.20.1.P.Seq | F | M00063462D:D07 | CH21PRN |
| 80 | 549934 | RTA22200253F.n.10.1.P.Seq | F | M00063826A:D03 | CH21PRN |
| 81 | 819149 | RTA22200255F.e.16.1.P.Seq | F | M00063938B:H07 | CH21PRN |
| 82 | 817455 | RTA22200267F.n.24.1.P.Seq | F | M00064723D:H03 | CH22PRC |
| 83 | 377696 | RTA22200251F.j.03.1.P.Seq | F | M00063482A:F07 | CH21PRN |
| 84 | 830146 | RTA22200260F.b.07.1.P.Seq | F | M00063578B:E02 | CH22PRC |
| 85 | 194490 | RTA22200264F.l.07.1.P.Seq | F | M00064481C:F03 | CH22PRC |
| 86 | 819460 | RTA22200257F.m.15.3.P.Seq | F | M00064200D:E08 | CH21PRN |
| 87 | 819018 | RTA22200257F.p.01.3.P.Seq | F | M00064212D:E04 | CH21PRN |
| 88 | 830620 | RTA22200259F.p.24.1.P.Seq | F | M00063571B:G03 | CH22PRC |
| 89 | 141079 | RTA22200262F.k.19.1.P.Seq | F | M00064354A:A10 | CH22PRC |
| 90 | 376588 | RTA22200256F.e.04.1.P.Seq | F | M00064083D:E05 | CH21PRN |
| 91 | 380604 | RTA22200264F.g.05.1.P.Seq | F | M00064460C:B01 | CH22PRC |
| 92 | 413138 | RTA22200260F.b.05.1.P.Seq | F | M00063577C:C02 | CH22PRC |
| 93 | 818544 | RTA22200265F.e.12.1.P.Seq | F | M00064527A:H07 | CH22PRC |
| 94 | 647435 | RTA22200257F.h.08.1.P.Seq | F | M00064172C:A02 | CH21PRN |
| 95 | 551785 | RTA22200266F.c.09.1.P.Seq | F | M00064593A:A05 | CH22PRC |
| 96 | 17092 | RTA22200261F.f.17.1.P.Seq | F | M00064002C:F06 | CH22PRC |
| 97 | 818326 | RTA22200251F.i.06.1.P.Seq | F | M00063478C:D01 | CH21PRN |
| 98 | 377944 | RTA22200262F.e.03.2.P.Seq | F | M00064328B:H04 | CH22PRC |
| 99 | 745559 | RTA22200262F.m.04.1.P.Seq | F | M00064359B:H12 | CH22PRC |
| 100 | 818326 | RTA22200265F.d.08.1.P.Seq | F | M00064524A:A09 | CH22PRC |
| 101 | 379879 | RTA22200264F.b.23.1.P.Seq | F | M00064446A:D11 | CH22PRC |
| 102 | 819640 | RTA22200257F.f.24.1.P.Seq | F | M00064165A:B12 | CH21PRN |
| 103 | 818326 | RTA22200265F.a.14.1.P.Seq | F | M00064514D:F11 | CH22PRC |
| 104 | 243524 | RTA22200265F.g.04.1.P.Seq | F | M00064532D:G06 | CH22PRC |
| 105 | 43995 | RTA22200261F.l.02.1.P.Seq | F | M00064294D:F01 | CH22PRC |
| 106 | 597854 | RTA22200262F.g.06.2.P.Seq | F | M00064337D:F01 | CH22PRC |
| 107 | 268290 | RTA22200260F.p.14.1.P.Seq | F | M00063981D:A06 | CH22PRC |
| 108 | 818043 | RTA22200256F.p.10.2.P.Seq | F | M00064138A:F11 | CH21PRN |
| 109 | 830930 | RTA22200267F.b.03.1.P.Seq | F | M00064652B:D09 | CH22PRC |
| 110 | 389627 | RTA22200260F.j.01.1.P.Seq | F | M00063957A:E02 | CH22PRC |
| 111 | 378730 | RTA22200260F.i.07.1.P.Seq | F | M00063955C:F07 | CH22PRC |
| 112 | 819037 | RTA22200260F.n.09.1.P.Seq | F | M00063972C:E10 | CH22PRC |
| 113 | 830397 | RTA22200261F.g.14.1.P.Seq | F | M00064005D:A08 | CH22PRC |
| 114 | 450247 | RTA22200261F.e.10.1.P.Seq | F | M00063998C:E09 | CH22PRC |
| 115 | 819273 | RTA22200252F.b.09.1.P.Seq | F | M00063517A:A04 | CH21PRN |
| 116 | 587779 | RTA22200257F.i.11.3.P.Seq | F | M00064175B:B09 | CH21PRN |
| 117 | 818639 | RTA22200256F.j.09.1.P.Seq | F | M00064115B:E12 | CH21PRN |
| 118 | 615617 | RTA22200261F.o.13.1.P.Seq | F | M00064309C:H09 | CH22PRC |
| 119 | 79309 | RTA22200257F.j.13.3.P.Seq | F | M00064180A:G03 | CH21PRN |
| 120 | 748994 | RTA22200261F.o.20.1.P.Seq | F | M00064310C:A10 | CH22PRC |
| 121 | 818682 | RTA22200258F.h.07.1.P.Seq | F | M00064271B:D03 | CH21PRN |
| 122 | 373061 | RTA22200253F.j.09.1.P.Seq | F | M00063795C:D09 | CH21PRN |
| 123 | 484413 | RTA22200253F.g.09.1.P.Seq | F | M00063781B:B10 | CH21PRN |
| 124 | 819273 | RTA22200258F.h.04.1.P.Seq | F | M00064270B:B03 | CH21PRN |
| 125 | 569532 | RTA22200252F.h.18.1.P.Seq | F | M00063613D:C11 | CH21PRN |
| 126 | 170313 | RTA22200255F.g.20.1.P.Seq | F | M00063949D:A05 | CH21PRN |
| 127 | 818682 | RTA22200253F.p.14.1.P.Seq | F | M00063841A:B09 | CH21PRN |
| 128 | 377188 | RTA22200255F.l.06.1.P.Seq | F | M00064043D:C09 | CH21PRN |
| 129 | 518848 | RTA22200257F.j.22.3.P.Seq | F | M00064186C:B03 | CH21PRN |
| 130 | 45592 | RTA22200259F.l.08.1.P.Seq | F | M00063557D:C07 | CH22PRC |
| 131 | 819273 | RTA22200255F.n.19.1.P.Seq | F | M00064053C:G04 | CH21PRN |
| 132 | 397284 | RTA22200251F.a.06.1.P.Seq | F | M00063151D:B10 | CH21PRN |
| 133 | 818326 | RTA22200258F.e.14.1.P.Seq | F | M00064260C:E05 | CH21PRN |
| 134 | 819037 | RTA22200251F.c.15.1.P.Seq | F | M00063452A:F08 | CH21PRN |
| 135 | 817417 | RTA22200253F.m.14.1.P.Seq | F | M00063818C:A09 | CH21PRN |
| 136 | 819640 | RTA22200254F.i.11.1.P.Seq | F | M00063891A:F11 | CH21PRN |
| 137 | 818771 | RTA22200254F.i.19.1.P.Seq | F | M00063892B:G02 | CH21PRN |
| 138 | 389627 | RTA22200254F.k.10.1.P.Seq | F | M00063898A:A10 | CH21PRN |
| 139 | 379067 | RTA22200260F.e.20.1.P.Seq | F | M00063593A:D03 | CH22PRC |
| 140 | 818544 | RTA22200251F.f.02.1.P.Seq | F | M00063463D:B05 | CH21PRN |
| 141 | 819440 | RTA22200251F.j.22.1.P.Seq | F | M00063485A:E05 | CH21PRN |
| 142 | 817417 | RTA22200251F.k.10.1.P.Seq | F | M00063487C:C02 | CH21PRN |
| 143 | 385307 | RTA22200262F.k.11.1.P.Seq | F | M00064352C:H01 | CH22PRC |
| 144 | 611440 | RTA22200263F.d.24.2.P.Seq | F | M00064386B:C02 | CH22PRC |
| 145 | 376056 | RTA22200259F.e.16.1.P.Seq | F | M00063538D:B01 | CH22PRC |
| 146 | 611440 | RTA22200263F.d.24.1.P.Seq | F | M00064386B:C02 | CH22PRC |
| 147 | 820061 | RTA22200264F.f.09.1.P.Seq | F | M00064457D:C09 | CH22PRC |
| 148 | 617825 | RTA22200264F.p.06.1.P.Seq | F | M00064508A:B09 | CH22PRC |
| 149 | 819440 | RTA22200257F.h.17.1.P.Seq | F | M00064173B:E01 | CH21PRN |
| 150 | 819145 | RTA22200266F.m.08.1.P.Seq | F | M00064631C:H11 | CH22PRC |
| 151 | 817653 | RTA22200265F.p.07.1.P.Seq | F | M00064579A:C06 | CH22PRC |
| 152 | 611440 | RTA22200263F.e.01.1.P.Seq | F | M00064386B:C02 | CH22PRC |
| 153 | 375958 | RTA22200264F.j.22.1.P.Seq | F | M00064476D:C04 | CH22PRC |
| 154 | 611440 | RTA22200257F.a.20.1.P.Seq | F | M00064144D:A07 | CH21PRN |
| 155 | 831049 | RTA22200266F.0.13.1.P.Seq | F | M00064637B:F03 | CH22PRC |
| 156 | 818162 | RTA22200266F.g.18.1.P.Seq | F | M00064610D:H01 | CH22PRC |
| 157 | 553200 | RTA22200263F.p.02.1.P.Seq | F | M00064429D:B07 | CH22PRC |
| 158 | 139677 | RTA22200254F.o.07.1.P.Seq | F | M00063910D:A12 | CH21PRN |
| 159 | 139677 | RTA22200252F.c.11.1.P.Seq | F | M00063520D:E11 | CH21PRN |
| 160 | 397284 | RTA22200262F.i.22.2.P.Seq | F | M00064346C:B09 | CH22PRC |
| 161 | 385810 | RTA22200256F.m.04.2.P.Seq | F | M00064126C:F12 | CH21PRN |
| 162 | 404624 | RTA22200261F.e.07.1.P.Seq | F | M00063997C:B12 | CH22PRC |
| 163 | 375958 | RTA22200262F.b.14.2.P.Seq | F | M00064322C:A10 | CH22PRC |
| 164 | 616555 | RTA22200265F.b.24.1.P.Seq | F | M00064520A:E04 | CH22PRC |
| 165 | 616555 | RTA22200265F.c.01.1.P.Seq | F | M00064520A:E04 | CH22PRC |
| 166 | 295694 | RTA22200260F.o.20.1.P.Seq | F | M00063978B:B06 | CH22PRC |
| 167 | 36113 | RTA22200265F.e.06.1.P.Seq | F | M00064526D:F05 | CH22PRC |
| 168 | 831812 | RTA22200263F.f.05.1.P.Seq | F | M00064390A:C05 | CH22PRC |
| 169 | 817653 | RTA22200252F.g.23.1.P.Seq | F | M00063610D:C11 | CH21PRN |
| 170 | 397284 | RTA22200252F.m.15.1.P.Seq | F | M00063636A:E01 | CH21PRN |
| 171 | 817979 | RTA22200253F.p.15.1.P.Seq | F | M00063841A:E08 | CH21PRN |
| 172 | 817653 | RTA22200255F.m.18.1.P.Seq | F | M00064048C:G12 | CH21PRN |
| 173 | 611440 | RTA22200253F.f.03.1.P.Seq | F | M00063774A:D09 | CH21PRN |
| 174 | 386014 | RTA22200261F.f.06.1.P.Seq | F | M00064001A:B03 | CH22PRC |
| 175 | 549981 | RTA22200255F.b.10.1.P.Seq | F | M00063925B:F04 | CH21PRN |
| 176 | 193373 | RTA22200255F.l.21.1.P.Seq | F | M00064046A:G02 | CH21PRN |
| 177 | 400619 | RTA22200255F.g.14.1.P.Seq | F | M00063947D:D01 | CH21PRN |
| 178 | 831149 | RTA22200261F.o.21.1.P.Seq | F | M00064310D:F03 | CH22PRC |
| 179 | 36113 | RTA22200255F.d.16.1.P.Seq | F | M00063932D:G08 | CH21PRN |
| 180 | 817503 | RTA22200253F.l.16.1.P.Seq | F | M00063805D:E05 | CH21PRN |
| 181 | 376588 | RTA22200260F.i.11.1.P.Seq | F | M00063955D:F05 | CH22PRC |
| 182 | 141079 | RTA22200252F.f.23.1.P.Seq | F | M00063606C:B04 | CH21PRN |
| 183 | 818063 | RTA22200253F.p.04.1.P.Seq | F | M00063839A:F01 | CH21PRN |
| 184 | 455264 | RTA22200253F.n.14.1.P.Seq | F | M00063828A:H12 | CH21PRN |
| 185 | 189234 | RTA22200251F.f.17.1.P.Seq | F | M00063466C:C11 | CH21PRN |
| 186 | 295694 | RTA22200265F.j.05.1.P.Seq | F | M00064550A:A07 | CH22PRC |
| 187 | 648679 | RTA22200260F.f.06.1.P.Seq | F | M00063594B:H07 | CH22PRC |
| 188 | 830930 | RTA22200264F.e.10.1.P.Seq | F | M00064452D:E11 | CH22PRC |
| 189 | 818497 | RTA22200256F.d.07.1.P.Seq | F | M00064079C:A10 | CH21PRN |
| 190 | 373928 | RTA22200256F.d.19.1.P.Seq | F | M00064082A:A08 | CH21PRN |
| 191 | 385307 | RTA22200263F.j.12.1.P.Seq | F | M00064406B:H06 | CH22PRC |
| 192 | 403453 | RTA22200266F.e.10.1.P.Seq | F | M00064601D:B05 | CH22PRC |
| 193 | 730318 | RTA22200264F.c.09.1.P.Seq | F | M00064447B:A07 | CH22PRC |
| 194 | 44183 | RTA22200271F.a.01.1.P.Seq | F | M00021929A:D03 | CH03MAH |
| 195 | 373928 | RTA22200255F.d.22.1.P.Seq | F | M00063934B:E04 | CH21PRN |
| 196 | 404624 | RTA22200255F.d.23.1.P.Seq | F | M00063934C:C10 | CH21PRN |
| 197 | 403173 | RTA22200253F.a.21.1.P.Seq | F | M00063685A:C02 | CH21PRN |
| 198 | 372700 | RTA22200253F.c.06.1.P.Seq | F | M00063689D:E12 | CH21PRN |
| 199 | 374343 | RTA22200261F.h.04.1.P.Seq | F | M00064008A:B01 | CH22PRC |
| 200 | 597854 | RTA22200255F.j.03.1.P.Seq | F | M00064033D:B01 | CH21PRN |
| 201 | 817417 | RTA22200255F.a.23.1.P.Seq | F | M00063922B:A12 | CH21PRN |
| 202 | 818497 | RTA22200257F.k.05.3.P.Seq | F | M00064188B:G08 | CH21PRN |
| 203 | 377696 | RTA22200255F.f.15.1.P.Seq | F | M00063943B:G12 | CH21PRN |
| 204 | 379105 | RTA22200252F.n.19.1.P.Seq | F | M00063642B:A08 | CH21PRN |
| 205 | 831188 | RTA22200267F.o.02.1.P.Seq | F | M00064723D:H11 | CH22PRC |
| 206 | 376056 | RTA22200253F.m.09.1.P.Seq | F | M00063810C:E03 | CH21PRN |
| 207 | 124863 | RTA22200255F.n.15.1.P.Seq | F | M00064053B:D09 | CH21PRN |
| 208 | 376056 | RTA22200254F.i.03.1.P.Seq | F | M00063890A:F11 | CH21PRN |
| 209 | 831812 | RTA22200266F.j.10.1.P.Seq | F | M00064620C:D01 | CH22PRC |
| 210 | 141079 | RTA22200260F.i.14.1.P.Seq | F | M00063956A:F05 | CH22PRC |
| 211 | 19148 | RTA22200265F.o.18.1.P.Seq | F | M00064577C:B12 | CH22PRC |
| 212 | 124396 | RTA22200252F.a.14.1.P.Seq | F | M00063514C:E08 | CH21PRN |
| 213 | 831026 | RTA22200265F.c.03.1.P.Seq | F | M00064520A:F08 | CH22PRC |
| 214 | 819037 | RTA22200263F.i.23.1.P.Seq | F | M00064405B:C04 | CH22PRC |
| 215 | 380207 | RTA22200263F.i.19.1.P.Seq | F | M00064404C:G05 | CH22PRC |
| 216 | 819460 | RTA22200255F.c.13.1.P.Seq | F | M00063928A:G09 | CH21PRN |
| 217 | 379067 | RTA22200253F.g.23.1.P.Seq | F | M00063784C:E10 | CH21PRN |
| 218 | 403173 | RTA22200252F.p.23.1.P.Seq | F | M00063682A:C04 | CH21PRN |
| 219 | 3856 | RTA22200269F.a.05.1.P.Seq | F | M00003773D:H02 | CH01COH |
| 220 | 378551 | RTA22200263F.d.17.1.P.Seq | F | M00064385D:C11 | CH22PRC |
| 221 | 456089 | RTA22200272F.a.09.1.P.Seq | F | M00043134A:A05 | CH19COP |
| 222 | 549981 | RTA22200267F.a.22.1.P.Seq | F | M00064650B:B07 | CH22PRC |
| 223 | 378551 | RTA22200265F.m.21.1.P.Seq | F | M00064568A:H06 | CH22PRC |
| 224 | 819201 | RTA22200256F.n.23.2.P.Seq | F | M00064132B:B07 | CH21PRN |
| 225 | 374826 | RTA22200251F.c.20.1.P.Seq | F | M00063453B:F08 | CH21PRN |
| 226 | 389409 | RTA22200253F.l.23.1.P.Seq | F | M00063807A:D12 | CH21PRN |
| 227 | 819149 | RTA22200260F.a.17.1.P.Seq | F | M00063575B:G02 | CH22PRC |
| 228 | 389409 | RTA22200255F.e.18.1.P.Seq | F | M00063939C:D06 | CH21PRN |
| 229 | 818165 | RTA22200254F.h.15.1.P.Seq | F | M00063888D:F02 | CH21PRN |
| 230 | 817757 | RTA22200252F.i.15.1.P.Seq | F | M00063617D:F09 | CH21PRN |
| 231 | 553242 | RTA22200263F.i.20.1.P.Seq | F | M00064404D:A06 | CH22PRC |
| 232 | 385615 | RTA22200265F.b.08.1.P.Seq | F | M00064517B:F10 | CH22PRC |
| 233 | 819102 | RTA22200258F.h.19.1.P.Seq | F | M00064272C:G01 | CH21PRN |
| 234 | 817757 | RTA22200255F.o.16.1.P.Seq | F | M00064057C:H10 | CH21PRN |
| 235 | 385615 | RTA22200265F.b.07.1.P.Seq | F | M00064517B:F04 | CH22PRC |
| 236 | 385615 | RTA22200253F.l.06.1.P.Seq | F | M00063804C:A11 | CH21PRN |
| 237 | 827355 | RTA22200266F.n.23.1.P.Seq | F | M00064636B:A04 | CH22PRC |
| 238 | 817629 | RTA22200259F.a.13.1.P.Seq | F | M00063165A:C09 | CH22PRC |
| 239 | 817514 | RTA22200260F.h.02.1.P.Seq | F | M00063600C:C09 | CH22PRC |
| 240 | 817514 | RTA22200252F.p.21.1.P.Seq | F | M00063681B:C02 | CH21PRN |
| 241 | 680563 | RTA22200265F.f.13.1.P.Seq | F | M00064530B:H02 | CH22PRC |
| 242 | 827355 | RTA22200255F.e.20.1.P.Seq | F | M00063939C:H01 | CH21PRN |
| 243 | 377286 | RTA22200254F.a.04.1.P.Seq | F | M00063843B:D07 | CH21PRN |
| 244 | 680563 | RTA22200258F.g.18.1.P.Seq | F | M00064268D:G03 | CH21PRN |
| 245 | 819156 | RTA22200255F.h.06.1.P.Seq | F | M00064021D:H01 | CH21PRN |
| 246 | 220584 | RTA22200261F.f.22.1.P.Seq | F | M00064003B:C10 | CH22PRC |
| 247 | 616555 | RTA22200263F.o.12.1.P.Seq | F | M00064428B:A12 | CH22PRC |
| 248 | 819498 | RTA22200254F.o.14.1.P.Seq | F | M00063912A:D06 | CH21PRN |
| 249 | 817508 | RTA22200257F.h.01.1.P.Seq | F | M00064171D:E05 | CH21PRN |
| 250 | 817690 | RTA22200257F.e.05.1.P.Seq | F | M00064159A:H03 | CH21PRN |
| 251 | 819156 | RTA22200256F.h.13.1.P.Seq | F | M00064106C:G03 | CH21PRN |
| 252 | 830904 | RTA22200266F.j.12.1.P.Seq | F | M00064620D:G05 | CH22PRC |
| 253 | 819498 | RTA22200253F.b.04.1.P.Seq | F | M00063686B:E07 | CH21PRN |
| 254 | 817508 | RTA22200257F.g.24.1.P.Seq | F | M00064171D:E05 | CH21PRN |
| 255 | 817508 | RTA22200252F.a.19.1.P.Seq | F | M00063515B:F06 | CH21PRN |
| 256 | 831160 | RTA22200267F.h.01.1.P.Seq | F | M00064690A:C04 | CH22PRC |
| 257 | 817762 | RTA22200252F.k.13.1.P.Seq | F | M00063627C:F06 | CH21PRN |
| 258 | 377286 | RTA22200266F.k.07.1.P.Seq | F | M00064624C:B03 | CH22PRC |
| 259 | 831160 | RTA22200267F.g.24.1.P.Seq | F | M00064690A:C04 | CH22PRC |
| 260 | 819994 | RTA22200256F.k.11.1.P.Seq | F | M00064119C:D12 | CH21PRN |
| 261 | 819994 | RTA22200256F.k.09.1.P.Seq | F | M00064119B:H10 | CH21PRN |
| 262 | 373298 | RTA22200259F.c.19.1.P.Seq | F | M00063533A:C12 | CH22PRC |
| 263 | 819894 | RTA22200256F.m.03.2.P.Seq | F | M00064126C:C02 | CH21PRN |
| 264 | 372718 | RTA22200260F.b.22.1.P.Seq | F | M00063580D:B06 | CH22PRC |
| 265 | 827355 | RTA22200262F.1.20.1.P.Seq | F | M00064358A:G03 | CH22PRC |
| 266 | 819894 | RTA22200255F.d.09.1.P.Seq | F | M00063931B:E10 | CH21PRN |
| 267 | 827355 | RTA22200266F.e.07.1.P.Seq | F | M00064601C:G07 | CH22PRC |
| 268 | 372718 | RTA22200256F.1.03.1.P.Seq | F | M00064122C:B06 | CH21PRN |
| 269 | 647435 | RTA22200251F.b.10.1.P.Seq | F | M00063156D:H10 | CH21PRN |
| 270 | 450262 | RTA22200265F.a.10.1.P.Seq | F | M00064514A:G10 | CH22PRC |
| 271 | 484703 | RTA22200255F.i.20.1.P.Seq | F | M00064032D:G04 | CH21PRN |
| 272 | 819498 | RTA22200256F.f.12.1.P.Seq | F | M00064089B:F09 | CH21PRN |
| 273 | 406043 | RTA22200263F.i.12.1.P.Seq | F | M00064404A:B05 | CH22PRC |
| 274 | 817500 | RTA22200255F.f.24.1.P.Seq | F | M00063945A:C03 | CH21PRN |
| 275 | 818180 | RTA22200264F.o.18.1.P.Seq | F | M00064506A:C07 | CH22PRC |
| 276 | 818143 | RTA22200251F.a.03.1.P.Seq | F | M00063151A:G06 | CH21PRN |
| 277 | 819756 | RTA22200267F.a.18.1.P.Seq | F | M00064649A:E04 | CH22PRC |
| 278 | 406908 | RTA22200257F.i.18.3.P.Seq | F | M00064176D:H10 | CH21PRN |
| 279 | 124863 | RTA22200256F.o.21.2.P.Seq | F | M00064136C:D12 | CH21PRN |
| 280 | 429009 | RTA22200257F.e.24.1.P.Seq | F | M00064161B:G04 | CH21PRN |
| 281 | 402586 | RTA22200257F.i.24.3.P.Seq | F | M00064178B:A05 | CH21PRN |
| 282 | 400475 | RTA22200254F.i.04.1.P.Seq | F | M00063890A:H04 | CH21PRN |
| 283 | 403453 | RTA22200264F.d.12.1.P.Seq | F | M00064450C:E07 | CH22PRC |
| 284 | 383021 | RTA22200259F.d.06.1.P.Seq | F | M00063534C:A02 | CH22PRC |
| 285 | 394913 | RTA22200254F.p.10.1.P.Seq | F | M00063915C:E01 | CH21PRN |
| 286 | 831361 | RTA22200263F.k.19.1.P.Seq | F | M00064414D:D06 | CH22PRC |
| 287 | 646020 | RTA22200267F.n.21.1.P.Seq | F | M00064723C:H04 | CH22PRC |
| 288 | 831361 | RTA22200263F.1.03.1.P.Seq | F | M00064415B:G03 | CH22PRC |
| 289 | 831580 | RTA22200261F.f.18.1.P.Seq | F | M00064002C:H09 | CH22PRC |
| 290 | 402586 | RTA22200257F.j.01.3.P.Seq | F | M00064178B:A05 | CH21PRN |
| 291 | 400475 | RTA22200262F.j.21.1.P.Seq | F | M00064349D:H01 | CH22PRC |
| 292 | 818937 | RTA22200262F.h.14.2.P.Seq | F | M00064341A:C02 | CH22PRC |
| 293 | 557697 | RTA22200261F.j.20.1.P.Seq | F | M00064018C:E07 | CH22PRC |
| 294 | 831361 | RTA22200265F.m.24.1.P.Seq | F | M00064569B:A09 | CH22PRC |
| 295 | 194490 | RTA22200252F.c.10.1.P.Seq | F | M00063520D:D08 | CH21PRN |
| 296 | 818143 | RTA22200254F.b.18.1.P.Seq | F | M00063848C:G11 | CH21PRN |
| 297 | 377286 | RTA22200259F.a.10.1.P.Seq | F | M00063163A:G04 | CH22PRC |
| 298 | 831361 | RTA22200265F.n.01.1.P.Seq | F | M00064569B:A09 | CH22PRC |
| 299 | 385307 | RTA22200255F.p.07.1.P.Seq | F | M00064060B:D03 | CH21PRN |
| 300 | 378447 | RTA22200251F.c.01.1.P.Seq | F | M00063158A:E11 | CH21PRN |
| 301 | 378447 | RTA22200251F.b.24.1.P.Seq | F | M00063158A:E11 | CH21PRN |
| 302 | 817514 | RTA22200260F.m.17.1.P.Seq | F | M00063968D:G08 | CH22PRC |
| 303 | 818942 | RTA22200255F.f.03.1.P.Seq | F | M00063941B:C12 | CH21PRN |
| 304 | 818942 | RTA22200267F.e.23.1.P.Seq | F | M00064678D:F05 | CH22PRC |
| 305 | 817363 | RTA22200266F.f.04.1.P.Seq | F | M00064605C:G05 | CH22PRC |
| 306 | 818942 | RTA22200255F.i.02.1.P.Seq | F | M00064025D:E07 | CH21PRN |
| 307 | 818942 | RTA22200265F.g.23.1.P.Seq | F | M00064534D:F06 | CH22PRC |
| 308 | 817457 | RTA22200267F.e.15.1.P.Seq | F | M00064675C:E09 | CH22PRC |
| 309 | 831968 | RTA22200263F.f.23.1.P.Seq | F | M00064393B:H04 | CH22PRC |
| 310 | 530941 | RTA22200253F.h.05.1.P.Seq | F | M00063785C:F03 | CH21PRN |
| 311 | 763446 | RTA22200257F.j.05.3.P.Seq | F | M00064179A:C04 | CH21PRN |
| 312 | 763446 | RTA22200255F.n.21.1.P.Seq | F | M00064053D:F02 | CH21PRN |
| 313 | 819219 | RTA22200256F.f.16.1.P.Seq | F | M00064090C:A02 | CH21PRN |
| 314 | 763446 | RTA22200258F.b.19.2.P.Seq | F | M00064248A:E02 | CH21PRN |
| 315 | 10154 | | | | |
| 316 | 10154 | | | | |

TABLE 2

| | Nearest | | | Nearest Neighbor | | |
|---|---|---|---|---|---|---|
| | Neighbor | | | (BlastX vs. Non- | | |
| | (BlastN vs. | | | Redundant | | |
| SEQ | Genbank) | | | Proteins) | | |
| ID | ACCESSION | DESCRIPTION | P VALUE | ACCESSION | DESCRIPTION | P VALUE |
| 19 | <NONE> | <NONE> | <NONE> | 1077580 | hypothetical | 7 |
| | | | | | protein | |
| | | | | | YDR125c - | |
| | | | | | yeast | |
| 20 | <NONE> | <NONE> | <NONE> | 4585925 | (AC007211) | 6 |
| | | | | | unknown protein | |
| 21 | <NONE> | <NONE> | <NONE> | 1085306 | EVI1 protein - | 4.3 |
| | | | | | human | |
| 22 | <NONE> | <NONE> | <NONE> | 3876587 | (Z81521) | 0.85 |
| | | | | | predicted using | |
| | | | | | Genefinder; | |
| | | | | | cDNA EST | |
| | | | | | yk233g4.5 | |
| | | | | | comes from this | |
| | | | | | gene; cDNA | |
| | | | | | EST yk233g4.3 | |
| | | | | | comes from this | |
| | | | | | gene | |
| | | | | | [Caenorhabditis | |
| | | | | | elegans] | |
| 23 | <NONE> | <NONE> | <NONE> | 1086591 | (U41007) | 0.34 |
| | | | | | similar to S. | |
| | | | | | cervisiae nuclear | |
| | | | | | protein SNF2 | |
| 24 | <NONE> | <NONE> | <NONE> | 157272 | (L11345) DNA - | 0.29 |
| | | | | | binding protein | |
| | | | | | [Drosophila | |
| | | | | | melanogaster] | |
| 25 | <NONE> | <NONE> | <NONE> | 2633160 | (Z99108) | 0.19 |
| | | | | | similar to | |
| | | | | | surface adhesion | |
| | | | | | YfiQ [Bacillus | |
| | | | | | subtilis] | |
| 26 | <NONE> | <NONE> | <NONE> | 755468 | (U19879) | 0.042 |
| | | | | | transmembrane | |
| | | | | | protein | |
| | | | | | [Xenopus laevis] | |
| 27 | <NONE> | <NONE> | <NONE> | 4507339 | T brachyury | 0.029 |
| | | | | | (mouse) | |
| | | | | | homolog protein | |
| | | | | | [Homo sapiens] | |
| 28 | <NONE> | <NONE> | <NONE> | 729711 | PROTEASE | 0.004 |
| | | | | | DEGS | |
| | | | | | PRECURSOR | |
| | | | | | 3.4.21.—) hhoB - | |
| | | | | | Escherichia coli> gi\| | |
| | | | | | 558913 | |
| | | | | | (U15661) HhoB | |
| | | | | | [Escherichia | |
| | | | | | coli] > gi\| | |
| | | | | | 606174 | |
| | | | | | (U18997) | |
| | | | | | ORF_o355 coli] > gi\| | |
| | | | | | 1789630 | |
| | | | | | (AE000402) | |
| | | | | | protease | |
| | | | | | [Escherichia | |
| | | | | | coli] | |
| 29 | <NONE> | <NONE> | <NONE> | 3168911 | (AF068718) No | 8e−013 |
| | | | | | definition line | |
| | | | | | found | |
| | | | | | [Caenorhabditis | |
| | | | | | elegans] | |
| 30 | <NONE> | <NONE> | <NONE> | 2832777 | (AL021086)/ | 3e−040 |
| | | | | | prediction = (method:; | |
| | | | | | comes | |
| | | | | | from the 5′ | |
| | | | | | UTR | |
| | | | | | [Drosophila | |
| | | | | | melanogaster] | |
| 31 | X78712 | H. sapiens | 2.1 | 2852449 | (D88207) | 9.1 |
| | | mRNA for | | | protein kinase | |
| | | glycerol kinase | | | [Arabidopsis | |
| | | testis specific 2 | | | thaliana] > gi\| | |
| | | | | | 2947061 | |
| | | | | | (AC002521) | |
| | | | | | putative protein | |
| | | | | | kinase | |
| 32 | X60760 | L. esculentum | 2.1 | 157272 | (L11345) DNA - | 5 |
| | | TDR8 mRNA | | | binding protein | |
| | | | | | [Drosophila | |
| | | | | | melanogaster] | |
| 33 | U40853 | Oryctolagus | 2 | <NONE> | <NONE> | <NONE> |
| | | cuniculus | | | | |
| | | pulmonary | | | | |
| | | surfactant | | | | |
| | | protein B (SP-B) | | | | |
| | | gene, complete | | | | |
| | | cds | | | | |
| 34 | AF083655 | Homo sapiens | 2 | <NONE> | <NONE> | <NONE> |
| | | procollagen C- | | | | |
| | | proteinase | | | | |
| | | enhancer protein | | | | |
| | | (PCOLCE) | | | | |
| | | gene, 5′ | | | | |
| | | flanking region | | | | |
| | | and complete | | | | |
| | | cds | | | | |
| 35 | AJ223776 | Staphylococcus | 2 | <NONE> | <NONE> | <NONE> |
| | | warneri hld gene | | | | |
| 36 | U40853 | Oryctolagus | 2 | <NONE> | <NONE> | <NONE> |
| | | cuniculus | | | | |
| | | pulmonary | | | | |
| | | surfactant | | | | |
| | | protein B (SP-B) | | | | |
| | | gene, complete | | | | |
| | | cds | | | | |
| 37 | X04436 | Clostridium | 2 | <NONE> | <NONE> | <NONE> |
| | | tetani gene for | | | | |
| | | tetanus toxin | | | | |
| 38 | Z35787 | S. cerevisiae | 2 | 157272 | (L11345) DNA - | 8.4 |
| | | chromosome II | | | binding protein | |
| | | reading frame | | | [Drosophila | |
| | | ORF YBL026w | | | melanogaster] | |
| 39 | X78712 | H. sapiens | 2 | 2852449 | (D88207) | 8.2 |
| | | mRNA for | | | protein kinase | |
| | | glycerol kinase | | | [Arabidopsis | |
| | | testis specific 2 | | | thaliana] > gi\| | |
| | | | | | 2947061 | |
| | | | | | (AC002521) | |
| | | | | | putative protein | |
| | | | | | kinase | |
| 40 | Z15056 | B. subtilis genes | 2 | 477124 | P3A2 DNA | 2.8 |
| | | spoVD, murE, | | | binding protein | |
| | | mraY, murD | | | homolog EWG - | |
| | | | | | fruit fly | |
| | | | | | (Drosophila | |
| | | | | | melanogaster) | |
| 41 | S65623 | cAMP-regulated | 2 | 119266 | PROTEIN | 0.55 |
| | | enhancer- | | | GRAINY- | |
| | | binding protein | | | HEAD (DNA- | |
| | | 1 of 3] | | | BINDING | |
| | | | | | PROTEIN ELF- | |
| | | | | | 1) (ELEMENT | |
| | | | | | I-BINDING | |
| | | | | | ACTIVITY) | |
| | | | | | regulatory | |
| | | | | | protein elf-1 - | |
| | | | | | fruit fly | |
| | | | | | (Drosophila | |
| | | | | | melanogaster) > gi\| | |
| | | | | | 7939\|emb\| | |
| | | | | | CAA33692\| | |
| | | | | | (X15657) Elf-1 | |
| | | | | | protein (AA 1-1063) | |
| | | | | | [Drosophila | |
| | | | | | melanogaster] | |
| 42 | NM_0044151 | Homo sapiens | 2 | 2649177 | (AE001008) | 0.2 |
| | | desmoplakin | | | conserved | |
| | | (DPI, DPII) | | | hypothetical | |
| | | (DSP) mRNA | | | protein | |
| | | mRNA, | | | [Archaeoglobus | |
| | | complete cds | | | fulgidus] | |
| 43 | AF031552 | Vibrio cholerae | 2 | 2088714 | (AF003139) | 2e−013 |
| | | magnesium | | | strong similarity | |
| | | transporter | | | to NADPH | |
| | | (mgtE) gene, | | | oxidases; partial | |
| | | partial cds; | | | CDS, the gene | |
| | | sensor kinase | | | begins in the | |
| | | (vieS), response | | | neighboring | |
| | | regulator, | | | clone | |
| | | (vieA), and | | | | |
| | | response | | | | |
| | | regulator (vieB) | | | | |
| | | genes, complete | | | | |
| | | cds; and | | | | |
| | | collagenase | | | | |
| | | (vcc) gene, | | | | |
| | | (vcc) gene, | | | | |
| | | partial cds | | | | |
| 44 | AF116852.1 | Danio rerio | 2 | 3800951 | (AF100657) No | 2e−019 |
| | | dickkopf-1 | | | definition line | |
| | | (dkk1) mRNA, | | | found | |
| | | complete cds | | | [Caenorhabditis | |
| | | | | | elegans] | |
| 45 | X82595 | P. sativum fuc | 1.9 | <NONE> | <NONE> | <NONE> |
| | | gene | | | | |
| 46 | AF008216 | Homo sapiens | 1.9 | <NONE> | <NONE> | <NONE> |
| | | candidate tumor | | | | |
| | | suppressor | | | | |
| | | pp32r1 | | | | |
| 47 | AF130672.1 | Felis catus clone | 1.9 | <NONE> | <NONE> | <NONE> |
| | | Fca603 | | | | |
| | | microsatellite | | | | |
| | | sequence | | | | |
| 48 | AJ007044 | Oryctolagus | 1.9 | 388055 | (L22981) | 7.8 |
| | | Cuniculus sod | | | merozoite | |
| | | gene | | | surface protein- | |
| | | | | | 1 [Plasmodium | |
| | | | | | chabaudi] | |
| 49 | AC004497 | Homo sapiens | 1.9 | 160925 | (M94346) | 7.7 |
| | | chromosome 21, | | | A.1.12/9 | |
| | | P1 clone | | | antigen | |
| | | LBNL#6 | | | [Schistosoma | |
| | | | | | mansoni] | |
| 50 | U30290 | Rattus | 1.9 | 3024079 | GALECTIN-4 | 4.5 |
| | | norvegicus | | | (LACTOSE | |
| | | galanin receptor | | | BINDING | |
| | | GALR1 mRNA, | | | LECTIN 4) (L- | |
| | | complete cds | | | 36 LACTOSE | |
| | | | | | BINDING | |
| | | | | | PROTEIN) | |
| | | | | | (L36LBP) | |
| | | | | | >gi\|2281707 | |
| | | | | | sapiens] | |
| | | | | | >gi\|2623387 | |
| | | | | | (U82953) | |
| | | | | | galectin-4 | |
| | | | | | [Homo sapiens] | |
| 51 | Y13234 | Chironomus | 1.9 | 4567068 | (AF125568) | 3.4 |
| | | tentans mRNA | | | tumor | |
| | | for chitinase, | | | suppressing STF | |
| | | 1695 bp | | | cDNA 4 [Homo sapiens] | |
| 52 | NM_003644.1 | Homo sapiens | 1.9 | 125560 | PROTEIN | 0.53 |
| | | growth arrest- | | | KINASE C, | |
| | | specific 7 | | | GAMMA TYPE | |
| | | (GAS7) mRNA > :: | | | C (EC 2.7.1.—) | |
| | | emb\|AJ224876\| | | | gamma - rabbit | |
| | | HSAJ4876 | | | >gi\|165652 | |
| | | Homo sapience | | | (M19338) | |
| | | mRNA for | | | protein kinase | |
| | | GAS7 protein | | | delta | |
| | | | | | [Oryctolagus | |
| | | | | | cuniculus] | |
| 53 | AB013448.1 | Oryza sativa | 1.8 | <NONE> | <NONE> | <NONE> |
| | | gene for Pib, | | | | |
| | | complete cds | | | | |
| 54 | D63854 | Human | 1.8 | <NONE> | <NONE> | <NONE> |
| | | cytomegalovirus | | | | |
| | | DNA, replication | | | | |
| | | origin | | | | |
| 55 | AB002340 | Human mRNA | 1.8 | <NONE> | <NONE> | <NONE> |
| | | for KIAA0342 | | | | |
| | | gene, complete | | | | |
| | | cds | | | | |
| 56 | AF017779 | Mus musculus | 1.8 | <NONE> | <NONE> | <NONE> |
| | | vitamin D | | | | |
| | | receptor gene, | | | | |
| | | promoter region | | | | |
| 57 | D63854 | Human | 1.8 | <NONE> | <NONE> | <NONE> |
| | | cytomegalovirus | | | | |
| | | DNA, replication | | | | |
| | | origin | | | | |
| 58 | M24102 | Bovine | 1.8 | <NONE> | <NONE> | <NONE> |
| | | ADP/ATP | | | | |
| | | translocase T1 | | | | |
| | | mRNA, | | | | |
| | | complete cds. | | | | |
| 59 | AC004497 | Homo sapiens | 1.8 | <NONE> | <NONE> | <NONE> |
| | | chromosome 21, | | | | |
| | | P1 clone | | | | |
| | | LBNL#6 | | | | |
| 60 | M37394 | Rat epidermal | 1.8 | <NONE> | <NONE> | <NONE> |
| | | growth factor | | | | |
| | | receptor mRNA. | | | | |
| 61 | AF006304 | Saccharomyces | 1.8 | <NONE> | <NONE> | <NONE> |
| | | cerevisiae | | | | |
| | | protein tyrosine | | | | |
| | | phosphatase | | | | |
| | | (PTP3) gene, | | | | |
| | | complete cds | | | | |
| 62 | D13454 | Candida | 1.8 | <NONE> | <NONE> | <NONE> |
| | | albicans | | | | |
| | | CACHS3 gene | | | | |
| | | for chitin | | | | |
| | | synthase III | | | | |
| 63 | Y00354 | Xenopus laevis | 1.8 | 1077580 | hypothetical | 7.5 |
| | | gene encoding | | | protein | |
| | | vitellogenin A2 | | | YDR125c - | |
| | | | | | yeast | |
| 64 | U90936 | Aspergillus | 1.8 | 4337033 | (AF124138) | 7.3 |
| | | niger px27 | | | transcriptional | |
| | | gene, promoter | | | activator protein | |
| | | region | | | CdaR | |
| | | | | | [Streptomyces | |
| | | | | | coelicolor] | |
| | | | | | transcriptional | |
| | | | | | regulator | |
| | | | | | [Streptomyces | |
| | | | | | coelicolor] | |
| 65 | D84448 | Cavia cobaya | 1.8 | 4704603 | (AF109916) | 7.1 |
| | | mRNA for | | | putative | |
| | | Na+, K+- | | | dehydrin | |
| | | ATPase beta-3 | | | | |
| | | subunit, | | | | |
| | | complete cds | | | | |
| 66 | AF039948 | Xenopus laevis | 1.8 | 1695839 | (U58151) | 5.6 |
| | | clone H-0 | | | envelope | |
| | | transcription | | | glycoprotein | |
| | | elongation factor | | | [Human | |
| | | S-II (TFIIS) | | | immunodeficien | |
| | | precursor RNA, | | | cy virus type 1] | |
| | | isoform | | | | |
| | | TFIIS.h, partial | | | | |
| | | cds | | | | |
| 67 | M18061 | Xenopus laevis | 1.8 | 780502 | (U18466) AP | 3.1 |
| | | vitelloginin | | | endonuclease | |
| | | gene, complete | | | class II [African | |
| | | cds. | | | swine fever | |
| | | | | | virus] > gi\| | |
| | | | | | 1097525\|prf\|\| | |
| | | | | | 2113434ET | |
| | | | | | AP | |
| | | | | | endonuclease: IS | |
| | | | | | OTYPE = class | |
| | | | | | II [African | |
| | | | | | swine fever | |
| | | | | | virus] | |
| 68 | U61112 | Mus musculus | 1.8 | 3043646 | (AB011133) | 1.9 |
| | | Eya3 homolog | | | KIAA0561 | |
| | | mRNA, | | | protein [Homo | |
| | | complete cds | | | sapiens] | |
| 69 | AB018442 | Oryza sativa | 1.8 | 4455041 | (AF116463) | 0.49 |
| | | mRNA for | | | unknown | |
| | | phytochrome C, | | | [Streptomyces | |
| | | complete cds | | | lincolnensis] | |
| 70 | D63854 | Human | 1.8 | 1169200 | DNA- | 0.22 |
| | | cytomegalovirus | | | DAMAGE- | |
| | | DNA, replication | | | REPAIR/TOLE | |
| | | origin | | | RATION | |
| | | | | | PROTEIN | |
| | | | | | DRT111 | |
| | | | | | PRECURSOR > gi\| | |
| | | | | | 421829\|pir\|\| | |
| | | | | | S33706 | |
| | | | | | DNA-damage | |
| | | | | | resistance | |
| | | | | | protein - | |
| | | | | | Arabidopsis | |
| | | | | | thaliana and | |
| | | | | | DNA-damage | |
| | | | | | resistance | |
| | | | | | protein | |
| | | | | | (DRT111) | |
| | | | | | mRNA, | |
| | | | | | complete cds.], | |
| | | | | | gene product | |
| | | | | | [Arabidopsis | |
| | | | | | thaliana] | |
| 71 | D26549 | Bovine mRNA | 1.8 | 755468 | (U19879) | 0.042 |
| | | for adseverin, | | | transmembrane | |
| | | complete cds | | | protein | |
| | | | | | [Xenopus laevis] | |
| 72 | J05211 | Human | 1.8 | 728867 | ANTER- | 0.015 |
| | | desmoplakin | | | SPECIFIC | |
| | | mRNA, 3′ end. | | | PROLINE- | |
| | | | | | RICH | |
| | | | | | PROTEIN APG | |
| | | | | | PRECURSOR > gi\| | |
| | | | | | 99694\|pir\|\| | |
| | | | | | S21961 | |
| | | | | | proline-rich | |
| | | | | | protein APG - | |
| | | | | | Arabidopsis | |
| | | | | | thaliana > gi\| | |
| | | | | | 22599\|emb\| | |
| | | | | | CAA42925\| | |
| 73 | NM_004415.1 | Homo sapiens | 1.8 | 728867 | ANTER- | 0.015 |
| | | desmoplakin | | | SPECIFIC | |
| | | (DPI, DPII) | | | PROLINE- | |
| | | (DSP) mRNA | | | RICH | |
| | | mRNA, | | | PROTEIN APG | |
| | | complete cds | | | PRECURSOR | |
| | | | | | > gi\|99694\|pir\|\| | |
| | | | | | S21961 | |
| | | | | | proline-rich | |
| | | | | | protein APG - | |
| | | | | | Arabidopsis | |
| | | | | | thaliana > gi\| | |
| | | | | | 22599\|emb\| | |
| | | | | | CAA42925\| | |
| 74 | AF038604 | Caenorhabditis | 1.8 | 3877951 | (Z81555) | 3e−008 |
| | | elegans cosmid | | | predicted using | |
| | | B0546 | | | Genefinder | |
| 75 | AF038604 | Caenorhabditis | 1.8 | 3877951 | (Z81555) | 2e−011 |
| | | elegans cosmid | | | predicted using | |
| | | B0546 | | | Genefinder | |
| 76 | U23551 | Prochlorothrix | 1.8 | 2828280 | (AL021687) | 2e−013 |
| | | hollandica | | | putative protein | |
| | | phosphomannomutase | | | [Arabidopsis | |
| | | | | | thaliana] > gi\| | |
| | | | | | 2832633\|emb\| | |
| | | | | | CAA16762\| | |
| | | | | | (AL021711) | |
| | | | | | putative protein | |
| | | | | | [Arabidopsis | |
| | | | | | thaliana] | |
| 77 | S60150 | ORF1 . . . ORF6 | 1.8 | 1065454 | (U40410) | 2e−019 |
| | | {3′ terminal | | | C54G7.2 gene | |
| | | reigon} | | | product | |
| | | [chrysanthemum | | | [Caenorhabditis | |
| | | virus B CVB, | | | elegans] | |
| | | Genomic RNA, | | | | |
| | | 6 genes, 3426 | | | | |
| | | nt] | | | | |
| 78 | AB014558 | Homo sapiens | 1.8 | 3850072 | (AL033385) | 6e−027 |
| | | mRNA for | | | dna-directed rna | |
| | | KIAA0658 | | | polymerase iii | |
| | | protein, partial | | | subunit | |
| | | cds | | | [Schizosaccharomyces | |
| | | | | | pombe] | |
| 79 | X17191 | E. gracilis | 1.7 | <NONE> | <NONE> | <NONE> |
| | | chloroplast | | | | |
| | | RNA | | | | |
| | | polymerase | | | | |
| | | rpoB-rpoC1- | | | | |
| | | rpoC2 operon | | | | |
| 80 | X07729 | R. norvegicus | 1.7 | 4584544 | (AL049608) | 8.8 |
| | | gene encoding | | | extensin-like | |
| | | neuron-specific | | | protein | |
| | | enolase, exons | | | | |
| | | 8-12 | | | | |
| 81 | D38178 | Human gene for | 1.7 | 73714 | infected cell | 1.1 |
| | | cytosolic | | | protein ICP34.5 - | |
| | | phospholipase | | | human | |
| | | A2, exon 1 | | | herpesvirus 1 | |
| | | | | | (strain F) > gi\| | |
| | | | | | 330123 | |
| | | | | | (M12240) | |
| | | | | | infected cell | |
| | | | | | protein [Herpes | |
| | | | | | simplex virus | |
| | | | | | type 1] | |
| 82 | U23551 | Prochlorothrix | 1.7 | 2828280 | (AL021687) | 2e−010 |
| | | hollandica | | | putative protein | |
| | | phosphomannomutase | | | [Arabidopsis | |
| | | | | | thaliana] > gi\| | |
| | | | | | 2832633\|emb\| | |
| | | | | | CAA16762\| | |
| | | | | | (AL021711) | |
| | | | | | putative protein | |
| | | | | | [Arabidopsis | |
| | | | | | thaliana] | |
| 83 | Y00525 | Klebsiella | 1.6 | 3800951 | (AF100657) No | 6e−013 |
| | | pneumoniae | | | definition line | |
| | | nifL gene for | | | found | |
| | | regulatory | | | [Caenorhabditis | |
| | | protein | | | elegans] | |
| 84 | AF100170.1 | Bos taurus | 1.5 | 463552 | (U05877) AF-1 | 0.074 |
| | | major fibrous | | | [Homo sapiens] | |
| | | sheath protein | | | | |
| | | precursor, | | | | |
| | | mRNA, | | | | |
| | | complete cds | | | | |
| 85 | Y13441 | Homo sapiens | 0.74 | <NONE> | <NONE> | <NONE> |
| | | Rox gene, exon 2 | | | | |
| 86 | L46792 | Actinidia | 0.73 | 3170252 | (AF043636) | 0.001 |
| | | deliciosa clone | | | circumsporozoite | |
| | | AdXET-5 | | | protein | |
| | | xyloglucan | | | [Plasmodium | |
| | | endotransglycos | | | chabaudi] | |
| | | ylase precursor | | | | |
| | | (XET) mRNA, | | | | |
| | | complete cds | | | | |
| 87 | U73489 | Drosophila | 0.7 | 3915994 | HYPOTHETIC | 3e−005 |
| | | melanogaster | | | AL 53.2 KD | |
| | | Nem (nem) | | | PROTEIN IN | |
| | | mRNA, | | | PRC-PRPA | |
| | | complete cds | | | INTERGENIC | |
| | | | | | REGION | |
| 88 | U95097 | Xenopus laevis | 0.68 | 157272 | (L11345) DNA- | 8.5 |
| | | mitotic | | | binding protein | |
| | | phosphoprotein | | | [Drosophila | |
| | | 43 mRNA, | | | melanogaster] | |
| | | partial cds | | | | |
| 89 | AF082012 | Caenorhabditis | 0.67 | 2494313 | PUTATIVE | 8.4 |
| | | elegans UDP-N- | | | TRANSLATION | |
| | | acetylglucosamine: | | | INITIATION | |
| | | a-3-D- | | | FACTOR EIF- | |
| | | mannoside b- | | | 2B SUBUNIT 1 | |
| | | 1,2-N- | | | (EIF-2B GDP- | |
| | | acetylglucosaminyltransferase I | | | GTP | |
| | | (gly-14) mRNA, | | | EXCHANGE | |
| | | complete cds | | | FACTOR) eIF- | |
| | | | | | 2B, subunit | |
| | | | | | alpha - | |
| | | | | | Methanococcus | |
| | | | | | jannaschii aIF- | |
| | | | | | 2B, subunit | |
| | | | | | delta (aIF2BD) | |
| | | | | | [Methanococcus | |
| | | | | | jannaschii] | |
| 90 | U04354 | Mus musculus | 0.67 | 4755188 | (AC007018) | 8e−026 |
| | | ADSEVERIN | | | unknown protein | |
| | | mRNA, | | | | |
| | | complete cds | | | | |
| 91 | M68881 | S. pombe cigl + gene, | 0.67 | 2078441 | (U56964) weak | 2e−030 |
| | | complete | | | similarity to S. | |
| | | cds. | | | cerevisiae | |
| | | | | | intracellular | |
| | | | | | protein transport | |
| | | | | | protein US)1 | |
| | | | | | (SP: P25386) | |
| 92 | U95097 | Xenopus laevis | 0.66 | 2829685 | PROTEIN- | 6.2 |
| | | mitotic | | | TYROSINE | |
| | | phosphoprotein | | | PHOSPHATASE X | |
| | | 43 mRNA, | | | PRECURSOR | |
| | | partial cds | | | (R-PTP-X) | |
| | | | | | (PTP IA- | |
| | | | | | 2BETA) | |
| | | | | | (PROTEIN | |
| | | | | | TYROSINE | |
| | | | | | PHOSPHATASE-NP) | |
| | | | | | (PTP- | |
| | | | | | NP) > gi\| | |
| | | | | | 1515425 | |
| | | | | | (U57345) | |
| | | | | | protein tyrosine | |
| | | | | | phosphatase-NP | |
| | | | | | [Mus musculus] | |
| 93 | Z15056 | B. subtilis genes | 0.66 | 477124 | P3A2 DNA | 2.1 |
| | | spoVD, murE, | | | binding protein | |
| | | mra Y, murD | | | homolog EWG - | |
| | | | | | fruit fly | |
| | | | | | (Drosophila | |
| | | | | | melanogaster) | |
| 94 | M86808 | Human pyruvate | 0.65 | <NONE> | <NONE> | <NONE> |
| | | dehydrogenase | | | | |
| | | complex | | | | |
| | | (PDHA2) gene, | | | | |
| | | complete cds. | | | | |
| 95 | J03754 | Rat plasma | 0.65 | 4507549 | transmembrane | 8e−006 |
| | | membrane | | | protein with | |
| | | Ca2+ ATPase- | | | EGF-like and | |
| | | isoform 2 | | | two follistatin- | |
| | | mRNA, | | | like domains 1 > gi\| | |
| | | complete cds. | | | 755466 | |
| 96 | NM_000887.1 | Homo sapiens | 0.64 | <NONE> | <NONE> | <NONE> |
| | | integrin, alpha | | | | |
| | | X (antigen | | | | |
| | | CD11C | | | | |
| | | emb\|Y00093\|H | | | | |
| | | SP15095 | | | | |
| | | H. sapiens | | | | |
| | | mRNA for | | | | |
| | | leukocyte | | | | |
| | | adhesion | | | | |
| | | glycoprotein | | | | |
| | | p150,95 | | | | |
| 97 | L27080 | Human | 0.64 | <NONE> | <NONE> | <NONE> |
| | | melanocortin 5 | | | | |
| | | receptor | | | | |
| | | (MC5R) gene, | | | | |
| | | complete cds. | | | | |
| 98 | U07890 | Mus musculus | 0.64 | <NONE> | <NONE> | <NONE> |
| | | C57BL/6J | | | | |
| | | epidermal | | | | |
| | | surface antigen | | | | |
| | | (mesa) mRNA, | | | | |
| | | complete cds. | | | | |
| 99 | AF079139 | Streptomyces | 0.64 | 3041869 | (U96109) | 2.8 |
| | | venezuelae | | | proline-rich | |
| | | pikCD operon, | | | transcription | |
| | | complete | | | factor ALX3 | |
| | | sequence | | | [Mus musculus] | |
| 100 | M16140 | Chicken | 0.64 | 123984 | ACROSIN | 4e−008 |
| | | ovoinhibitor | | | INHIBITORS | |
| | | gene, exon 15. | | | IIA AND IIB | |
| 101 | NM_000887.1 | Homo sapiens | 0.63 | <NONE> | <NONE> | <NONE> |
| | | integrin, alpha | | | | |
| | | X (antigen | | | | |
| | | CD11C | | | | |
| | | emb\|Y00093\|H | | | | |
| | | SP15095 | | | | |
| | | H. sapiens | | | | |
| | | mRNA for | | | | |
| | | leukocyte | | | | |
| | | adhesion | | | | |
| | | glycoprotein | | | | |
| | | p150,95 | | | | |
| 102 | Z17316 | Kluyveromyces | 0.63 | <NONE> | <NONE> | <NONE> |
| | | lactis for gene | | | | |
| | | encoding | | | | |
| | | phosphofructoki | | | | |
| | | nase beta | | | | |
| | | subunit | | | | |
| 103 | Z25470 | H. sapiens | 0.63 | <NONE> | <NONE> | <NONE> |
| | | melanocortin 5 | | | | |
| | | receptor gene, | | | | |
| | | complete CDS | | | | |
| 104 | L19954 | Bacillus subtilis | 0.63 | <NONE> | <NONE> | <NONE> |
| | | feuA, B, and C | | | | |
| | | genes, 3 ORFs, | | | | |
| | | 2 complete cds's | | | | |
| | | and 5′ end. | | | | |
| 105 | U44405 | Spiroplasma | 0.63 | 2499642 | SERINE/THREONINE- | 7.7 |
| | | citri | | | PROTEIN | |
| | | chromosome | | | KINASE STE20 | |
| | | pre-inversion | | | HOMOLOG > gi\| | |
| | | border, SPV1- | | | 1737181 | |
| | | like sequences, | | | (U73457) | |
| | | transposase | | | Cst20p [Candida | |
| | | gene, partial | | | albicans] | |
| | | cds, adhesin-like | | | | |
| | | protein P58 | | | | |
| | | gene, complete | | | | |
| | | cds. | | | | |
| 106 | Z28264 | S. cerevisiae | 0.63 | 3880930 | (AL021481) | 2e−014 |
| | | chromosome XI | | | similar to | |
| | | reading frame | | | Phosphoglucomutase | |
| | | ORF YKR039w | | | and | |
| | | | | | phosphomannomutase | |
| | | | | | phosphoserine; | |
| | | | | | cDNA EST | |
| | | | | | EMBL: D36168 | |
| | | | | | comes from this | |
| | | | | | gene; cDNA | |
| | | | | | EST | |
| | | | | | EMBL: D70697 | |
| | | | | | comes from this | |
| | | | | | gene; cDNA | |
| | | | | | EST yk373h9.5 | |
| | | | | | comes from this | |
| | | | | | gene; cDNA | |
| | | | | | EST | |
| | | | | | EMBL: T00805 | |
| | | | | | . . . | |
| 107 | AE001107 | Archaeoglobus | 0.62 | <NONE> | <NONE> | <NONE> |
| | | fulgidus section | | | | |
| | | 172 of 172 of | | | | |
| | | the complete | | | | |
| | | genome | | | | |
| 108 | Z14112 | B. firmus TopA | 0.62 | 310115 | (L02530) | 0.026 |
| | | gene encoding | | | Drosophila | |
| | | DNA | | | polarity gene | |
| | | topoisomerase I | | | (frizzled) | |
| | | | | | homologue | |
| 109 | AF118101 | Toxoplasma | 0.62 | 726403 | (U23175) | 4e−018 |
| | | gondii protein | | | similar to anion | |
| | | kinase 6 (tpk6) | | | exchange | |
| | | mRNA, | | | protein | |
| | | complete cds | | | [Caenorhabditis | |
| | | | | | elegans] | |
| 110 | M59743 | Rabbit cardiac | 0.61 | <NONE> | <NONE> | <NONE> |
| | | muscle Ca-2 + release | | | | |
| | | channel | | | | |
| 111 | M12036 | Human tyrosine | 0.61 | 61962 | (X58484) gag | 7.5 |
| | | kinase-type | | | [Simian foamy | |
| | | receptor (HER2) | | | virus] | |
| | | gene, partial | | | | |
| | | cds. | | | | |
| 112 | AF043195 | Homo sapiens | 0.61 | 1572629 | (U69699) | 7.5 |
| | | tight junction | | | unknown protein | |
| | | protein ZO (ZO- | | | precursor [Mus | |
| | | 2) gene, | | | musculus] | |
| | | alternative splice | | | | |
| | | products, | | | | |
| | | promoter and | | | | |
| | | exon A | | | | |
| 113 | U18178 | Human HLA | 0.61 | 1336688 | (S81116) | 5.7 |
| | | class I genomic | | | properdin | |
| | | survey | | | [guinea pigs, | |
| | | sequence. | | | spleen, Peptide, | |
| | | | | | 470 aa] [Cavia] | |
| 114 | U44405 | Spiroplasma | 0.61 | 2827531 | (AL021633) | 3.3 |
| | | citri | | | hypothetical | |
| | | chromosome | | | protein | |
| | | pre-inversion | | | | |
| | | border, SPV1- | | | | |
| | | like sequences, | | | | |
| | | transposase | | | | |
| | | gene, partial | | | | |
| | | cds, adhesin-like | | | | |
| | | protein P58 | | | | |
| | | gene, complete | | | | |
| | | cds. | | | | |
| 115 | Z33011 | M. capricolum | 0.61 | 3915729 | HYPERPLASTIC | 0.26 |
| | | DNA for | | | DISCS | |
| | | CONTIG | | | PROTEIN | |
| | | MC008 | | | (HYD | |
| | | | | | PROTEIN) > gi\| | |
| | | | | | 2673887 | |
| | | | | | (L14644) | |
| | | | | | hyperplastic | |
| | | | | | discs protein | |
| 116 | NM_001429.1 | Homo sapiens | 0.61 | 4204294 | (AC003027) | 5e−005 |
| | | E1A binding | | | lcl\|prt_seq No | |
| | | protein p300 | | | definition line | |
| | | mRNA, | | | found | |
| | | complete cds. > :: | | | | |
| | | gb\|I62297\|I622 | | | | |
| | | 97 Sequence 1 | | | | |
| | | from patent US | | | | |
| | | 5658784 | | | | |
| 117 | Z25418 | C. familiaris | 0.61 | 3877493 | (Z48583) | 1e−007 |
| | | MHC class Ib | | | similar to | |
| | | gene (DLA-79) | | | ATPases | |
| | | gene, complete | | | associated with | |
| | | CDS | | | various cellular | |
| | | | | | activities | |
| | | | | | (AAA); cDNA | |
| | | | | | EST | |
| | | | | | EMBL: Z14623 | |
| | | | | | comes from this | |
| | | | | | gene; cDNA | |
| | | | | | EST | |
| | | | | | EMBL: D75090 | |
| | | | | | comes from this | |
| | | | | | gene; cDNA | |
| | | | | | EST | |
| | | | | | EMBL: D72255 | |
| | | | | | comes from this | |
| | | | | | gene; cDNA | |
| | | | | | EST yk200e4.5 | |
| | | | | | . . . | |
| 118 | AB002150 | Bacillus subtilis | 0.6 | <NONE> | <NONE> | <NONE> |
| | | DNA for FeuB, | | | | |
| | | FeuA, YbbB, | | | | |
| | | YbbC, YbbD, | | | | |
| | | YbzA, YbbE, | | | | |
| | | YbbF, YbbH, | | | | |
| | | YbbI, YbbJ, | | | | |
| | | YbbK, YbbL, | | | | |
| | | YbbM, YbbP, | | | | |
| | | complete cds | | | | |
| 119 | Y07786 | V. cholerae | 0.6 | <NONE> | <NONE> | <NONE> |
| | | ORF's involved | | | | |
| | | in | | | | |
| | | lipopolysaccharide | | | | |
| | | synthese | | | | |
| 120 | Z17316 | Kluyveromyces | 0.6 | <NONE> | <NONE> | <NONE> |
| | | lactis for gene | | | | |
| | | encoding | | | | |
| | | phosphofructokinase | | | | |
| | | beta | | | | |
| | | subunit | | | | |
| 121 | Z71403 | S. cerevisiae | 0.6 | <NONE> | <NONE> | <NONE> |
| | | chromosome | | | | |
| | | XIV reading | | | | |
| | | frame ORF | | | | |
| | | YNL127w | | | | |
| 122 | L34641 | Homo sapiens | 0.6 | 1147634 | (U42213) | 9.6 |
| | | platelet/endothelial | | | micronemal | |
| | | cell adhesion | | | TRAP-C1 | |
| | | molecule-1 | | | protein homolog | |
| | | (PECAM-1) | | | | |
| | | gene, exon 10. | | | | |
| 123 | AF070572 | Homo sapiens | 0.6 | 399034 | N- | 2.5 |
| | | clone 24778 | | | ACETYLMUR | |
| | | unknown | | | AMOYL-L- | |
| | | mRNA | | | ALANINE | |
| | | | | | AMIDASE | |
| | | | | | AMIB | |
| | | | | | PRECURSOR > gi\| | |
| | | | | | 628763\|pir\|\| | |
| | | | | | S41741 N- | |
| | | | | | acetylmuramoyl- | |
| | | | | | L-alanine | |
| | | | | | amidase (EC | |
| | | | | | 3.5.1.28) - | |
| | | | | | Escherichia coli > gi\| | |
| | | | | | 304914 | |
| | | | | | (L19346) N- | |
| | | | | | acetylmuramoyl- | |
| | | | | | L-alanine | |
| | | | | | amidase | |
| | | | | | [Escherichia | |
| | | | | | coli] N- | |
| | | | | | acetylmuramoyl- | |
| | | | | | l-alanine | |
| | | | | | amidase II; a | |
| 124 | X75627 | C. burnetii trxB, | 0.6 | 3036833 | (AJ003163) | 0.28 |
| | | spoIIIE and serS | | | apsB | |
| | | genes | | | [Emericella | |
| | | | | | nidulans] | |
| 125 | Z99765 | Flaveria pringlei | 0.59 | <NONE> | <NONE> | <NONE> |
| | | gdcsH gene | | | | |
| 126 | U02538 | Mycoplasma | 0.59 | <NONE> | <NONE> | <NONE> |
| | | hyopneumoniae | | | | |
| | | J ATCC 25934 | | | | |
| | | 23S rRNA gene, | | | | |
| | | partial sequence | | | | |
| 127 | Z71403 | S. cerevisiae | 0.59 | <NONE> | <NONE> | <NONE> |
| | | chromosome | | | | |
| | | XIV reading | | | | |
| | | frame ORF | | | | |
| | | YNL127w | | | | |
| 128 | X03942 | Mouse simple | 0.59 | <NONE> | <NONE> | <NONE> |
| | | repetitive DNA | | | | |
| | | (sqr family) | | | | |
| | | transcript (clone | | | | |
| | | pmlc 2) with | | | | |
| | | conserved | | | | |
| | | GACA/GATA | | | | |
| | | repeats | | | | |
| 129 | U11844 | Mus musculus | 0.59 | <NONE> | <NONE> | <NONE> |
| | | glucose | | | | |
| | | transporter | | | | |
| | | (GLUT3) gene, | | | | |
| | | exon 1 | | | | |
| 130 | D63395 | Homo sapiens | 0.59 | 4433616 | (AF107018) | 1.8 |
| | | mRNA for | | | alpha- | |
| | | NOTCH4, | | | mannosidase IIx | |
| | | partial cds | | | [Mus musculus] | |
| 131 | Z33011 | M. capricolum | 0.59 | 3915729 | HYPERPLASTIC | 0.27 |
| | | DNA for | | | DISCS | |
| | | CONTIG | | | PROTEIN | |
| | | MC008 | | | (HYD | |
| | | | | | PROTEIN) > gi\| | |
| | | | | | 2673887 | |
| | | | | | (L14644) | |
| | | | | | hyperplastic | |
| | | | | | discs protein | |
| 132 | U05670 | Haemophilus | 0.58 | <NONE> | <NONE> | <NONE> |
| | | influenzae DL42 | | | | |
| | | Lex2A and | | | | |
| | | Lex2B genes, | | | | |
| | | complete cds. | | | | |
| 133 | L27080 | Human | 0.58 | 123984 | ACROSIN | 2e−006 |
| | | melanocortin 5 | | | INHIBITORS | |
| | | receptor | | | IIA AND IIB | |
| | | (MC5R) gene, | | | | |
| | | complete cds. | | | | |
| 134 | AF043195 | Homo sapiens | 0.57 | 1572629 | (U69699) | 6.7 |
| | | tight junction | | | unknown protein | |
| | | protein ZO (ZO- | | | precursor [Mus | |
| | | 2) gene, | | | musculus] | |
| | | alternative splice | | | | |
| | | products, | | | | |
| | | promoter and | | | | |
| | | exon A | | | | |
| 135 | U57707 | Bos taurus | 0.57 | 807646 | (M17294) | 0.068 |
| | | activin receptor | | | unknown protein | |
| | | type IIB | | | [Human | |
| | | precursor | | | herpesvirus 4] | |
| 136 | Z17316 | Kluyveromyces | 0.56 | <NONE> | <NONE> | <NONE> |
| | | lactis for gene | | | | |
| | | encoding | | | | |
| | | phosphofructokinase | | | | |
| | | beta | | | | |
| | | subunit | | | | |
| 137 | M21535 | Human erg | 0.56 | <NONE> | <NONE> | <NONE> |
| | | protein (ets- | | | | |
| | | related gene) | | | | |
| | | mRNA, | | | | |
| | | complete cds. | | | | |
| 138 | M64932 | Candida maltosa | 0.56 | 3219524 | (AF069428) | 1.3 |
| | | cyclohexamide | | | NADH | |
| | | resistance | | | dehydrogenase | |
| | | protein | | | subunit IV | |
| | | | | | [Alligator | |
| | | | | | mississippiensis] > gi\| | |
| | | | | | 3367630\|emb\| | |
| | | | | | CAA73570\| | |
| | | | | | (Y13113) | |
| | | | | | NADH | |
| | | | | | dehydrogenase | |
| | | | | | subunit 4 | |
| | | | | | [Alligator | |
| | | | | | mississippiensis] | |
| 139 | AE000342 | Escherichia coli | 0.56 | 3874685 | (Z78539) | 0.088 |
| | | K-12 MG1655 | | | Similarity to | |
| | | section 232 of | | | S. pombe | |
| | | 400 of the | | | hypothetical | |
| | | complete | | | protein | |
| | | genome | | | C4G8.04 | |
| | | | | | (SW: YAD4_SC | |
| | | | | | HPO); cDNA | |
| | | | | | EST | |
| | | | | | EMBL: D27846 | |
| | | | | | comes from this | |
| | | | | | gene; cDNA | |
| | | | | | EST | |
| | | | | | EMBL: D27845 | |
| | | | | | comes from this | |
| | | | | | gene; cDNA | |
| | | | | | EST yk202h7.3 | |
| | | | | | comes from this | |
| | | | | | gene; cDNA | |
| | | | | | EST yk202h7.5 | |
| | | | | | come . . . | |
| 140 | Z15056 | B. subtilis genes | 0.55 | 477124 | P3A2 DNA | 3.7 |
| | | spoVD, murE, | | | binding protein | |
| | | mraY, murD | | | homolog EWG - | |
| | | | | | fruit fly | |
| | | | | | (Drosophila | |
| | | | | | melanogaster) | |
| 141 | Z58167 | H. sapiens CpG | 0.53 | <NONE> | <NONE> | <NONE> |
| | | island DNA | | | | |
| | | genomic Mse1 | | | | |
| | | fragment, clone | | | | |
| | | 30e10, forward | | | | |
| | | read | | | | |
| | | cpg30e10.ft1b | | | | |
| 142 | M27159 | Rat potassium | 0.53 | 1850920 | (U21247) Bet | 0.9 |
| | | channel-Kv2 | | | [Human | |
| | | gene, partial | | | spumaretrovirus] | |
| | | cds. | | | | |
| 143 | M15555 | Mouse Ig | 0.24 | <NONE> | <NONE> | <NONE> |
| | | germline V- | | | | |
| | | kappa-24 chain | | | | |
| | | (VK24C) gene, | | | | |
| | | exons 1 and 2. | | | | |
| 144 | U95097 | Xenopus laevis | 0.24 | 399109 | TRANSCRIPTION | 4 |
| | | mitotic | | | FACTOR | |
| | | phosphoprotein | | | BF-1 (BRAIN | |
| | | 43 mRNA, | | | FACTOR 1) | |
| | | partial cds | | | (BF1) > gi\| | |
| | | | | | 92020\|pir\|\| | |
| | | | | | JH0672 brain | |
| | | | | | factor 1 protein - | |
| | | | | | rat > gi\| | |
| | | | | | 203135 | |
| | | | | | (M87634) BF-1 | |
| | | | | | [Rattus | |
| | | | | | norvegicus] | |
| 145 | AJ002014 | Crythecodinium | 0.24 | 416704 | BALBIANI | 0.36 |
| | | cohnii mRNA | | | RING | |
| | | for nuclear | | | PROTEIN 3 | |
| | | protein JUS1 | | | PRECURSOR | |
| | | | | | balbiani ring 3 | |
| | | | | | (BR3) | |
| | | | | | [Chironomus | |
| | | | | | tentans] | |
| 146 | L35330 | Rattus | 0.23 | 1388158 | (U58204) | 8.8 |
| | | norvegicus | | | myomesin | |
| | | glutathione S- | | | [Gallus gallus] | |
| | | transferase Yb3 | | | | |
| | | subunit gene, | | | | |
| | | complete cds. | | | | |
| 147 | NM_001432.1 | Homo sapiens | 0.23 | 2851520 | TRANSFORMING | 2e−008 |
| | | epiregulin | | | GROWTH | |
| | | (EREG) mRNA > :: | | | FACTOR | |
| | | dbj\|D30783\|D30783 | | | ALPHA | |
| | | Homo | | | PRECURSOR | |
| | | sapiens mRNA | | | (TGF-ALPHA) | |
| | | for epiregulin, | | | (EGF-LIKE | |
| | | complete cds | | | TGF) (ETGF) | |
| | | | | | (TGF TYPE 1) | |
| | | | | | precursor - rat > gi\| | |
| | | | | | 207282 | |
| | | | | | (M31076) | |
| | | | | | transforming | |
| | | | | | growth factor | |
| | | | | | alpha precursor | |
| | | | | | [Rattus | |
| | | | | | norvegicus] | |
| 148 | U57043 | Cebus apella | 0.22 | <NONE> | <NONE> | <NONE> |
| | | gamma globin | | | | |
| | | (gamma1) gene, | | | | |
| | | complete cds | | | | |
| 149 | AB023188.1 | Homo sapiens | 0.22 | <NONE> | <NONE> | <NONE> |
| | | mRNA for | | | | |
| | | KIAA0971 | | | | |
| | | protein, | | | | |
| | | complete cds | | | | |
| 150 | M18105 | Yeast | 0.22 | <NONE> | <NONE> | <NONE> |
| | | (S. cerevisiae) | | | | |
| | | SST2 gene | | | | |
| | | encoding | | | | |
| | | desensitization | | | | |
| | | to alpha-factor | | | | |
| | | pheromone, | | | | |
| | | complete cds. | | | | |
| 151 | AJ001113 | Homo sapiens | 0.22 | 3122961 | ENHANCER | 8.5 |
| | | UBE3A gene, | | | OF SPLIT | |
| | | exon 16 | | | GROUCHO- | |
| | | | | | LIKE PROTEIN | |
| | | | | | 1 > gi\|2408145 | |
| | | | | | (U18775) | |
| | | | | | enhancer of split | |
| | | | | | groucho | |
| 152 | L35330 | Rattus | 0.22 | 1388158 | (U58204) | 8.1 |
| | | norvegicus | | | myomesin | |
| | | glutathione S- | | | [Gallus gallus] | |
| | | transferase Yb3 | | | | |
| | | subunit gene, | | | | |
| | | complete cds. | | | | |
| 153 | D42042 | Human mRNA | 0.22 | 4827063 | zinc finger | 6.1 |
| | | for KIAA0085 | | | protein 142 | |
| | | gene, partial cds | | | (clone pHZ-49) > gi\| | |
| | | | | | 3123312\|sp\| | |
| | | | | | P52746\|Z142_{—} | |
| | | | | | HUMAN | |
| | | | | | ZINC FINGER | |
| | | | | | PROTEIN 142 | |
| | | | | | (KIAA0236) | |
| | | | | | (HA4654) > gi\| | |
| | | | | | 1510147\|dbj\| | |
| | | | | | BAA13242\| | |
| 154 | L35330 | Rattus | 0.22 | 2853301 | (AF007194) | 1.6 |
| | | norvegicus | | | mucin [Homo | |
| | | glutathione S- | | | sapiens] | |
| | | transferase Yb3 | | | | |
| | | subunit gene, | | | | |
| | | complete cds. | | | | |
| 155 | Z11653 | H. sapiens DBH | 0.22 | 3819705 | (AL032824) | 1.2 |
| | | gene complex | | | syntaxin binding | |
| | | repeat | | | protein 1; sec1 | |
| | | polymorphism | | | family secretory | |
| | | DNA | | | protein | |
| | | | | | [Schizosaccharomyces | |
| | | | | | pombe] | |
| 156 | L29063 | Candida | 0.22 | 3046871 | (AB003753) | 0.32 |
| | | albicans fatty | | | high sulfur | |
| | | acid synthase | | | protein B2E | |
| | | alpha subunit | | | [Rattus | |
| | | (FAS2) gene, | | | norvegicus] | |
| | | complete cds. | | | | |
| 157 | M64865 | Horse alcohol | 0.22 | 2213909 | (AF004874) | 0.037 |
| | | dehydrogenase- | | | latent TGF-beta | |
| | | S-isoenzyme | | | binding protein- | |
| | | mRNA, | | | 2 [Mus | |
| | | complete cds. | | | musculus] | |
| 158 | Y09472 | B. taurus gene | 0.21 | 2909874 | (AF047829) | 7.6 |
| | | encoding | | | melatonin- | |
| | | preprododecapeptide | | | related receptor | |
| | | | | | [Ovis aries] | |
| 159 | Y09472 | B. taurus gene | 0.21 | 2909874 | (AF047829) | 7.5 |
| | | encoding | | | melatonin- | |
| | | preprododecapeptide | | | related receptor | |
| | | | | | [Ovis aries] | |
| 160 | X80301 | N. tabacum axi 1 | 0.21 | 2832715 | (AJ003066) | 6 |
| | | gene | | | subunit beta of | |
| | | | | | the | |
| | | | | | mitochondrial | |
| | | | | | fatty acid beta- | |
| | | | | | oxydation | |
| | | | | | multienzyme | |
| | | | | | complex [Bos | |
| | | | | | taurus] | |
| 161 | AF073485 | Homo sapiens | 0.21 | 2224559 | (AB002307) | 3.3 |
| | | MHC class I- | | | KIAA0309 | |
| | | related protein | | | [Homo sapiens] | |
| | | MR1 precursor | | | | |
| | | (MR1) gene, | | | | |
| | | partial cds | | | | |
| 162 | S78251 | growth hormone | 0.21 | 729381 | DYNAMIN-1 | 2 |
| | | receptor | | | (DYNAMIN | |
| | | {alternatively | | | BREDNM19) | |
| | | spliced, exon | | | | |
| | | 1B} [sheep, | | | | |
| | | Merino, skeletal | | | | |
| | | muscle, mRNA | | | | |
| | | Partial, 438 nt] | | | | |
| 163 | U16135 | Synechococcus | 0.21 | 135514 | T-CELL | 0.02 |
| | | sp. Clp protease | | | RECEPTOR | |
| | | proteolytic | | | BETA CHAIN | |
| | | subunit | | | PRECURSOR | |
| | | | | | precursor (ANA | |
| | | | | | 11) - rabbit | |
| 164 | X95601 | M. hominis lmp3 | 0.21 | 2995445 | (Y10496) CDV- | 0.005 |
| | | and lmp4 genes | | | 1 protein [Mus | |
| | | | | | musculus] | |
| 165 | X95601 | M. hominis lmp3 | 0.21 | 2995447 | (Y10495) CDV- | 0.005 |
| | | and lmp4 genes | | | 1R protein [Mus | |
| | | | | | musculus] | |
| 166 | AF124249.1 | Homo sapiens | 0.21 | 423456 | epidermal | 8e−010 |
| | | SH2-containing | | | growth factor- | |
| | | protein Nsp1 | | | receptor-binding | |
| | | mRNA, | | | protein GRB-4 - | |
| | | complete cds | | | mouse | |
| | | | | | (fragment) | |
| 167 | AF030282 | Danio rerio | 0.21 | 3928083 | (AC005770) | 2e−014 |
| | | homeobox | | | unknown protein | |
| | | protein Six7 | | | [Arabidopsis | |
| | | (six7) mRNA, | | | thaliana] | |
| | | complete cds | | | | |
| 168 | X83427 | O. anatinus | 0.21 | 132575 | RIBONUCLEASE | 3e−021 |
| | | mitochondrial | | | INHIBITOR | |
| | | DNA, complete | | | | |
| | | genome | | | | |
| 169 | AJ001113 | Homo sapiens | 0.2 | <NONE> | <NONE> | <NONE> |
| | | UBE3A gene, | | | | |
| | | exon 16 | | | | |
| 170 | AF081533.1 | Anopheles | 0.2 | <NONE> | <NONE> | <NONE> |
| | | gambiae | | | | |
| | | putative gram | | | | |
| | | negative bacteria | | | | |
| | | binding protein | | | | |
| | | gene, complete | | | | |
| | | cds | | | | |
| 171 | U70316 | Dictyostelium | 0.2 | <NONE> | <NONE> | <NONE> |
| | | discoideum | | | | |
| | | IonA (iona) | | | | |
| | | gene, partial cds | | | | |
| 172 | AF009341 | Homo sapiens | 0.2 | <NONE> | <NONE> | <NONE> |
| | | E6-AP | | | | |
| | | ubiquitin-protein | | | | |
| | | ligase | | | | |
| 173 | L35330 | Rattus | 0.2 | 3702275 | (AC005793) | 2.5 |
| | | norvegicus | | | KIAA0561 | |
| | | glutathione S- | | | protein [AA 1-593] | |
| | | transferase Yb3 | | | [Homo | |
| | | subunit gene, | | | sapiens] | |
| | | complete cds. | | | | |
| 174 | AE000573.1 | Helicobacter | 0.2 | 3947855 | (AL034381) | 2.5 |
| | | pylori 26695 | | | putative Golgi | |
| | | section 51 of | | | membrane | |
| | | 134 of the | | | protein | |
| | | complete | | | | |
| | | genome | | | | |
| 175 | X83230 | G. gallus | 0.2 | 3258596 | (U95821) | 0.81 |
| | | hsp90beta gene | | | putative | |
| | | | | | transmembrane | |
| | | | | | GTPase | |
| | | | | | [Drosophila | |
| | | | | | melanogaster] | |
| 176 | X57157 | Chicken mRNA | 0.2 | 108325 | insulin-like | 0.17 |
| | | for Hsp47, heat | | | growth factor- | |
| | | shock protein 47 | | | binding protein 6 | |
| 177 | M58748 | Chicken alpha- | 0.2 | 1086863 | (U41272) | 4e−005 |
| | | globin gene | | | T03G11.6 gene | |
| | | domain with | | | product | |
| | | structural matrix | | | [Caenorhabditis | |
| | | attachment sites. | | | elegans] | |
| 178 | AB016815 | Anthocidaris | 0.2 | 423456 | epidermal | 1e−012 |
| | | crassispina | | | growth factor- | |
| | | mRNA for Src- | | | receptor-binding | |
| | | type protein | | | protein GRB-4 - | |
| | | tyrosine kinase, | | | mouse | |
| | | complete cds | | | (fragment) | |
| 179 | AF030282 | Danio rerio | 0.2 | 3928083 | (AC005770) | 3e−014 |
| | | homeobox | | | unknown protein | |
| | | protein Six7 | | | [Arabidopsis | |
| | | (six7) mRNA, | | | thaliana] | |
| | | complete cds | | | | |
| 180 | AL035559 | Streptomyces | 0.2 | 2088714 | (AF003139) | 3e−022 |
| | | coelicolor | | | strong similarity | |
| | | cosmid 9F2 | | | to NADPH | |
| | | | | | oxidases; partial | |
| | | | | | CDS, the gene | |
| | | | | | begins in the | |
| | | | | | neighboring | |
| | | | | | clone | |
| 181 | S79641 | SDH = succinate | 0.2 | 4755188 | (AC007018) | 2e−022 |
| | | dehydrogenase | | | unknown protein | |
| | | flavoprotein | | | | |
| | | subunit Mutant, | | | | |
| | | 387 nt] | | | | |
| 182 | X75383 | H. sapiens | 0.19 | <NONE> | <NONE> | <NONE> |
| | | mRNA for | | | | |
| | | TFIIA-alpha | | | | |
| 183 | U53901 | Hippopotamus | 0.19 | <NONE> | <NONE> | <NONE> |
| | | amphibius b- | | | | |
| | | casein gene, | | | | |
| | | exon 7, partial | | | | |
| | | cds | | | | |
| 184 | J05265 | Mouse | 0.19 | 77356 | hypothetical | 0.0005 |
| | | interferon | | | 70K protein - | |
| | | gamma receptor | | | eggplant mosaic | |
| | | mRNA, | | | virus | |
| | | complete cds. | | | | |
| 185 | U72353 | Rattus | 0.19 | 3880857 | (AL031633) | 2e−006 |
| | | norvegicus | | | cDNA EST | |
| | | lamin B1 | | | yk404d1.5 | |
| | | mRNA, | | | comes from this | |
| | | complete cds | | | gene; cDNA | |
| | | | | | EST yk404d1.3 | |
| | | | | | comes from this | |
| | | | | | gene | |
| 186 | AB016815 | Anthocidaris | 0.19 | 3930217 | (AF047487) | 2e−007 |
| | | crassispina | | | Nck-2 [Homo | |
| | | mRNA for Src- | | | sapiens] | |
| | | type protein | | | | |
| | | tyrosine kinase, | | | | |
| | | complete cds | | | | |
| 187 | D10911 | Mus musculus | 0.19 | 2662366 | (D86332) | 5e−011 |
| | | DNA for MS2 | | | membrane type- | |
| | | protein, | | | 2 matrix | |
| | | complete cds | | | metalloproteinase | |
| | | | | | [Mus | |
| | | | | | musculus] | |
| 188 | AB015345 | Homo sapiens | 0.075 | 3877417 | (Z66564) | 6.4 |
| | | HRIHFB2216 | | | similar to anion | |
| | | mRNA, partial | | | exchange | |
| | | cds | | | protein | |
| 189 | AF086410 | Homo sapiens | 0.075 | 3023371 | PHEROMONE | 4.9 |
| | | full length insert | | | B BETA 1 | |
| | | cDNA clone | | | RECEPTOR | |
| | | ZD77B03 | | | | |
| 190 | K02024 | Human T-cell | 0.075 | 2791527 | (AL021246) | 0.11 |
| | | lymphotropic | | | PE_PGRS | |
| | | virue type II env | | | [Mycobacterium | |
| | | gene encoding | | | tuberculosis] | |
| | | envelope | | | | |
| | | glycoprotein, | | | | |
| | | complete cds. | | | | |
| 191 | M10188 | X. laevis | 0.074 | 4753163 | huntingtin | 2.8 |
| | | mitochondrial | | | DISEASE | |
| | | DNA containing | | | PROTEIN) (HD | |
| | | the D-loop, and | | | PROTEIN) > gi\| | |
| | | the 12S rRNA, | | | 454415 | |
| | | apocytochrome | | | (L12392) | |
| | | b, Glu-tRNA, | | | Huntington's | |
| | | Thr-tRNA, Pro- | | | Disease protein | |
| | | tRNA and Phe- | | | [Homo sapiens] | |
| | | tRNA genes. | | | | |
| 192 | X85525 | G. gallus AG | 0.073 | 984339 | (U20966) Rev | 3.6 |
| | | repeat region | | | [Simian | |
| | | (GgaMU130) | | | immunodeficiency | |
| | | | | | virus] | |
| 193 | AJ238394.1 | Homo sapiens | 0.07 | 4240219 | (AB020672) | 2 |
| | | AML2 gene | | | KIAA0865 | |
| | | (partial) | | | protein [Homo | |
| | | | | | sapiens] | |
| 194 | AF039704 | Homo sapiens | 0.069 | 2894106 | (Z78279) | 0.39 |
| | | lysosomal | | | Collagen alpha1 | |
| | | pepstatin | | | [Rattus | |
| | | insensitive | | | norvegicus] | |
| | | protease (CLN2) | | | | |
| | | gene, complete | | | | |
| | | cds | | | | |
| 195 | K02024 | Human T-cell | 0.068 | 4504857 | potassium | 0.5 |
| | | lymphotropic | | | intermediate/sm | |
| | | virue type II env | | | all conductance | |
| | | gene encoding | | | calcium- | |
| | | envelope | | | activated | |
| | | glycoprotein, | | | channel, | |
| | | complete cds. | | | subfamily N, | |
| | | | | | member 3 > gi\| | |
| | | | | | 3309531 | |
| | | | | | (AF031815) | |
| | | | | | calcium- | |
| | | | | | activated | |
| | | | | | potassium | |
| | | | | | channel [Homo | |
| | | | | | sapiens] | |
| 196 | Z60719 | H. sapiens CpG | 0.068 | 4826874 | nucleoporin | 0.044 |
| | | island DNA | | | 214 kD (CAIN) | |
| | | genomic Mse1 | | | PROTEIN | |
| | | fragment, clone | | | NUP214 | |
| | | 33a11, forward | | | (NUCLEOPORIN | |
| | | read | | | NUP214) | |
| | | cpg33a11.ft1m | | | (214 KD | |
| | | | | | NUCLEOPORIN) | |
| | | | | | transforming | |
| | | | | | protein (can) - | |
| | | | | | human sapiens] | |
| 197 | AF053994 | Lycopersicon | 0.068 | 2842699 | PUTATIVE | 9e−009 |
| | | esculentum | | | UBIQUITIN | |
| | | Hcr2-0A (Hcr2- | | | CARBOXYL- | |
| | | 0A) gene, | | | TERMINAL | |
| | | complete cds | | | HYDROLASE | |
| | | | | | C6G9.08 | |
| | | | | | (UBIQUITIN | |
| | | | | | THIOLESTERASE) | |
| | | | | | (UBIQUITIN- | |
| | | | | | SPECIFIC | |
| | | | | | PROCESSING | |
| | | | | | PROTEASE) | |
| 198 | AJ233650.1 | Equus caballus | 0.067 | <NONE> | <NONE> | <NONE> |
| | | endogenous | | | | |
| | | retroviral | | | | |
| | | sequence ERV- | | | | |
| | | L pol gene, | | | | |
| | | clone ERV-L | | | | |
| | | Horse1 | | | | |
| 199 | M10188 | X. laevis | 0.067 | 4753163 | huntingtin | 2.5 |
| | | mitochondrial | | | DISEASE | |
| | | DNA containing | | | PROTEIN) (HD | |
| | | the D-loop, and | | | PROTEIN) > gi\| | |
| | | the 12S rRNA, | | | 454415 | |
| | | apocytochrome | | | (L12392) | |
| | | b, Glu-tRNA, | | | Huntington's | |
| | | Thr-tRNA, Pro- | | | Disease protein | |
| | | tRNA and Phe- | | | [Homo sapiens] | |
| | | tRNA genes. | | | | |
| 200 | U14646 | Murine hepatitis | 0.067 | 3880930 | (AL021481) | 1e−019 |
| | | virus Y strain S | | | similar to | |
| | | glycoprotein | | | Phosphoglucomutase | |
| | | gene, complete | | | and | |
| | | cds. | | | phosphomannomutase | |
| | | | | | phosphoserine; | |
| | | | | | cDNA EST | |
| | | | | | EMBL: D36168 | |
| | | | | | comes from this | |
| | | | | | gene; cDNA | |
| | | | | | EST | |
| | | | | | EMBL: D70697 | |
| | | | | | comes from this | |
| | | | | | gene; cDNA | |
| | | | | | EST yk373h9.5 | |
| | | | | | comes from this | |
| | | | | | gene; cDNA | |
| | | | | | EST | |
| | | | | | EMBL: T00805 | |
| | | | | | . . . | |
| 201 | X15373 | Mouse | 0.066 | 164507 | (M81771) | 9.4 |
| | | cerebellum | | | immunoglobulin | |
| | | mRNA for P400 | | | gamma-chain | |
| | | protein | | | [Sus scrofa] | |
| 202 | AF086410 | Homo sapiens | 0.066 | 3023371 | PHEROMONE | 4.2 |
| | | full length insert | | | B BETA 1 | |
| | | cDNA clone | | | RECEPTOR | |
| | | ZD77B03 | | | | |
| 203 | AL034492 | Streptomyces | 0.066 | 3800951 | (AF100657) No | 3e−015 |
| | | coelicolor | | | definition line | |
| | | cosmid 6C5 | | | found | |
| | | | | | [Caenorhabditis | |
| | | | | | elegans] | |
| 204 | L13377 | Staphylococcus | 0.065 | <NONE> | <NONE> | <NONE> |
| | | aureus | | | | |
| | | enterotoxin | | | | |
| | | gene, 3′ end. | | | | |
| 205 | U83478 | Thelephoraceae | 0.065 | 3877335 | (Z92786) | 9.1 |
| | | sp. ‘Taylor #13’ | | | predicted using | |
| | | ITS1, 5.8S | | | Genefinder | |
| | | ribosomal RNA | | | | |
| | | gene, and ITS2, | | | | |
| | | complete | | | | |
| | | sequence | | | | |
| 206 | AJ002014 | Crythecodinium | 0.065 | 1213283 | (U40576) SIM2 | 0.47 |
| | | cohnii mRNA | | | [Mus musculus] | |
| | | for nuclear | | | | |
| | | protein JUS1 | | | | |
| 207 | AB016804 | Aloe | 0.065 | 2832777 | (AL021086)/ | 5e−036 |
| | | arborescens | | | prediction = (method:; | |
| | | mRNA for | | | comes | |
| | | NADP-malic | | | from the 5′ | |
| | | enzyme, | | | UTR | |
| | | complete cds | | | [Drosophila | |
| | | | | | melanogaster] | |
| 208 | AJ002014 | Crythecodinium | 0.063 | 1213283 | (U40576) SIM2 | 0.45 |
| | | cohnii mRNA | | | [Mus musculus] | |
| | | for nuclear | | | | |
| | | protein JUS1 | | | | |
| 209 | AB023143.1 | Homo sapiens | 0.024 | 132575 | RIBONUCLEASE | 8e−026 |
| | | mRNA for | | | INHIBITOR | |
| | | KIAA0926 | | | | |
| | | protein, | | | | |
| | | complete cds | | | | |
| 210 | U72966 | Human | 0.022 | <NONE> | <NONE> | <NONE> |
| | | hepatocyte | | | | |
| | | nuclear factor 4- | | | | |
| | | alpha gene, | | | | |
| | | exon 7 | | | | |
| 211 | X02801 | Mouse gene for | 0.022 | 2231607 | (U85917) nef | 7 |
| | | glial fibrillary | | | protein [Human | |
| | | acidic protein | | | immunodeficiency | |
| | | | | | virus type 1] | |
| 212 | AF017636 | Mesocricetus | 0.022 | 2723362 | (AF023459) | 0.097 |
| | | auratus 3-keto- | | | lustrin A | |
| | | steroid reductase | | | [Haliotis | |
| | | | | | rufescens] | |
| 213 | Z36879 | F. pringlei | 0.008 | <NONE> | <NONE> | <NONE> |
| | | gdcsPA gene for | | | | |
| | | P-protein of the | | | | |
| | | glycine cleavage | | | | |
| | | system | | | | |
| 214 | X73150 | P. sativum | 0.008 | 1572629 | (U69699) | 8.6 |
| | | GapC1 gene | | | unknown protein | |
| | | | | | precursor [Mus | |
| | | | | | musculus] | |
| 215 | AJ239031.1 | Homo sapiens | 0.008 | 4508019 | zinc finger | 0.01 |
| | | LSS gene, | | | protein 231 | |
| | | partial, exons | | | protein [Homo | |
| | | 22, 23 and | | | sapiens] | |
| | | joined CDS | | | | |
| 216 | U76602 | Human 180 kDa | 0.007 | 3170252 | (AF043636) | 0.0001 |
| | | bullous | | | circumsporozoite | |
| | | pemphigoid | | | protein | |
| | | antigen 2/type | | | [Plasmodium | |
| | | XVII collagen | | | chabaudi] | |
| | | (BPAG2/COL17 | | | | |
| | | A1) gene, exons | | | | |
| | | 49, 50, 51 and | | | | |
| | | 52 | | | | |
| 217 | M11283 | Aplysia | 0.007 | 3874685 | (Z78539) | 9e−013 |
| | | californica | | | Similarity to | |
| | | FMRFamide | | | S. pombe | |
| | | mRNA, partial | | | hypothetical | |
| | | cds, clone | | | protein | |
| | | FMRF-2. | | | C4G8.04 | |
| | | | | | (SW: YAD4_SC | |
| | | | | | HPO); cDNA | |
| | | | | | EST | |
| | | | | | EMBL: D27846 | |
| | | | | | comes from this | |
| | | | | | gene; cDNA | |
| | | | | | EST | |
| | | | | | EMBL: D27845 | |
| | | | | | comes from this | |
| | | | | | gene; cDNA | |
| | | | | | EST yk202h7.3 | |
| | | | | | comes from this | |
| | | | | | gene; cDNA | |
| | | | | | EST yk202h7.5 | |
| | | | | | come . . . | |
| 218 | J03998 | P. falciparum | 0.003 | <NONE> | <NONE> | <NONE> |
| | | glutamic acid- | | | | |
| | | rich protein | | | | |
| | | gnen, complete | | | | |
| | | cds. | | | | |
| 219 | Z23143 | M. musculus | 0.002 | 2393890 | (AF006064) | 1e−011 |
| | | ALK-6 mRNA, | | | protein kinase | |
| | | complete CDS | | | homolog | |
| | | | | | [Fowlpox virus] | |
| 220 | AB007914 | Homo sapiens | 0.001 | 2136964 | cysteine-rich | 1.9 |
| | | mRNA for | | | hair keratin | |
| | | KIAA0445 | | | associated | |
| | | protein, | | | protein - rabbit > gi\| | |
| | | complete cds | | | 510541\|emb\| | |
| | | | | | CAA56339\| | |
| | | | | | (X80035) | |
| | | | | | cysteine rich | |
| | | | | | hair keratin | |
| | | | | | associated | |
| | | | | | protein | |
| 221 | AB012105 | Brassica rapa | 0.0008 | 3687246 | (AC005169) | 5.5 |
| | | mRNA for | | | putative | |
| | | SLG45, | | | suppressor | |
| | | complete cds | | | protein | |
| | | | | | [Arabidopsis | |
| | | | | | thaliana] | |
| 222 | L41608 | Methylobacterium | 0.0008 | 3024235 | NERVOUS- | 5.1 |
| | | extorquens | | | SYSTEM | |
| | | (clone pDN9, | | | SPECIFIC | |
| | | HINDIIIAB) | | | OCTAMER- | |
| | | mxaS gene 3′ | | | BINDING | |
| | | end, mxaA, | | | TRANSCRIPTION | |
| | | mxaC, mxaK, | | | FACTOR | |
| | | mxaL and mxaD | | | N-OCT 3 | |
| | | genes, complete | | | PROTEIN) | |
| | | cds. | | | | |
| 223 | AB007914 | Homo sapiens | 0.0008 | 2136964 | cysteine-rich | 2.5 |
| | | mRNA for | | | hair keratin | |
| | | KIAA0445 | | | associated | |
| | | protein, | | | protein - rabbit > gi\| | |
| | | complete cds | | | 510541\|emb\| | |
| | | | | | CAA56339\| | |
| | | | | | (X80035) | |
| | | | | | cysteine rich | |
| | | | | | hair keratin | |
| | | | | | associated | |
| | | | | | protein | |
| 224 | AC002293 | Genomic | 0.0008 | 2789557 | (AF034316) | 0.0002 |
| | | sequence from | | | MHC class I | |
| | | Human 9q34, | | | antigen [Triakis | |
| | | complete | | | scyllium] | |
| | | sequence [Homo | | | scyllium] | |
| | | sapiens] | | | | |
| 225 | L16013 | Rattus | 9e−005 | <NONE> | <NONE> | <NONE> |
| | | norvegicus Q- | | | | |
| | | like gene | | | | |
| | | sequence | | | | |
| 226 | AF148512.1 | Homo sapiens | 9e−005 | <NONE> | <NONE> | <NONE> |
| | | hexokinase II | | | | |
| | | gene, promoter | | | | |
| | | region | | | | |
| 227 | U94776 | Human muscle | 9e−005 | 4759138 | solute carrier | 5.4 |
| | | glycogen | | | family 7 | |
| | | phosphorylase | | | transporter 3 | |
| | | (PYGM) gene, | | | [Homo sapiens] | |
| | | exons 6 through | | | | |
| | | 17 | | | | |
| 228 | X56030 | H. sapiens IAPP | 1e−005 | <NONE> | <NONE> | <NONE> |
| | | gene for | | | | |
| | | amyloid | | | | |
| | | polypeptide, | | | | |
| | | exon 1 | | | | |
| 229 | U36515 | Human CT | 4e−007 | 2435616 | (AF026215) No | 0.85 |
| | | microsatellite, | | | definition line | |
| | | clone GM5927- | | | found | |
| | | CT-2-3, from | | | [Caenorhabditis | |
| | | the tandernly | | | elegans] | |
| | | repeated genes | | | | |
| | | encoding U2 | | | | |
| | | small nuclear | | | | |
| | | RNA (RNU2 | | | | |
| | | locus) | | | | |
| 230 | AB011119 | Homo sapiens | 4e−007 | 4758508 | airway trypsin- | 3e−031 |
| | | mRNA for | | | like protease | |
| | | KIAA0547 | | | protease [Homo | |
| | | protein, | | | sapiens] | |
| | | complete cds | | | | |
| 231 | NM_000521.1 | Homo sapiens | 5e−008 | 2119379 | slow muscle | 2.8 |
| | | hexosaminidase | | | troponin T - | |
| | | B (beta | | | chicken T | |
| | | polypeptide) | | | [Gallus gallus] | |
| | | (HEXB) mRNA | | | | |
| 232 | X13895 | Human serum | 4e−008 | 699405 | (U18682) novel | 7.7 |
| | | amyloid A | | | antigen receptor | |
| | | (GSAA1) gene, | | | [Ginglymostoma | |
| | | complete cds | | | cirratum] | |
| 233 | AB009288.1 | Homo sapiens | 4e−008 | 4520342 | (AB008893) N- | 3e−006 |
| | | mRNA for N- | | | copine [Mus | |
| | | copine, | | | musculus] | |
| | | complete cds | | | | |
| 234 | AB011119 | Homo sapiens | 4e−008 | 4758508 | airway trypsin- | 1e−028 |
| | | mRNA for | | | like protease | |
| | | KIAA0547 | | | protease [Homo | |
| | | protein, | | | sapiens] | |
| | | complete cds | | | | |
| 235 | X13895 | Human serum | 5e−009 | 699405 | (U18682) novel | 7.8 |
| | | amyloid A | | | antigen receptor | |
| | | (GSAA1) gene, | | | [Ginglymostoma | |
| | | complete cds | | | cirratum] | |
| 236 | X13895 | Human serum | 2e−009 | 699405 | (U18682) novel | 7.2 |
| | | amyloid A | | | antigen receptor | |
| | | (GSAA1) gene, | | | [Ginglymostoma | |
| | | complete cds | | | cirratum] | |
| 237 | U64997 | Bos taurus | 2e−009 | 3914810 | RIBONUCLEASE | 3e−018 |
| | | ribonuclease K6 | | | K6 | |
| | | gene, partial cds | | | PRECURSOR | |
| | | | | | (RNASE K6) > gi\| | |
| | | | | | 2745760 | |
| | | | | | (AF037086) | |
| | | | | | ribonuclease k6 | |
| | | | | | precursor | |
| 238 | J02635 | Rat liver alpha- | 2e−009 | 112913 | ALPHA-2- | 4e−019 |
| | | 2-macroglobulin | | | MACROGLOBULIN | |
| | | mRNA, | | | PRECURSOR | |
| | | complete cds. | | | precursor - rat > gi\| | |
| | | | | | 202592 | |
| | | | | | (J02635) | |
| | | | | | prealpha-2- | |
| | | | | | macroglobulin | |
| | | | | | [Rattus | |
| | | | | | norvegicus] | |
| 239 | Z78141 | M. musculus | 5e−010 | 3219569 | (AL023893)/ | 4e−009 |
| | | partial cochlear | | | prediction = | |
| | | mRNA (clone | | | (method:; | |
| | | 29C9) | | | | |
| 240 | AF060917 | Gambusia | 2e−010 | 3874618 | (Z48241) | 0.096 |
| | | affinis | | | similar to coiled | |
| | | microsatellite | | | coil domains; | |
| | | Gafu6 | | | cDNA EST | |
| | | | | | yk302g12.5 | |
| | | | | | comes from this | |
| | | | | | gene; cDNA | |
| | | | | | EST | |
| | | | | | yk365d10.5 | |
| | | | | | comes from this | |
| | | | | | gene; cDNA | |
| | | | | | EST yk461c1.5 | |
| | | | | | comes from this | |
| | | | | | gene | |
| | | | | | [Caenorhabditis | |
| | | | | | elegans] coil | |
| | | | | | domains; cDNA | |
| | | | | | EST | |
| | | | | | yk302g12.5 | |
| | | | | | comes from this | |
| | | | | | gene; cDNA | |
| | | | | | EST | |
| 241 | U68138 | Human PSD-95 | 2e−010 | 4521241 | (AB024927) | 2e−022 |
| | | mRNA, partial | | | CsENDO-3 | |
| | | cds | | | [Ciona savignyi] | |
| 242 | U88827 | Aotus trivirgatus | 6e−011 | 3914810 | RIBONUCLEASE | 1e−016 |
| | | ribonuclease | | | K6 | |
| | | precursor gene, | | | PRECURSOR | |
| | | complete cds | | | (RNASE K6) > gi\| | |
| | | | | | 2745760 | |
| | | | | | (AF037086) | |
| | | | | | ribonuclease k6 | |
| | | | | | precursor | |
| 243 | AF045573 | Mus musculus | 2e−012 | 3025718 | (AF045573) | 3e−016 |
| | | FLI-LRR | | | FLI-LRR | |
| | | associated | | | associated | |
| | | protein-1 | | | protein-1 [Mus | |
| | | mRNA, | | | musculus] | |
| | | complete cds | | | | |
| 244 | NM_001365.1 | Homo sapiens | 2e−012 | 4521241 | (AB024927) | 5e−020 |
| | | discs, large | | | CsENDO-3 | |
| | | (Drosophila) | | | [Ciona savignyi] | |
| | | homolog 4 | | | | |
| | | (DLG4) mRNA > :: | | | | |
| | | gb\|U83192\|HS | | | | |
| | | U83192 Homo | | | | |
| | | sapiens post- | | | | |
| | | synaptic density | | | | |
| | | protein 95 | | | | |
| | | (PSD95) | | | | |
| | | mRNA, | | | | |
| | | complete cds | | | | |
| 245 | U28049 | Human TBX2 | 7e−013 | 2501115 | TBX2 | 2e−011 |
| | | (TXB2) mRNA, | | | PROTEIN (T- | |
| | | complete cds. | | | BOX PROTEIN | |
| | | | | | 2) | |
| 246 | M23404 | Chicken | 2e−013 | 726403 | (U23175) | 1e−025 |
| | | erythrocyte | | | similar to anion | |
| | | anion transport | | | exchange | |
| | | protein (band3) | | | protein | |
| | | mRNA, | | | [Caenorhabditis | |
| | | complete cds. | | | elegans] | |
| 247 | AF005963 | Homo sapiens | 1e−014 | 104270 | Ig heavy chain - | 1.9 |
| | | XY homologous | | | clawed frog | |
| | | region, partial | | | | |
| | | sequence | | | | |
| 248 | M29863 | Human farnesyl | 9e−015 | 182405 | (M29863) | 0.005 |
| | | pyrophosphate | | | farnesyl | |
| | | synthetase | | | pyrophosphate | |
| | | mRNA | | | synthetase | |
| | | | | | [Homo sapiens] | |
| 249 | D28126 | Human gene for | 3e−015 | <NONE> | <NONE> | <NONE> |
| | | ATP synthase | | | | |
| | | alpha subunit, | | | | |
| | | complete cds | | | | |
| | | (exon 1 to 12) | | | | |
| 250 | Z80150 | H. sapiens | 3e−015 | 3387914 | (AF070550) | 3.5 |
| | | CACNL1A4 | | | cote 1 [Homo | |
| | | gene, exons 41 | | | sapiens] | |
| | | and 42 > :: | | | | |
| | | emb\|A70716.1\| | | | | |
| | | A70716 | | | | |
| | | Sequence 37 | | | | |
| | | from Patent | | | | |
| | | WO9813490 | | | | |
| 251 | U28049 | Human TBX2 | 4e−016 | 2501116 | TBX2 | 6e−009 |
| | | (TXB2) mRNA, | | | PROTEIN (T- | |
| | | complete cds. | | | BOX PROTEIN | |
| | | | | | 2) tbx gene | |
| | | | | | [Mus musculus] | |
| 252 | U31629 | Mus musculus | 1e−017 | 3024998 | HYPOTHETICAL | 3e−017 |
| | | C2C12 unknown | | | HEART | |
| | | mRNA, partial | | | PROTEIN | |
| | | cds. | | | | |
| 253 | J05262 | Human farnesyl | 1e−018 | 182405 | (M29863) | 0.0001 |
| | | pyrophosphate | | | farnesyl | |
| | | synthetase | | | pyrophosphate | |
| | | mRNA, | | | synthetase | |
| | | complete cds. | | | [Homo sapiens] | |
| 254 | D28126 | Human gene for | 5e−019 | <NONE> | <NONE> | <NONE> |
| | | ATP synthase | | | | |
| | | alpha subunit, | | | | |
| | | complete cds | | | | |
| | | (exon 1 to 12) | | | | |
| 255 | D28126 | Human gene for | 5e−019 | 3219984 | HYPOTHETICAL | 5.7 |
| | | ATP synthase | | | PROTEIN | |
| | | alpha subunit, | | | MJ1597.1 | |
| | | complete cds | | | region | |
| | | (exon 1 to 12) | | | MJ1597.1 | |
| | | | | | [Methanococcus | |
| | | | | | jannaschii] | |
| 256 | NM_004587.1 | Homo sapiens | 2e−019 | 4759056 | ribosome | 0.004 |
| | | ribosome | | | binding protein | |
| | | binding protein | | | 1 (dog 180 kD | |
| | | 1 (dog 180 kD | | | homolog) > gi\| | |
| | | homolog) | | | 3299885 | |
| | | (RRBP1) | | | (AF006751) | |
| | | mRNA > :: | | | ES/130 [Homo | |
| | | gb\|AF006751\| | | | sapiens] | |
| | | AF006751 | | | | |
| | | Homo sapiens | | | | |
| | | ES/130 mRNA, | | | | |
| | | complete cds | | | | |
| 257 | U89915 | Mus musculus | 5e−020 | 3462455 | (U89915) | 2e−005 |
| | | junctional | | | junctional | |
| | | adhesion | | | adhesion | |
| | | molecule (Jam) | | | molecule [Mus | |
| | | mRNA, | | | musculus] | |
| | | complete cds | | | | |
| 258 | AF045573 | Mus musculus | 5e−020 | 3025718 | (AF045573) | 9e−025 |
| | | FLI-LRR | | | FLI-LRR | |
| | | associated | | | associated | |
| | | protein-1 | | | protein-1 [Mus | |
| | | mRNA, | | | musculus] | |
| | | complete cds | | | | |
| 259 | NM_004587.1 | Homo sapiens | 2e−020 | 4759056 | ribosome | 0.0008 |
| | | ribosome | | | binding protein | |
| | | binding protein | | | 1 (dog 180 kD | |
| | | 1 (dog 180 kD | | | homolog) > gi\| | |
| | | homolog) | | | 3299885 | |
| | | (RRBP1) | | | (AF006751) | |
| | | mRNA > :: | | | ES/130 [Homo | |
| | | gb\|AF006751\| | | | sapiens] | |
| | | AF006751 | | | | |
| | | Homo sapiens | | | | |
| | | ES/130 mRNA, | | | | |
| | | complete cds | | | | |
| 260 | AF051098 | Mus musculus | 2e−021 | 3858883 | (U67056) | 0.002 |
| | | seven | | | myosin I heavy | |
| | | transmembrane | | | chain kinase | |
| | | domain orphan | | | [Acanthamoeba | |
| | | receptor mRNA, | | | castellanii] > gi\| | |
| | | complete cds | | | 4206769 | |
| | | | | | (AF104910) | |
| | | | | | myosin I heavy | |
| | | | | | chain kinase | |
| | | | | | [Acanthamoeba | |
| | | | | | castellanii] | |
| 261 | AF051098 | Mus musculus | 2e−021 | 3858883 | (U67056) | 0.001 |
| | | seven | | | myosin I heavy | |
| | | transmembrane | | | chain kinase | |
| | | domain orphan | | | [Acanthamoeba | |
| | | receptor mRNA, | | | castellanii] > gi\| | |
| | | complete cds | | | 4206769 | |
| | | | | | (AF104910) | |
| | | | | | myosin I heavy | |
| | | | | | chain kinase | |
| | | | | | [Acanthamoeba | |
| | | | | | castellanii] | |
| 262 | M13519 | Human N- | 2e−021 | 4504373 | hexosaminidase | 2e−007 |
| | | acetyl-beta- | | | B (beta | |
| | | glucosaminidase | | | polypeptide) > gi\| | |
| | | (HEXB) | | | 123081\|sp\| | |
| | | mRNA, 3′ end. | | | P07686\| | |
| | | | | | HEXB_{—} | |
| | | | | | HUMAN | |
| | | | | | BETA- | |
| | | | | | HEXOSAMINIDASE | |
| | | | | | BETA | |
| | | | | | CHAIN | |
| | | | | | PRECURSOR | |
| | | | | | beta-N- | |
| | | | | | acetylhexosaminidase | |
| | | | | | (EC | |
| | | | | | 3.2.1.52) beta | |
| | | | | | chain - human > gi\| | |
| | | | | | 386770 | |
| | | | | | (M23294) beta- | |
| | | | | | hexosaminidase | |
| | | | | | beta-subunit | |
| | | | | | [Homo sapiens] | |
| 263 | Z81014 | Human DNA | 2e−022 | <NONE> | <NONE> | <NONE> |
| | | sequence from | | | | |
| | | cosmid U65A4, | | | | |
| | | between | | | | |
| | | markers | | | | |
| | | DXS366 and | | | | |
| | | DXS87 on | | | | |
| | | chromosome X* | | | | |
| 264 | AF147311.1 | Homo sapiens | 2e−022 | 3875904 | (Z70207) | 0.07 |
| | | full length insert | | | predicted using | |
| | | cDNA clone | | | Genefinder; | |
| | | YA82F10 | | | similar to | |
| | | | | | collagen; cDNA | |
| | | | | | EST | |
| | | | | | EMBL: D65905 | |
| | | | | | comes from this | |
| | | | | | gene; cDNA | |
| | | | | | EST | |
| | | | | | EMBL: D65858 | |
| | | | | | comes from this | |
| | | | | | gene; cDNA | |
| | | | | | EST | |
| | | | | | EMBL: D69306 | |
| | | | | | comes from this | |
| | | | | | gene; cDNA | |
| | | | | | EST | |
| | | | | | EMBL: D65755 | |
| | | | | | comes from this | |
| | | | | | gen . . . | |
| 265 | AF037088 | Gorilla gorilla | 9e−024 | 3914791 | RIBONUCLEASE | 3e−019 |
| | | ribonuclease k6 | | | K6 | |
| | | precursor, gene, | | | PRECURSOR | |
| | | complete cds | | | (RNASE K6) > gi\| | |
| | | | | | 2745752 | |
| | | | | | (AF037082) | |
| | | | | | ribonuclease k6 | |
| | | | | | precursor | |
| 266 | Z81014 | Human DNA | 8e−024 | <NONE> | <NONE> | <NONE> |
| | | sequence from | | | | |
| | | cosmid U65A4, | | | | |
| | | between | | | | |
| | | markers | | | | |
| | | DXS366 and | | | | |
| | | DXS87 on | | | | |
| | | chromosome X* | | | | |
| 267 | AF037088 | Gorilla gorilla | 9e−025 | 3914810 | RIBONUCLEASE | 4e−018 |
| | | ribonuclease k6 | | | K6 | |
| | | precursor, gene, | | | PRECURSOR | |
| | | complete cds | | | (RNASE K6) > gi\| | |
| | | | | | 2745760 | |
| | | | | | (AF037086) | |
| | | | | | ribonuclease k6 | |
| | | | | | precursor | |
| 268 | AF147311.1 | Homo sapiens | 1e−026 | 131413 | PULMONARY | 0.059 |
| | | full length insert | | | SURFACTANT- | |
| | | cDNA clone | | | ASSOCIATED | |
| | | YA82F10 | | | PROTEIN A | |
| | | | | | PRECURSOR | |
| | | | | | (SP-A) (PSP-A) | |
| | | | | | (PSAP) | |
| | | | | | precursor - | |
| | | | | | rabbit > gi\| | |
| | | | | | 165706 | |
| | | | | | (J03542) | |
| | | | | | apoprotein of | |
| | | | | | surfactant | |
| | | | | | [Oryctolagus | |
| | | | | | cuniculus] | |
| 269 | Z46786 | D. melanogaster | 1e−027 | 1079042 | acetyl-CoA | 4e−025 |
| | | mRNA for | | | synthetase - fruit | |
| | | acetyl-CoA | | | fly | |
| | | synthetase | | | | |
| 270 | NM_004039.1 | Homo sapiens | 4e−028 | 450448 | (M33322) | 0.1 |
| | | annexin II | | | calpactin I | |
| | | (lipocortin II) | | | heavy chain | |
| | | for lipocortin II, | | | [Mus musculus] | |
| | | complete cds | | | | |
| 271 | X53064 | Homo sapiens | 1e−028 | 134846 | SMALL | 0.005 |
| | | SPRR2A gene | | | PROLINE- | |
| | | encoding small | | | RICH | |
| | | proline rich | | | PROTEIN II | |
| | | protein | | | rich protein | |
| | | | | | [Homo sapiens] | |
| 272 | M29863 | Human farnesyl | 1e−028 | 4503685 | farnesyl | 2e−008 |
| | | pyrophosphate | | | diphosphate | |
| | | synthetase | | | synthase | |
| | | mRNA | | | dimethylallyltranstransferase, | |
| | | | | | geranyltranstransferase) | |
| | | | | | bp313 | |
| | | | | | to bp1374 is | |
| | | | | | almost identical | |
| | | | | | to human | |
| | | | | | farnesyl | |
| | | | | | pyrophosphate | |
| | | | | | synthetase | |
| | | | | | mRNA. [Homo | |
| | | | | | sapiens] | |
| 273 | Z18950 | H. sapiens genes | 5e−029 | 2493898 | DOPAMINE- | 1.4 |
| | | for S100E | | | BETA- | |
| | | calcium binding | | | MONOOXYGENASE | |
| | | protein, CAPL, | | | PRECURSOR | |
| | | and S100D | | | (DOPAMINE | |
| | | calcium binding | | | BETA- | |
| | | protein EF- | | | HYDROXYLASE) | |
| | | Hand patent U.S. | | | (DBH) | |
| | | Pat. No. | | | 1.14.17.1) | |
| | | 5789248 | | | precursor - | |
| | | | | | mouse > gi\| | |
| | | | | | 260873\|bbs\| | |
| | | | | | 119249 621 | |
| | | | | | aa] [Mus sp.] | |
| 274 | M19481 | Human | 5e−030 | <NONE> | <NONE> | <NONE> |
| | | follistatin gene, | | | | |
| | | exon 6. | | | | |
| 275 | AF007155 | Homo sapiens | 2e−032 | 4502641 | chemokine (C- | 1.6 |
| | | clone 23763 | | | C) receptor 7 | |
| | | unknown | | | TYPE 7 | |
| | | mRNA, partial | | | PRECURSOR | |
| | | cds | | | (C-C CKR-7) | |
| | | | | | (CC-CKR-7) | |
| | | | | | (CCR-7) (MIP-3 | |
| | | | | | BETA | |
| | | | | | RECEPTOR) | |
| | | | | | (EBV- | |
| | | | | | INDUCED G | |
| | | | | | PROTEIN- | |
| | | | | | COUPLED | |
| | | | | | RECEPTOR 1) | |
| | | | | | (EBI1) (BLR2) > gi\| | |
| | | | | | 1082381\|pir\|\| | |
| | | | | | B55735 | |
| | | | | | lymphocyte- | |
| | | | | | specific G- | |
| | | | | | protein-coupled | |
| | | | | | receptor EBI1 - | |
| | | | | | human > gi\| | |
| | | | | | 468316 | |
| | | | | | (L3158 | |
| 276 | M99624 | Human | 8e−034 | 294845 | (L13655) | 9e−014 |
| | | epidermal | | | membrane | |
| | | growth factor | | | protein | |
| | | receptor-related | | | [Saccharum | |
| | | gene, 5′ end. | | | hybrid cultivar | |
| | | | | | H65-7052] | |
| 277 | U49082 | Human | 8e−035 | 1840045 | (U49082) | 1e−014 |
| | | transporter | | | transporter | |
| | | protein (g17) | | | protein [Homo | |
| | | mRNA, | | | sapiens] | |
| | | complete cds | | | | |
| 278 | D50369 | Homo sapiens | 9e−036 | 3024781 | UBIQUINOL- | 0.0002 |
| | | mRNA for low | | | CYTOCHROME C | |
| | | molecular mass | | | REDUCTASE | |
| | | ubiquinone- | | | COMPLEX | |
| | | binding protein, | | | UBIQUINONE- | |
| | | complete cds | | | BINDING | |
| | | | | | PROTEIN QP- | |
| | | | | | C PROTEIN) | |
| | | | | | (COMPLEX III | |
| | | | | | SUBUNIT VII) | |
| | | | | | ubiquinone- | |
| | | | | | binding protein | |
| | | | | | [Homo sapiens] | |
| 279 | AF086313 | Homo sapiens | 9e−036 | 2832777 | (AL021086)/ | 1e−039 |
| | | full length insert | | | prediction = (method: | |
| | | cDNA clone | | | ; comes | |
| | | ZD52B10 | | | from the 5′ | |
| | | | | | UTR | |
| | | | | | [Drosophila | |
| | | | | | melanogaster] | |
| 280 | NM_004074.1 | Homo sapiens | 1e−038 | 2499854 | PROBABLE | 2 |
| | | cytochrome c | | | PEPTIDASE | |
| | | oxidase subunit | | | Y4SO > gi\| | |
| | | VIII (COX8), | | | 2182630 | |
| | | nuclear gene | | | | |
| | | encoding | | | | |
| | | mitochondrial | | | | |
| | | protein, mRNA > :: | | | | |
| | | gb\|J04823\|HU | | | | |
| | | MCOX8A | | | | |
| | | Human | | | | |
| | | cytochrome c | | | | |
| | | oxidase subunit | | | | |
| | | VIII (COX8) | | | | |
| | | mRNA, | | | | |
| | | complete cds. | | | | |
| 281 | AB024436.1 | Homo sapiens | 2e−041 | 3132900 | (AF038662) | 4e−016 |
| | | mRNA for beta- | | | beta-1,4- | |
| | | 1,4- | | | galactosyltransferase | |
| | | galactosyltransferase | | | [Homo | |
| | | IV, | | | sapiens] beta- | |
| | | complete cds | | | 1,4- | |
| | | | | | galactosyltransfe | |
| | | | | | galactosyltransferase | |
| | | | | | IV [Homo | |
| | | | | | sapiens] | |
| 282 | AF057734 | Homo sapiens | 2e−043 | 2842416 | (AL008730) | 3e−062 |
| | | 17-beta- | | | dJ487J7.1.1 | |
| | | hydroxysteroid | | | (putative protein | |
| | | dehydrogenase | | | dJ487J7. 1 | |
| | | IV (HSD17B4) | | | isoform 1) | |
| | | gene, exon 16 | | | [Homo sapiens] | |
| 283 | Z69650.1 | Human DNA | 2e−044 | 1872200 | (U22376) | 1e−008 |
| | | sequence from | | | alternatively | |
| | | cosmid L69F7B, | | | spliced product | |
| | | Huntington's | | | using exon 13 A | |
| | | Disease Region, | | | | |
| | | chromosome | | | | |
| | | 4p16.3 contains | | | | |
| | | Huntington | | | | |
| | | Disease (HD) | | | | |
| | | gene | | | | |
| 284 | NM_003938.1 | Homo sapiens | 2e−044 | 3478639 | (AC005545) | 3e−016 |
| | | adaptin, delta | | | delta-adaptin, | |
| | | (ADTD) mRNA > :: | | | partial CDS | |
| | | gb\|U91930\|HS | | | [Homo sapiens] | |
| | | U91930 Homo | | | | |
| | | sapiens AP-3 | | | | |
| | | complex delta | | | | |
| | | subunit mRNA, | | | | |
| | | complete cds | | | | |
| 285 | AF026029 | Homo sapiens | 8e−045 | 1916930 | (U88570) | 7.6 |
| | | poly(A) binding | | | CREB-binding | |
| | | protein II | | | protein homolog | |
| | | (PABP2) gene, | | | [Drosophila | |
| | | complete cds | | | melanogaster] | |
| 286 | AB006622 | Homo sapiens | 1e−045 | 73404 | E2 protein - | 0.11 |
| | | mRNA for | | | human | |
| | | KIAA0284 | | | papillomavirus | |
| | | gene, partial cds | | | type 5 | |
| 287 | U90918 | Human clone | 1e−048 | 3877568 | (Z70208) | 0.042 |
| | | 23654 mRNA | | | similar to | |
| | | sequence | | | collagen | |
| 288 | AB006622 | Homo sapiens | 1e−049 | 73404 | E2 protein - | 0.11 |
| | | mRNA for | | | human | |
| | | KIAA0284 | | | papillomavirus | |
| | | gene, partial cds | | | type 5 | |
| 289 | AL049258.1 | Homo sapiens | 1e−050 | <NONE> | <NONE> | <NONE> |
| | | mRNA; cDNA | | | | |
| | | DKFZp564E173 | | | | |
| | | (from clone | | | | |
| | | DKFZp564E173) | | | | |
| 290 | AF022367 | Homo sapiens | 5e−051 | 3132900 | (AF038662) | 6e−019 |
| | | beta-1,4- | | | beta-1,4- | |
| | | galactosyltransferase | | | galactosyltransferase | |
| | | mRNA, | | | [Homo | |
| | | complete cds | | | sapiens] beta- | |
| | | | | | 1,4- | |
| | | | | | galactosyltransferase | |
| | | | | | IV [Homo | |
| | | | | | sapiens] | |
| 291 | AF057734 | Homo sapiens | 7e−053 | 2842416 | (AL008730) | 6e−055 |
| | | 17-beta- | | | dJ487J7.1.1 | |
| | | hydroxysteroid | | | (putative protein | |
| | | dehydrogenase | | | dJ487J7.1 | |
| | | IV (HSD17B4) | | | isoform 1) | |
| | | gene, exon 16 | | | [Homo sapiens] | |
| 292 | AF097709 | Homo sapiens | 8e−055 | 4506141 | protease, serine, | 2e−017 |
| | | serine protease | | | 11 (IGF | |
| | | (PRSS11) | | | binding) > gi\| | |
| | | mRNA, partial | | | 1513059\|dbj\| | |
| | | cds | | | BAA13322\| | |
| | | | | | (D87258) serin | |
| | | | | | protease with | |
| | | | | | IGF-binding | |
| | | | | | motif [Homo | |
| | | | | | sapiens] | |
| | | | | | protease, | |
| | | | | | PRSS11 [Homo | |
| | | | | | sapiens] | |
| 293 | U31629 | Mus musculus | 9e−057 | 3025215 | HYPOTHETICAL | 5e−033 |
| | | C2C12 unknown | | | 81.0 KD | |
| | | mRNA, partial | | | PROTEIN | |
| | | cds. | | | C35D10.4 IN | |
| | | | | | CHROMOSOME | |
| | | | | | III > gi\| | |
| | | | | | 2146877\|pir\|\| | |
| | | | | | S72572 | |
| | | | | | probable ABC1 | |
| | | | | | protein homolog - | |
| | | | | | Caenorhabditis | |
| | | | | | elegans protein | |
| | | | | | (Swiss-Prot | |
| | | | | | Acc: P27697) | |
| | | | | | [Caenorhabditis | |
| | | | | | elegans] | |
| 294 | AB006622 | Homo sapiens | 8e−057 | 73404 | E2 protein - | 1.7 |
| | | mRNA for | | | human | |
| | | KIAA0284 | | | papillomavirus | |
| | | gene, partial cds | | | type 5 | |
| 295 | AF025439 | Homo sapiens | 4e−059 | <NONE> | <NONE> | <NONE> |
| | | Opa-interacting | | | | |
| | | protein OIP3 | | | | |
| | | mRNA, partial | | | | |
| | | cds | | | | |
| 296 | M99624 | Human | 1e−060 | 123364 | SEGMENTATION | 5.3 |
| | | epidermal | | | PROTEIN | |
| | | growth factor | | | EVEN- | |
| | | receptor-related | | | SKIPPED fly | |
| | | gene, 5′ end. | | | (Drosophila sp.) > gi\| | |
| | | | | | 157387 | |
| | | | | | (M14767) even- | |
| | | | | | skipped gene | |
| | | | | | [Drosophila | |
| | | | | | melanogaster] | |
| 297 | AF045573 | Mus musculus | 5e−061 | 3025718 | (AF045573) | 7e−029 |
| | | FLI-LRR | | | FLI-LRR | |
| | | associated | | | associated | |
| | | protein-1 | | | protein-1 [Mus | |
| | | mRNA, | | | musculus] | |
| | | complete cds | | | | |
| 298 | AB006622 | Homo sapiens | 2e−062 | 2119133 | ribosomal | 2e−015 |
| | | mRNA for | | | proiein S17 —cat | |
| | | KIAA0284 | | | (fragment) | |
| | | gene, partial cds | | | musculus] | |
| 299 | M30702 | Human | 2e−063 | 4502199 | amphiregulin | 0.0002 |
| | | amphiregulin | | | (schwannoma- | |
| | | (AR) gene, exon | | | derived growth | |
| | | 5, clones | | | factor) > gi\| | |
| | | lambda- | | | 113754\|sp\| | |
| | | ARH(6, 12). | | | P15514\|AMPR_{—} | |
| | | | | | HUMAN | |
| | | | | | AMPHIREGULIN | |
| | | | | | PRECURSOR | |
| | | | | | (AR) | |
| | | | | | (COLORECTUM | |
| | | | | | CELL- | |
| | | | | | DERIVED | |
| | | | | | GROWTH | |
| | | | | | FACTOR) | |
| | | | | | (CRDGF) > gi\| | |
| | | | | | 107391\|pir\|\| | |
| | | | | | A34702 | |
| | | | | | amphiregulin | |
| | | | | | precursor - | |
| | | | | | human > gi\| | |
| | | | | | 178890 | |
| | | | | | (M30703) | |
| | | | | | amphiregulin | |
| | | | | | [Homo sapien | |
| 300 | L38847 | Mus musculus | 6e−064 | 3861228 | (AJ235272) | 2.9 |
| | | hepatoma | | | unknown | |
| | | transmembrane | | | [Rickettsia | |
| | | kinase ligand | | | prowazekii] | |
| | | Sequence 1 from | | | | |
| | | patent | | | | |
| | | U.S. Pat. No. 5624899 | | | | |
| 301 | L38847 | Mus musculus | 6e−064 | 3861228 | (AJ235272) | 2.9 |
| | | hepatoma | | | unknown | |
| | | transmembrane | | | [Rickettsia | |
| | | kinase ligand | | | prowazekii] | |
| | | Sequence 1 from | | | | |
| | | patent | | | | |
| | | U.S. Pat. No. 5624899 | | | | |
| 302 | Z78141 | M. musculus | 8e−066 | 1490324 | (Z78141) | 8e−019 |
| | | partial cochlear | | | unknown [Mus | |
| | | mRNA (clone | | | musculus] | |
| | | 29C9) | | | | |
| 303 | X12650 | Mus musculus | 2e−072 | 833602 | (X54277) | 7e−022 |
| | | gene for beta- | | | cardiac | |
| | | tropomyosin | | | tropomyosin | |
| | | | | | [Coturnix | |
| | | | | | coturnix] | |
| 304 | M87635 | Mouse beta- | 2e−084 | 1216293 | (L35239) | 5e−019 |
| | | tropomyosin 2 | | | cardiac | |
| | | mRNA, | | | tropomyosin | |
| | | complete cds. | | | [Xenopus laevis] | |
| 305 | M13364 | Rabbit calcium- | 2e−084 | 115611 | CALCIUM- | 1e−058 |
| | | dependent | | | DEPENDENT | |
| | | protease, small | | | PROTEASE, | |
| | | subunit mRNA, | | | SMALL | |
| | | complete cds. | | | NEUTRAL | |
| | | | | | PROTEINASE) | |
| | | | | | (CANP) > gi\| | |
| | | | | | 108563\|pir\|\| | |
| | | | | | A34466 | |
| | | | | | calpain (EC | |
| | | | | | 3.4.22.17) II | |
| | | | | | light chain - | |
| | | | | | bovine | |
| | | | | | 3.4.22.17) [Bos | |
| | | | | | taurus] | |
| 306 | M87635 | Mouse beta- | 3e−088 | 1216293 | (L35239) | 9e−028 |
| | | tropomyosin 2 | | | cardiac | |
| | | mRNA, | | | tropomyosin | |
| | | complete cds. | | | [Xenopus laevis] | |
| 307 | M87635 | Mouse beta- | 5e−092 | 1216293 | (L35239) | 2e−035 |
| | | tropomyosin 2 | | | cardiac | |
| | | mRNA, | | | tropomyosin | |
| | | complete cds. | | | [Xenopus laevis] | |
| 308 | X85992 | M. musculus | 8e−097 | 2137756 | semaphorin C - | 2e−048 |
| | | mRNA for | | | mouse | |
| | | semaphorin C | | | (fragment) | |
| | | | | | musculus] | |
| 309 | M24103 | Bovine | e−103 | 113463 | ADP, ATP | 2e−035 |
| | | ADP/ATP | | | CARRIER | |
| | | translocase T2 | | | PROTEIN, | |
| | | mRNA, | | | LIVER | |
| | | complete cds. | | | ISOFORM T2 | |
| | | | | | (ADP/ATP | |
| | | | | | TRANSLOCASE | |
| | | | | | 3) | |
| | | | | | (ADENINE | |
| | | | | | NUCLEOTIDE | |
| | | | | | TRANSLOCATOR | |
| | | | | | 3) (ANT 3) > gi\| | |
| | | | | | 86757\|pir\|\| | |
| | | | | | S03894 | |
| | | | | | ADP, ATP | |
| | | | | | carrier protein | |
| | | | | | T2 - human | |
| 310 | U48852 | Cricetulus | e−107 | 1216486 | (U48852) HT | 3e−057 |
| | | griseus HT | | | protein | |
| | | protein mRNA, | | | [Cricetulus | |
| | | complete cds. | | | griseus] | |
| 311 | X76168 | R. norvegicus | e−112 | 544118 | GAP | 1e−063 |
| | | mRNA for | | | JUNCTION | |
| | | connexin 30.3 | | | BETA-5 | |
| | | | | | PROTEIN | |
| | | | | | (CONNEXIN | |
| | | | | | 30.3) (CX30.3) > gi\| | |
| | | | | | 481577\|pir\|\| | |
| | | | | | S38891 | |
| | | | | | connexin 30.3 - | |
| | | | | | rat > gi\| | |
| | | | | | 431204\|emb\| | |
| | | | | | CAA53762\| | |
| | | | | | (X76168) | |
| | | | | | connexin 30.3 | |
| 312 | X76168 | R. norvegicus | e−115 | 461864 | GAP | 7e−064 |
| | | mRNA for | | | JUNCTION | |
| | | connexin 30.3 | | | BETA-5 | |
| | | | | | PROTEIN | |
| | | | | | junction protein | |
| | | | | | Cx30.3 - mouse > gi\| | |
| | | | | | 192647 | |
| | | | | | (M91443) | |
| | | | | | connexin 30.3 | |
| | | | | | [Mus musculus] | |
| 313 | AJ009634.1 | Mus musculus | e−137 | 4138203 | (AJ009634) | 5e−065 |
| | | fjx1 gene | | | Fjx1 [Mus | |
| | | | | | musculus] | |
| 314 | X76168 | R. norvegicus | e−130 | 544118 | GAP | 2e−074 |
| | | mRNA for | | | JUNCTION | |
| | | connexin 30.3 | | | BETA-5 | |
| | | | | | PROTEIN | |
| | | | | | (CONNEXIN | |
| | | | | | 30.3) (CX30.3) > gi\| | |
| | | | | | 481577\|pir\|\| | |
| | | | | | S38891 | |
| | | | | | connexin 30.3 - | |
| | | | | | rat > gi\| | |
| | | | | | 431204\|emb\| | |
| | | | | | CAA53762\| | |
| | | | | | (X76168) | |
| | | | | | connexin 30.3 | |

TABLE 4

| SEQ | | | CLONES | CLONES | RATIO | RATIO |
|---|---|---|---|---|---|---|
| ID | CLUST | PairAB-text | in A | in B | PLUS | MINUS |
| 4 | 819498 | _21,22 (Normal Prostate vs. Cancerous Prostate) | 6 | 0 | 5.9 | |
| 8 | 728115 | _15,16 (Normal Colon vs. Colon Tumor) | 0 | 7 | | 6.62 |
| | | _16,17 (Colon Tumor vs. Colon Metastasis) | 7 | 0 | 7.11 | |
| 9 | 372700 | _08,09 (Lung, High Metastatic Potential vs. Lung, Low Metastatic Potential) | 3 | 50 | | 11.93 |
| | | _19,20 (Colon Tumor vs. Colon Tumor Metastasis) | 8 | 0 | 5.98 | |
| 12 | 729832 | _15,16 (Normal Colon vs. ColonTumor) | 0 | 11 | | 10.41 |
| | | _16,17 (Colon Tumor vs. Colon Metastasis) | 11 | 0 | 11.17 | |
| 13 | 505514 | _23,24 (Normal Lung vs. Lung Tumor) | 26 | 10 | 2.63 | |
| 17 | 549934 | _21,22 (Normal Prostate vs. Cancerous Prostate) | 8 | 0 | 7.87 | |
| | | _16,17 (Colon Tumor vs. Colon Metastasis) | 3 | 20 | | 6.56 |
| | | _15,16 (Normal Colon vs. Colon Tumor) | 11 | 3 | 3.88 | |
| 25 | 450399 | _15,16 (Normal Colon vs. Colon Tumor) | 28 | 68 | | 2.3 |
| | | _15,17 (Normal Colon vs. Colon Metastasis) | 28 | 117 | | 3.89 |
| 26 | 450982 | _16,17 (Colon Tumor vs. Colon Metastasis) | 14 | 32 | | 2.25 |
| 28 | 379302 | _21,22 (Normal Prostate vs. Cancerous Prostate) | 8 | 1 | 7.87 | |
| 43 | 817503 | _21,22 (Normal Prostate vs. Cancerous Prostate) | 18 | 4 | 4.43 | |
| 48 | 830085 | _21,22 (Normal Prostate vs. Cancerous Prostate) | 0 | 9 | | 9.15 |
| 52 | 830931 | _21,22 (Normal Prostate vs. Cancerous Prostate) | 0 | 7 | | 7.12 |
| 55 | 819046 | _21,22 (Normal Prostate vs. Cancerous Prostate) | 2 | 13 | | 6.61 |
| 58 | 728115 | _15,16 (Normal Colon vs. Colon Tumor) | 0 | 7 | | 6.62 |
| | | _16,17 (Colon Tumor vs. Colon Metastasis) | 7 | 0 | 7.11 | |
| 65 | 553242 | _16,17 (Colon Tumor vs. Colon Metastasis) | 0 | 6 | | 5.91 |
| 71 | 820061 | _21,22 (Normal Prostate vs. Cancerous Prostate) | 1 | 20 | | 20.33 |
| 78 | 220584 | _08,09 (Lung, High Metastatic Potential vs. Lung, Low Metastatic Potential) | 1 | 12 | | 8.59 |
| 80 | 549934 | _16,17 (Colon Tumor vs. Colon Metastasis) | 3 | 20 | | 6.56 |
| | | _15,16 (Normal Colon vs. Colon Tumor) | 11 | 3 | 3.88 | |
| | | _21,22 (Normal Prostate vs. Cancerous Prostate) | 8 | 0 | 7.87 | |
| 86 | 819460 | _21,22 (Normal Prostate vs. Cancerous Prostate) | 18 | 1 | 17.7 | |
| 95 | 551785 | _21,22 (Normal Prostate vs. Cancerous Prostate) | 0 | 6 | | 6.1 |
| 96 | 17092 | _03,04 (Breast, High Metastatic Potential vs. Breast, Non-Metastatic) | 0 | 25 | | 25.62 |
| 99 | 745559 | _21,22 (Normal Prostate vs. Cancerous Prostate) | 1 | 9 | | 9.15 |
| 101 | 379879 | _21,22 (Normal Prostate vs. Cancerous Prostate) | 0 | 9 | | 9.15 |
| | | _08,09 (Lung, High Metastatic Potential vs. Lung, Low Metastatic Potential) | 0 | 13 | | 9.3 |
| 107 | 268290 | _21,22 (Normal Prostate vs. Cancerous Prostate) | 33 | 69 | | 2.13 |
| 108 | 818043 | _21,22 (Normal Prostate vs. Cancerous Prostate) | 6 | 0 | 5.9 | |
| 114 | 450247 | _21,22 (Normal Prostate vs. Cancerous Prostate) | 23 | 8 | 2.83 | |
| 115 | 819273 | _21,22 (Normal Prostate vs. Cancerous Prostate) | 7 | 0 | 6.88 | |
| 116 | 587779 | _21,22 (Normal Prostate vs. Cancerous Prostate) | 6 | 0 | 5.9 | |
| 118 | 615617 | _21,22 (Normal Prostate vs. Cancerous Prostate) | 0 | 7 | | 7.12 |
| 121 | 818682 | _21,22 (Normal Prostate vs. Cancerous Prostate) | 11 | 2 | 5.41 | |
| 123 | 484413 | _21,22 (Normal Prostate vs. Cancerous Prostate) | 7 | 0 | 6.88 | |
| 124 | 819273 | _21,22 (Normal Prostate vs. Cancerous Prostate) | 7 | 0 | 6.88 | |
| 127 | 818682 | _21,22 (Normal Prostate vs. Cancerous Prostate) | 11 | 2 | 5.41 | |
| 131 | 819273 | _21,22 (Normal Prostate vs. Cancerous Prostate) | 7 | 0 | 6.88 | |
| 147 | 820061 | _21,22 (Normal Prostate vs. Cancerous Prostate) | 1 | 20 | | 20.33 |
| 153 | 375958 | _21,22 (Normal Prostate vs. Cancerous Prostate) | 2 | 11 | | 5.59 |
| | | _08,09 (Lung, High Metastatic Potential vs. Lung, Low Metastatic Potential) | 0 | 9 | | 6.44 |
| 155 | 831049 | _21,22 (Normal Prostate vs. Cancerous Prostate) | 0 | 11 | | 11.18 |
| 157 | 553200 | _21,22 (Normal Prostate vs. Cancerous Prostate) | 0 | 6 | | 6.1 |
| 158 | 139677 | _21, 22 (Normal Prostate vs. Cancerous Prostate) | 6 | 0 | 5.9 | |
| 159 | 139677 | _21, 22 (Normal Prostate vs. Cancerous Prostate) | 6 | 0 | 5.9 | |
| 163 | 375958 | _08, 09 (Lung, High Metastatic Potential vs. Lung, Low Metastatic Potential) | 0 | 9 | | 6.44 |
| | | _21, 22 (Normal Prostate vs. Cancerous Prostate) | 2 | 11 | | 5.59 |
| 168 | 831812 | _21, 22 (Normal Prostate vs. Cancerous Prostate) | 0 | 7 | | 7.12 |
| 176 | 193373 | _21, 22 (Normal Prostate vs. Cancerous Prostate) | 6 | 0 | 5.9 | |
| 177 | 400619 | _08, 09 (Lung, High Metastatic Potential vs. Lung, Low Metastatic Potential) | 6 | 0 | 8.38 | |
| 178 | 831149 | _21, 22 (Normal Prostate vs. Cancerous Prostate) | 0 | 7 | | 7.12 |
| 180 | 817503 | _21, 22 (Normal Prostate vs. Cancerous Prostate) | 18 | 4 | 4.43 | |
| 187 | 648679 | _23, 24 (Normal Lung vs. Lung Tumor) | 11 | 1 | 11.11 | |
| | | _16, 17 (Colon Tumor vs. Colon Metastasis) | 79 | 0 | 80.23 | |
| | | _15, 17 (Normal Colon vs. Colon Metastasis) | 7 | 0 | 7.51 | |
| | | _15, 16 (Normal Colon vs. Colon Tumor) | 7 | 79 | | 10.68 |
| 190 | 373928 | _21, 22 (Normal Prostate vs. Cancerous Prostate) | 7 | 0 | 6.88 | |
| 195 | 373928 | _21, 22 (Normal Prostate vs. Cancerous Prostate) | 7 | 0 | 6.88 | |
| 198 | 372700 | _19, 20 (Colon Tumor vs. Colon Tumor Metastasis) | 8 | 0 | 5.98 | |
| | | _08, 09 (Lung, High Metastatic Potential vs. Lung, Low Metastatic Potential) | 3 | 50 | | 11.93 |
| 204 | 379105 | _15, 16 (Normal Colon vs. Colon Tumor) | 0 | 8 | | 7.57 |
| 205 | 831188 | _21, 22 (Normal Prostate vs. Cancerous Prostate) | 0 | 8 | | 8.13 |
| 209 | 831812 | _21, 22 (Normal Prostate vs. Cancerous Prostate) | 0 | 7 | | 7.12 |
| 213 | 831026 | _21, 22 (Normal Prostate vs. Cancerous Prostate) | 0 | 10 | | 10.17 |
| 215 | 380207 | _21, 22 (Normal Prostate vs. Cancerous Prostate) | 0 | 6 | | 6.1 |
| | | _08, 09 (Lung, High Metastatic Potential vs. Lung, Low Metastatic Potential) | 0 | 8 | | 5.72 |
| 216 | 819460 | _21, 22 (Normal Prostate vs. Cancerous Prostate) | 18 | 1 | 17.7 | |
| 224 | 819201 | _21, 22 (Normal Prostate vs. Cancerous Prostate) | 6 | 0 | 5.9 | |
| 225 | 374826 | _15, 17 (Normal Colon vs. Colon Metastasis) | 5 | 20 | | 3.73 |
| | | _08, 09 (Lung, High Metastatic Potential vs. Lung, Low Metastatic Potential) | 38 | 132 | | 2.49 |
| | | _15, 16 (Normal Colon vs. Colon Tumor) | 5 | 18 | | 3.41 |
| 231 | 553242 | _16, 17 (Colon Tumor vs. Colon Metastasis) | 0 | 6 | | 5.91 |
| 246 | 220584 | _08, 09 (Lung, High Metastatic Potential vs. Lung, Low Metastatic Potential) | 1 | 12 | | 8.59 |
| 248 | 819498 | _21, 22 (Normal Prostate vs. Cancerous Prostate) | 6 | 0 | 5.9 | |
| 253 | 819498 | _21, 22 (Normal Prostate vs. Cancerous Prostate) | 6 | 0 | 5.9 | |
| 256 | 831160 | _21, 22 (Normal Prostate vs. Cancerous Prostate) | 0 | 12 | | 12.2 |
| 259 | 831160 | _21, 22 (Normal Prostate vs. Cancerous Prostate) | 0 | 12 | | 12.2 |
| 262 | 373298 | _15, 17 (Normal Colon vs. Colon Metastasis) | 126 | 42 | 3.22 | |
| | | _15, 16 (Normal Colon vs. Colon Tumor) | 126 | 59 | 2.26 | |
| 270 | 450262 | _21, 22 (Normal Prostate vs. Cancerous Prostate) | 0 | 8 | | 8.13 |
| 271 | 484703 | _21, 22 (Normal Prostate vs. Cancerous Prostate) | 28 | 0 | 27.54 | |
| 272 | 819498 | _21, 22 (Normal Prostate vs. Cancerous Prostate) | 6 | 0 | 5.9 | |
| 273 | 406043 | _21, 22 (Normal Prostate vs. Cancerous Prostate) | 0 | 6 | | 6.1 |
| 274 | 817500 | _21, 22 (Normal Prostate vs. Cancerous Prostate) | 2 | 18 | | 9.15 |
| 275 | 818180 | _21, 22 (Normal Prostate vs. Cancerous Prostate) | 2 | 10 | | 5.08 |
| 280 | 429009 | _21, 22 (Normal Prostate vs. Cancerous Prostate) | 8 | 1 | 7.87 | |
| 284 | 383021 | _21, 22 (Normal Prostate vs. Cancerous Prostate) | 3 | 12 | | 4.07 |
| 289 | 831580 | _21, 22 (Normal Prostate vs. Cancerous Prostate) | 0 | 6 | | 6.1 |
| 311 | 763446 | _21, 22 (Normal Prostate vs. Cancerous Prostate) | 11 | 1 | 10.82 | |
| 312 | 763446 | _21, 22 (Normal Prostate vs. Cancerous Prostate) | 11 | 1 | 10.82 | |
| 314 | 763446 | _21, 22 (Normal Prostate vs. Cancerous Prostate) | 11 | 1 | 10.82 | |
| 315 | 10154 | _3, 4 (Breast, High Metastatic Potential vs. Breast, Low Metastatic) | 3 | 317 | | 108.1 |

TABLE 7

| Library No. | Clones |
|---|---|
| es75 | M00063947D: D01 |
| | M00063158A: A01 |
| | M00063517A: A04 |
| | M00063520D: E11 |
| | M00063638C: G12 |
| | M00063642B: A08 |
| | M00063686B: E07 |
| | M00063689D: E12 |
| | M00063781B: B10 |
| | M00063826A: D03 |
| es76 | M00063838B: G08 |
| | M00063838B: G08 |
| | M00063841A: B09 |
| | M00063886A: B06 |
| | M00063910D: A12 |
| | M00063912A: D06 |
| | M00063920D: H05 |
| | M00063928A: G09 |
| | M00063934B: E04 |
| | M00063945A: C03 |
| es77 | M00064032D: G04 |
| | M00064046A: G02 |
| | M00064053C: G04 |
| | M00064053D: F02 |
| | M00064082A: A08 |
| | M00064089B: F09 |
| | M00064132B: B07 |
| | M00064138A: F11 |
| | M00064161B: G04 |
| | M00064175B: B09 |
| es78 | M00064178C: C04 |
| | M00064179A: C04 |
| | M00064200D: E08 |
| | M00064248A: E02 |
| | M00064270B: B03 |
| | M00064271B: D03 |
| | M00063580C: A06 |
| | M00063594B: H07 |
| | M00064002C: F06 |
| | M00064002C: H09 |
| es79 | M00064003B: C10 |
| | M00064302A: D10 |
| | M00064309C: H09 |
| | M00064310D: F03 |
| | M00064322C: A10 |
| | M00064359B: H12 |
| | M00064390A: C05 |
| | M00064404A: B05 |
| | M00064404C: G05 |
| | M00064404D: A06 |
| es80 | M00064429D: B07 |
| | M00064446A: D11 |
| | M00064457D: C09 |
| | M00064476D: C04 |
| | M00064506A: C07 |
| | M00064514A: G10 |
| | M00064520A: F08 |
| | M00064579D: E11 |
| | M00064620C: D01 |
| | M00064624D: C09 |
| es81 | M00064633C: A03 |
| | M00064637B: F03 |
| | M00064690A: C04 |
| | M00064690A: C04 |
| | M00064714A: G03 |
| | M00064723D: H11 |
| | GKC10154-1 |
| | GKC10154-3 |
| es82 | M00063151A: G06 |
| | M00063151D: B10 |
| | M00063152C: B07 |
| | M00063156D: H10 |
| | M00063158A: E11 |
| | M00063158A: E11 |
| | M00063452A: F08 |
| | M00063453B: F08 |
| | M00063462D: D07 |
| | M00063463D: B05 |
| | M00063466C: C11 |
| | M00063467D: H07 |
| | M00063478C: D01 |
| | M00063482A: A08 |
| | M00063482A: F07 |
| | M00063485A: E05 |
| | M00063487C: C02 |
| | M00063514C: D03 |
| | M00063514C: E08 |
| | M00063515B: F06 |
| | M00063515B: H02 |
| | M00063518D: A01 |
| | M00063520D: D08 |
| | M00063604A: B11 |
| | M00063606C: B04 |
| | M00063610D: C11 |
| | M00063613D: C11 |
| | M00063617D: F09 |
| | M00063627C: F06 |
| | M00063636A: E01 |
| | M00063681B: C02 |
| | M00063682A: C04 |
| | M00063685A: C02 |
| | M00063774A: D09 |
| | M00063784A: H12 |
| | M00063784C: E10 |
| | M00063785C: F03 |
| | M00063795C: D09 |
| | M00063801B: D04 |
| | M00063804C: A11 |
| | M00063805D: E05 |
| | M00063807A: D12 |
| | M00063810C: E03 |
| | M00063852D: F07 |
| | M00063888D: D05 |
| | M00063888D: F02 |
| | M00063890A: F11 |
| | M00063890A: H04 |
| | M00063891A: F11 |
| | M00063892B: G02 |
| | M00063898A: A10 |
| | M00063915C: E01 |
| | M00063919C: E07 |
| | M00063920D: H02 |
| | M00063922B: A12 |
| | M00063925B: F04 |
| | M00063926A: H04 |
| | M00063931B: E10 |
| | M00063931B: F07 |
| | M00063932D: G08 |
| | M00063934C: C10 |
| | M00063938B: H07 |
| | M00063939C: D06 |
| | M00063939C: H01 |
| | M00063940D: F09 |
| | M00063940D: F09 |
| | M00063941B: C12 |
| | M00063943B: G12 |
| | M00063949D: A05 |
| | M00064021D: H01 |
| | M00064025D: E07 |
| | M00064025D: H12 |
| | M00064033C: C11 |
| | M00064033D: B01 |
| | M00063843B: D07 |
| | M00063848C: G11 |
| | M00063852B: D08 |
| | M00063818C: A09 |
| | M00063828A: H12 |
| | M00063828D: E05 |
| | M00063839A: F01 |
| | M00063841A: E08 |
| es83 | M00064043D: C09 |
| | M00064048C: G12 |
| | M00064053B: D09 |
| | M00064057C: H10 |
| | M00064059A: C11 |
| | M00064060B: D03 |
| | M00064079C: A10 |
| | M00064082D: D10 |
| | M00064083D: E05 |
| | M00064086C: E01 |
| | M00064090C: A02 |
| | M00064090D: D09 |
| | M00064105B: A03 |
| | M00064106C: G03 |
| | M00064113B: C04 |
| | M00064115B: E12 |
| | M00064119B: H10 |
| | M00064119C: D12 |
| | M00064122C: B06 |
| | M00064126C: C02 |
| | M00064126C: F12 |
| | M00064136C: D12 |
| | M00064144D: A07 |
| | M00064151B: C07 |
| | M00064159A: H03 |
| | M00064165A: B12 |
| | M00064171D: E05 |
| | M00064171D: E05 |
| | M00064172C: A02 |
| | M00064173B: E01 |
| | M00064176D: H10 |
| | M00064178B: A05 |
| | M00064178B: A05 |
| | M00064180A: G03 |
| | M00064186C: B03 |
| | M00064188B: G08 |
| | M00064194C: D02 |
| | M00064212D: E04 |
| | M00064260C: E05 |
| | M00064268D: G03 |
| | M00064272C: G01 |
| | M00063163A: G04 |
| | M00063165A: C09 |
| | M00063577C: C02 |
| | M00063578B: E02 |
| | M00063578C: A06 |
| | M00063580D: B06 |
| | M00063593A: D03 |
| | M00063600C: C09 |
| | M00063955C: F07 |
| | M00063955D: F05 |
| | M00063956A: F05 |
| | M00063957A: E02 |
| | M00063957A: E02 |
| | M00063967C: A12 |
| | M00063967D: G02 |
| | M00063968D: G08 |
| | M00063972C: E10 |
| | M00063978B: B06 |
| | M00063981D: A06 |
| | M00063990A: D05 |
| | M00063990A: D05 |
| | M00063997C: B12 |
| | M00063998C: E09 |
| | M00064000B: C03 |
| | M00064001A: B03 |
| | M00064005D: A08 |
| | M00064008A: B01 |
| | M00064009A: C01 |
| | M00064014D: H05 |
| | M00064018C: E07 |
| | M00064293D: B12 |
| | M00064294D: F01 |
| | M00063557D: C07 |
| | M00063559D: G03 |
| | M00063571B: G03 |
| | M00063575B: G02 |
| | M00063555B: D01 |
| | M00063533A: C12 |
| | M00063534C: A02 |
| | M00063538D: B01 |
| | M00063539C: C11 |
| es84 | M00064307B: G02 |
| | M00064307C: G03 |
| | M00064310C: A10 |
| | M00064328B: H04 |
| | M00064328B: H09 |
| | M00064337D: F01 |
| | M00064341A: C02 |
| | M00064345A: A03 |
| | M00064346C: B09 |
| | M00064349D: H01 |
| | M00064352C: H01 |
| | M00064354A: A10 |
| | M00064358A: G03 |
| | M00064358C: D09 |
| | M00064375B: G07 |
| | M00064376A: A05 |
| | M00064385D: C11 |
| | M00064386B: C02 |
| | M00064386B: C02 |
| | M00064393B: H04 |
| | M00064399A: E01 |
| | M00064405B: C04 |
| | M00064406B: H06 |
| | M00064414D: D06 |
| | M00064415B: G03 |
| | M00064424B: C12 |
| | M00064428B: A12 |
| | M00064447B: A07 |
| | M00064447B: C06 |
| | M00064450C: E07 |
| | M00064452D: E11 |
| | M00064454A: H10 |
| | M00064454C: B06 |
| | M00064460C: B01 |
| | M00064467B: D06 |
| | M00064481C: F03 |
| | M00064508A: B09 |
| | M00064514D: F11 |
| | M00064517B: F04 |
| | M00064517B: F10 |
| | M00064517C: F11 |
| | M00064564A: C02 |
| | M00064568A: H06 |
| | M00064569B: A09 |
| | M00064569B: A09 |
| | M00064571C: C04 |
| | M0064577C: B120 |
| | M00064579A: C06 |
| | M00064593A: A05 |
| | M00064593D: C01 |
| | M00064601C: G07 |
| | M00064601D: B05 |
| | M00064605C: G05 |
| | M00064610D: H01 |
| | M00064620D: G05 |
| | M00064624C: B03 |
| | M00064631A: C07 |
| | M00064631A: C07 |
| | M00064631C: H11 |
| | M00064636B: A04 |
| | M00064649A: E04 |
| | M00064650B: B07 |
| | M00064652B: D09 |
| | M00064675C: E09 |
| | M00064678D: F05 |
| | M00064693D: F08 |
| | M00064723C: H04 |
| | M00064723D: H03 |
| | M00064723D: H03 |
| | M00003773D: H02 |
| | M00021929A: D03 |
| | M00043134A: A05 |
| | M00064534D: F06 |
| | M00064550A: A07 |
| | M00064554D: A03 |
| | M00064526D: F05 |
| | M00064527A: H07 |
| | M00064530B: H02 |
| | M00064532D: G06 |
| | M00064520A: E04 |
| | M00064520A: E04 |
| | M00064524A: A09 |

TABLE 8

| | Path | | Primary | Primary | | | | Incidence | Regional | | Descrip | | |
|---|---|---|---|---|---|---|---|---|---|---|---|---|---|
| | Report | Anatomical | Tumor | Tumor | Histopath | | Lymphnode | Lymphnode | Lymphnode | Distant | Distant | Dist Met | |
| PatientID | ID | Loc | Size | Grade | Grade | Local Invasion | Met | Met | Grade | Met & Loc | Met | Grade | Comment |
| 15 | 21 | Ascending | 4.0 | T3 | G2 | extending into | positive | 3/8 | N1 | negative | | MX | invasive |
| | | colon | | | | subserosal | | | | | | | adenocarcinoma, |
| | | | | | | adipose tissue | | | | | | | moderately |
| | | | | | | | | | | | | | differentiated; |
| | | | | | | | | | | | | | focal |
| | | | | | | | | | | | | | perineural |
| | | | | | | | | | | | | | invasion is |
| | | | | | | | | | | | | | seen |
| 52 | 71 | Ascending | 9.0 | T3 | G3 | Invasion | negative | 0/12 | N0 | negative | | MO | Hyperplastic |
| | | colon | | | | through | | | | | | | polyp in |
| | | | | | | muscularis | | | | | | | appendix. |
| | | | | | | propria, | | | | | | | |
| | | | | | | subserosal | | | | | | | |
| | | | | | | involvement; | | | | | | | |
| | | | | | | ileocec. valve | | | | | | | |
| | | | | | | involvement | | | | | | | |
| 121 | 140 | Sigmoid | 6 | T4 | G2 | Invasion of | negative | 0/34 | N0 | negative | | M0 | Perineural |
| | | | | | | muscularis | | | | | | | invasion; |
| | | | | | | propria into | | | | | | | donut |
| | | | | | | serosa, | | | | | | | anastomosis |
| | | | | | | involving | | | | | | | negative. |
| | | | | | | submucosa of | | | | | | | One |
| | | | | | | urinary bladder | | | | | | | tubulovillous |
| | | | | | | | | | | | | | and one |
| | | | | | | | | | | | | | tubular |
| | | | | | | | | | | | | | adenoma |
| | | | | | | | | | | | | | with no high |
| | | | | | | | | | | | | | grade |
| | | | | | | | | | | | | | dysplasia. |
| 125 | 144 | Cecum | 6 | T3 | G2 | Invasion | negative | 0/19 | N0 | negative | | M0 | patient |
| | | | | | | through the | | | | | | | history of |
| | | | | | | muscularis | | | | | | | metastatic |
| | | | | | | propria into | | | | | | | melanoma |
| | | | | | | suserosal | | | | | | | |
| | | | | | | adipose tissue. | | | | | | | |
| | | | | | | Ileocecal | | | | | | | |
| | | | | | | junction. | | | | | | | |
| 128 | 147 | Transverse | 5.0 | T3 | G2 | Invasion of | positive | 1/5 | N1 | negative | | M0 | |
| | | colon | | | | muscularis | | | | | | | |
| | | | | | | propria into | | | | | | | |
| | | | | | | percolonic fat | | | | | | | |
| 130 | 149 | Splenic | 5.5 | T3 | | through wall | positive | 10/24 | N2 | negative | | M1 | |
| | | flexure | | | | and into | | | | | | | |
| | | | | | | surrounding | | | | | | | |
| | | | | | | adipose tissue | | | | | | | |
| 133 | 152 | Rectum | 5.0 | T3 | G2 | Invasion | negative | 0/9 | N0 | negative | | M0 | Small |
| | | | | | | through | | | | | | | separate |
| | | | | | | muscularis | | | | | | | tubular |
| | | | | | | propria into | | | | | | | adenoma |
| | | | | | | non- | | | | | | | (0.4 cm) |
| | | | | | | peritonealized | | | | | | | |
| | | | | | | pericolic tissue; | | | | | | | |
| | | | | | | gross | | | | | | | |
| | | | | | | configuration is | | | | | | | |
| | | | | | | annular. | | | | | | | |
| 141 | 160 | Cecum | 5.5 | T3 | G2 | Invasion of | positive | 7/21 | N2 | positive | adenocarcinoma | M1 | Perineural |
| | | | | | | muscularis | | | | (Liver) | consistant | | invasion |
| | | | | | | propria into | | | | | with | | identified |
| | | | | | | pericolonic | | | | | primary | | adjacent to |
| | | | | | | adipose tissue, | | | | | | | metastatic |
| | | | | | | but not through | | | | | | | adenocarcinoma. |
| | | | | | | serosa. Arising | | | | | | | |
| | | | | | | from tubular | | | | | | | |
| | | | | | | adenoma. | | | | | | | |
| 156 | 175 | Hepatic | 3.8 | T3 | G2 | Invasion | positive | 2/13 | N1 | negative | | M0 | Separate |
| | | flexure | | | | through | | | | | | | tubolovillous |
| | | | | | | mucsularis | | | | | | | and tubular |
| | | | | | | propria into | | | | | | | adenomas |
| | | | | | | subserosa/pericolic | | | | | | | |
| | | | | | | adipose, noserosal | | | | | | | |
| | | | | | | involvement. | | | | | | | |
| | | | | | | Gross | | | | | | | |
| | | | | | | configuration | | | | | | | |
| | | | | | | annular. | | | | | | | |
| 228 | 247 | Rectum | 5.8 | T3 | G2 to G3 | Invasion | positive | 1/8 | N1 | negative | | MX | Hyperplastic |
| | | | | | | through | | | | | | | polyps |
| | | | | | | muscularis | | | | | | | |
| | | | | | | propria to | | | | | | | |
| | | | | | | involve | | | | | | | |
| | | | | | | subserosal, | | | | | | | |
| | | | | | | perirectoal | | | | | | | |
| | | | | | | adipose, and | | | | | | | |
| | | | | | | serosa | | | | | | | |
| 264 | 283 | Ascending | 5.5 | T3 | G2 | Invasion | negative | 0/10 | N0 | negative | | M0 | Tubulovillous |
| | | colon | | | | through | | | | | | | adenoma |
| | | | | | | muscularis | | | | | | | with high |
| | | | | | | propria into | | | | | | | grade |
| | | | | | | subserosal | | | | | | | dysplasia |
| | | | | | | adipose tissue. | | | | | | | |
| 266 | 285 | Transverse | 9 | T3 | G2 | Invades | negative | 0/15 | N1 | positive | 0.4 cm, | MX | |
| | | colon | | | | through | | | | (Mesenteric | may | | |
| | | | | | | muscularis | | | | deposit | represent | | |
| | | | | | | propria to | | | | | lymphnode | | |
| | | | | | | involve | | | | | completely | | |
| | | | | | | pericolonic | | | | | replaced | | |
| | | | | | | adipose, | | | | | by | | |
| | | | | | | extends to | | | | | tumor | | |
| | | | | | | serosa. | | | | | | | |
| 268 | 287 | Cecum | 6.5 | T2 | G2 | Invades full | negative | 0/12 | N0 | negative | | M0 | |
| | | | | | | thickness of | | | | | | | |
| | | | | | | muscularis | | | | | | | |
| | | | | | | propria, but | | | | | | | |
| | | | | | | mesenteric | | | | | | | |
| | | | | | | adipose free of | | | | | | | |
| | | | | | | malignancy | | | | | | | |
| 278 | 297 | Rectum | 4 | T3 | G2 | Invasion into | positive | 7/10 | N2 | negative | | M0 | Descending |
| | | | | | | perirectal | | | | | | | colon |
| | | | | | | adipose tissue. | | | | | | | polyps, no |
| | | | | | | | | | | | | | HGD or |
| | | | | | | | | | | | | | carcinoma |
| | | | | | | | | | | | | | identified. |
| 295 | 314 | Ascending | 5.0 | T3 | G2 | Invasion | negative | 0/12 | N0 | negative | | M0 | Melanosis |
| | | colon | | | | through | | | | | | | coli and |
| | | | | | | muscularis | | | | | | | diverticular |
| | | | | | | propria into | | | | | | | disease. |
| | | | | | | percolic | | | | | | | |
| | | | | | | adipose tissue. | | | | | | | |
| 339 | 358 | Rectosigmoid | 6 | T3 | G2 | Extends into | negative | 0/6 | N0 | negative | | M0 | 1 |
| | | | | | | perirectal fat | | | | | | | hyperplastic |
| | | | | | | but does not | | | | | | | polyp |
| | | | | | | reach serosa | | | | | | | identified |
| 341 | 360 | Ascending | 2 cm | T3 | G2 | Invasion | negative | 0/4 | N0 | negative | | MX | |
| | | colon | invasive | | | through | | | | | | | |
| | | | | | | muscularis | | | | | | | |
| | | | | | | propria to | | | | | | | |
| | | | | | | involve | | | | | | | |
| | | | | | | pericolonic fat. | | | | | | | |
| | | | | | | Arising from | | | | | | | |
| | | | | | | villous | | | | | | | |
| | | | | | | adenoma. | | | | | | | |
| 356 | 375 | Sigmoid | 6.5 | T3 | G2 | Through colon | negative | 0/4 | N0 | negative | | M0 | |
| | | | | | | wall into | | | | | | | |
| | | | | | | subserosal | | | | | | | |
| | | | | | | adipose tissue. | | | | | | | |
| | | | | | | No serosal | | | | | | | |
| | | | | | | spread seen. | | | | | | | |
| 360 | 412 | Ascending | 4.3 | T3 | G2 | Invasion thru | positive | 1/5 | N1 | negative | | M0 | Two |
| | | colon | | | | muscularis | | | | | | | mucosal |
| | | | | | | propria to | | | | | | | polyps |
| | | | | | | pericolonic fat | | | | | | | |
| 392 | 444 | Ascending | 2 | T3 | G2 | Invasion | positive | 1/6 | N1 | positive | Macrovesicular | M1 | Tumor |
| | | colon | | | | through | | | | (Liver) | and | | arising at |
| | | | | | | muscularis | | | | | microvesicular | | priorileocolic |
| | | | | | | propria into | | | | | steatosis | | surgical |
| | | | | | | subserosal | | | | | | | anastomosis. |
| | | | | | | adipose tissue, | | | | | | | |
| | | | | | | not serosa. | | | | | | | |
| 393 | 445 | Cecum | 6.0 | T3 | G2 | Cecum, invades | negative | 0/21 | N0 | negative | | M0 | |
| | | | | | | through | | | | | | | |
| | | | | | | muscularis | | | | | | | |
| | | | | | | propria to | | | | | | | |
| | | | | | | involve | | | | | | | |
| | | | | | | subserosal | | | | | | | |
| | | | | | | adipose tissue | | | | | | | |
| | | | | | | but not serosa. | | | | | | | |
| 413 | 465 | Ascending | 4.8 | T3 | G2 | Invasive | negative | 0/7 | N0 | positive | adenocarcinoma | M1 | rediagnosis |
| | | colon | | | | through | | | | (Liver) | in | | of |
| | | | | | | muscularis to | | | | | multiple | | oophorecto |
| | | | | | | involve | | | | | slides | | my path to |
| | | | | | | periserosal fat; | | | | | | | metastatic |
| | | | | | | abutting | | | | | | | colon |
| | | | | | | ileocecal | | | | | | | cancer. |
| | | | | | | junction. | | | | | | | |
| 505 | 383 | | 7.5 cm | T3 | G2 | Invasion | positive | 2/17 | N1 | positive | moderately | M1 | Anatomical |
| | | | max dim | | | through | | | | (Liver) | differentiated | | location of |
| | | | | | | muscularis | | | | | adenocarcinoma, | | primary not |
| | | | | | | propria | | | | | consistant | | notated in |
| | | | | | | involving | | | | | with | | report. |
| | | | | | | pericolic | | | | | primary | | Evidence of |
| | | | | | | adipose, serosal | | | | | | | chronic |
| | | | | | | surface | | | | | | | colitis. |
| | | | | | | uninvolved | | | | | | | |
| 517 | 395 | Sigmoid | 3 | T3 | G2 | penetrates | positive | 6/6 | N2 | negative | | M0 | No mention |
| | | | | | | muscularis | | | | | | | of distant |
| | | | | | | propria, | | | | | | | met in report |
| | | | | | | involves | | | | | | | |
| | | | | | | pericolonic fat. | | | | | | | |
| 534 | 553 | Ascending | 12 | T3 | G3 | Invasion | negative | 0/8 | N0 | negative | | M0 | Omentum |
| | | colon | | | | through the | | | | | | | with fibrosis |
| | | | | | | muscularis | | | | | | | and fat |
| | | | | | | propria | | | | | | | necrosis. |
| | | | | | | involving | | | | | | | Small bowel |
| | | | | | | pericolic fat. | | | | | | | with acute |
| | | | | | | Serosa free of | | | | | | | and chronic |
| | | | | | | tumor. | | | | | | | serositis, |
| | | | | | | | | | | | | | focal |
| | | | | | | | | | | | | | abscess and |
| | | | | | | | | | | | | | adhesions. |
| 546 | 565 | Ascending | 5.5 | T3 | G2 | Invasion | positive | 6/12 | N2 | positive | metastatic | M1 | |
| | | colon | | | | through | | | | (Liver) | adenocarcinoma | | |
| | | | | | | muscularis | | | | | | | |
| | | | | | | propria | | | | | | | |
| | | | | | | extensively | | | | | | | |
| | | | | | | through | | | | | | | |
| | | | | | | submucosal and | | | | | | | |
| | | | | | | extending to | | | | | | | |
| | | | | | | serosa. | | | | | | | |
| 577 | 596 | Cecum | 11.5 | T3 | G2 | Invasion | negative | 0/58 | N0 | negative | | M0 | Appendix |
| | | | | | | through the | | | | | | | dilated and |
| | | | | | | bowel wall, | | | | | | | fibrotic, but |
| | | | | | | into suberosal | | | | | | | not involved |
| | | | | | | adipose. | | | | | | | by tumor |
| | | | | | | Serosal surface | | | | | | | |
| | | | | | | free of tumor. | | | | | | | |
| 695 | 714 | Cecum | 14 | T3 | G2 | extending | negative | 0/22 | N0 | negative | | MX | tubular |
| | | | | | | through bowel | | | | | | | adenoma |
| | | | | | | wall into | | | | | | | and |
| | | | | | | serosal fat | | | | | | | hyperplstic |
| | | | | | | | | | | | | | polyps |
| | | | | | | | | | | | | | present, |
| | | | | | | | | | | | | | moderately |
| | | | | | | | | | | | | | differentiated |
| | | | | | | | | | | | | | adenoma |
| | | | | | | | | | | | | | with |
| | | | | | | | | | | | | | mucinous |
| | | | | | | | | | | | | | diferentiation |
| | | | | | | | | | | | | | (% not |
| | | | | | | | | | | | | | stated) |
| 784 | 803 | Ascending | 3.5 | T3 | G3 | through | positive | 5/17 | N2 | positive | | M1 | invasive |
| | | colon | | | | muscularis | | | | (Liver) | | | poorly |
| | | | | | | propria into | | | | | | | differentiated |
| | | | | | | pericolic soft | | | | | | | adenosquamous |
| | | | | | | tissues | | | | | | | carcinoma |
| 786 | 805 | Descending | 9.5 | T3 | G2 | through | negative | 0/12 | N0 | positive | | M1 | moderately |
| | | colon | | | | muscularis | | | | (Liver) | | | differentiated |
| | | | | | | propria into | | | | | | | invasive |
| | | | | | | pericolic fat, | | | | | | | adenocarcinoma |
| | | | | | | but not at | | | | | | | |
| | | | | | | serosal surface | | | | | | | |
| 791 | 810 | Ascending | 5.8 | T3 | G3 | through the | positive | 13/25 | N2 | positive | | M1 | poorly |
| | | colon | | | | muscularis | | | | (Liver) | | | differentiated |
| | | | | | | propria into | | | | | | | invasive |
| | | | | | | pericolic fat | | | | | | | colonic |
| | | | | | | | | | | | | | adenocarcinoma |
| 888 | 908 | Ascending | 2.0 | T2 | G1 | into muscularis | positive | 3/21 | N0 | positive | | M1 | well-to |
| | | colon | | | | propria | | | | (Liver) | | | moderately- |
| | | | | | | | | | | | | | differentiated |
| | | | | | | | | | | | | | adenocarcinoma; |
| | | | | | | | | | | | | | this |
| | | | | | | | | | | | | | patient has |
| | | | | | | | | | | | | | tumors of |
| | | | | | | | | | | | | | the |
| | | | | | | | | | | | | | ascending. |
| | | | | | | | | | | | | | colon and |
| | | | | | | | | | | | | | the sigmoid |
| | | | | | | | | | | | | | colon |
| 889 | 909 | Cecum | 4.8 | T3 | G2 | through | positive | 1/4 | N1 | positive | | M1 | moderately |
| | | | | | | muscularis | | | | (Liver) | | | differentiated |
| | | | | | | propria int | | | | | | | adenocarcinoma |
| | | | | | | subserosal | | | | | | | |
| | | | | | | tissue | | | | | | | |

We claim:

1. An isolated polynucleotide comprising a nucleotide sequence which hybridizes under stringent conditions to a sequence selected from the group consisting of SEQ ID NOS: 1-316.

2. An isolated polynucleotide comprising at least 15 contiguous nucleotides of a nucleotide sequence having at least 90% sequence identity to a sequence selected from the group consisting of:
SEQ ID NOS:1-316, a degenerate variant of SEQ ID NOS:1-316, an antisense of SEQ ID NOS:1-316, and a complement of SEQ ID NOS:1-316.

3. A polynucleotide comprising a nucleotide sequence of an insert contained in a clone deposited as clone number xx of ATCC Deposit Number xx.

4. An isolated cDNA obtained by the process of amplification using a polynucleotide comprising at least 15 contiguous nucleotides of a nucleotide sequence of a sequence selected from the group consisting of SEQ ID NOS:1-316.

5. The isolated cDNA of claim 4, wherein amplification is by polymerase chain reaction (PCR) amplification.

6. An isolated recombinant host cell containing the polynucleotide according to claims 1, 2, 3, or 4.

7. An isolated vector comprising the polynucleotide according to claims 1, 2, 3, or 4.

8. A method for producing a polypeptide, the method comprising the steps of:
culturing a recombinant host cell containing the polynucleotide according to claims 1, 2, 3, or 4, said culturing being under conditions suitable for the expression of an encoded polypeptide;
recovering the polypeptide from the host cell culture.

9. An isolated polypeptide encoded by the polynucleotide according to claims 1, 2, 3, or 4.

10. An antibody that specifically binds the polypeptide of claim 9.

11. A method of detecting differentially expressed genes correlated with a cancerous state of a mammalian cell, the method comprising the step of:
detecting at least one differentially expressed gene product in a test sample derived from a cell suspected of being cancerous, where the gene product is encoded by a gene comprising an identifying sequence of at least one of SEQ ID NOS:1-316;
wherein detection of the differentially expressed gene product is correlated with a cancerous state of the cell from which the test sample was derived.

12. A library of polynucleotides, wherein at least one of the polynucleotides comprises the sequence information of the polynucleotide according to claims 1, 2, 3, or 4

13. The library of claim 12, wherein the library is provided on a nucleic acid array.

14. The library of claim 12, wherein the library is provided in a computer-readable format.

15. A method of inhibiting tumor growth by modulating expression of a gene product, the gene product being encoded by a gene identified by a sequence selected from the group consisting of SEQ ID NOS:1-316.


SEQUENCE LISTING (324 sequences)

SEQ ID NO: 1 - DNA, 214 bp, Homo sapiens
ttagtactgcatatgtaaatactaccttttcaatgagctatataaacaatgatagcacatccttccttttactatgtctcacctcctttaggagagaacttccttaagtaagtgctaaacatacatatacggaacttgaaagctttggttagccttgccttaggtaatcagactagtttacactgtttccagggagtagttgaattactataag

SEQ ID NO: 2 - DNA, 353 bp, Homo sapiens
  misc_feature (1)...(353): n = A,T,C or G
ggcacgaggagagaactagaaaatatgtatattggatatactatgtgccaggcacgattccaagcccctgatacattctctannnnnnnnnnnnnnnnnnnnnnnnnnnnnnnnnnnnnnnnnnnnnnnnnnnnnnnnnnnnaaagtggaacactgggatttgaacaaggttttggttgggcatcttttcctatgggagctcagaaatatctgttgtctagccctttctcagcctcccaaccttctcggttccttacctatgtcacagctgactttgagctaaagtcatctcggggcagctaggtgcctatgtgagctggcgttcatttctcactgtttctccttccaaatac

SEQ ID NO: 3 - DNA, 399 bp, Homo sapiens
ggcacgagcccaccaagagctgcatagagcacgtttagctagagtaggagtttgcagtgctcatatgggaaatgctgctgctatacttttaggaatttctgagtgcaatttagaaacatctagcacacttgaaacactgcgtatcattttcctcactcatgaatatagtcatcagaattcataaatagtttacctgagccctttaacaacctcaaataggccatatttctctctctggttgatggcatggaccctacaggaaaaaccacaccttaccgcttctgaccagcatcactacaaaaaggagtgctgaagccaatcaccatgtaagcaagataaaagcaaagggggtcttgcctgcccatctctgttccatacattcttaccaggcactgagag

SEQ ID NO: 4 - DNA, 389 bp, Homo sapiens
  misc_feature (1)...(389): n = A,T,C or G
ggcacgaggagagggtggtgggtccctgagttggtggaaagggatagagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagtagaggatgcctggtatgaggcaatatttgggatagggaagggaagcttgggattttagctacgtagagacacttgaaaattggagggaggaaaggagtgggtggctttggagn

SEQ ID NO: 5 - DNA, 279 bp, Homo sapiens
  misc_feature (1)...(279): n = A,T,C or G
cctctccccttggaacccaaagaggaacggggccgaactttataaactttaggcaagggcaaagggcgtgnnnnnnnnnnnnggggccaaggggcatttcccaagcgattaaaatttgggaacctttggttacaaaaattgcggggaaaatttatttcgggagcaattttccctttaaaaatttgagaattcttacccggagagtgtgacataatttaaggcgcctctgcccaaagaggccatgtgcgtgaggggaataccgcgtttaattatcacaaa

SEQ ID NO: 6 - DNA, 388 bp, Homo sapiens
ggcacgaggcagaggcctccctgcactggtcctggcctcactcttttccctgacccttggggcccagggccatggagggacccttaggagttcaatgagagagaccatgaggccactgggctttccccttcccaggcctcctgggtgccacccccttacgttattcttgggcctctaataagtgtcccacaggtgcctggccaggcccacctgctgcagatgtggtctgtgtgtgtgcatgtgtgggtgtgtgtgggcacaggtgtgagtgtgtgagcaacagtaccccattccagtcgtttcctgctgtgactaagtcagcaacacagttcctctgacatgggccttggctgtgcttctttgggggtgaagagattgcggaggaagt

SEQ ID NO: 7 - DNA, 410 bp, Homo sapiens
  misc_feature (1)...(410): n = A,T,C or G
ggcacgaggggaagtcgcgcatgcgcgagtgtacgcgttgccggcgaagaggggagcctgacgactcggaaatttgaataccacagtagcatggagtgtgacctcatggagactgacatcttggagtcgttggaagatctaggttacaagggcccattgttggaagatggagcgctctctcaggcagtctctgctggagccagttcccccgagtttaccaaactctgtgcttggctggtgtctgaattaagagtgctctgtaaactagaggaaaacgtgcaagcaactaacagtccgagtgaagctgaagaattccagcttgaggtgagtgggctactaggggagatgaactgcccgtatctttcactgacatctgnngatgtgaccaagcgccttctcattcagaaaaa

SEQ ID NO: 8 - DNA, 229 bp, Homo sapiens
ctaacaaaaaacactaaaaaaaaataaaagaaattaattgaaactgacctaactcgtggcagggggaactcggctataagacccacaaaccctgctgactcataacaaactgagttgtaagacattcatcgccgcgatatccttgagtaaagaatgaactctggaagcccacccacggacaatgcaccttcacaaagattctgcactaatctgagtgaaggtctttggt

SEQ ID NO: 9 - DNA, 380 bp, Homo sapiens
  misc_feature (1)...(380): n = A,T,C or G
ggcacgagagtagttgggaaatcttttataaatccacctattactacctattggtaggggagattaaatttctacaggtatggagagtcggcttgactacactgtgtggagcaagttttaaagaagcaaaggtatagcagttccaagtannnnnnnnnnnnnnnnagaccaaactctagatcttgcccaaaatggacggccgcggcatttaaatgaagaaagatttatttttccttttttcttttaagaaaaatttttttaaaaaattttgattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 10 - DNA, 317 bp, Homo sapiens
  misc_feature (1)...(317): n = A,T,C or G
cacacacacacatccactctctctttttgctctcttctcacacacacatatctcccttactcacacactctctctcacacaccccctctttcttttcccccgcactttctttctctcacgcgcgcgcgcactcactctcttttttcttctctctctcactctctctctccgcgcgctctctcacacgctttatatctctctctctgagggacttctctctcctctcactcttatttttttgttgtgttttatagcgtctctctcttccctnnnnnnnnnnntctatatatacagagagagatctctctgctctctcc

SEQ ID NO: 11 - DNA, 391 bp, Homo sapiens
ggcacgagagaattagctgaaacccaccaagagctgcatagagcacgtttagctagagtaggagtttgcagtgctcatatgggaaatgctgctgctatacttttaggaatttctgagtgcaatttagaaacatctagcacacttgaaacactgcgtatcattttcctcactcatgaatatagtcatcagaattcataaatagtttacctgagccctttaacaacctcaaataggccatatttctctctctggttgatggcatggaccctacaggaaaaaccacaccttaccgcttctgaccagcatcactacaaaaaggagtgctgaagccaatcaccatgtaagcaagataaaagcaaagggggtcttgcctgcccatctctgttccata

SEQ ID NO: 12 - DNA, 280 bp, Homo sapiens
  misc_feature (1)...(280): n = A,T,C or G
tgtgcgcgcccccccggggcgctctctctctacactcgtgcgctcccccctctgtctgtctctctctctagagtcacggtctcctacacggcgcgcacatgcgaggggcactnnnnnnnnnnngctcnnnnnnnnnnnnnnnnnnnnnnncgnnnnnnnnnnnnnnntcccttgtatactctctgtgtgcgcggggacannnnnnnnnnnnngtgcgcgcgcgagagcgcgcgcgccacacaagagagagcgcgccctnnnnnnnnnnnnnaccgcgaac

SEQ ID NO: 13 - DNA, 311 bp, Homo sapiens
cgcttttttgggaacccaaaccttttttggctggccggaaaaaatttccacgggaagggtaaaggggtttattaatttttttggcaaaacaggggttaagaaaccttccctcccggcctaagggtgggctaggctttggaaaggctaaaagggggaaatttctggcccttgttccaagggaaacatgggctagggggaaaccccaccccttcagggccctttaaaagggcccccaaaaaaagaacccctttattaagggttaaaaaaggttaaaaaaggtgggaacctcatgggccaaggcaaatttttgt

SEQ ID NO: 14 - DNA, 387 bp, Homo sapiens
  misc_feature (1)...(387): n = A,T,C or G
ggcacgaggtcttttctgcccacatctcacacaattgaggtgtctgaacaagcttggggagggtctataaggggtaggctcnnnnnnnnnnnncccatttggaaagggcgttttgccaacccaagggcttttttaagccgatttttnnnnnnnnnnccggacttggtaattggcttttggctttttaaagcccaaaaaataataattaaggggcccaaaataaggaagggcaaaaaaagcctttactccccctgcctttcaaaaagaaaaggaaaaaccggcccccccttaataattggcacccctaaaaaaaggggttttaaaaaaagccaaaaacaaaagggcctggaaaaaaattttgactttttttaacccggaacctgggaa

SEQ ID NO: 15 - DNA, 273 bp, Homo sapiens
  misc_feature (1)...(273): n = A,T,C or G
ctgtctctctctctccccccctctccctcccgcgcgcgcacgctctttcatctctctctctacagacaggggggggtgttctctctccctctcgagagggaccgctttttttttctccccctctctcacactcggggtgtgcgcgctccctttgggggcttttctatagggcgcgctctaaagaaagcccgcctttctcctctgggtgcctcctcccacacccgggttttctcccccgctgtttttgaagaaactcctcctggtctccttatn

SEQ ID NO: 16 - DNA, 283 bp, Homo sapiens
  misc_feature (1)...(283): n = A,T,C or G
ctctctctctggccccccccctctctttacacacactttctctcctctctctcgctctctctttttttttctctctcccctcgctctctctgtgtgtctctatctcgtgtctctctctgcgtgtccctcacacacactcgcgcgagagatctctctctatatctctcctttgtctgtgtctctctctcgcgcgcccacacatctatatatttttgcgcgcacacgcgagagtgtgtccctctctctctctgcacnnnnnnnnnnnnnnnncacaccctccccc

SEQ ID NO: 17 - DNA, 392 bp, Homo sapiens
  misc_feature (1)...(392): n = A,T,C or G
ggcacgagatgactccttccttaaaatccagctcaaatctccccccttttggtggctttctctgacactccatcataaagctaattgtttaagtatgatccagtggcacagtttattcctacttcataacttttatctcactatgttgtaagatattaggtatgtttcttctactaccagtaattttcaaagagttaaggaagaaggatagaagacagcagtataggtgaatgtgtgcatgtgttnnnnnnnnnnnnngccatattggccaaaatttttggactggctggtaaaacaaaggcttttcaaattttcaaatacctttaaaaaaaacctggaaattgttttgtnnnnnnnnnncgcccaaaaaaaaattttgggcctggggggga

SEQ ID NO: 18 - DNA, 385 bp, Homo sapiens
ggcacgaggcagaggcctccctgcactggtcctggcctcactcttttccctgacccttggggcccagggccatggagggacccttaggagttcaatgagagagaccatgaggccactgggctttccccttcccaggcctcctgggtgccacccccttacgttattcttgggcctctaataagtgtcccacaggtgcctggccaggcccacctgctgcagatgtggtctgtgtgtgtgcatgtgtgggtgtgtgtgggcacaggcgtgagtgtgtgagcaacagtaccccattccagtcgtttcctgctgtgactaagtcagcaacacagttcctctgacatgggccttggctgtgcttctttgggggtgaagagattgctgaggc

SEQ ID NO: 19 - DNA, 383 bp, Homo sapiens
gaaggcttgcggagagaaaaccctggagccatcttcataggaagaggaaaggaaactgtatgacaggagaatgaatcaagtttggggctcaaggtgccggccactgggaaaaacagctgccccgagttgcaaaactctgggtcctatatgtataaactatgccctgaggaaggaatctcaggcgtatcttaggagaaaatgttctagcttgggaaacaaacacaacaggaccgtgaatccaaatatttcaagtgggtttagaggactggagttctaaacgctgcttttactgtaagtgatcacgccccggaatgtgctgaagaaaggaaaatgagccagtatcggcgaggactatgggcaaggaaaacgagagtgtgcgatgt

SEQ ID NO: 20 - DNA, 313 bp, Homo sapiens
ctctcccccgcgctcttgagatatgcgcgccccttttttcttctacacgggggggggcgcgcctctttttctcgcgcgcccccctctctctcttttgtgcgcacgcgcgcgcgcgggggggttctttttttgtgcggagagagagtctgtctcaggggtttttttgttttctttcacgacacacactttctcccctgtgcatgtgttttgatgctctctcgagatatgtctctctctctctgtgtgtgtgtgttgtgcgccccccctggggagagcgctcttctctctctcctcatatagcgcgcgcgcgcga

SEQ ID NO: 21 - DNA, 396 bp, Homo sapiens
ggcacgaggggacccccttcacctctgtctagagagctgggtagatcagaaacttggtgacacctggctagcacagagcaggctcacttgtcttggtcccactacccagattcctgcagacattgcaaaccaaatgaaggttgttgaatgacccctgtccccagccacttgttttgttatcatctgctctgcagtggaatgcctgtgtgtttgagttcactctgcatctgtatatttgagtatagaaaccgagtcaagtgatcatgtgcatccagacacactgtgtcacctgagccacagagcaaatcaccttaacgatctggaatgaaactgtgaccagtgccgccctgggtggttctggagagactgccgtcttcttgtttggccataggtgcg

SEQ ID NO: 22 - DNA, 310 bp, Homo sapiens
  misc_feature (1)...(310): n = A,T,C or G
tgaatatcagggccctgaacatctctcacgcccgtcttctaaaagagaagaaaaaaacgcgcgcgggctttttctctctctcagaggggtgaaacacacaatatctcggggggccgggggagagcccgctctctctgcctgtaaaacacacagaagtgcgctcacgccctgcgcgggagcccacagacttttttttaaaacaaaaagtatattggggtgtgttttaatctccctctccgctcctagagggggggcgnnnnnnnnnnnnnnntttttaaataggggggcccgagtctcacccaatagaagg

SEQ ID NO: 23 - DNA, 375 bp, Homo sapiens
ggcacgagccggcgaagaggggagcctgacgactcggaaatttgaataccacagtagcatggagtgtgacctcatggagactgacatcttggagtcgttggaagatctaggttacaagggcccattgttggaagatggagcgctctctcaggcagtctctgctggagccagttcccccgagtttaccaaactctgtgcttggctggtgtctgaattaagagtgctctgtaaactagaggaaaacgtgcaagcaactaacagtccgagtgaagctgaagaattccagcttgaggtgagtgggctactaggggagatgaactgcccgtatctttcactgacatctggggatgtgaccaagcgccttctcattcagaa

SEQ ID NO: 24 - DNA, 477 bp, Homo sapiens
  misc_feature (1)...(477): n = A,T,C or G
gctccttcttcttnttgttgatcccatcgatccgaattcggcacgagagcacctctgtgcctctctgagagcactcacagccaaaagtacacagctgcccccaggctgagagtgcttgatacacccttgaatcccctcttatatgatgccccagcccaggagagataaaagcatcagcaccatgagattcacctgcctctggtcgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactcttagacagcaaaaatgctttctcccagtcttgttcccttgttctcagttcccaccctgcctggataactactgttcttggtttnnnnnnnnnnnnnnnnnnnnnnnnagtctcgtaccagattcataaatcagccg

SEQ ID NO: 25 - DNA, 265 bp, Homo sapiens
cgcgcgggggggacccctctctctctctctgttgcgcgcgctctctcaccccgtgtgtcgcccccgatattgtcagagagaccccctatttttttctcccgccccacacacatctatgtgtaaaatgtgcgtgtctgtcgcgcacacccacacactctccccggggggtttataaaatactcgcgcgctatattttcgcccccctttttgtgtgtgggcgccacaaaaacaccacacgctctcccccctgtctctcgcgggtgtt

SEQ ID NO: 26 - DNA, 388 bp, Homo sapiens
  misc_feature (1)...(388): n = A,T,C or G
ggcacgagggaggctctttgttatagatgcttttgcccccttaatacagcaatgagagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaggcctttgtgaagaattttcatcaaggcatctgtagagatcagtgagcccaaaattaaagttttcagatgaaacaacaaaacttgtcaagctgactn

SEQ ID NO: 27 - DNA, 431 bp, Homo sapiens
  misc_feature (1)...(431): n = A,T,C or G
ggcacgagagaggggctactttagatgcaaaggggacaattagaaggctactgaggtaatccggacaaaaagttgtaaataaatcacggtggcagtatggtgaatagtggaaggggtgtatttgaagaaactggggaggccgtgggagaggctggctagtgagaaatgggccgaaggtgaaagcagcttaggggctggtttccagttttctggcactgcagactgggtagtgggaggtggctttctcaagaggagaggtgagtgggaaggagcagggctgcaggggaggtcatggtcttgggagtggtgctcagtctgacttgcacataggggagattattttagatttccgcaagaaaatgtccagcatgtagtcatatcaatgnnnnnnnnnnnnnnnnnnnnnnnnnnntgagatttacccaaaaaga

SEQ ID NO: 28 - DNA, 389 bp, Homo sapiens
ggcacgagccacccccaagagtgtggccatctggggccgtgtggtatttgccactcaggagacatgtccctatgacatagcagtggtgagcctggaggaggacctggatgatgtccccatccctgtgcccgctgagcacttccatgaaggcgaggctgtgagtgtggtgggctttggcgtctttggccagtcttgcgggccctcggtgacctcaggcatcctttccgctgtggtgcaggtgaatggcacgcccgtaatgctgcagaccacgtgtgctgtgcacagcggctccagtgggggacccctcttctccaaccactcaggaaacctccttggcataatcaccagcaacacccgggacaataatacgggggccacctacccccacc

SEQ ID NO: 29 - DNA, 431 bp, Homo sapiens
ggacgaggctccagcgcacttttccaacacatcactgcattatttgaatgcaccatggcagctattgtcaccttacttgggagtgatccagttggagctctttatattcggacatgtcgagtattgatgctttctgactgggacacgatgctttacaacccaaggccagattacggtaccacagtgcactgtactcatgaagccggctacccactatataccatcgtatttatctattacgcattctgcttggtattaatgatgctgctccgacctcttctggtgaagaagaatgcatgtgggttagggaaatctgatcgatataaaagtatttatgctgcactttacttcttcccaaatgtaaccgtgcttcaggcagctgtgggaggccttttatattacgccttcccatacattatattagtggtatc

SEQ ID NO: 30 - DNA, 393 bp, Homo sapiens
ggcacgagactacacccgcttcgatgactggtacctgtgggttcagatgtacaaggggactgtgtccatgccagtcttccagtccttggaggcctactggcctggtcttcagagcctcattggagacattgacaatgccatgaggaccttcctcaactactacactgtatggaagcagtttggggggctcccggaattctacaacattcctcagggatacacagtggagaagcgagagggctacccacttcggccagaacttattgaaagcgcaatgtacctctaccgtgccacgggggatcccaccctcctagaactcggaagagatgctgtggaatccattgaaaaaatcagcaaggtggagtgcggatttgcaacaatcaaagatctgcg

SEQ ID NO: 31 - DNA, 459 bp, Homo sapiens
  misc_feature (1)...(459): n = A,T,C or G
gcaatcgcattgtctttttgaggatnnnatnaatgtcaattcggcacgagctttgtggatgtttccagctgccatcgtcacccttctgtctgctccctggaccagcttcaggacttgaaggccctcgtggctgagatcatcacacatttgcaggggctgcagagggacttatctctagcagtctcctacagcaggctccattcctcagactggaatctgtgtactgtatttgggatcctcctgggctatcctgttccctatacctttcacctgaaccagggagatgacaactgcttagctctgactccactacgagtattcactgcccggatctcatggttgctaggtcaacccccaatcctgctctattcttttagtgtcccagagagtttgttcccacgcctgagggacattctgaacacctgggagaaagacctcagaacccgatttatgactcac

SEQ ID NO: 32 - DNA, 445 bp, Homo sapiens
  misc_feature (1)...(445): n = A,T,C or G
ggcacgagatggagagcacctctgtgcctctctgagagcactcacagccaaaagtacacagctgcccccaggctgagagtgcttgatacacccttgaatcccctcttatatgatgccccagcccaggagagataaaagcatcagcaccatgagattcacctgcctctggtcgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactcttagacagcaaaaatgctnttctccagtctttgttccttggtctcaaggtccacccttgctggataactactggtcttgggttcctggggtaaagatggaacttgagtaagctcgacccaaatccaaaatcaatccg

SEQ ID NO: 33 - DNA, 429 bp, Homo sapiens
  misc_feature (1)...(429): n = A,T,C or G
ggcacgagcgcctgccctgcatcagggagacatgtcagctgaggagtaattgaccagatttctgctttagaaatatggcagtggaggcaggagatggcatctgaggcccaggctggggagaagggtgctgggatgagaacctggagttcagaccagggaagggatgagagcctaagaagaggagctctcaccctgagacaggctggtgcaggagtctgctcgatccaggcctgggtccctggttccctctgagcttgggaggactatgtgagacagaacaggaccaggggcctgcattcccccttgtattattcatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgatggccc

SEQ ID NO: 34 - DNA, 439 bp, Homo sapiens
  misc_feature (1)...(439): n = A,T,C or G
gttctgtgggaatagagggtccctggtgacagggcagggctagatctggagcctgcacttggcctgtgacatactgtcttgtttctgagaatcctcccctacttctctagttaatctccagagacttctgtgactacttaatcacaaaggaaattttcaggaatattatcaaatactattttagaaaaaaaaagagaagggatttgaatgttttcagttcagtttagntatcnnnnnnnnnnnnnnnccccaaacttcaaaatggaggcccccccctcctttaacccccctaaaaaaaattctgatgtttgaggtttggttgccaattaaccaaacccccaaaaaaaaagggggttaaaccccattggaaagttttcctaattttggggggtgccctttgaggtggacccggttccctgccctgggaaaggccccaaag

SEQ ID NO: 35 - DNA, 440 bp, Homo sapiens
  misc_feature (1)...(440): n = A,T,C or G
ggcacgaggtgaagtcctggttccagactcccctttttgccgggacatgatggatctgtcagctggtgcctatagtcctagagagctagagatggagggaaattcagatcatctaaacccttcagcccttcactggacagaagaggaaactgaggctccatctgcatgacgttcccagagtcacggcacaaattcatggaagaagcagcaggaaactcagttctccagtctgggtccaatgtgtgttttagaaatatctccacagggttaatgactcaatttttcatgcatgattgctagtaatgacaatcatgttatgtttgtttctgtagctttggaaatcactccttccacttgagtttcaggtcccaactgtccacacctgcaggagtgaggttttgctgagactgataaggcactcacattntgtgggagttgaa

SEQ ID NO: 36 - DNA, 423 bp, Homo sapiens
  misc_feature (1)...(423): n = A,T,C or G
acgagcgcnnnncctgcatcagggagacatgtcagctgaggagtaattgaccagatttctgctttagaaatatggcagtggaggcaggagatggcatctgaggcccaggctggggagaagggtgctgggatgagaacctggagttcagaccagggaagggatgagagcctaagaagaggagctctcaccctgagacaggctggtgcaggagtctgctcgatccaggcctgggtccctggttccctctgagcttgggaggactatgtgagacagaacaggaccaggggcctgcattcccccttgtattattcatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgatgg

SEQ ID NO: 37 - DNA, 424 bp, Homo sapiens
  misc_feature (1)...(424): n = A,T,C or G
ggcttgtaganctcggaggttngcaagaatcgcattcggcacgagctgggacacagtgngctctcttatatttgttgctggaataaatgaatgaactaaggcagtcttgtagggatttactgttaaccaccatgggaaaattaaataaatgcggggaaggaaaacgttctaaaattagaagactactttctactctcagcttctgattccctctgagctaagaaccagacagccttaggctggtaactcctataagctggtcctcctcccatgctgaccccatctttactgtacaattcacttttcatggactgaaggcaccaccaagatagatccaggagtgacaactccagtgtaggtgtccactgttcccttaatctctgtcctgctccaagtataaataaatcggggccatttccttaga

SEQ ID NO: 38 - DNA, 434 bp, Homo sapiens
  misc_feature (1)...(434): n = A,T,C or G
ggcacgaggtacacagctgcccccaggctgagagtgcttgatacacccttgaatcccctcttatatgatgccccagcccaggagagataaaagcatcagcaccatgagattcacctgcctctggtcgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactcttagacagcaaaaatgctttctcccagtcttgttcccttgttctcagttcccaccctgcctggataactactgttcttggtttnnnnnnnnnnnnnnnnnnnnnnnnagtctcgtaccagattcaaaaatcagtcaactacttcaaaaacaatgacatgctggctacttaa

SEQ ID NO: 39 - DNA, 428 bp, Homo sapiens
ggcacgagctttgtggatgtttccagctgccagcgtcacccttctgtctgctccctggaccagcttcaggacttgaaggccctcgtggctgagatcatcacacatttgcaggggctgcagagggacttatctctagcagtctcctacagcaggctccattcctcagactggaatctgtgtactgtatttgggatcctcctgggctatcctgttccctatacctttcacctgaaccagggagatgacaactgcttagctctgactccactacgagtattcactgcccggatctcatggttgctaggtcaacccccaatcctgctctattcttttagtgtcccagagagtttgttcccaggcctgagggacattctaaacacctgggagaaggacctcagaacccgatttaggactcagaatgactttgc

SEQ ID NO: 40 - DNA, 429 bp, Homo sapiens
  misc_feature (1)...(429): n = A,T,C or G
ggcacgagtggagagcacctctgtgcctctctgagagcactcacagccaaaagtacacagctgcccccaggctgagagtgcttgatacacccttgaatcccctcttatatgatgccccagcccaggagagataaaagcatcagcaccatgagattcacctgcctctggtcgttagggaacaatggaggcctgcgatttggagttaaactctcagtgatctctgtgttgacaacaccaaagctagaggaatccagtaggatgtgggcatggttttcccggaaggctgactgagcagttctgcaaatgtttgcaagtacagggcagaatttcatccagcctcagaaccttgagccaagactcagcatcagcaaagccaaaagtttcattttcttgactgtgggagtgctagtcccaacctttagatggccn

SEQ ID NO: 41 - DNA, 430 bp, Homo sapiens
actctgcaaacagctacttgtgctgattgcaggagacccataaattcgaacgaggaacaaccgagacctgaaggggctgacgaacgcgatttctgataagtatggggtccctgaagagaacatttaccaagcctacaataaatgcacgcgaggaatcttatgcaacatggacaacaacatcattcagcattacagcaaccacgtcgccttcctgctggacatggcggagctggacggcaaaattcagatcatccttaaggagctggaaggcctctcgagcatacaaaccctcacgacctgcatggggccagcagggacgtggccccacgccacacacaacctctccacatgcctcaacgctgttacttgaatgccttccctgagggaagaggcccttgagtcacagacccacagacgtcaggaccatggg

SEQ ID NO: 42 - DNA, 437 bp, Homo sapiens
  misc_feature (1)...(437): n = A,T,C or G
ggcacgaggcgccctctgcccccctcagagggtctctcctctcgacccccaaattcccccagcatctcaatcccttgcatggggagcaaggcctcgagcccccatggtttgggctccccgctggtggcttctccaagactggagaagcggctgggaggcctggccccacagcggggcagcaggatctctgtgctgtcagccagcccagtgtctgatgtcagctatatgtttggaagcagccagtccctcctgcactccagcaactccagccatcagtcatcttccagatccttggaaagtccagccaactcttcctccagcctccacagccttggctcagtgtccctgtgtacaagacccagtgacttccaggctcccagaaaccccaccctaaccatgggccaacccagaacaccccactctccaccactgggcan

SEQ ID NO: 43 - DNA, 432 bp, Homo sapiens
  misc_feature (1)...(432): n = A,T,C or G
ggnncagtgaccaccaggacctggtgtctgtgcacatctacatcacccagctggctgagaagttcgacctcaggaccactatgctgtacatctgtgagcggcacttccagaaggttctgaaccggagtctattcacaggcctgcgctccatcacccactttggccgtcccccctttgagcccttcttcaactccctgcaggaggtccacccccaggtccggaagatcggggtgtttagctgtggcccccctggcatgaccaagaatgtggaaaaggcctgtcagctcatcaacaggcaggaccggactcacttctcccaccattatgagaacttctaggccccttcccgggggttctgcccactgtccagttgagcagaggtttgagcccacacctcacctctgttcttcctatttctggctgcctcagccc

SEQ ID NO: 44 - DNA, 436 bp, Homo sapiens
  misc_feature (1)...(436): n = A,T,C or G
ggcacgagccgaggcgcgcgtgttccgtggccgcttccagggccgcgcggcggtgatcaagcaccgcttccccaagggctaccggcacccggcgctggaggcgcggcttggcagacggcggacggtgcaggaggcccgggcgctcctccgctgtcgccgcgctggaatatctgccccagttgtcttttttgtggactatgcttccaactgcttatatatggaagaaattgaaggctcagtgactgttcgagattatattcagtccactatggagactgaaaaaactccccagggtctctccaacttagccaagacaattgggcaggttttggctcgaatgcacgatgaagacctcattcatggtgatctcaccacctccaacatgctcctgaaacccccccttgaacagctgaacattgtgctcatagactntggg

SEQ ID NO: 45 - DNA, 300 bp, Homo sapiens
tctctctctctctctctcacagacacttttaccccatatatacacataaaatgtgtgcgcgagagagagagagccctctcgctctatatatatccccgcgggggggagataaaaatatatatccccacactttatagggcgggctcccccctctatcctgtgtgtagagagaaatatatatatatctgtggggggagagagagatctctcacccccccgcacacgcgagctctttcttaagatgtgtgagcgccccccccctgtttttgtaaaaaagagaggggtatatatattgggggg

SEQ ID NO: 46 - DNA, 191 bp, Homo sapiens
caaaacaaaaccatgttcccactggtgatgcctgtctgacacgttttggtatttagtaggaaatgaagggtcttcaagcttcgagagaaccttcaaaattgtcacaattgctgaaaacagaatgaatcgggaacattatctcaatattttgcataatagacaacaccacagtgttttggttccctgacctg

SEQ ID NO: 47 - DNA, 302 bp, Homo sapiens
  misc_feature (1)...(302): n = A,T,C or G
gcccgggcgtgtgtgtatgtgtgtacacgcccccgtgggctctctgtcgcatcttgnnnnnnnnnnnnnnnnnnnnnnnnnntgtannnnnnnnnncacatagcgcgcgcgctcgcgcgcacggagctatagagacaccactctctctctgagatacacgcgcgcgcacacactctgcgcgcgcgcgctcttctttgtctcgcgcgcgcgcccgctatgtggagggtatatgtgggggaaaatagcgaggtgtgcgcgcacccgcgcacgcgcgctctatatctctatatcttcagcgcgcg

SEQ ID NO: 48 - DNA, 411 bp, Homo sapiens
ggcacgaggcttgcggggcattaggactagagggttggtgaaaattcagacagaatgtaacttgacaaagagaagacagcaacaactgtaacaattatcttatgaatatttgcgaaactcaaagggatctgattggtgacctctgggctttatcaaattaacatcacaacttctagaagaaagtcaaccttcatcttttacaatagaaatcatatgttttgctaacccattcctatttaggctgaaaacaattaagagttatgggtacttaaaaaaatcattatgtttataaaattagtgatagaaggagcatagtgttctatacagtcacacacatacacttccttatttcttttatttaaactttgagtaacatagcagtctatgtttgggtcagttttcccttttttg

SEQ ID NO: 49 - DNA, 408 bp, Homo sapiens
ggcacgaggcacacaaagccaagggcataccctatagagtaaagctgcagccaccctgtgtctcatgtgcagctgaaatagtgatctgcttctgtcactgtcacatagacagccctgcatgccccctgtctcacacagtttgtaatgaagacagctccttctcatctttccataagcctgagatacaagttcagggactcagcaatgcactttaggactgagctaggaggcaaatatctgaagcttgctatgctgttctttccattccttttccctctgaaacacacaaaataccaaaggaacttacgcatcacaccactgagtcctctaactaatcatatgtgctcagacacagctcaagcacaccccttagttaagagagaacctccatatacattaatttttttc

SEQ ID NO: 50 - DNA, 407 bp, Homo sapiens
  misc_feature (1)...(407): n = A,T,C or G
agagaaacatccactcgaattcggcacgaggacagggcagggctagatcttttttctgcacttggcctgtgacatactgtctggtgtctgagaatcctcccctacttctctagttaatctccagagacttctgtgactacttaatcacaaaggaaattttcaggaatattatcaaatactattttagaaaaaaaaagagaagggatttgaatgttttcagttcagtttagttatcnnnnnnnnnnnnnnnncccaaactcaagtatggaggcccccccctctttaaacccaccaaaaaaattttttggggttcagggtgggttggccaactacccaaacccccaaagaaaatgggggttaacccccttgaaaaagttttcttactttggggggctgcccttgagccg

SEQ ID NO: 51 - DNA, 312 bp, Homo sapiens
ccccgggggcgctctctttttttttccccccaagtgagagagccccgcgcgcgtctctctctcgcattttttcgacaccccccttgtgtggggcggggcgcgcgtctgtgtgtgatacacagaatgtgcgtggtgtgtctgagagacactcttcgcgcttgtgtgtgagacacgagactttctctttttagggggcggggggggagttttatgtgtgccacatgttttctgtgtataaaaagagcgcacagagtgttttttatatctgtgagagagacctctctgtatatatacacgctcagaggggagagg

SEQ ID NO: 52 - DNA, 420 bp, Homo sapiens
  misc_feature (1)...(420): n = A,T,C or G
acgagggnnnnnaagcaccgcgggtaccccatgagggcctacaagctggccaccctggccatgacccatctcaacctgagctacaatcaggacacacaccctgccattaatgatgttttgtgggcctgtgcgcttagccactcccttggtaaaaatgagcttgcagctataatacctctggtggtcaagagtgtcaagtgtgcaacggtactgtcagacattttgcgcagatgcactctgaccactcctggcatggtgggacttcatgggaggaggaactctggtaagctcatgtcactggacaaagcccccttgaggcaactcttggatgccacgatcggggcctacatcaacacaacgcactcacggctcacacacatcagtcctcggcactatagtgagtttatagagttcctcagc

SEQ ID NO: 53 - DNA, 394 bp, Homo sapiens
  misc_feature (1)...(394): n = A,T,C or G
ggcacgaggtgtggatgacagagcgagaccctgcctcatnnnnnnnnnnnnnnnnnnnncccccccnnnnnnnnnnnaaaaacccggttgggccccggctgttctttagggccctaaaaattgccccaaaaaaaattggccgggccctaaaaaaaccccggttttttggggagaattcaaaaaagggtcggtnnnnnnnnnntttttaaacttccaaccggcctcagggggaaaaaacctggaaaactcaatgggggttggaacaaaatcaatatttggtcctaccggaaagcgttaagattttaaaccagtaaaaatggccaannnnnnnnnnnnnnnnnnnnaacagggcccccggggtaagggctaaaaattttcagatttgaaccttttt

SEQ ID NO: 54 - DNA, 390 bp, Homo sapiens
ggcacgagattttcttggcaataagcggactctgggactccggctccctaccccaaactgaagcgcttccgtgaacacccccgtcctccgtagggggaggggagcaggcgggatcctgggtccctcataagcactttggttttaccgcctgcaacctcactgtgcccgccccgcaccatgccctagccccaggtctagccgggcccattgcagggggcagcacttgggggcatctccggcacttgggtgggaccaaggagatgccaccatagacctttccctcgccttcttcctccctagtccgggttccattcttttcaccagcacccatcgcccaaggggtaccgagggggggcagggggtggtcaattcaaacccaacccccgctcg

SEQ ID NO: 55 - DNA, 280 bp, Homo sapiens
  misc_feature (1)...(280): n = A,T,C or G
tctctctctctctctgcgcccacacctctctcannnnnnnnnngcacgtgtatatctnnnnnnnnnnnnnttttttttagagagacatctcgcgcgtgtctctctttttcccgcccgccgctcttttctcgcgcgcgcgcgcaccccccctgtgtggggcgcgcgctctctttttttttgtgcgcgcgannnnnnnnnntctctctctgtggcgnnnnnnnnnnnntctcttattttatattttggggggcggggggcctcccctcccccctgtgtgcct

SEQ ID NO: 56 - DNA, 398 bp, Homo sapiens
  misc_feature (1)...(398): n = A,T,C or G
ggcacgaggtccacctcagctcagcaatctcatgccggttggcaattagtcagcataagccgatgcctgcccatcagttctttactctgaggtgttagagtggaataaaaatataaatacttacnnnnnnnnnnnnnncaatacccaaccccctcccattnnnnnnnnnnnnngcccgcccccctaaaattcatggagaggcctatttcgtagccagccactatataaaccctgctggttgggcggnnnnnnnnnnnngtgaaggggggaaaaaaaagcctttttttgaaaaaattagtcattttttgctttttttggacacattttgcgggacaaagaaccctgtaaaacccccctattcnnnnnnnnnnnnnnnaacctcaacgaggggggggcgg

SEQ ID NO: 57 - DNA, 386 bp, Homo sapiens
ggcacgagattttcttggcaataagcggactctgggactccggctccctaccccaaactgaagcgcttccgtgaacacccccgtcctccgtagggggaggggagcaggcgggatcctgggtccctcataagcactttggttttaccgcctgcaacctcactgtgcccgccccgcaccatgccctagccccaggtctagccgggcccattgcagggggcagcacttgggggcatctccggcacttgggtgggaccaaggagatgccaccatagacctttccctcgccttcttcctccctagtccgggttccattcttttcaccagcacccatcgcccaaggggtaccgagggggggcaggggggggtcaagtccaggcccacccccg

SEQ ID NO: 58 - DNA, 202 bp, Homo sapiens
cactttttctatatgaatatcttggccgtatcatagactcaaaaaagaaattatgcaagttctttctgcccccacctgcgccaggggagaagtttaccttcgggaactccagagttaaagcagttgtggtgataattttttatgctgaacacaccacgatataaaaaacaacattcacgtgctttatttttgttatgtgttt

SEQ ID NO: 59 - DNA, 394 bp, Homo sapiens
ggcacgagtctgcttctgtcactgtcacatagacagccctgcatgccccctgtctcacacagtttgtaatgaagacagctccttctcatctttccataagcctgagatacaagttcagggactcagcaatgcactttaggactgagctaggaggcaaatatctgaagcttgctatgctgttctttccattccttttccctctgaaacacacaaaataccaaaggaacttacgcaacacaccactgagtcctctaactaatcatatgtgctcagacacagctcaagcacaccccttagttaagaaagaacctccatatacattaatttttttctgcctaaaaataaaattgcgttgtggcagcaatttggaaactacagcaaagtctccaaaaaa

SEQ ID NO: 60 - DNA, 246 bp, Homo sapiens
  misc_feature (1)...(246): n = A,T,C or G
cccctccttttttaggcctgaatacaaagtagaagatcactttccttcactgtgctgagaatttctagatactacagntcttactcctctcttccctttgttattcagggtgaccaggatggcgggaggggatctgtgtcactgtaggtactgtgcccaggaaggctgggtgaagtgaccatctaaattgcaggatggtgaaattatccccatctgtcctaatgggcttacctcctctttgccttn

SEQ ID NO: 61 - DNA, 395 bp, Homo sapiens
  misc_feature (1)...(395): n = A,T,C or G
ggcacgagcttgcttccctctcaccctctgcagtttccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttccatatcgtaaactgccttggaaccaattaccactaccagggagacaaactattgcttagaggatgctgacaggagcagcatgccaaaattggaagaaggagaaagtttaagctctcctcactatgagttttcaagtataaaagactttttcttccacgattttgagaacaactgaggactcttgtgaccaggacaacagggaagcttgcagcaagatagctccaggttggattcatgcttcgcaccccaaaggct

SEQ ID NO: 62 - DNA, 387 bp, Homo sapiens
ggcacgaggcttgcggggcattatgactagagggttggtgaaaattcagacagaatgtaacttgacaaagagaagacagcaacaactgtaacaattatcttatgaatatttgcgaaactcaaagggatctgattggtgacctctgggctttatcaaattaacatcacaacttctagaagaaagtcaaccttcatcttttacaatagaaatcatatgttttgctaacccattcctatttaggctgaaaacaattaagagttatgggtacttaaaaaaatcattatgtttataaaattagtgatagaaggagcatagtgttctatacagtcacacacatacacttccttatttcttttatttaaactttgagtaacatagcagtctatg

SEQ ID NO: 63 - DNA, 401 bp, Homo sapiens
ggcacgagggaaactgtatgacaggagaatgaatcaggtttggggctcaaggtgccggccactgggaaaaacagctgccccgagttgcaaaactctgggtcctatatgtataaactatgccctgaggaaggaatctcaggcgtatcttaggagaaaatgttctagcttgggaaacaaacacaacaggaccgtgaatccaaatatttcaagtgggtttagaggactggagttctaaacgctgcttttactgtaagtgatcacgccccggaatgtgctgaagaaaggaaaatgagccagtatcggcgaggactatgggcaaggaaaacgagagtgtgcgatgtgtcaaagcaagacatctgtgtatagtaatataaccaagtaatagatagtcatagaatcaa

SEQ ID NO: 64 - DNA, 274 bp, Homo sapiens
  misc_feature (1)...(274): n = A,T,C or G
cacgcacccgcctgtgtgtgtgcgcacacacgctccctctctctatagacagacacacactgcgcgctcgctctctcttttgtgtgcgctctccgtgctccccccctctctctcttttttctctatatnnnnnnnnnnnnnnnnntctgagagctcgcgcgctcagcgttctattcacacgcgcgttttttttatatatatattttgtgcgcgccggggggggcgcacacactctctcttttttgtgggttcgctgtccgcgtccctccttttg

SEQ ID NO: 65 - DNA, 279 bp, Homo sapiens
  misc_feature (1)...(279): n = A,T,C or G
cccttttttttatacacccccccttgtctgtctgttttgtgtgtctgccccccttctctcgttgtgatctccctctctcttttttctccccccgcgctctctctctcttgcggggaggctcacataccccctctctctctcttttttgaaccacacattccgtttctcttttttttatctctacccctctctcgtctgtaccccccacannnnnnnnnnnnnnnnnnnnnnnnagagtagagttgcgttccccactctccnnnnnnnnnngtggggtgc

SEQ ID NO: 66 - DNA, 311 bp, Homo sapiens
caaaacaaaaattaaaaatgacccccctttaaaattttagggggtccatttttaaaaaccttaacagtttaaaggttcttggtcagtttggggaaccccaccttgagatgggagcaaaaaaggggatttttttccaacatagcgagcggtttagattttttttgtcccgttagagttgccctgtgcaccacgccaaaacctccagaggtcttctttttttacacaccctgtctgggggtgtttctcagaagattaacacagcgcctgggggtttaagggaggggtgacctccgcaggacattatggggctt

SEQ ID NO: 67 - DNA, 386 bp, Homo sapiens
ggcacgagggaatctcaggcgtatcttatgagaaaatgttctagcttgggaaacaaacacaacaggaccgtgaatccaaatatttcaagtgggtttagaggactggagttctaaacgctgcttttactgtaagtgatcacgccccggaatgtgctgaagaaaggaaaatgagccagtatcggcgaggactatgggcaaggaaaacgagagtgtgcgatgtgtcaaagcaagacatctgtgtatagtaatataatcaagtaatagatagtcatagaatcaagctgatgtatttggcaggggccgcgggaggatgaggcaactcccatcagattagaaagatgttaacactgtaacaaaagtggggctcgaggaaggggaaaagcgca

SEQ ID NO: 68 - DNA, 396 bp, Homo sapiens
  misc_feature (1)...(396): n = A,T,C or G
ggcacgaggaggcagctgcctttgtttgccatggatgggtaggggctgcactgagcagcaccggtgttcttcatccggctgcacccccaacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctgggtgcagagtgagcccatgggacagccctctgaaattatactgcttacaaccatgctgagtctgcaaggan

SEQ ID NO: 69 - DNA, 397 bp, Homo sapiens
ggcacgagtcttagtcaacatggacaacaacatcattcagcattacagcaaccacgtcgccttcctgctggacatgggggagctggacggcaaaattcagatcatccttaaggagctgtaaggcctctcgagcatccaaaccctcacgacctgcaaggggccagcagggacgtggccccacgccacacacaacctctccacatgcctcagcgctgttacttgaatgccttccctgagggaagaggcccttgagtcacagacccacagacgtcagggccagggagagacctagggggtcccctggcctggatccccatggtatgcttgaatctgctccctgaacttcctgccagtgcctccccgtaccccaaaacaatgtcaccatggttaccaccta

SEQ ID NO: 70 - DNA, 394 bp, Homo sapiens
ggcacgagccaaacctagcacaaaacggggttcacaagccatggtcggggtccgggggggacagaaatggattttcttggcaataagcggactctgggactccggctccctaccccaaactgaagcgcttccgtgaacacccccgtcctccgtagggggaggggagcaggcgggatcctgggtccctcataagcactttggttttaccgcctgcaacctcactgtgcccgccccgcaccatgccctagccccaggtctagccgggcccattgcagggggcagcacttgggggcatctccggcacttgggtgggaccaaggagatgccaccatagacctttccctcgccttcttcctccctagtccgggttccattcttttcaccagcacccatc

SEQ ID NO: 71 - DNA, 389 bp, Homo sapiens
ggcacgaggaaagttaagcatctacaggttatggctttgggagttccaatatcagtctatcttttattcaacgcaatgacagcactgaccgaagaggcagccgtgactgtaacacctccaatcacagcccagcaaggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgtctaaaattgaaatcgccttacaatgtctgttctggagaaagacgaccactgtgaagcctttgtgaagaattttcatcaaggcatctgtagagatcagtgag

SEQ ID NO: 72 - DNA, 396 bp, Homo sapiens
  misc_feature (1)...(396): n = A,T,C or G
ggcacgaggcctggccccacagcggggcagcaggatctctgtgctgtcagccagcccagtgtctgatgtcagctatatgtttggaagcagccagtccctcctgcactccagcaactccagccatcagtcatcttccagatccttggaaagtccagccaactcttcctccagcctccacagccttggctcagtgtccctgtgtacaagacccagtgacttccaggctcccagaaaccccaccctaaccatgggccaacccagaacaccccactctccaccactggccaaagaacatgccagcagctgccccccatccatcaccaactccatggtggacatacccattgtgctgatcaacggctgcccagaaccagggtcttctccaccccagcggan

SEQ ID NO: 73 - DNA, 386 bp, Homo sapiens
ggcacgaggccacctgttgccctaacaccctgtctgactctctcccgctgcagcagccagtccctcctgcactccagcaactccagccatcagtcatcttccagatccttggaaagtccagccaactcttcctccagcctccacagccttggctcagtgtccctgtgtacaagacccagtgacttccaggctcccagaaaccccaccctaaccatgggccaacccagaacaccccactctccaccactggccaaagaacatgccagcagctgccccccatccatcaccaactccatggtggacatacccattgtgctgatcaacggctgcccagaaccagggtcttctccaccccagcggaccccaggacaccagaactccgttca

SEQ ID NO: 74 - DNA, 390 bp, Homo sapiens
ggcacgagctcagatccggggactgcggataaatggccttaggccgcgggcagcgagatgttgcgttccggtgtgggtgtgggtgtgcctccgacggcgtctcggtgccagtgtcgaggttctttctgcttagctacccggagccgactacggaggaggacacctgagtttacgtctcttccatctgctgctcgcctcagctgcctgggtccccgacgagagccaggtgacacttaactccgccatctgcgttttgagcactgttctcataatggagtttcctgatttggggaagcattgttcagaaaagacttgcaagcagctagattttcttccagtaaaatgtgatgcatgtaaacaagatttctgtaaagatcattttccatacgg

SEQ ID NO: 75 - DNA, 399 bp, Homo sapiens
ggcacgagaaatggccttaggccgcgggcagcgagatgttgcgttccggtgtgggtgtgggtgtgcctccgacggcgtctcggtgccagtgtcgaggttctttctgcttagctacccggagccgactacggaggaggacacctgagtttacgtctcttccatctgctgctcgcctcagctgcctgggtccccgacgagagccaggtgacacttaactccgccatctgcgttttgagcactgttctcataatggagtttcctgatttggggaagcattgttcagaaaagacttgcaagcagctagattttcttccagtaaaatgtgatgcatgtaaacaagatttctgtaaagatcattttccatacgctgcacataagtgtccgtttgcattccagaag

SEQ ID NO: 76 - DNA, 386 bp, Homo sapiens
ggcacgagcaaaggctcgcagcggccagaaacccggctccgagcggcggcggcccggcttccgctgcccgtgagctaaggacggtccgctccctctatccagctccgaatcctgatccaggcgggggccaggggcccctcgcctcccctctgaggaccgaagatgagcttcctcttcagcagccgctcttctaaaacattcataccaaagaagaatatccctgatggatctcatcagtatgaactcttaaaacatgcagaagcaactctaggaagagggaatctgagacaagctgctatgttgcctgagggagaggatctcaatgaatggagtgctgcgaacacctggggattcttttaccagcaacaacatggtttttgggaact

SEQ ID NO: 77 - DNA, 395 bp, Homo sapiens
ggcacgaggccatctccaaatactgcggttgttcagaagctcttagtttgtgggctgtccttgttatttcacttgaccatctgtacaacattacctgtggagtacaacattgatgagcattttcaagctacagcttcgtggccaacaaagattatctatctgtatatctctcttttggctgccagacccaaatactattttgcatggacgctagctgatgccattaataatgctgcaggctttggtttcagagggtatgacgaaaatggagcagctcgctgggacttaatttccaatttgagaattcaacaaatagagatgtcaacaagtttcaagatgtttcttgataattggaatattcagacagctctttggctcaaaagggtgtgttatga

SEQ ID NO: 78 - DNA, 389 bp, Homo sapiens
ggcacgaggcaggccgggatgttcgtcctggtggaaatggtggacaccgtccggatccccccttggcagtttgagaggaagctcaacgactccattgccgaggagctgaacaagaagttggccaacaaggtcgtgtacaacgtgggactctgcatttgtctgtttgatatcaccaaactggaggatgcctatgtattccctggggatggcgcatcacacaccaaagtccattttcgctgcgtggtgtttcatccattcctagatgagattctcattgggaagatcaaaggctgcagcccagaaggagtgcacgtctctctaggcttcttcgatgacattctcatccccccagagtcactgcagcagccagccaagttcgacgaagcgga

SEQ ID NO: 79 - DNA, 365 bp, Homo sapiens
  misc_feature (1)...(365): n = A,T,C or G
ggcacgagaaaacatttcatcttgatttttattaaggtgatatgtatgttacttaacagctgtataatacacatttgcatgcattaggaagttttttttgggttttattcatcctgtagtgatgtatctgtgacctcaacgagtaggcacttctgtactgtactggtttcttaaagtttcttttatcccgcccccacccccaacctcagcctcaagtatgtaannnnnnnnnnnnnnnnnnnnnnnnnnnnnnnnnnnnnnnnnnaaaacaaagccccgttttgtccccaggctggataacaggggcggaatctgggttaattgaaccctttgcttttggggttaaggcaattttcctgcctcac

SEQ ID NO: 80 - DNA, 376 bp, Homo sapiens
ggcacgagctggaaaccagcccctaagctgctttcaccttcggcccatttctcactagccagcctctcccacggcctccccagtttcttcaaatacaccccctccactattcaccatactgccaccgtgatttatttacaactttttgtccggattacctcagtagccttctaattgtcccctttgcatctaaagtagcccctctcatcccccaaatcttaccatgtcactcttctacataattctggctttccatgacccataaaccacatttctcaagtgtgctctatgctggcttgaatatgttaatgatcttaattctacttttagtgcaattttcttagagctggcatcactttcatcatgacgtgagaac

SEQ ID NO: 81 - DNA, 384 bp, Homo sapiens
ggcacgagaggattgtgtgaaattgtgcaaatgcatgaatgtgggctgggatagtaaaagggagggccccggagcagcccacctggggtcctatctagtagacgcgcccggtgcccacccattgctgtgatgccagcagcccactgcaagcatcctcttcctttccaaggttctgtctggtacatgaataggtgtggcaggggtgggggctcctgaagaccaactaggggtactagggaccttagactcttgcgagagcctgcaccccatatcaggtggggtcaatagataaatacccctgcctccttgccccttagttctggtgtggtgggcaagtcagaggaactgttcttctcacactttcacgtgctctcggtggagatc

SEQ ID NO: 82 - DNA, 383 bp, Homo sapiens
ggcacgagcaaaggctcgcagcggccagaaacccggctccgagcggcggcggcccggcttccgctgcccgtgagctaaggacggtccgctccctctagccagctccgaatcctgatccaggcgggggccaggggcccctcgcctcccctctgaggaccgaagatgagcttcctcttcagcagccgctcttctaaaacattcaaaccaaagaagaatatccctgaaggatctcatcagtatgaactcttaaaacatgcagaagcaactctaggaagtgggaatctgagacaagctgttatgttgcctgagggagaggatctcaatgaatggattgctgtgaacaactggggatttctttaccaggatcacaatggtaatatggg

SEQ ID NO: 83 - DNA, 358 bp, Homo sapiens
ggcacgagcagggccgcgcggcggtgatcaagcaccgcttccccaagggctaccggcacccggcgctggaggcgcggcttggcagacggcggacggtgcaggaggcccgggcgctcctccgctgtcgccgcgctggaatatctgccccagttgtcttttttgtggactatgcttccaactgcttatatatggaagaaattgaaggctcagtgactgttcgagattatattcagtccactatggagactgaaaaaaactccccagggtctctccaacttagccaagacaattgggcaggttttggctcgaatgcacgatgaagacctcattcatggtgatctcaccacctccaacatgc

SEQ ID NO: 84 - DNA, 338 bp, Homo sapiens
aagatggctgagagggacagaatgctttattttggagagaaacaatgttctaggtcaaactgagtctaccaaatgcacactttcacaatgggtctagaagaaatctggacaagtcttttcatgtggtttttctacgcattgattacatgtttgctcacagatgaagtggccattctgcctgcccctcagaacctctctgtactctcaaccaacatgaagcatctcttgatgtggagcccagtgatcgcgcctggagagacagtgtactattctgtcgaataccagggggagtacgagagcctgtacacgagccacatctggattcccagcagctggtg

SEQ ID NO: 85 - DNA, 475 bp, Homo sapiens
  misc_feature (1)...(475): n = A,T,C or G
gtcgctcaataggcaggagtccatcgattcgaattcggcacgagnnnnnnnnnnnnnnnnnnnnnnnnnnnnnnnnnnnnnnnnnnnnnnnnnnnnnnnnnnnnnnnnnnnnnnnnnnnnnnnnnnnnncgctccactgtgcactcctgacacatactttccccgctacactctctattctccccctcttgtgttctctctctatagcggtagatagagaggcctgtgtgtagataataaacgtgtgtgtgtgtgtaagaaaggagacacaaacacgcccacnnnnnnnnnnnttggggcctttttttcttttgagccctttggggaaaaaacccggggaaaacagcccatacccactatttggggcgcgccaaaaaaccttctttaaaaaaaatgtgttaaatgttaaattttttaggaacannnnnnnnnnngcaaaaaatagcaccccaaaagcaggggttttacatttttg

SEQ ID NO: 86 - DNA, 467 bp, Homo sapiens
gagcgattttctgcaggattctatcgattcgaattcggcacgagccatggtctcagtgagggctggaatttacagagaagtttggccagggggtccaccatgctgccagtcagtttgggaaggaaacagagaagctcggccatggggtccaccatggggttaatgaggcctggaaggaagcagagaagtttggccagggtgtccaccatgctgcctcgcaggtggggaaggaggaagacagagtggtccaaggcctccatcatggcgttagtcaggctggaagggaggcggggcagtttggccacgacattcaccacacagcagggcaggctgggaaagagggagacatagcagttcatggtgtccaacctggggtccacgaggccgggaaggaggcagggcaatttggccagggagttcaccatacccttgaacaggccgggaaggaagcaaacaaagcggtccag

SEQ ID NO: 87 - DNA, 449 bp, Homo sapiens
  misc_feature (1)...(449): n = A,T,C or G
cggggtgggaaaccngannttnannaancggacggattctcccgttccgaatagccttttacagaagattcttcacagctatgtgcctgaagagatcanggatggaaatcaagttcgagttacctcatgggatggcaggaaatggggagaactggagggggacacctatgaccgggtgctggtggatgtgccctgtaccacagaccgccactcccttcatgaggaggagaacaacatctttaagcggtcaaggaagaaggagcgacagatattgcctgtgctgcaagtgcagcttcttgcggctggactccttgccaccaaaccaggaggccatgttgtctattctacctgctcactctcacacttacagaacgagtatgtggtgcaaggtgccattgagctcctgggcaatcaatacagcatccaggtacaggtggaagatctgactg

SEQ ID NO: 88 - DNA, 439 bp, Homo sapiens
  misc_feature (1)...(439): n = A,T,C or G
gtagtgtatgtgcagcctcccatcgattcgaattcggcacgagatcccctcttatatgatgccccagcccaggagagataaaagcatcagcaccatgagattcacctgcctctggtcgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactcttagacagcaaaaatgctttctcccagtcttgttcccttgttctcagttcccaccctgcctggataactactgttcttggtttnnnnnnnnnnnnnnnnnnnnnnnnagtctcgtaccagattcaaaaatcagtcaactacttcaaaaacaatgacatgctggctacttagatagaagaggaggc

SEQ ID NO: 89 - DNA, 436 bp, Homo sapiens
  misc_feature (1)...(436): n = A,T,C or G
ggcacgagcatcaaatagtaaatatagatcttatgctggaaatgtcaacctccctggcagctgtaacgcccatcattgaaagggaaagcggaggacaccattatgttaatatgactttacctgtcgatgcagttatatctgttgctccagaagaaacatggggaaaagttcgtaaactcctggttgatgcaattcataatcaactaactgacatggaaaaatgtattttgaaatatatgaaaggaacatctattgtggtccctgaaccactgcactttttattaccagggaaaaaaaatcttgtaacaatttcatatccttcaggaataccagatggccagctgcaggcctataggaaggagttacatgatcttttcaatctgcctcacgacagaccctatttcaaaaggtctaatgcttatcactttccagatgn

SEQ ID NO: 90 - DNA, 437 bp, Homo sapiens
  misc_feature (1)...(437): n = A,T,C or G
ggcacgagagatcatgcactaccacatgcagcacgagcagtaccggcaggtcatcagcgtgtgtgagcgccatggggagcaggacccctccttgtgggagcaggccctcagctacttcgctcgcaaggaggaggactgcaaggagtatgtggcagctgtcctcaagcatatcgagaacaagaacctcatgccacctcttctagtggtgcagaccctggcccacaactccacagccacactctccgtcatcagggactacctggtccaaaaactacagaaacagagccagcagattgcacaggatgagctgcgggtgcggcggtaccgagaggagaccacccgtatccgccaggagatccaagagctcaaggccagtcctaagattttccaaaagaccaagtgcagcatctgtaacagtgccttggagttgccctcan

SEQ ID NO: 91 - DNA, 437 bp, Homo sapiens
ggcacgagcttcagtcttatgtcatttactctttaggacaacctcttgaaaaactaaatcatttctttgaaggtgttgaagctcgcgtggcacagggcataagggaggaggaagtaagttaccaacttgcatttaacaaacaagaacttcgtaaagtcattaaggagtaccctggaaaggaagtaaaaaaaggtctagataacctctacaagaaagttgataaacatttatgtgaagaagagaacttacttcaggtggtgtggcactccatgcaagatgaatttatacgccagtataagcactttgaaggtttgatagctcgctgttatcctggatctggtgttacaatggaattcactattcaggacattctggattattgttccagcattgcacagtcccactaaaccttgtgaaagaagaaaagataactgaat

SEQ ID NO: 92 - DNA, 427 bp, Homo sapiens
  misc_feature (1)...(427): n = A,T,C or G
aacggctcttctncttttgaggagcccatcgagtcgaattcggcacgaggcgagtctctgggtcgcgacgggaaggagtgaaacacctctctgcgcctgcgcgctccgtgcctgcgaagcaaacccggcctcaccttttcctgcccgaagcagaagattctcgcaggcctggtttctccctccagaagaccccccacccaaatcctctgtagctcctgggagtgccctgacccctgctgccaccgtccttcagagagcaacggaagagcttcccggagggcgaggaaaagagggaaagtagccagcaatgtcgaacgcagtgtataataagatgtggcatcagacccaagaagccctcggtgctttactcgatgaagagcctcagacgatgattgaaccacacagaaatcaggttttcatctttcaa

SEQ ID NO: 93 - DNA, 429 bp, Homo sapiens
  misc_feature (1)...(429): n = A,T,C or G
gtgacgatcccatcattcaattcggcacgagctcacagccaaagttccttctgcccccaggctgagagtgcttgatacacccttgaatcccctcttatatgatgccccagcccaggagagataaaagcatcagcaccatgagattcacctgcctctggtcgttagggaacaatggaggcctgcgatttggagttaaactctcagtgatctctgtgttgacaacaccaaagctagaggaatccagtaggatgtgggcatggttttcccggaaggctgactgagcagttctgcaaatgtttgcaagtacagggcagaatttcatccagcctcagaaccttgagccaagactcagcatcagcaaagccaaaagtttcatttcttcgactgtgggagtgctagtcccaacctttagatggccattcagttnta

SEQ ID NO: 94 - DNA, 421 bp, Homo sapiens
  misc_feature (1)...(421): n = A,T,C or G
ggcacgagattatttacttggtgtgtggtcaccactgttttttaaatgagtgttttcatttgtatcaaactggacctgctttcctcaaggattgcccaaaaggagacacaaatttactaaacacttatcaataatagaacaccgtgctaggcaatttccatatactattaatttaatcctcacaataactttggaagacagaaagtattttctctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcctctgtctccaaagcctgtacttcattcaggacactttcccccacatttagaaaagctgtaattatcttccagtgagacagcatagcacatgtgatcactgtcccttc

SEQ ID NO: 95 - DNA, 421 bp, Homo sapiens
ggcacgagatgagaagataaaattcagcgttggcctttagactttgccatccttaaggagtgatggaagccaagtgaacaagcctcagtgacacaagtcaaattcatagtttcactctgggttttttgttgttgtgtggttattattctcactacagaaagactgagtttcatgctcctggctatgtcagatgtgaattttcatgggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtctaaaattgaaatcgccttacaatgtctgttctgga

SEQ ID NO: 96 - DNA, 418 bp, Homo sapiens
  misc_feature (1)...(418): n = A,T,C or G
tggatccatcgattcaattcggcacgaggttatttttaagaacttttgcttactatattggatttacctgcggtgtgagtagctttaaatgtttgtgtttatacagataagaaatgctatttctttctggttcctgcagccattgaaaaacctttttccttgcaaattataatgtttttgatagatttttatcaactgtgggaaaccaaacacaaagctgataacctttcttaaaaacgacccagtcacagtaaagaagacacaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnng

SEQ ID NO: 97 - DNA, 418 bp, Homo sapiens
atgctacattgctactttgttgcattgatcgccagacgaccactagattcgaaactgagcgcgagatgatgaatctgtgtgttatgaaaatgcatgctaccagtataaaatgacattctctattaataacatctgcggtgcgacacacataattgtcccaatttttaatattgatggggagcatgaagcatttttttaatgtgttggcaggccccattaaatgcataaactgcataggactcatgtggtctgaatgtattttagggctttctgggaattgtcttgacagagaacctcagctggacaaagcagccttgatctgagtgagctaactgacacaatgaaactgtcaggcatgtttctgctcctctctctggctcttttctgctttttaacaggtgtcttcagtcagggaggg

SEQ ID NO: 98 - DNA, 417 bp, Homo sapiens
  misc_feature (1)...(417): n = A,T,C or G
catcgattcgaattcggcacgaggccaagtggacaggccatagcccccacagactggagggacgcggctagggaatgtcccacagagtggccagttatccctgagagaaagagcaggttttagcggagactctgaggctgctttagaatatggtgggtgtgtggggcaaaagggacacccaggggtgtatcaagaggtcatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 99 - DNA, 416 bp, Homo sapiens
ggcacgagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtgaacatt

SEQ ID NO: 100 - DNA, 417 bp, Homo sapiens
ggcacgagggaaaatgtaggctaccagtagaaaatgacattctctattaataagatctgaggtgcgacacacataattgtcccaatttttaagattgatggggagcatgaagcatttttttaatgtgttggcaggccccattaaatgcataaactgcataggactcatgtggtctgaatgtattttagggctttctgggaattgtcttgacagagaacctcagctggacaaagcagccttgatctgagtgagctaactgacacaatgaaactgtcaggcatgtttctgctcctctctctggctcttttctgctttttaacaggtgtcttcagtcaaggaggacaggttgactgtggtgagttccaggacaccaaggtctactgcactcgggaatctaacccacactgtggctctgat

SEQ ID NO: 101 - DNA, 412 bp, Homo sapiens
  misc_feature (1)...(412): n = A,T,C or G
ggcacgaggaaagtaaacgtgtatctcttgttcatttttatagaacttttgcatactatattggatttacctgcggtgtgactagctttaaatgtttgtgtttatacagataagaaatgctatttctttctggttcctgcagccattgaaaaacctttttccttgcaaattataatgtttttgatagatttttatcaactgtgggaaaccaaacacaaagctgataacctttcttaaaaacgacccagtcacagtaaagaagacacaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 102 - DNA, 414 bp, Homo sapiens
ggcacgaggtcttgctcacatgttgtactactctctcctggatgtcacttgtcacctctaccagccctcctttctccagatggcttcttcataaccaccaggtcagaagaggatccgttccaatgattttcctaaaacaatggaagtgttttccaaagagcttataaggcattgtaggatctggcctgccctgactccactttaccagaaccatctgctgctcttctctcttgtgttactcaaggtattagctgctgtggcaaatcaactctgaaatctccgtgacttaatacaagagaggtttatttcttactcacgctgggtgcactgccacttggtaacagaggagctatggaaacttgagacctaagcagaaatgagttcaataatattgctacactctaggactttctc

SEQ ID NO: 103 - DNA, 410 bp, Homo sapiens
  misc_feature (1)...(410): n = A,T,C or G
ggcacgaggaagagccgggaggatgtattggttgttaggaaaatgtaggctaccagtagaaaatgacattctctattaataagatctgaggtgcgacacacataattgtcccaatttttaagattgatggggagcatgaagcatttttttaatgtgttggcaggccccattaaatgcataaactgcataggactcatgtggtctgaatgtattttagggctttctgggaattgtcttgacagagaacctcagctggacaaagcagccttgatctgagtgagctaactgacacaatgaaactgtcaggcatgtttctgctcctctctctggctcttttctgctttttaacaggtgtcttcagtcaaggaggacaggttgactgtggtgagttccaagacacccaaggctan

SEQ ID NO: 104 - DNA, 411 bp, Homo sapiens
ggcacgagatacgaatggggtgtatttttcgactgctcgcaggcacccccaggttatgtggacagagctaagcccaaagttgtgattttccactctgttctgtccatgtcgagggaagataagtagaaagtgacacagtaagagccagaatacaccaggtgaaggagagaattgcattgtgttttgagaagtttcactgacaagttatcctgggctgtgggacatcactagctttgaaagtgtagctggcacctcgtccatctaatttgatgggtgtgtgtggggtgttgtgcacgcgtcggtctaacatatctgaacccaggtgatttctgttctcaggacgcttttaggtgacaaggatcaggcatgtgaacaaataaccatactgtaaagctggctgtgctgggtctc

SEQ ID NO: 105 - DNA, 413 bp, Homo sapiens
  misc_feature (1)...(413): n = A,T,C or G
ggcacgaggaagattctcgcagtcctggtttctccctccagaagaccccccacccaaatgctctgtagctcctggtagtgccctgacccctgctgccaccgtccttcagagagcaacggaagagcttcccggagggcgaggaaaagagggaaagtagccagcaatgtcgaacgcaatgtataataagatgtggcatcagacccaagaagccctcggtgctttactcgataaagagcctcagaagatgattgaaccacaaagaaatcaggttttcatctttcaaacattagccaccttctacgtaaagtatgtgcagatctttagaaacctagagaatgtctacgaccagttcgtccacccccagaaacgaatactgatcaggaaagtcctggacggngtgatgggccgcatcc

SEQ ID NO: 106 - DNA, 412 bp, Homo sapiens
aggatcccatcgattctaattcggcacgagctccataaggcagaggtctatgcgaggacgcccggctggaccacgagaccgcccattgattgcgctgggacaagaattccttatctttggaggcagtgaaacgactaatagctaaaggtaatacagaagaactacgaaaatgttttggggtccgaatggagtttgtgacagctggcctccgagctgctatgggacctggaatttctcgtatgaatgacttgaccatcatccagactacacagggattttgcagatacctggaaaaacaattcagtgacttatagcagaaaggcatccggatcagttatgacgcccgagctcatccatccagagggggtagcatcaaaaggtttgcccgacttgctgcaaccacatttatcag

SEQ ID NO: 107 - DNA, 408 bp, Homo sapiens
  misc_feature (1)...(408): n = A,T,C or G
ggcacgaggaaaaaccagtttctcttttattgtctgttactaatctctattctaaagattcagctcaattctcaaccatactccaaactctctcttttccagctacctttactccctctccttcaattccactttcctctgcttacnnnnnnnnnnncnnnnnnnnnnnnnnnnnnnnnnnnnnnnnnnnnnnnnnnnnnnnnnnnnnnnnnnnnnnnnnnnnnnnnnnnnnnnnnggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatgttttttttcattaaagagagaaatcacctattcaggaccggcccccacctttg

SEQ ID NO: 108 - DNA, 405 bp, Homo sapiens
ggcacgaggcttacaggggtgaccagggcccttcctaactcgaccgcatgtggattggtggctggcttgggagggaggctgtccgatgctgacattccccttaacatggccctgaccgtggctgtcaggggccaccttgcctcaccaggccagccccactgggaatggggtcagtcacagcagaaccgtccaaaggtggacctgatgtgggccctgccgggggcgcttggcctcagcgggccatgggagacccagtgaaacgactctagtgtgaggcagtggtcctgccactgactgacaaaccctctttgtaagcaaacttgacaaataatgaatctactgaactctgttatagaacaagctcattctgcatgaacttctcttattgaagcagaagccacgtca

SEQ ID NO: 109 - DNA, 403 bp, Homo sapiens
  misc_feature (1)...(403): n = A,T,C or G
ggatcccatcgnttcgnattcggcacgaggcaaccagctcgtccagcgcgtggccctgctgctcaaggagcagactgcgtaccccccgacacactacatccggagggtgccccagaggaagatccactacttcacgggcctgcaggcgcttcagctgctgctgctgtgtgccttcggcatgagctccctgccctacatgaagatgatctttcccctcatcatgatcgccatgatccccatccgctatatcctgctgccccgaatcattgaagccaagtacttggatgtcatggacgctgagcacaggccttgactggcagaccctgcccacgccccattcgccagccctccacgtactcccaagctggctctggaactgtgaggggaaggggaagatgtgtgg

SEQ ID NO: 110 - DNA, 397 bp, Homo sapiens
ggcacgagtctgcttctgtcactgtcacatagacagccctgcatgccccctgtctcacacaggttgtaatgaagacagctccttctcatctttccataagcctgagatacaagttcagggactcagcaatgcactttaggactgagctaggaggcaaatatctgaagcttgctatgctgttctttccattccttttccctctgaaacacacaaaataccaaaggaacttacgcaacacaccactgagtcctctaactaatcatatgtgctcagacacagctcaagcacaccccttagttaagaaagaacctccatatacattaatttttttctgcctaaaaataaaattgcgttgtggcagcaatttggaaactacagcaaagtctccaaaaaaatc

SEQ ID NO: 111 - DNA, 401 bp, Homo sapiens
  misc_feature (1)...(401): n = A,T,C or G
ggcacgagagccgttgccttcaccgccctttctccttttatccttttttaaacgctcttgggggttatgtccgctgcttcttgggtgccgagacatatagatggtggtctcgggccagcccctcctctccccgccttctgggaggaggaggtcacacgctgatgggcactggagaggccagaagagactcacaggagcgggctgccttccgcctggggctccctgtgacctctcagtcccctggcccggccagccaccgtccccagcacccaagcatgcaattgcctgtcccccccggccagcctccccaacttgatgtttgcgttttgtttggggggatatttttcataattatttnnnnnnnnnnnnnnnnnnnnnnnnnnnnnnnnnnnnnnnnnnnc

SEQ ID NO: 112 - DNA, 401 bp, Homo sapiens
ggcacgagggcagtccagcaacaagcctttcatttacattaaattataacttttcattcattcctaaaccaaacttaaaattctgctttcctttgagtagaaggtatttaacttgttttgtttttccttcagaaggaatttaatgcaaacggattgcagtcagcactttctgaatgttttcacacagtatgcaaagcttacatcataccaaggagtggagagttgaagtttcctcccagtgactccagtgacagaccacacctagaaagcgtttctcttcctgagtatttcaaaaagatgtaaaagagctggggagagtatgggaagaaacaatacaggattgcctttaattaattaagaattgcctcctgataaaaggaaaaagaaattaatgctggagg

SEQ ID NO: 113 - DNA, 401 bp, Homo sapiens
  misc_feature (1)...(401): n = A,T,C or G
ggcacgaggccccacgggccccatctccccacaggcattgagggtaactggggtaggctcctggagcaggtgggcaccatggctttgtgggccagccaaagggaaaaggaggtgcttaggagggaaagggcagtggaatggcgggagagggctgtggaaaaaagggagcgagccctggaggaggtggaaagggccatcctggagatgaagtggaaggtgagggctgagaaggaggcatgccagcgggagaaagagctgcctgcagcagtacatcccttccattttgtttaaattgggcttggagaatctattctgaaaacattgactctagacttgtagaanagagccattttaattttcaccttcaatggtaaaagcaagggtaatttggttgacatttt

SEQ ID NO: 114 - DNA, 399 bp, Homo sapiens
ggcacgagagcagaagattctctcagtcctggtttctccctccagaagaccccccacccaaatcctctgtagctcctggtagtgccctgacccctgctgccaccgtccttcagagagcaacggaagagcttcccggagggcgaggaaaagagggaaagtagccagcaatgtcgaacgcaatgtataataagatgtggcatcagacccaagaagccctcggtgctttactcgataaagagcctcagaagatgattgaaccacaaagaaatcaggttttcatctttcaaacattagccaccttctacgtaaagtatgtgcagatctttagaaacctagagaatggctacgaccaggtcgtccacccccagaaacgaatactgatcaggaaagtcctggacg

SEQ ID NO: 115 - DNA, 399 bp, Homo sapiens
  misc_feature (1)...(399): n = A,T,C or G
ggcacgaggctttttccaacttttaaggatatcaggagagaagacactcttgatgtggaggtttctgccagtggctacacaaaggaaatgcaggcagatgatgaactgcttcatccattaggtccagatgataaaaatattgaaacaaaagagggatctgaattctcattttcagatggagaagtggcagaaaaagcagaggtttacaggtcagaaaatgaaagtgaacggaactgtctagaagaatcagagggctgctattgcagatcatctggagaccctgaacaaataaaggaagacagtttatcagaagagagtgctgatgcacggagttttgaaatgactgaattcaatcaagctttataagaaataaaagggcaggttgttgaaaacaactcn

SEQ ID NO: 116 - DNA, 400 bp, Homo sapiens
ggcacgagcggaccgggccgagccgggccgcccgggcgcagtctttaaccatggcgtccctcttcaagaagaaaactgtggatgatgtaataaaggaacagaatcgagagttacgaggtacacagagggctataatcagagatcgagcagctttagagaaacaagaaaaacagctggaattagaaattaagaaaatggccaagattggtaataaggaagcttgcaaagttttagccaaacaacttgtgcatctacggaaacagaagacgagaacttttgctgtaagttcaaaagttacttctatgtctacacaaacaaaagtgatgaattcccaaatgaagatggctggagcaatgtctaccacagcaaaaacaatgcaggcagttaacaagaagatggg

SEQ ID NO: 117 - DNA, 402 bp, Homo sapiens
ggcacgaggggagatcgctcagctggccgtgtcctggcaggccacggcatatgcctccaaggacggggtcctcactgaggccatgatggacgcctgtgtgcaagatgctgtccagcagtaccgacagaagatgcgctggctgaaggcggaggggcctgggcgcggggtcgagcaccccctatccggagtccaaggcgagaccctcacctcatggagcctggccacggacccctcctacccctgccttgccggcccctgcacatttaggatatgctcctggatggggactgggctgtgcccagggcctctgtcccccaggatgtcttgtggtggcggtcggccgttctgccccccagggcaccccctgttgtaggcactggctctaggagggcaggcctcctt

SEQ ID NO: 118 - DNA, 395 bp, Homo sapiens
ggcacgaggtagagatacgaatggggtgtagtagccgactgctcgcaggcacccccaggttatgtggacagagctaagcccaaagttgtgattttccactctgttctgtccatgtcgagggaagataagtagaaagtgacacagtaagagccagaatacaccaggtgaaggagagaattgcattgtgttttgagaagtttcactgacaagttatcctgggctgtgggacatcactagctttgaaagtgtagctggcacctcgtccatctaatttgatgggtgtgtgtggggtgttgggcacgcgtcggcctagcagatctgaacccaggtgatttctgttctcaggaagcttttaggtgacaaggatcaggcatgtgaacaaataaccatactgg

SEQ ID NO: 119 - DNA, 144 bp, Homo sapiens
  misc_feature (1)...(144): n = A,T,C or G
ccggtaaggaatatacttcttctgatactaaatatgccaatatttaaaatgtaatattcagggattacaactgtgagggctaaacacacggaattacccaccaattcctctgtagttctctactaattcaattttgcatcctcn

SEQ ID NO: 120 - DNA, 392 bp, Homo sapiens
ggcacgagaccaggtcataagaggatccgttccaatgattttcctaaaacaatggaagtgttttccaaagagcttataaggcattgtaggatctggcctgccctgactccactttaccagaaccatctgctgctcttctctcttgtgttactcaaggtattagctgctgtggcaaatcaactctgaaatctccgtgacttaatacaagagaggtttatttcttactcacgctgggtgcactgccacttggtaacagaggagctatggaaacttgagacctaagcagaaatgagttcaataatattgctacactctaggactttctccaaaattaacaacagaacaaaagtgcaaggcagtgataacccatctgacagcatttggggagtgtt

SEQ ID NO: 121 - DNA, 395 bp, Homo sapiens
ggcacgagatcaatcacaaaagtttatccttaagacttcccttcagctgctggaaggcagtcatcacatctgtgaaaagagtgctagttataacaaatgagatcacaaatttgaccattttattagacaccctctattagtgttaacagacaaagatgaaggttaagttgaaatcaaattgaaatcatcttccctctgtacagattgcaatatctgataataccctcaactttcttggtgcaaattaattgcctggtactcacagtccagtgttaacaggcaataatggtgtgattccagaggagaggactaggtggcaggaaaataaatgagattagcagtatttgattggagccataagcataatttggttccggcggcggccaggtttaaaa

SEQ ID NO: 122 - DNA, 288 bp, Homo sapiens
  misc_feature (1)...(288): n = A,T,C or G
cgcccgcgcctctctgttctctctcgcgcgcggtgtctctctcgatagagtgcgcgacctgcacaccctctgtgtggggttctcgctccccgtgtgcgcgcgcgcgcgctctctgtgggactcgcacacaccgcgcgcgcgcgcgctctctgtgggggggccctccccgcaccttgtgtgtgtgtgtctgtgttatctctgtgagatgtgcgtgnnnnnnnnnnntctgtgtgtgtgtctgccctccgcgccgtgtctgttatatatgcgctcgctcgctggggcgcg

SEQ ID NO: 123 - DNA, 393 bp, Homo sapiens
ggcacgaggatccattcttcgacccccagatgtgactctaaagaaggctgaaaatttttgtccaaattgccatgcagatatcttgaacagcaggacatttgcaggccttgtctactggacttttctcccaaacaggacaagcccaggcagggctgcatggagaggaatggaacctggagctagaattaattgcccactctcccaccctaccagtgcagcccggcaagggcaggaattgggaggcctaaggtgggcatgaaagcttgggaagcactgtcgtctctcagacaggcgtcctaaagacctctaggctggaagcttgggcttgcaagtggatccgggaccgagggtggtctcttggacaaccccaggaacttggaccaaggcagagcc

SEQ ID NO: 124 - DNA, 394 bp, Homo sapiens
ccgcgacgagatgatgatctgcttcttccattatgcccagatgataaaaaggattgatacaaaagagggatctgaattctcattttcagatggagaagtggccgaaaaagcagaggtttacaggtcagaaaatgaaagtgaacggaactgcctagaagaatcagagggctgctattgcagatcatctggagaccctgaacaaataaaggacgacagtttatcagaagagagtgctgatgcacggagttttgaaatgactgaactcaatcaagctttagaagaaataaaagggcaggctgttgaaaacacctctgtaactgaattttctgaggagaaacaccgaacttgaaattcacaccggcctaatgtccaagaattcaagggggggtccctc

SEQ ID NO: 125 - DNA, 390 bp, Homo sapiens
  misc_feature (1)...(390): n = A,T,C or G
ggcacgagcccttatacaaacatatatgaacatatatactttttttgttgtataaaaacaggatcacattatagatattattctgtaactttctgttttcacccaaaatacagcagagcactattttccagaagcacgtagttctaacttnnnnnnnnnnnnnnnnnnnnnnnnnnnnnnnnnnnnnnnnnnnnnaactttattcaagtacttcacattttaagtggacattccatttgtctgctataatttacaattatagcaatactttgagaaaggtctttgcaagtatatccatatgaactaatgtctatgtagaagatatgctggctcaaatattatgtacatttaatgtcttaataaacaccgctagattactttccaggaagc

SEQ ID NO: 126 - DNA, 388 bp, Homo sapiens
ggcacgaggtcagcacacattactttaacactttggacttgaaattctgaaagatcagaaattccttactgtttgagatgattaggttttagggactagccattttatctcacatgactcaggccttaatgctccattgctaatagctaaatgtggaaaagtttagaattacatttaatttagtcaactgttaggctgcaatcatttttttttaaaaatctgcttatggcattattcgagataacttgaccaactctaaaatatatatgtaattacttctagatgtaagtagtttttcatattaacaacacaatcaggctctgtttcagttagttcttagagtggtgaaaaaaaatctttacagtaagtgcaaaattataatccaagg

SEQ ID NO: 127 - DNA, 388 bp, Homo sapiens
ggcacgagagttaatccaaaagacttcccttcagctgctggaaatcagtcatcacatctgtgaaaagagtgctagttataacaaatgagatcacaaatttgaccattttattagacaccctctattagtgttaacagacaaagatgaaggttaagttgaaatcaaattgaaatcatcttccctctgtacagattgcaatatctgataataccctcaactttcttggtgcaaattaattgcctggtactcacagtccagtgttaacaggcaataatggtgtgattccagaggagaggactaggtggcaggaaaataaatgagattagcagtatttgacttggagccataggcatcaattctgctccagctgtcgaccaggttctaaaaa

SEQ ID NO: 128 - DNA, 267 bp, Homo sapiens
  misc_feature (1)...(267): n = A,T,C or G
actgtgtgtgtgtctgttttctctctctctcttctcagtcacactttttttttgggacacaccctccatccgcgggggggtttttttcccggcgcgcgccctttttttttgtgtgtttctctgcgcgcctctcttttttctctctcttccccccccgcttannnnnnnnnnnnnnngcggggggggttttcgcgcgttcnnnnnnnnnnnnnctcttccgcccccccacaggggggtgctgtttattatctttctttctccctgagc

SEQ ID NO: 129 - DNA, 389 bp, Homo sapiens
ggcacgagcttgactgcaaacttgctgaaggtagggactgtttgtcttggacttcgctgccagtccttagaacagtgtctgggacacagtgtgttctcaaatatttgttgctggaataaatgaatgaactaaatcagtcttttagggatttactgttaaccaccatgggaaaattaaataaatgcggggaaggaaaacgttctaaaattagaagactactttctactctcagcttctgattccctctgagctaagaaccagacagccttaggctggtaactcctataagctggtcctcctcccatgctgaccccatctttactgtacaattcacttttcatggactgaaggcaccaccaagatagatccaggagtgacaactccagtgg

SEQ ID NO: 130 - DNA, 319 bp, Homo sapiens
tgttgtaactgggagtggaggcccagtggctggggagacattaggtggtggggcccagcccgacctccaggttcttccttctccctagctgttgctttggtctggccactcccagcccccttgtccccttggaagcttgccctgccctcatcttgcccatgccttctactgccaggagacttgcacccatttcaaccctagggcgggggcaagtggggcaaggatggaccagcagaaggggggtaaggctctgttcacttccccctgcctccacagaacgaagccacggattccgttatcttcctccagttttgttcct

SEQ ID NO: 131 - DNA, 385 bp, Homo sapiens
ggcacgagaaacgtttcagctacgaaagtgagctttttccaacttttaaggatatcaggagagaagacactcttgatgtggaggtttctgccagtggctacacaaaggaaatgcaggcagatgatgaactgcttcatccattaggtccagatgataaaaatattgaaacaaaagagggatctgaattctcattttcagatggagaagtggcagaaaaagcagaggtttacaggtcagaaaatgaaagtgaacggaactgtctagaagaatcagagggctgctattgcagatcatctggagaccctgaacaaataaaggaagacagtttatcagaagagagtgctgatgcacggagttttgaaatgactgaattcaatcaagcttt

SEQ ID NO: 132 - DNA, 383 bp, Homo sapiens
  misc_feature (1)...(383): n = A,T,C or G
ggcacgaggggaatagagggtccctggtgacagggcaaggctagatctggagcctgcacttggcctgtgacatactgtcttgtttctgagaatcctcccctacttctctagataatctccaaacacttctgtgactacttaatcacaaaggaaattttcaggagatataatcgaattctattttacaaaaaaaaagagaagggatctgaatgttttcagttcacgctagggatcnnnnnnnnnnnnnnncccaaacctgacgtttgaggacccgcctttttttcagccaatttaaaagattttttaaggtttagggttggttggccattaaaccatccccggaaagaaaatgggggtaaaagaccaagaaggaggtcgccaag

SEQ ID NO: 133 - DNA, 382 bp, Homo sapiens
ggcacgagataagatctgaggtgttacacacataattgtcccaatttttaagattgatggggagcatgaagcatttttttaatgtgttggcaggccccattaaatgcataaactgcataggactcatgtggtctgaatgtattttagggctttctgggaattgtcttgacagagaacctcagctggacaaagcagccttgatctgagtgagctaactgacacaatgaaactgtcaggcatgtttctgctcctctctctggctcttttctgctttttaacaggtgtcttcagtcagggaggacaggttgactgtggtgagttccaggacaccaaggtctactgcactcgggaatctaacccacactggggccttgaatggcca

SEQ ID NO: 134 - DNA, 375 bp, Homo sapiens
ggcacgagcaagcctttcatttacattaaattataacttttcattcattcctaaaccaaacttaaaattctgctttcctttgagtagaaggtatttaacttgttttgtttttccttcagaaggaatttaatgcaaacggattgcagtcagcactttctgaatgttttcacacagtatgcaaagcttacatcataccaaggagtggagagttgaagtttcctcccagtgactccagtgacagaccacacctagaaagcgtttctcttcctgagtatttcaaaaagatgtaaaagagctggggagagtatgggaagaaacaatacaggattgcctttaattaattaagaattgcctcctgataaaaggaaaaagaaa

SEQ ID NO: 135 - DNA, 376 bp, Homo sapiens
ggcacgagacctgtttgaggtggaactccaagcagctcgcaccttggagcgactggagctccagagtctggaggcagctgagatagagccggaggcccaggcccagaggtcgcccaggcccacgggctcagatctgctccctggagcccccatcctcagtctgcgcttctcctacatctgccctgaccggcagttgcgtcgctatttggtgctggagcctgatgcccacgcagctgtccaggagctgcttgccgtgttgaccccagtcaccaatgtggctgttcccctgcaggatctgagtggcatagagctgggcctggcaggccagagcctgcggctagagtgggcagctggggcgggccgctgtgtgctgctg

SEQ ID NO: 136 - DNA, 371 bp, Homo sapiens
ggcacgaggtcacctctaccagccctcctttctccagatggcttcttcataaccaccaggtcagaagaggatccgttccaatgattttcctaaaacaatggaagtgttttccaaagagcttataaggcattgtaggatctggcctgccctgactccactttaccagaaccatctgctgctcttctctcttgtgttactcaaggtattagctgctgtggcaaatcaactctgaaatctccgtgacttaatacaagagaggtttatttcttactcacgctgggtgcactgccacttggtaacagaggagctatggaaacttgagacctaagcagaaatgagttcaataatattgctacactctaggactttct

SEQ ID NO: 137 - DNA, 258 bp, Homo sapiens
  misc_feature (1)...(258): n = A,T,C or G
cagtttctttgtgcgcgcgcccccccttttttctctctctctccgcgcgggcgtgtccctccnnnnnnnnnnctgtgtgtgcgctctctccgccccatatatattgtgtttttctctgtggannnnnnnnnntctctctagagtcttttctctcccctcgcgcgcacattgttatacactcctcccctctctttctttttacacacacatatatattgcgcccctctccccccacacatttatatctctctcacatct

SEQ ID NO: 138 - DNA, 368 bp, Homo sapiens
ggcacgagacattttgagacttcttccaaattggtccctagaaagttacactggtttgtactctcacttatgtcactgtttataccaccactgactgctgcctgctttattatttctttaatgagttggactgaacagtggttaatcctgactctgtttttgactgacagttaacagttacatgaaccattcatattacagctcttacttaaatttgaccaagccaggatatatctgttaggccacattcatttagggatcatgttttccaaagcaggtttgggcaaaattaatccacaggactgaaaggtatacatctgtgagttttgttctcacttccacctctaatttgaagaacactttaattg

SEQ ID NO: 139 - DNA, 372 bp, Homo sapiens
acggcacgagctggctcctcgttttctttgtggacagtctcattaccaacatcctcgttcgggtctaggatgcctttctgctcgaggggaccaacgcggcgattcgctatgccttggccattatcttgtacaacgagaaggacatcttgaggctacagaatggcctggaaatctaccaggacctgcgcttcttcaccaataccaactccatcagccggaagctgatgaacattgccttcaatgacatgaaccccttccgcatgaaactattgcggcagctgtgcatggcccaccgtgagcggctggaggctgatctgccggagctggagcaacttaaggcaaagtacctggctaggcaggcatcccggcgca

SEQ ID NO: 140 - DNA, 365 bp, Homo sapiens
ggcacgaggctgagagtgcttgatacacccttgaatcccctcttatatgatgccccagcccaggagagataaaagcatcagcaccatgagattcacctgcctctggtcgttagggaacaatggaggcctgcgatttggagttaaactctcagtgatctctgtgttgacaacaccaaagctagaggaatccagtaggatgtgggcatggttttcccggaaggctgactgagcagttctgcaaatgtttgcaagtacagggcagaatttcatccagcctcagaaccttgagccaagactcagcatcagcaaagccaaaagtttcatttctttgactgtgggagtgctagtcccaacctttagatggc

SEQ ID NO: 141 - DNA, 353 bp, Homo sapiens
ggcacgagaaacaaaagagagcaagagagaagacagtgggtgaagtcctggttccagactcccctttttgccgggatatgatggatctgtcagctggtgaggcccctctaagaggggtggtatcttcgggccaggtgcctagagtcctagagagctagagatggagggaaattcagatcatctaaacccttcagcccttcactggacagaagaggaaactgaggctccatctgcatgacgttcccagagtcacggcacaaattcatggaagaagcagcaggaaactcagttctccagtctgggtccaatgtgtgttttagaaatatctccacagggttaatgactcaattttt

SEQ ID NO: 142 - DNA, 352 bp, Homo sapiens
ggcacgaggccactcgggggcccaggaacccctcagttagggcttctcagtcactgagcggaaggtgcccccagagggggcagccgcctgtgaggagcaggcgtgtctgggtaaccatgtggctcctgctggcctcccctgcctgtccccaaagcacagggctcagctccagagggagacgggctgggctgtcagtggtcccaggtgcatcccactttccagcagcacttggtgccagcagaggctgcaggtgtggcaggagggggcccagccgtgagggcaccaggttcaggcccggcatctcagggtggagagccagggctgtcctgaacctccagagggggtgagctgg

SEQ ID NO: 143 - DNA, 470 bp, Homo sapiens
gacttctgtctttttaggatcccatcgacttcaattcggcacgaggtcatgagaaaggaaccaatggagtatgagaagtttccagtgaaaaacagaaagaatccagtagaatttatttagggaagaggaaaagatgtgttcggggtggccttggaagtgaacgttgaaggactactgagattggttcaagaaactgtgaagggaaagaaagggttatactgagaaatggaagagataattttagaaacttgcgaaaaatggcttaatctaaatgagtgttaggggagatacagctgtgatgataggttgagctcacatggtggagagccacagttgcgggtgcttgcactgataatgtgagggcatggagacagacaataagttgaatgctctttttttaacaaaggaagctaaaagggagggggatgctaatttgatcaatacgtttgggaaaacttatattttcttgg

SEQ ID NO: 144 - DNA, 456 bp, Homo sapiens
  misc_feature (1)...(456): n = A,T,C or G
tagcacttttgtttaggaggaccccatcgattcgaattcggcacgagctgcactgagcagcaccggtgttcttcatccggctgcacccccaacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctggttgcagagtgagcccatgggacagccctctgaaattatactgcttacaaccatgctgagtctgcaaggacttcgtccaagcctttccgtccaggacctcaaacagatccaatcacaagaagagagatn

SEQ ID NO: 145 - DNA, 464 bp, Homo sapiens
atcgcccatacggcgagcccaccgacgcgaattcggcacgaggggaaacacaggcctcttctgcttttaggaccctccccctgccttgcagggggctcggggagagcaatatcaggagctagggcttgctgctgcccacactcctgctttttgggatatctaactgctaaggagggagttgacatcccccttctggctcatgtgtctgacaccaacaacatgggctctgtccctctctctttgactctccctttgtcctccccatacagctggggtggggtggatccctatacctggggcaggcagccccaaagtggtggagggggatggcaaagactgtataggcgccactggactctggcaaggcctttattacctttactccccttcctctcccatcaccagcctcaaggcctgaggtgtgcaggggctcctggcagctactgagtgagggttcctggtcg

SEQ ID NO: 146 - DNA, 448 bp, Homo sapiens
  misc_feature (1)...(448): n = A,T,C or G
ggcacgagctgcactgagcagcaccggtgttcttcatccggctgcacccccaacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctggttgcagagtgagcccatgggacagccctctgaaattatactgcttacaaccatgctgagtctgcaaggacttcgtccaagcctttccgtccaggacctcaaacagatccaatcacaagaagagagatttcaggaaagagaanattattcctatcatcgn

SEQ ID NO: 147 - DNA, 439 bp, Homo sapiens
ggcacgaggaaagttaagcaactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtgtctaaaattgaaatcgccttacaatg

SEQ ID NO: 148 - DNA, 334 bp, Homo sapiens
  misc_feature (1)...(334): n = A,T,C or G
ccccgcgcgcgctccctctctatcttttatacaaaatatagagagcgcacatctctgtgtgtgagagagtctgtgcgcgcgcgcatatatatatgggaggggtgtctccccccatctgtgtgtctctcctcttgcggggcatatgcgtgcgcacacccgcgcgctgtgtctcttttgtgccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgcgcgcacacacccacacaccgtgtgttctacagcgcgataaagagagacacaca

SEQ ID NO: 149 - DNA, 428 bp, Homo sapiens
ggcacgaggtcctgagcagcctcatgggaggtgaattagagaaaacaaaagagagcaagagagaagacagtgggtgaagtcctggttccagactcccctttttgccgggatatgatggatctgtcagctggtgcctagagtcctagagagctagagatggagggaaattcagatcatctaaacccttcagcccttcactggacagaagaggaaactgaggctccatctgcatgacgttcccagagtcacggcacaaattcatggaagaagcagcaggaaactcagttctccagtctgggtccaatgtgtgttttagaaatatctccacagggttaatgactcaatttttcatgcatgattgctagtaatgacaatcatgttatgtttggttctgtagctttggaaatcactccttccacttgagtttc

SEQ ID NO: 150 - DNA, 427 bp, Homo sapiens
  misc_feature (1)...(427): n = A,T,C or G
cgccccaaannnnaatctctaaaggggtaagggagatacctaccttgtctggtaggggagatgtttcgttttcatgctttaccagaaaatccacttccctgccgaccttagtttcaaagcttattcttaattagagacaagaaacctgtttcaacttgaagacaccgtatgaggtgaatggacagccagccaccacaatgaaagaaatcaaaccaggaataacctatgctgaacccacgcctcaatcgtccccaagtgtttcctgacacgcatctttgcttacagtgcatcacaactgaagaatggggttcaacttgacgcttgcaaaattaccaaataacgagctgcacggccaagagagtcacaattcaggcaacaggagcgacgggccaggaaagaacaccacccttcacaatgaatttgacac

SEQ ID NO: 151 - DNA, 437 bp, Homo sapiens
  misc_feature (1)...(437): n = A,T,C or G
ccgagccggatgnccttnnngagtatngcaangattccaattcggcacgagagacagtggcatggagctttgaaagacgagtaggtgttagcaaggaaataaggaggaacgggggttacgggcagaggagaaagcacatgccaagtcagcaaagaaaagtagaattcgaaaactttttaaaaatattactaaggattttcacaatgctgcactgggctagaaactgaagctaaaacagatacgtggtccctgctgctatggggcttacgttctacaggcaaggacaggttgtgatgagggttctgaaggatagagaccaagcatggagggtgttgaggaggcttctgcgagacctgaatgatgggaagccacgaagtgggaggggtgggggtccaggctggaggggcccaatgtatgtgtagagggactacagccct

SEQ ID NO: 152 - DNA, 425 bp, Homo sapiens
  misc_feature (1)...(425): n = A,T,C or G
ggcacgagctgcactgagcagcaccggtgttcttcatccggctgcacccccaacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctggttgcagagtgagcccatgggacagccctctgaaattatactgcttacaaccatgctgagtctgcaaggacttcgtccaagcctttccgtccagggacctcaacagatccaatcacaagaagagagatttcaggaan

SEQ ID NO: 153 - DNA, 421 bp, Homo sapiens
ggcacgagccgtggctgcctcgtgagcctcccagagcccaggcctccgtggcctcctcctgtgtgagtcccaccaggagccacgtgcccggccttgccctcaaggatttttgcttttctcctgtgcacctggcgaggctgaaggcgaggggtggaggaggccccagcacagcctcatctccatgtgtacacgtgtgtacgtgtgtatgcgtgtgtgtacgtgtgtatgcgtgtgtgtacgcgtgtgtacgtgcgtgtgtacacatgcgtggccgcctgtggtgtgcacgtgtgctctgggctccgaggcttctccagagctgggagctggctggcgtggcaagggcatgctctggggcagtgtgtccctcaggaaccagggtcctccctcccctttctgcctggtcagccccgtggcctct

SEQ ID NO: 154 - DNA, 423 bp, Homo sapiens
ggcatgagtggaagggaggcagctgcctttgtttgccatggatgggtaggggctgcactgagcagcagcggtgttcttcatccggctgcacccccaacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctggttgcagagtgagcccatgggacagccctctgaaattatactgcttacaaccatgctgagtctgcaaggacttccgccaagcctttccgtc

SEQ ID NO: 155 - DNA, 312 bp, Homo sapiens
tctgtcactcacaaaacacagtgcgcgcacatagcggggggggagcacacacacaagatgtgtgtgtatacaacccgcgcgcgagagagcgctctcttttgtgggggggaaaaaaactcttatacacacacgtgtgtgtgtgtcgctctccgaaaatacacactataacaaacgcactgtgtgtgtgagacacacactcctctctccgagtggggagagagagatcgcgctccactcttaaacacatatgcgctcacagagagcatatatatgttttttttgagagaagagagagatctctttgtggtttct

SEQ ID NO: 156 - DNA, 428 bp, Homo sapiens
tgaccttccaggctacctacgcaggtgtcggggccaacaagcacctgcaggagctggcccaggaggaggtgaagcagcatgcccaggaactctgggctgcctacaggggtctgctgcgagttgccttagagcgcaagggccaggccctggaggaggatgaagacacagagacaagggacctccaggtgcatggattggtgctgcccctcatgctgcccagcttctactcagagctcttcacgctctacctgctgcttcatgagcgggaggacagcttctacagccagggcattgccaacttgagcctctttcctgatacccaactgctcgagttcctggatgtgcagaagcacttgtggcccctcaaggacctcacgctgacgagcaatcagaggtactccctggtcagggacaagtgtttcctgtca

SEQ ID NO: 157 - DNA, 430 bp, Homo sapiens
ggcacgagaggactttgagcccagagagatgaagtcatttgctcaaggcagcagtcagtggaagggcttggagaaggagaaggggtctgaaggtggtgtgggacacatgagagtgatctcgcagcttggtttgctgcagcagactcggacaagcattgtttcagtgcctggtttctccctccacttgatgggggccaactccaacccaatgtcccattcctatcctgaaatgcttctaaaggcagtgccctgagaaccaccaacctcacagcctgtctccattttattgtcttctgggaacttctcccttctgtctagcacctgtttgcactgggattgtcctgtctgtccttcagttggatcctggtttgcacccgatgaggatttagcaattttaggctgtgcttcggcaaaggccaactcacaatgg

SEQ ID NO: 158 - DNA, 405 bp, Homo sapiens
ggcacgagggaagatttccagtggtctcaatggtgtgaatcctatgaaggtgtcttatttgttgaattagaggtgaaagcctccttcctcactcttttttagaaacagtttagttttattattatgcagaatttgttgagcaaattgcaacagcccaagccacagctagctccacaagagcccttccatgagccctcaacctgggatctcgtgtatctttgttggaatggacattaggtttccaagtccaggcctgtgatttagaagggtcaggttgggtaggagagaggagagtcttggaggggctgctccatgggggtcacacctctctcctgtgggttttcgctggtgattgagttctgaggcatttgctgcattgactgttgtagctttaactcgtgtgca

SEQ ID NO: 159 - DNA, 403 bp, Homo sapiens
ggcacgagcctgactcaaggggttttggaagatttccagtggtctcaatggtgtgaatcctatgaaggtgtcttatttgttgaattagaggtgaaagcctccttcctcactcttttttagaaacagtttagttttattattatgcagaatttgttgagcaaattgcaacagcccaagccacagctagctccacaagagcccttccatgagccctcaacctgggatctcgtgtatctttgttggaatggacattaggtttccaagtccaggcctgtgatttagaagggtcaggttgggtaggagagaggagagtcttggaggggctgctccatgggggtcacacctctctcctgtgggttttcgctggtgattgagttctgaggcatttgctgcattgactgtg

SEQ ID NO: 160 - DNA, 417 bp, Homo sapiens
  misc_feature (1)...(417): n = A,T,C or G
gttctgtgggaatagagggtccctggtgacagggcagggctagatctggagcctgcacttggcctgtgacatactgtcttgtttctgagaatcctcccctacttctctagttaatctccagagacttctgtgactacttaatcacaaaggaaattttcaggaatattatcaaatactattttagaaaaaaaaagagaagggatttgaatgttttcagttcagtttagttatcnnnnnnnnnnnnnnnccccaaactccagaatgggggcccccccttctttaaccccacctaaaaatttttcggaggttcagggttggttggcaaattacaaaaaccccaaaagaaaatgggggttaacccccttggaaaagttttcttactttggggggtggccctttgacgtnggcccgggttac

SEQ ID NO: 161 - DNA, 300 bp, Homo sapiens
  misc_feature (1)...(300): n = A,T,C or G
ctatatctctctgcgccctctccccctcttgtgttttcccccgcccctctagagatatctctctcactcgcgggcgcacacccccctttacaaaatagggggctctctgtgtgtggtgtttttcttgggcgccccctctttttttttctttttgcgggcccccccctgtgtgtctctctctagacacacccccccgcgcgtgttttttataaatatctgtctctcacacaccccctactgcccctctgtgtgtgggcgcgttcccccccacacacacagagtgtgtgnnnnnnnnnnnnn

SEQ ID NO: 162 - DNA, 411 bp, Homo sapiens
ggcacgagggcaccgagcctcctgtgggaggtcccgaggcagcttcgcctgctcggcctggctgcagccctcacctgccgcagccttagctgagcagccgccgccactgggcgccccccgctccccacttcgccagcgcccgctcctcggctcggcccggggtagtttgtagggacgcagctctccacgtgcgcgactgcgaggctggacgctacgggctcctggaaaggagcagacaccagcatttgccacaatgctgtcatccactgactttacatttgcttcctgggagcttgtggtccgcgttgaccatcccaatgaagagcaggcagaaagacgtccgcactgagaggattctggagacccttcacgttggaaggagtgatgctcaaggttagtagaacagatcaa

SEQ ID NO: 163 - DNA, 412 bp, Homo sapiens
gcacgatccatcattcaattcggacagccactccaactgacctgttccgtggctgcctcgagagcctcccatagcccaggcctccgtaggcctcctcctgtgtgagtcccaccaggagccacgtgcccggccttgccctcaagggtttttgcttttctcctgtgcacctggctaggctgaaggcgaggggtggaggaggccccagcacagcctcatctccatgtgtacacgtgtgtacgtgtgtatgcgtgtgtgtacgcgtgtacgcgtgtgtgtacgcgtgagtacgtgctgtgtgtacacatgcgtggccgcctgtggtgtgcacgtgtgctctgggctccgaggcttctccagagctgggagctggctggcgtggcaagggcatgctctggggcagtgtgtccctcag

SEQ ID NO: 164 - DNA, 411 bp, Homo sapiens
  misc_feature (1)...(411): n = A,T,C or G
ggcacgagaggatatggtgcaaaaaaatatgattttgttaaccacaacaaaaagaaaggtaagaaatgctaggagaaagctaaaagctccatactaaaataatggtcctaatattaagcaaagtaaaatgtggtatgattttgagtggtcagcagagtgtaagaataatctatttgcacttgatactttcagctgtcacagaggtcatagaattgggcttattgagaaggaaaggtaaatgctagtacactacttggctcagaagtgaacaaaattgcagtttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 165 - DNA, 415 bp, Homo sapiens
  misc_feature (1)...(415): n = A,T,C or G
ggcacgagaggatatggtgcaaaaaaatatgattttgttaaccacaacaaaaagaaaggtaagaaatgctaggagaaagctaaaagctccatactaaaataatggtcctaatattaagcaaagtaaaatgtggtatgattttgagtggtcagcagagtgtaagaataatctatttgcacttgatactttcagctgtcacagaggtcatagaattgggcttattgagaaggaaaggtaaatgctagtacactacttggctcagaagtgaacaaaattgcagtttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtn

SEQ ID NO: 166 - DNA, 403 bp, Homo sapiens
ggcacgaggaaggtgtcaggagcatcccatttgtgtctctctctctacctctgtgaagggcgcgaatgggcagagcagaacttctagaagggaagatgagcacccaggatccctcagatctgtggagcagatccgatggagaggctgagctgctccaggacttggggtggtatcacggcaacctcacacgccatgctgctgaagctcttctcctctcaaatggatgtgacggcagctaccttctgagggacagcaatgagaccaccgggctgtactctctctctgtgagggccaaagattctgttaaacactttcatgttgaatatactggatattcatttaaatttggctttaatgaattctcatctttgaaggattttgccaagcattttgcaaatcagcg

SEQ ID NO: 167 - DNA, 407 bp, Homo sapiens
ggcacgaggggcgacaagctgttggagctgcaatgggccgcggctggggattcttgtttggcctcctgggcgccgtgtggctgctcagctcgggccacggagaggagcagcccccggagacagcggcacagaggtgcttctgccaggttagtggttacttggatgattgtacctgtgatgttgaaaccattgatagatttaataactacaggcttttcccaagactacaaaaacttcttgaaagtgactactttaggtattacaaggtaaacctgaagaggccgtgtcctttctggaatgacatcagccagtgtggaagaagggactgtgctgtcaaaccatgtcaatctgatgaagttcctgatggaattaaatctgcgagctacaagtattctgaagaagccaat

SEQ ID NO: 168 - DNA, 416 bp, Homo sapiens
ggcacgagacacaactttgagacaccccaagtgctttctgcagaggttgtcgttggaaaactgtcaccttacagaagccaattgcaaggaccttgctgctgtgttggttgtcagccgggagctgacacacctgtgcttggccaagaaccccattgggaatacaggggtgaagtttctgtgtgagggcttgaggtaccccgagtgtaaactgcagaccttggtgctttggaactgcgacataactagcgatggctgctgcgatctcacaaagcttctccaagaaaaatcaagcctgttgtgtttggatctggggctgaatcacataggagttaagggaatgaagttcctgtgtgaggctttgaggaaaccactgtgcaacttgagatgtctgtggttgtggggatgttccatccctc

SEQ ID NO: 169 - DNA, 386 bp, Homo sapiens
  misc_feature (1)...(386): n = A,T,C or G
ggcacgaggaatctcgcctctgtctggtgtgttacctactgggggcacaggaacaatttcctcaaggagacagtggcatggagctttgaaagacgagtaggtgttagcaaggaaataaggaggaacgggggttacgggcagaggagaaagcacatgccaagtcagcaaagaaaagtagaattcgaaaactttttaaaaatattactaaggattttcacaatgctgcactgggctagaaactgaagctaaaacagatacgtggtccctgctgctatggggcttccgttctagaggcaaggacaggttgtgatgagggttctgaaggatagagaccaagcagggagggtgttgaggaggcttctgcgagacctgaaggatgggaagcn

SEQ ID NO: 170 - DNA, 391 bp, Homo sapiens
  misc_feature (1)...(391): n = A,T,C or G
ggcacgagaatagagggtccctggtgacagggcagggctagatctggagcctgcacttggcctgtgacatactgtcttgtttctgagaatcctcccctacttctctagttaatctccagagacttctgtgactacttaatcacaaaggaaattttcaggaatattatcaaatactattttagaaaaaaaaagagaagggatttgaatgttttcagttcagtttagttatcnnnnnnnnnnnnnnncccaaaactcaagattggggcccccccctcctttaaccccgctaaaaagttttttgggggtttagggtgggttggcaaataacaaaacccccaaaagaaaaggggggtaaacccccttggaaaagtttcctaactttggggggcgc

SEQ ID NO: 171 - DNA, 391 bp, Homo sapiens
  misc_feature (1)...(391): n = A,T,C or G
ggcacgagcctgcatcgacccatttttcctcatgacaaactattggtgcannnnnnnnnnnnnnnnnnnnnnnnnnnnnnnnnnnnnnnnnnnnnnnnnnnnnnnnnacttagggccactcatctgtcatggaaccagaatctaaatccaaataggctgttgccagtacagatggtaagtacatgtacttctggcaggaaagcagaataaaagttgactgaacctgaaagtctcggaaatggtcttctcatttctattctgtaaagtgtcacgtcttctaggcctacctctgtcaatattgaaatacaaaattaactttttctgctttttatttcacaaatcaacgggaacagtcttagtcatttgtgttttatgagttttaattaggccn

SEQ ID NO: 172 - DNA, 385 bp, Homo sapiens
  misc_feature (1)...(385): n = A,T,C or G
ggcacgaggacagtggcatggagctttgaaagacgagtaggtgttagcaaggaaataaggaggaacgggggttacgggcagaggagaaagcacatgccaagtcagcaaagaaaagtagaattcgaaaactttttaaaaatattactaaggattttcacaatgctgcactgggctagaaactgaagctaaaacagatacgtggtccctgctgctatggggcttccgttctagaggcaaggacaggttgtgatgagggttctgaaggatagagaccaagcagggagggtgttgaggaggcttctgcgagacctgaaggatgggaagccaggaagtgggaggggtgggggtncaggctggaggggcccaatgtangtgtaaagggact

SEQ ID NO: 173 - DNA, 392 bp, Homo sapiens
ggcacgagaaaggctggaagggaggcagctgcctttgtttgccatggatgggtaggggctgcactgagcagcaccggtgttcttcatccggctgcacccccgacagagctctttcttccccagatcccttttacagttggattctccctcttggatctggctctgccttagtccgacctagagggatcagcttcgcccacgcccactctcacccggaacctttcatctcttattgaagccttttaggcccattgggatgttcattagaactctgaaaactacagttctcccctttatgaggactgcaccacagctcgccctctcctgggttccgcctggttgcagagtgagcccatgggacagccctctgaaattatactgcttacaaccat

SEQ ID NO: 174 - DNA, 394 bp, Homo sapiens
ggcacgagatggaatgacagctttttttagtagcatatccttgcgctgtgttagatggagtctttgccctgatttccgtcttttgaaaatttatctgggatgtggacatcagtgggccagatgtacaaaaaggaccttgaactcttaaattggaccagcaaactgctgcagcgcaactctcatgcagatttacatttgactgttggagcaatgaaagtaaacgtgtatctcttgttcatttttatagaacttttgcatactatattggatttacctgcggtgtgactagctttaaatgtttgtgtttatacagataagaaatgctatttctttctgggtcctgcagccattggaaaaacttttttctttggaaataataaggtttttgatagat

SEQ ID NO: 175 - DNA, 387 bp, Homo sapiens
ggcacgagggcagttagggctgccatgtgctgggagctgtgtgtctgctctccttcgtccgctcccccagggcagtgtggtagcacatcccattgtagagatgagggcaccgaggcttcctggagcataccacctggtcccgttcatgagtggtggcaaagctagcactctcacttgtccattctgccttcctggagaccagtgggatgggtcagtacagcccaccacaccattagccccaggaacataaggctgtggctagacagcaggggtctcaggttcatacatgaggactggcttgtccttgagcacccactcacctgtctatgtggggaggaatcctacaataggtcaccatggcaggctggggcttgctgaccctgcccc

SEQ ID NO: 176 - DNA, 395 bp, Homo sapiens
ggcacgagcagacctccattacctccatccctgttggattatttaaagaaagcctcagacagtaagggctttttttaaaagaataaaatgacttggtttgcgcttggaagcaggggaagcattcagatgagcggtttctgcattaaccctgcctatcacgcatctcgtgtcctgtgtggctggcgagccccccttggaaggttctggtgcttcagctggctcctgcagagtccaccccgcctcgtggtgggaatgcagagccctttgctttccttcttgccgcctgcttcctgttcctggggacccgctgggcctttggtctgcatcccctggccaggtccctcagggctgatgcgcgtagaaggactttgagcagtggtggcagcacttgccct

SEQ ID NO: 177 - DNA, 388 bp, Homo sapiens
ggcacgagggacgctgcggagcccgctcacccgctccctgtacgtgaacatgactagcggcccgggtgggccggcggcggccgcgggcggcaggaaggagaaccaccagtggtatgtgtgcaacagagagaaattatgcgaatcactccaggctgtctttgttcagagttaccttgatcaaggaacacagatcttcttaaacaacagcattgagaaatcgggctggctatttatccaattatatcattcttttgtgtcatctgtttttagcctgtttatgtctagaacatctatcaatgggttgctaggaagaggctcaatgtttgtgttttcaccagatcagtttcagagactgcttaaaattaatccagactggaaaacccacaga

SEQ ID NO: 178 - DNA, 397 bp, Homo sapiens
ggcacgagcaggatccctcagatctgtggagcagatccgatggagaggctgagctgctccaggacttggggtggtatcacggcaacctcacacgccatgctgctgaagctcttctcctctcaaatggatgtgacggcagctaccttctgagggacagcaatgagaccaccgggctgtactctctctctgtgagggccaaagattctgttaaacactttcatgttgaatatactggatattcatttaaatttggctttaatgaattctcatctttgaaggattttgtcaagcattttgcaaatcagcctttgattggaagcgagacaggcactctgatggttctaaaacatccctacccaagaaaagtggaagaaccctccatttatgaatctgtccg

SEQ ID NO: 179 - DNA, 397 bp, Homo sapiens
ggcacgaggcgtggggcgacaagctgccggagctgcaatgggccgcggctggggattcttgtttggcctcctgggcgccgtgtggctgctcagctcgggccacggagaggagcagcccccggagacagcggcacagaggtgcttctgccaggttagtggttacttggatgattgtacctgtgatgttgaaaccattgatagatttaataactacaggcttttcccaagactacaaaaacttcttgaaagtgactactttaggtattacaaggtaaacctgaagaggccgtgtcctttctggaatgacatcagccagtgtggaagaagggactgtgctgtcaaaccatgtcaatctgatgaagttcctgatggaattaaatctgcgagctacaagtat

SEQ ID NO: 180 - DNA, 399 bp, Homo sapiens
  misc_feature (1)...(399): n = A,T,C or G
ggcacgaggtcaccccttttgcctccatcctcaaagacctggtcttcaagtcatccgtcagctgccaagtgttctgtaagaagatctacttcatctgggtgacgcggacccagcgtcagtttgagtggctggctgacatcatccgagaggtggaggagaatgaccaccaggacctggtgtctgtgcacatctacatcacccagctggctgagaagttcgacctcaggaccactatgctgtacatctgtgagcggcacttccagaaggttctgaaccggagtctattcacaggcctgcgctccatcacccactttggccgtcccccctttgagcccttcttcaactccctgcaggaggtccacccccacgtccggaagatcggagtgtttagctgtggcn

SEQ ID NO: 181 - DNA, 402 bp, Homo sapiens
ggcacgaggctacttcgctcgcaaggattagtactgcaaggagtatgtggcagctgtcctggagcatatcgagaacaagaacctcatgccacctcttctagtggtgcagaccctggcccacatctccacagccacactctgcgtcatcagggactacctggtccaaaaactacagaaacagagccagcagattgcacaggatgagctgcgggtgcggcggtaccgagaggagaccacccgtatccgccaggagatccaagagctcaaggccagtcctaagattttccaaaagaccaagtgcagcatctgtaacagtgccttggagatgccctcagtccacttcctgtgtggccactccttccaccaacactgctttgagagttactcggaaagcgaagctga

SEQ ID NO: 182 - DNA, 384 bp, Homo sapiens
  misc_feature (1)...(384): n = A,T,C or G
ggcacgagagcaactcaggcctgctgggttaactgcttacaccattttccttcccctcctcttccttgccttcgacactcttaacctggaaaaagcactaatttgtcctccatatctgtggttttgtcatttggaaaggttgtagaaatcctagagtatgtgaccttttaagatgcactttttagaaaactcaacatgttgctcttgtgttaatagtttgttctttttagtgttcggtattctcttgtgtggtcatgccccagtttatttaaccatcccatagatgtttattttcccttgtaaagttggttagcatgtannnnnnnnnnnnnnnnngggaaactcattctcnnnnnnnnnnnnnnnnnnnnnnnntgccccttg

SEQ ID NO: 183 - DNA, 384 bp, Homo sapiens
ggcacgagggaaggtgagggctgagaaggaggcatgccagcgggagaaagagctgcctgcagcagtacatcccttccattttgtttaaattgggcttggagaatctattctgaaaacattgactctagacttgtagaaaagagccattttagtttcaactcaaatgtaaagcaaggtagtttggtgacattttgcttttatgtgaaatagtgcacagtatgagttaatctgagcaggtctgaattgaccaaatgcttatctacgaggttcctagagctctgctgacccttggccgaaactctaaaatgtacctattaaagataaatgcttctaccaaagtaaaactctgtgagttgtttcagggcagaatgtaccagccagtca

SEQ ID NO: 184 - DNA, 379 bp, Homo sapiens
ggcacgagcttcctccagcctccacagccttggctcagtgtccctgtgtacaagacccagtgacttccaggctcccagaaaccccaccctaaccatgggccaacccagaacaccccactctccaccactggccaaagaacatgccagcagctgccccccatccatcaccaactccatggtggacatacccattgtgctgatcaacggctgcccagaaccagggtcttctccaccccagcggaccccaggacaccagaactccgttcaacctggagctgcttctcccagcaacccctgtccagccaccaggagcaacagccagaccctgtcagatgccccctttaccacatgcccagagggtacgtcgtaaaccaatatt

SEQ ID NO: 185 - DNA, 368 bp, Homo sapiens
ggcacgagacccggtccaggtgccctacgtcggcgcgagcgcgcggcaggtggagcacgtgttgtcgctgctgcgaggacgccccggaaaaacggtggatctgggctctggcgacggcaggatcgtgctggcggcccacaggtgcggcctccgcccggccgtgggctacgagctgaacccctggctggtggcgctggcgcggctgcacgcctggagggccggctgtgccggcagcgtctgctatcgccgcaaggatctctggaaggtaacctggggatccctggccacccgctgacagcccaaggtgcggctgacacctgcgagggctgggggccgggactcggaagctgcgatgacccggtgcccac

SEQ ID NO: 186 - DNA, 375 bp, Homo sapiens
ggcacgaggtctcacagagcgagaaggtgtcaggagcagcccatttgtgtctctctctctacctctgtgaagggcgcgaatgggcagagcagaacttctagaagggaagatgagcacccaggatccctcagatctgtggagcagatccgatggagaggctgagctgctccaggacttggggtggtatcacggcaacctcacacgccatgctgctgaagctcttctcctctcaaatggatgtgacggcagctaccttctgagggacagcaatgagaccaccgggctgtactctctctctgtgagggccaaagattctgttaaacactttcatgttgaatatactggatattcatttaaatttggctgtaatgaatt

SEQ ID NO: 187 - DNA, 368 bp, Homo sapiens
ggcacgaggccgtgcagagcctgtatggtaagcccctagggggctcaaaggccggccagctcccaggaaagatgtgcactgactttgaaacctgggactcctacagcccccaaggaaggcgccctgaaacgcagggccctaaatactgccactcttccttcgatgccatcactgtagacaggcaacagcaactgtacatttttaaagggagccatttctgggaggcggcagctgatggcaacgactcagagccccgtccactgcaggaaagatgggtcgggctgccccccaacattgaggctgcggcagcgtcattgaatgatggagatttctacttcttcaaaggggggcgatgctggaggatccgg

SEQ ID NO: 188 - DNA, 436 bp, Homo sapiens
ggcacgagaaggggctggggtgggctcaggcaaggcctggggccctggccttcttcctggcagggggaggcaggggactgtgcaggggctcagggaggcctcccccacctgccccctgaccacacccactctgatgaggctcatggcctcctggcaggtcgacggaggagatcatcgccctcttcatttccatcacgtttgtgctggatgccgtcaagggcacggttaaaatcttctggaagtactactatgggcattacttggacgactatcacacaaaaaggacttcatcccttgtcagcctgtcaggcctcggcgccagcctcaacgccagcctccacactgccctcaatgccagcttcctcgccagccccacggagctgccctcggccacacactcaggccaggcgaccgccgtgctcagcctcctcatcat

SEQ ID NO: 189 - DNA, 435 bp, Homo sapiens
  misc_feature (1)...(435): n = A,T,C or G
ggcacgagacagaccctttcttcctaaaggctttgtggcatcagacacataaagggtatatgtagtgtggagcactaaccatggcagggtaatttattccaggcacagagtcataattctggaaacatctagactcactgcattaacagagcattttgtttctaaagtagacctcttatgtcatccagatttcactcattctgaccacagccaggaagctgagggtgaagccagaattagctgaaacccaccaagagctgcatagagcacgtttagctagagtaggagtttgcagtgctcatatgggaaatgctgctgctatacttttaggaatttctgagtgcaatttagaaacatctagcacacttgaaacactgcgtatcattntcctcactcatgaatatagtcatcagaattcataaatagtttacctga

SEQ ID NO: 190 - DNA, 437 bp, Homo sapiens
  misc_feature (1)...(437): n = A,T,C or G
ggcacgagattaggacccttccttggcacaggggtgagaaagagcttggggaacgcttggcattatggagggctggaaggggctcaaccccgatttggagagaagtttgggatggagtgggcgagagattgagagagcgagcaggaaaagaggtcttggagcctgggactgatggtggataaggcctggaaagaagatgacgaggaggaggagagagggaagtggggtggatgaggagcaggctgacacctgggctgccctcaatccccaaggccagggagggcggngctggcccctgggaagaactgggtctctgggctccctatgcactgcccaaactggctgagccaggagtggggcaggaagtgagagtcaaggcccagcaaaaggagggggaggagctgccaattataaccttgtgganggaccggtttgng

SEQ ID NO: 191 - DNA, 434 bp, Homo sapiens
ggcacgagaagaaactgtgaagggaaagaaaggtttatactgagaaatggaagagataattttagaaacttgtgaaaaatggcttaatctaaatgagtgttaggggagatacagttgtgatgataggttgagctcacatggtggagagccacagttgcgggtgcttgcactgataatgtgagggcatggagacagacaataggttgaatgctctttttttacaaaaggaagtagaaagggagggggatgtaaatttgataaataggttggtgaaaacttatattttcttgtaaagagagagaactgagcatgttgtaggtataaggtaaaaaggcgtgaagaggaatatttcgttgataatgaaagtgagcagctagggaagaaaactcccagaggaagagggaggcaaggaaatcaagaacacacttaaagtg

SEQ ID NO: 192 - DNA, 323 bp, Homo sapiens
  misc_feature (1)...(323): n = A,T,C or G
gggtctctcgcccccctctctctcttttgtgtgtctctctctctgtcccgtgtgtgnnnnnnnnnnnnntctctctatatctcgcgcgcgcgcactcccgtgtgtgtgtgtgaccccgccccctcatgcgctctctcatttgtggagagagagaccgctatctatctctctctcccccgccctatacacatctccctctctgtgaaagagacgtgtgtgtgtctccacaccccttgggcgcgcgcgcgccaccccctctcctggggggggtgtcctctctgtatatatatatgtgcacacacgcgcgcgcgctctgtgttgtt

SEQ ID NO: 193 - DNA, 412 bp, Homo sapiens
ggcacgagaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgactttta

SEQ ID NO: 194 - DNA, 405 bp, Homo sapiens
  misc_feature (1)...(405): n = A,T,C or G
cgttgctgtcggtcagcaatgaaataaatatcttgtagaatgttcnnnnnnnnnnnnnnnnngaaccctcgggggcccttttttcccgaaacccccactggaaaaaaacccttggggggttggcaaaaccccccaataaaagggggggaaaaaaaggctttttttggaaaaatgggggggtctttgctttttttggacccctttaaagcggggaaaaccaggttaaccccccccaggggcnnnnnnnnnngtttcagggccnnnnnnnnnnnnnnnnnnttttttccctntctcccttctgtctcgccctgctgcgctgccgttttctcgttccactccccccgtttttgtactccccccgtgccgttgagcgtccaccctattctttcgcgccggtgcaccccc

SEQ ID NO: 195 - DNA, 400 bp, Homo sapiens
  misc_feature (1)...(400): n = A,T,C or G
ggcacgagattaggacccttccttggcacaggggtgagaaagagcttggggaacgcttggcattatggagggctggaaggggctcaaccccgatttggagagaagtttgggatggagtgggcgagagattgagagagcgagcaggaaaagaggtcttggagcctgggactgatggtggataaggccttgaaagaagatgacgaggaggaggagagagggaagtggggtggatgaggagcangctgacacctgggctgccctcaatccccaaggccagggagggcggngctggcccctgggaagaactgggtctctgggctccctaggcactgcccaaactggctgagccaggagtggggcaagaaatgagagttcaggcccaacacaaggagggggaggg

SEQ ID NO: 196 - DNA, 402 bp, Homo sapiens
ggcacgagattaggacccttccttggctcaggggtgagaaagagcttggggaacgcttggcattatggagggctggaaggggctcaaccccgatttggagagaagtttgggatggagtgggcgagagattgatagagcgagcaggaaaagaggtcttggagcctgggactgatggtggataaggcctggaaagaagatactaggaggaggagagagggaagtggggtggatgaggagcaggctgacacctgggctgccctcaatccccaaggccagggagggcggggctggcccctgggaagaactgggtctctgggctccctaggcactgcccaaactggctgaaccaggagtggggcaagaagtgagagtcaaggcccaacaaaaggagggggaggagct

SEQ ID NO: 197 - DNA, 401 bp, Homo sapiens
ggcacgagctctcagcggccggtttctgcgtccgctgccgcaggttccaccgcgctccaggtatttttttttctgaaggaaagctgcttcctcatatgtttcaagaatggctctccctatcattgtaaaatggggtggacaggagtattcagtgaccacactttcagaagatgatactgtgctcgatctcaaacagtttctcaagacccttacaggagttcttccagaacgccaaaagttacttggactcaaagttaaaggcaaacctgcagaaaatgatgttaagcttggagctctcaaactgaaaccaaatactaaaatcatgatgatgggaactcgtgaggagagcttggaagatgtcttaggtccaccccctgacaatgatgatgttgttaatgact

SEQ ID NO: 198 - DNA, 397 bp, Homo sapiens
  misc_feature (1)...(397): n = A,T,C or G
tgcatattagacattcttaacagggcggcagtctagtgttgaaagttttatttttccatttttcttttaagcaaattttttttaaaaaattctgattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctgatttaattcttttatttatcataaggggtttaattcctgaagtaaaggtttgcacctattaaacttaaaactgccaaatgatttttgttcttttatgtgcgcgatagaaatacaaagaatggagtggccacctcctccctttcaagctagggcagcagggacg

SEQ ID NO: 199 - DNA, 398 bp, Homo sapiens
ggcacgagaagaaaggtttatactgagaaatggaagagataattttagaaacttgtgaaaaatggcttaatctaaatgagtgttaggggagatacagttgtgatgataggttgagctcacatggtggagagccacagttgcgggtgcttgcactgataatgtgagggcatggagacagacaataggttgaatgctctttttttacaaaaggaagtagaaagggagggggatgtaaatttgataaataggttggtgaaaacttatattttcttgtaaagagagagaactgagcatgttgtaggtataaggtaaaaaggcgtgaagaggaatatttcgttgataatgaaagggagcaacttagggaaaaaaacttcccaaggaggaggggagcagggaaa

SEQ ID NO: 200 - DNA, 394 bp, Homo sapiens
ggcacgagcagaaggcagcggtctaggcgaggacgcccggctggaccaggagaccgcccagtggctgcgctgggacaagaattccttaactttggaggcagtgaaacgactaatagcagaaggtaataaagaagaactacgaaaatgttttggggcccgaatggagtttgggacagctggcctccgagctgctatgggacctggaatttctcgtatgaatgacttgaccatcatccagactacacagggattttgcagatacctggaaaaacaattcagtgacttaaagcagaaaggcatcgtgatcagttttgacgcccgagctcatccatccagtgggggtagcagcagaaggtttgcccgacttgctgcaaccacatttatcagtcagggg

SEQ ID NO: 201 - DNA, 391 bp, Homo sapiens
ggcacgagcaggcgtgtctgggtaaccatgtggctcctgctggcctcccctgcctgtccccaaagcacagggctcagctccagagggagacgggctgggctgtcagtggtcccaggtgcatcccactttccagcagcacttggtgccagcagaggctgcaggtgtggcaggagggggcccagccgtgagggcaccaggttcaggcccggcatctcagggtggagagccagggctgtcctgaacctccagagggggtgagctgggaacttgtgtgaaggggctttttccaaaaggaaaacgggagcttactggctcacggctgatgccccagacagcctcgaggatctgcaggtccccagacaccaagcctgggtgctctccagcagacggc

SEQ ID NO: 202 - DNA, 392 bp, Homo sapiens
ggcacgagattctcagtacactaaacacttgttaagagtgttgttaagagccagagtgagtatcatgtgggacacagaccctttcttcctaaaggctttgtggcatcagacacataaagggtatatgtagtgtggagcactaaccatggcagggtaatttattccaggcacagagtcataattctggaaacatctagactcactgcattaacagagcattttgtttctaaagtagacctcttatgtcatccagatttcactcattctgaccacagccaggaagctgagggtgaagccagaattagctgaaacccaccaagagctgcatagagcacgtttagctagagtaggagtttgcagtgctcatatgggaaatgctgctgctatacttt

SEQ ID NO: 203 - DNA, 392 bp, Homo sapiens
  misc_feature (1)...(392): n = A,T,C or G
ggcacgaggaggagcccgccccggaggctgaggctctggccgcagcccgggagcggagcagccgcttcttgagcggcctggagctggtgaagcagggtgccgaggcgcgcgtgttccgtggccgcttccagggccgcgcggcggtgatcaagcaccgcttccccaagggctaccggcacccggcgctggaggcgcggcttggcagacggcggacggtgcaggaggcccgggcgctcctccgctgtcgccgcgctggaatatctgccccagttgtcttttttgtggactatgcttccaactgcttatatatggaagaaattgaaggctcagtgactgttcgagattatattcagtccactatggagactgaaaaaactccccagggtctctcn

SEQ ID NO: 204 - DNA, 386 bp, Homo sapiens
ggcacgagaagccttaaaccgggaaatttccatgctatctagaggtttttgatgtcatcttaagaaacacacttaagagcatcagatttactgattgcattttatgctttaagtacgaaagggtttgtgccaatattcactacgtattatgcagtatttatatcttttgtatgtaaaactttaactgatttctgtcattcatcaatgagtagaagtaaatacattatagttgattttgctaaatcttaatttaaaagcctcattttcctagaaatctaattattcagttattcatgacaatatttttttaaaagtaagaaattctgagttgtcttcttggagctgtaggtcttgaagcagcaacgtctttcaggggttggagacag

SEQ ID NO: 205 - DNA, 295 bp, Homo sapiens
gcgctctcttcacacacaaaagatatatatatagaaagggagtgtggatatcccccctaaatatgtgagcgtgtctctctcgaccgtctcccccagagaaaatatctctagagagagcacaagtgtgttctctgtgtcttgtgtgtgagaaaaaataagtgcccgcgcacacatagatttttatatcgctcccccccgcgcctttatatatgtttttggtgtgtatatatattttatacaaaaacatgtttctttttgaggccccttacaacaaaaattttgttctttttgaacc

SEQ ID NO: 206 - DNA, 383 bp, Homo sapiens
ggcacgaggttacccatcagcccttgcaagtcccccactcaggcctctggaaggtccagggatgggctctgatgagagggtaaaagatgctcagggaaacacaggcctcagctgcctagaggaccctccccctgccttgcagtgggctcgggtagagcagtatcaggagctagggttgtctgctgcccacactcctgctttttgggatatctaactgctaaggagggagttgacatcccccttctggctcatgtgtctgacaccaacaacatggtctctgtccctctctctttgactctccctttgtcctccccatagagctggggtggggtggatccctatacctggggcaggcagccccaaagtgggggagggggatggca

SEQ ID NO: 207 - DNA, 385 bp, Homo sapiens
  misc_feature (1)...(385): n = A,T,C or G
ggcacgagcttcaggataagaagctcatggccatgttcctagagtataacaaagccatccggaactacacccgcttcgatgactggtacctgtgggttcagatgtacaaggggactgtgtccatgccagtcttccagtccttggaggcctactggcctggtcttcagagcctcattggagacattgacaatgccatgaggaccttcctcaactactacactgtatggaagcagtttggggggctcccggaattctacaacattcctcagggatacacagtggagaagcgagagggctacccacttcggccagaacttattgaaagcgcaatgtacctctaccgtgccacggnggatcccaccctcctagaactcggaagagatgg

SEQ ID NO: 208 - DNA, 374 bp, Homo sapiens
ggcacgagcctcagctgcctagaggaccctccccctgccttgcagtgggctcgggtagagcagtatcaggagctagggttgtctgctgcccacactcctgctttttgggatatctaactgctaaggagggagttgacatcccccttctggctcatgtgtctgacaccaacaacatggtctctgtccctctctctttgactctccctttgtcctccccatagagctggggtggggtggatccctatacctggggcaggcagccccaaagtgggggagggggatggcagagactgtaaaggcgccactggactctggcaaggcctttattacctttactcccctccctctcccatcaccagcctcaaggcctgagg

SEQ ID NO: 209 - DNA, 425 bp, Homo sapiens
ggcacgagcccaagtgctttctgcagaggttgtcgttggaaaactgtcaccttacagaagccaattgcaaggaccttgctgctgtgttggttgtcagccgggagctgacacacctgtgcttggccaagaaccccattgggaatacaggggtgaagtttctgtgtgagggcttgaggtaccccgagtgtaaactgcagaccttggtgctttggaactgcgacataactagcgatggctgctgcgatctcacaaagcttctccaagaaaaatcaagcctgttgtgtttggatctggggctgaatcacataggagttaagggaatgaagttcctgtgtgaggctttgaggaaaccactgtgcaacttgagatgtctgtggttgtggggatgttccatccctccgttcagttgtgaagacctctgctct

SEQ ID NO: 210 - DNA, 396 bp, Homo sapiens
ggcacgaggagcaaggaagtaatattgtcatatttgcagttgagaatgatccctgagtctcggttttcttatctatgaaatgaggctaagaataataaaatagagaattaaatgagataatgcctgtaaacagtgcctggcatatagcttattattcatccagctaagaggcccttccatatgtgaagctttgctctgtgaggtctgtattacaatcacattcagttatagctaattatttacttatgtagctatctctgaaacttagaaatgaaatcatcgaggaaaaaggccatttcttgatcctgtctgtgttccctgttcccagcataaagcctaacacgtattaggctaatgtcaccgagcaaagaaagcatcaaagtggcgggtcgggcc

SEQ ID NO: 211 - DNA, 267 bp, Homo sapiens
tctctagagacacacagagagggtgagcggctctctcacacgcaccccagagtcaggcgcgcacgctctctctctctctctctatccctcagaaagatcttcctttttccctctccctgtgatgtagtgagagtttgatgcatatttgtccgtgtccgcccccacagaccctctacctctctgtgctggccctatcttgtgtgtatgtttccctctctctctcgcgcgcccacacgatgtactttctttatatgtagtgccagttcc

SEQ ID NO: 212 - DNA, 396 bp, Homo sapiens
ggcacgagccaggaggaccctcgcttcctctccgccatgcttgccacctcttgcttctgagagtccatctcagttcgcagttctgtgacttgcattgacctggctccaatcaagctacaactcaagcagtcacggggagaaggattgtagatgggccagtgactcacagggtcaggcactcgggggagcctgagtcaggaggtcagtgggccctggaagggagggggcaagcctgggtgggtaaggttctgggccccaggcaagaaggcagagtttctccgcaggggtgtgtgcaagagctagctgcgcagaaggtctccgctggctctccaagccgggcttgtgaaataggaacgccaacatcctcctccacaggcagtggcaggcacctcctcc

SEQ ID NO: 213 - DNA, 284 bp, Homo sapiens
  misc_feature (1)...(284): n = A,T,C or G
tgggctgtctcgcccctcctccctctctctttgtactcacagtgaaaaattatagtgttcgcgtgcggggcgcgctctttacttttttttctctctcacacatatttatatatatagagagagcctccgagcgctctgcccccctcctctctctctctcttcacgtgtgtgcatcacccactcnnnnnnnnnnnctcttccagagatacgggggcttgtttcctccgctctctctcacacgtctgtgcagcagaggactatttttttctttcccccgcgtctcn

SEQ ID NO: 214 - DNA, 440 bp, Homo sapiens
  misc_feature (1)...(440): n = A,T,C or G
ggcacgagggattgcagtcagcactttctgaatgttttcacacagtatgcaaagcttacatcataccaaggagtggagagttgaagtttcctcccagtgactccagtgacagaccacacctagaaagcgtttctcttcctgagtatttcaaaaagatgtaaaagagctggggagagtatgggaagaaacaatacaggattgcctttaattaattaagaattgcctcctgataaaaggaaaaagaaattaatgctggagtatggaggggtgataaccttaaagattataaatatttgttgtctataaatacttataaattataaacacaatataattaaaattagaacatcaggaaaagaattaaaatcctcaggttgcaaaaccaaaatgttaaccaaaacaaatactcatgagattcaactttgttcacctatagaaan

SEQ ID NO: 215 - DNA, 439 bp, Homo sapiens
ggcacgagtgcacaggggacacttacggacacagaaatgcacaggggaggccgagcataaccaggggtgaggggcaggcagcagttgtagttactgccgcggggcactgctatgtgcagggacagccagcacccagcccatcaccactccctgggctggctggcaggtatggcaccctgggagcccggcatatacccagggcacccctacggctgccgccagtctcatgcccaggtgggtgctctgggctggagcgagggccaggttttgggccgaggcttccccaggcaatcctgtgagctcccttctagcctctgacccagtctggtctggcttgcatggatgtagggcttggggtgggaagttcaggtcctggctttgcctttgcctgatgtggatgagcagctcacatgctcagggccacctgagactgtcactg

SEQ ID NO: 216 - DNA, 392 bp, Homo sapiens
ggcacgaggagacagagaagtttggccagggggtccaccatactgctggtcaggttgggaaggaggcagagaagtttggccaggtggggaaggaggaagacagagtggtccaaggcctccatcatggcgttagtcaggctggaagggaggcggggcagtttggccacgacattcaccacacagcagggcaggctgggaaagagggagacatagcagttcatggtgtccaacctggggtccacgaggccgggaaggaggcagggcagtttggccagggagttcaccatacccttgaacaggccgggaaggaagcagacaaagcggtccaagggttccacactggggtccaccaggctgggaaggaagcagagaaacttggcccaggggtcaac

SEQ ID NO: 217 - DNA, 394 bp, Homo sapiens
ggcacgagcccatctggggcagcaccacgtggatctctccctcgtcaccttcaactggttcctcgtggtctttgcggacagtctcattagcaacatcctccttcgggtctgggatgccttcctgtacgaggggacgaaggtggtgtttcgctatgccttggccattttcaagtacaacgagaaggagatcttgaggctacagaatggcctggaaatctaccagtacctgcgcttcttcaccaagaccatctccaacagccggaagctgatgaacatcgccttcaatgacatgaaccccttccgcatgaaacagctgcggcagctgcgcatggtccaccgggagcggctggaggctgagctgcgggagctggagcagcttaaggcagagtacctg

SEQ ID NO: 218 - DNA, 432 bp, Homo sapiens
acacccacttgtttgaggacaccatcgattcgaattcggcacgagcctagccagcccctgacgtgccttacaggagttcttccagacacgccaaaagtgacttggactcaaagttaaaggcaaacctgcagaaaatgatgttaagcttgtagctctcaaactgaaaccacatactaatatcatgaggatggcatctcgagaggagagcttggaagatgtcttaggtccaccccctgacaatgatgatgttgttaatgactttgatattgaagatgaagtagttgaagtagaaaatagggaagaaaacctactgaaaatttctcgcagagcgaaagagtacaaagtggaaattttgaatcctcccagggaagggaaaaagcttttggtgctagatgttgattatacattatttgaccacaggtcttgtgcaag

SEQ ID NO: 219 - DNA, 395 bp, Homo sapiens
ggcacgagccctttactcctctacccaagatcttgcttgtttctttctaagttgcctctctatctagcttgcaggatttgagttgaggaaaacacagacttccatgagtttgggaactacgagagaaaagacagacagagtcaaatctacagcatatctctcacctcaggaactggaagatgtattttatcaatatgatgtaaagtctgaaatatacagctttggaatcgtcctctgggaaatcgccactggagatatcccgtttcaaggctgtaattctgagaagatccgcaagctggtggctgtgaagcggcagcaggagccactgggtgaagactgcccttcagagctgcgggagatcattgatgagtgccgggcccatgatccctctgtgc

SEQ ID NO: 220 - DNA, 487 bp, Homo sapiens
  misc_feature (1)...(487): n = A,T,C or G
tgctcttttgatgatgccatcgattcgaattcggcacgagcagctagctcagttcaaggtggaaatggcttaacgagaggaacggcaacagcaggtggctgaggactacgagctcagactggcccgggagcaagcgcgagtgtgcgaactgcagagtgggaaccagcagctggaggagcagcgggtggagctggtggaaagactgcaggccatgctgcaggcccactgggatgaggccaaccagctgctcagcaccactctcccgccgcccaaccctccagctcctcctgctggaccctccagccccgggcctcaggagcccgagaaggaggagaggagggtctggactatgcctcccatggccgtggccctgaagcctgtattgcagcagagccgggaagcaagggacgagctacctggagcgcctcctggtttttgcagntcctcctcagatcttagcctcctggtgggcccctcttttcagagc

SEQ ID NO: 221 - DNA, 365 bp, Homo sapiens
  misc_feature (1)...(365): n = A,T,C or G
ggatgccagtggtgaggctgtaagcgaaactcttcagtttaaagctcaagatctcttaagggcagtcccaagatccagagcagagatgtatgatgacgtccacagcgatggcagatactccctcagtggatctgtagctcactctagagatgccggaagagaaggcctgagaagtgacgtatttccagggccttccttcagatcaagcaacccttccatcagtgatgacagctactttcgcaaagaatgtggccgggatctggaattttctcactctgattctcgggaccaggtcattggccaccggaaattggggcatttccgttctcaggactggaaatttgcgctccgtggttcttgggaan

SEQ ID NO: 222 - DNA, 376 bp, Homo sapiens
  misc_feature (1)...(376): n = A,T,C or G
ggcacgaggagatttcccggcgggtcccggcctctgcgtgcacgcgcctgcgtgctcgcgctcgcggttctggcgctgctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgatggcagcagtggcctgcctgaagcagccactgccaagaatctagctgcagtnnnnnnnnnnnnnnnnnnnnncatgctccacacagccaccggaagccaagaacgcaccctcctgggtacagctgcaagccgccagccgaggctgcggacccgggcctccctggtgctctgggggttggg

SEQ ID NO: 223 - DNA, 399 bp, Homo sapiens
  misc_feature (1)...(399): n = A,T,C or G
ggcacgaggggtgacagagcggctggcgcatgctcagtagagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggtggaaagactgcaggccatgctgcaggcccactgggatgaggccaaccagctgctcagcaccactcttccgccgcccaaaccttcagcttcttctgcttgaccctccagccccgn

SEQ ID NO: 224 - DNA, 402 bp, Homo sapiens
ggcacgagggcagttcagtatcgatggacagatcttcctactctttgactcagagaagagaatgtgggcaacggttcatcctggagccagaaagatgaaagaaaagtgggagaatgacaaggatgtggccatgtccttccattacatctcaatgggagactgcataggatggcttgaggacttcttgatgggcatggacagcaccctggagccaagtgcaggagcaccactcgccatgtcctcaggcacaacccaactcagggccacagccaccaccctcatcctttgctgcctcctcatcatcctcccctgcttcatcctccctggcatctgaggagaatcctttagagtgacaggttaaagatgataccaaaaagcccctgtgagcacggtcttgatcag

SEQ ID NO: 225 - DNA, 270 bp, Homo sapiens
  misc_feature (1)...(270): n = A,T,C or G
ctctctttctttctccctccccccccgggcgcgctcatttatctcgtctcttatgtctctctctctgtgtctgtgacagacacactctttttcatatagcgcgctcccttttctttgctctcggggggggtctctctgtacgcgtgtgttctctctccagtgagtgtgcacgcctaggtgagagagagtnnnnnnnnnnnnnnnntgtgtgtgaattttatatatttctatatctctcactctctgggtgtcacactctccgtgtgtggg

SEQ ID NO: 226 - DNA, 404 bp, Homo sapiens
  misc_feature (1)...(404): n = A,T,C or G
ggcacgagaaccctcccaggctaagccccaatttggggctcgcctgccctgcatcagggagacatgtcagctgaggagtaattgaccagatttctgctttagaaatatggcagtggaggcaggagatggcatctgaggcccaggctggggagaagggtgctgggatgagaacctggagttcagaccagggaagggatgagagcctaagaagaggagctctcaccctgagacaggctggtgcaggagtctgctcgatccaggcctgggtccctggttccctctgagcttgggaggactatgtgagacagaacaggaccaggggcctgcattcccccttgtattattcatcttcnnnnnnnnnnnnnnnnnnnnnnnnnnnnnnnnnnnnnnnnnnnnnnnnnnnn

SEQ ID NO: 227 - DNA, 389 bp, Homo sapiens
  misc_feature (1)...(389): n = A,T,C or G
ggcacgagaagtcactcaacctctctgagccttttcttcacctataaagtggggatagtaactacctaccttatggaagcatatgaggattgtgtgaaatcatccatgtagcccttccaccgccacgtggagtttggcatggagcagtttctaaatggaagtcatcttgatcaggtgggctgccaacctctctgagcctcagtttgctcttctagggaatggggacaatgcaatgggaatctgaggattgtgtgaaattgtgcaaatgcatgaatgtgggctgggatagtaaaagggagggccccggagcagcccacctggggtcctatctagtggacgcgcccggtgcccacccattgctgtgatgccagcagcccactgcaagcatn

SEQ ID NO: 228 - DNA, 384 bp, Homo sapiens
  misc_feature (1)...(384): n = A,T,C or G
ggcacgagctgccacctctagaaagctgctttcttctatcaccgcttgcccttgaattattccctgaatgaagccaagaaccctcccaggctaagccccaatttggggctcgcctgccctgcatcagggagacatgtcagctgaggagtaattgaccagatttctgctttagaaatatggcagtggaggcaggagatggcatctgaggcccaggctggggagaagggtgctgggatgagaacctggagttcagaccagggaagggatgagagcctaagaagaggagctctcaccctgagacaggctggtgcaggagtctgctcgatccaggcctgggtccctggttccctctgagcttgggaggactatgtgagacagaacagn

SEQ ID NO: 229 - DNA, 292 bp, Homo sapiens
  misc_feature (1)...(292): n = A,T,C or G
ggtgtctctctctcgggggggccccccctctctctatttttttttgcgcgcacactcactctctctctcttttttcccccgcgcgcgcgcacgcgctttttttttcttttttctnnnnnnnnnnnactctctctcttttctcttttgtgtgggggtctccggcgcgcttctctctctctctctcacccacagacactctctctgtgtgcgcacctctctctctcggggggccggatctctctcccccctctctatctctgttattttgggggtcccctccgcgctctcctca

SEQ ID NO: 230 - DNA, 400 bp, Homo sapiens
ggcacgaggtgggacagaagtagaagagggtgaatggccctggcaggctagcctgcagtgggatgggagtcatcgctgtggagcaaccttaattaatgccacatggcttgtgagtgctgctcactgttttacaacatataagaaccctgccagatggactgcttcctttggagtaacaataaaaccttcgaaaatgaaacggggtctccggagaataattgtccatgaaaaatacaaacacccatcacatgactatgatatttctcttgcagagctttctagccctgttccctacacaaatgcagtacatagagtttgtctccctgatgcatcctgtgagtttcaaccaggtgatgtgatgtttgtgacaggatttggagcactgaaaaatgatggttac

SEQ ID NO: 231 - DNA, 332 bp, Homo sapiens
  misc_feature (1)...(332): n = A,T,C or G
tatatagacaccccgccttttttctctctctctctatacacacaccgtctctctcccccgtgtgtctctcccctctcttttgctcatacttatatacatctacacacttgtgtgggggactctctctagcgctccctctcttttgtgtgggcgctctcacacacacacacnnnnnnnnnnnnggagactcctttctctgtggagaatatgtgtgcgcaccatctctctctctcttatttttccctcgcgcgcgcgctctgtgagagagactctctgttctcacacatatgatatatatatccctcccctctctcacactcgtgccccgcgcn

SEQ ID NO: 232 - DNA, 407 bp, Homo sapiens
  misc_feature (1)...(407): n = A,T,C or G
ggcacgagaactccggctacgtttgctgtcccaacaaatagaccagggttccctaagtgtcgcttcctccaagaagccctccctgatgagttgagccactttagtttgtgctcaggctcaccctgcacgtcttggttgctctcatcactgtaatgatctaaaacacacgtctgctcatgagacccgcatcccacccccgatgctggggccgctcttggattttcatgcctgctgccagcacccagggggagctccggaaatgtctgctgggggctcggaatacccacctttctggtaatgcagcccagcgggtcccagcctcgttttccagccctcactcanaatggagtcgctctggttcgaacgcctctgancagtgtgtacctacgtgtcaggcccatccttcc

SEQ ID NO: 233 - DNA, 406 bp, Homo sapiens
ggcacgaggaaagacccacgtgctgcctcatgtggccgacatcctcagcaagtcttgcccggcacccaggtgagcctctggtgggggtgggtagtcaccactcggctctggaggatgaggcctgggccataatccagttgcagggacggatgatctccatctcgaaggtcccagaggtaactgcgttgtcccatcctccaggcatcccctgcggcgctggccaagtgcgtgctggccgaggtcccgaagcaggtggtggagtactacagccacagaggcctgcccccgagaagcctgggtgtccctgccggagaggccagcccaggctgcacaccgtgaaaatgtggagggcgtaaagggggggcccagaaagaaagtgtcccacacaacctctgtttgcacatgg

SEQ ID NO: 234 - DNA, 380 bp, Homo sapiens
ggcacgaggagggtgaatggccctggcaggctagcctgcagtgggatgggagtcatcgctgtggagcaaccttaattaatgccacatggcttgtgagtgctgctcactgttttacaacatataagaaccctgccagatggactgcttcctttggagtaacaataaaaccttcgaaaatgaaacggggtctccggagaataattgtccatgaaaaatacaaacacccatcacatgactatgatatttctcttgcagagctttctagccctgttccctacacaaatgcagtacatagagtttgtctccctgatgcatcctatgagtttcaaccaggtgatgtgatgtttgtgacaggatttggagcactgaaaaatgatggt

SEQ ID NO: 235 - DNA, 410 bp, Homo sapiens
  misc_feature (1)...(410): n = A,T,C or G
ggcacgagctgagcaggacttagaggaactccggctacgtttgctgtcccaacaaatagaccagggttccctaagtgtcgcttcctccaagaagccctccctgatgagttgagccactttagtttgtgctcaggctcaccctgcacgtcttggttgctctcatcactgtaatgatctaaaacacacgtctgctcatgagacccgcatcccacccccgatgctggggccgctcttggattttcatgcctgctgccagcacccagggggagctccggaaatgtctgctgggggctcggaatacccacctttctggtaatgcagcccagcgggtcccagcctcgttntccagccctcactcanaatggagtcgctctggttcgaacgcctctgacaagtgtgtacctacgtgt

SEQ ID NO: 236 - DNA, 394 bp, Homo sapiens
ggcacgagactccggctacgtttgctgtcccaacaaatagaccagggttccctaagtgtcgcttcctccaagaagccctccctgatgagttgagccactttagtttgtgctcaggctcaccctgcacgtcttggttgctctcatcactgtaatgatctaaaacacacgtctgctcatgagacccgcatcccacccccgatgctggggccgctcttggattttcatgcctgctgccagcacccagggggagctccggaaatgtctgctgggggctcggaatacccacctttctggtaatgcagcccagcgggtcccagcctcgttttccagccctcactcaaaatggagtcgctctggttcgaacgcctctgacaagtgtgtacctacgtgtcag

SEQ ID NO: 237 - DNA, 428 bp, Homo sapiens
  misc_feature (1)...(428): n = A,T,C or G
ttcggcacgannnaagaagaggccctcagagatctgacagcctatgagtgcgtggacaccacctcagcccactgagcaggagtcacagcacgaagaccaagcgcaaagcgacccctgccctccatcctgactgctcctcctaagagagatggcaccggccagagcaggattctgcccccttctgctgcttctgctgctggggctgtgggtggcagagatcccagtcagtgccaagcccaagggcatgacctcatcacagtggtttaaaattcagcacatgcagcccagccctcaagcatgcaactcagccatgaaaaacattaacaagcacacaaaacggtgcaaagacctcaacaccttcctgcacgagcctttctccagtgtggccgccacctgccagacccccaaaatagcctgcaagaatggcc

SEQ ID NO: 238 - DNA, 432 bp, Homo sapiens
tctcatggaggaacccatccattcgaattcggcacgaggatcaactggctatcatatctgtttaatacatttactggagccagaaacctaggccatcatcgaacgccagcccttggtctgagcctgcggctgtagatgtggaactcacagcatatgcattgttggcccagcttaccaagcccagcctgactcacaaggagatagcgaaggccactagcatataggcttggttggccaagcaacgcaatgcatatgggggcttctcttctactcacgatactgtagttgctgtacaagctcttgccaaatatgccactaccgcctacgtgccatctgaggagatcaacctggttgtaaaatccactgagaatttccagcgcacattcaacatacagccagctaacagattggtatttcagcaggataccctgc

SEQ ID NO: 239 - DNA, 373 bp, Homo sapiens
ggcacgaggcaggacctcctctcccagatcgcccagctgcaggaggagaacaagcagctcatgaccaacctctcccacaaggatgtcaacttctcagaggaggagttccagaagcatgaaggcatgtcagagcgggagcgacaggtgatgaacaagctgaaggaggtggtggacaaacaacgcgacgagatccgcgccaaggacagggagctgggcctgaaaaatgaggacgttgaggctttacagcagcagcagacacggctgatgaagatcaaccatgaccttcggcaccgggtcacggtggtggaggcccaggggaaagccctgatcgaacagaaggtggagctggaggcagacctgcagaccaaggagc

SEQ ID NO: 240 - DNA, 392 bp, Homo sapiens
ggcacgagagctgaccgagatggacgttttctacatcgcgtcgcttgtgggccacgagttcgagcgggtcattgaccagcacgggtgttaggccatcgcgcgcctcatgcccaaggtcgtgcgcgtgctggagatcttggaggtgctggtcagtcgcctccacgtcgcgcccgagctggacgatctgcgcctggagcaggacctcctctcccagatcgcccagctgctggaggagaacaagcagctcatgaccaacctctcccacaaggatgtcaacttctcagaggaggagttccagaagcatgaaggcatgtcagagcgggagcgacaggtgatgaagaagctgaaggaggtggtggacaaacaacgcgacgagatccgcgccaaggacg

SEQ ID NO: 241 - DNA, 434 bp, Homo sapiens
gatcccatccattcgaattcggcacgaggattgattcaccttcacctgtgctgcactccagctgacccaagtaggaagccggacgagctgtaaaacatgaacggaagagtggattatttggtcactgaggaagagatcaatcttaccagagggccctcagggctgggcttcaacatcgtcggtgggacagatcagcagtatgtctccaacgacagtggcatctacgtcagccgcatcaaagaaaatggggctgcggccctggatgggcggctccaggagggtgataagatcctttcggtaaatggccaagacctaaagaacctgctgcaccaggatgctgtagacctctttcgtaatgcaggctatgctgtgtctctgagagtgcagcacaggttacaggtgcagaatggacctataggacatcgaggtgaagg

SEQ ID NO: 242 - DNA, 385 bp, Homo sapiens
ggcacgaggagagcgcggacacctcctcaacccactgaacaggagtcacagcacgatgaccattcgcaaagcgacccctgccctccatcctgactgctcctcctaagagagatggcaccggccaaaacaggattatgcccccttctgctgcttctgctgctgccgctgagtgtggcagagatcccactcagtgccaaacccaagggcatgacctcatcacagtggtttagaattcagcacatgcagcccagccctcaagcatgcaactcagccatgaaaaacattaacaagcacacaaaacggtgcaaagacctcaacaccttcctgcacgagcctttctccagtgtggccgccacctgccagacccccaaaatagcctgcaaga

SEQ ID NO: 243 - DNA, 388 bp, Homo sapiens
ggcacgagagaaggcctgcggcaaagagatgagcttattgacaaacatggcttagttataatccccgatggcactcccaatggtgatgtcagtcatgaaccagtggctggagccatcactggtgcgtctcaggaagctgctcaggtcttggagtcaccaggagaagggccattacatgtttggctacgaaaacttgctggagagaaggaagaactactgtcacagattacaaaactgaagcttcagttagaggaggaacgacagaaatgctccatgactgatggcacagtgggtgacctggcaggactgcagaatggctcagacttgcaggtcatcgaaatgcagagagatgccaatagacaaattagcgaatacaaatttaagcttg

SEQ ID NO: 244 - DNA, 388 bp, Homo sapiens
  misc_feature (1)...(388): n = A,T,C or G
ggcacgaggtcactgttgaagagttcaatcttaccagagggccctcagggctgggcttcaacatcgccggtgggacagattaccagtatgtctccaacgacagtggcatctacgtcagccgcatcaaagaaaatggggctgcggccctggatgggcggctccaggagggggataagatcctttcggtaaatggccaagacctaaagaacctgctgcaccaggatgctgaacacctctttcgtaatgcaggctatgctgtgtctctgagagtgcagcacaggttacaggcgcagaatgtacctataggacatcgaggtgaaggggacccaagcggattcccatatttattgtgctggtgcccgnggctggcctctccctggtattcgcg

SEQ ID NO: 245 - DNA, 390 bp, Homo sapiens
ggcacgaggctgtgtgtctcttttctcaccccagggcctggccatgtcccctttgggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagaggactcgggctctgaactcaacagacgctcctccacgctctcctccagctccatgtccttgtcgcccag

SEQ ID NO: 246 - DNA, 397 bp, Homo sapiens
ggcacgagaccactgggacctcctgctcctcgccatcatcaacacagggctgtctctgtttgggctgccttggatccatgccgcctacccccactccccgctgcacgtgcgagccctggccttagtggaggagcgtgtggagaacggacacatctatgacacgattgtgaacgtgaaggagacgcggctgacctcgctgggcgccagcgtcctggtgggcctgtccctgttgctgctgccggtcccgcttcagtggatccccaagcccgtgctctatggcctcttcctctacatcgcgctcacctccctcgatggcaaccagctcgtccagcgcgtggccctggtggttcaggaaccaaactggggaaccccccgacacactacatcccggaggggg

SEQ ID NO: 247 - DNA, 471 bp, Homo sapiens
  misc_feature (1)...(471): n = A,T,C or G
ttacggcgcgtttgttaggggaccccaccgattcgaattcggcacgagctctttttattttcgctgatatctttcttttactaaatgccaccatccttacctgttcgggtgtctgcgtgcctaatttttcctggctgttacacaagaacccggattttagttgaactctggagcaaaaatcctgcatcatttgtaggtgggtgtcattgtgactggctgctacctccccatgagtcttctaaaataaaacctgcaaattcacatcttccccatgcttccagagaatgcatattcttcctttgaaaaaagaaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatg

SEQ ID NO: 248 - DNA, 403 bp, Homo sapiens
  misc_feature (1)...(403): n = A,T,C or G
ggcacgaggtacagacatctagttggcaggagccaaagatgttgccaaacatgtagtannnnnnnnnnnnnnnnnnnnnnnnnnnnnnnnnnnnnnnnnnnnnnnnnnnnnnnnnnnnnnnnnnnnnngagtagaggatgcctggtatgaggcaatatttgggatagggaagggaagcttgggattttagctacgtagagacacttgaaaattggagggaggaaaggagtgggtggctttggagatgttctggaatatgtgaatgaggggagtggaggggncctgnnngctctgnggaaggccangcccggtttcctgtctttcancctcttccaggaaaattacgggcagaaagaggctgagaaagtggtcccggggaaggcgctttatgaagagcttggtg

SEQ ID NO: 249 - DNA, 316 bp, Homo sapiens
  misc_feature (1)...(316): n = A,T,C or G
ccgcttaaaggcgccttcttttaatgcaatcattttgaacatgtgcgacagtcgagaatactaattggatcaatcttgatatactctacctaaagacagtctagaaacctgggggagaaagaactcacggcacaaaacattgggccgagaacggaattctctgtaagcctagttgctgaaacttcctgctgtaaccagaagccagttttatctatcggctactgaaacacccactgtgtgttgctcactccctcactcaccgaacanaacctgctacctccgcatgaatctactagtgccgataaactatatcaga

SEQ ID NO: 250 - DNA, 419 bp, Homo sapiens
ggcacgagatatcagtcaagggctcttcaagacacagcagaaacctcaccgggcctcgggctgcctcccactgggtcccatggccaccaccttgaccttggaaagctctgttatatggaaggtagggaggacactatttccctcaactacttctagtaaaaagctcagttctctccccagcagcaagagggcacctgtgaacacctgagtcacagcgcattcctcctctgcttagaacattcgatggctcccaccttacttgcagtaaatgctgaggtccttcctgtggcccccggggccctgcatgatctgatccatcccttacctaccctcatctctccactggcctccccacacttgctcccctccggacactctggactacttgctgctatctgaacataccaggcccctgcccc

SEQ ID NO: 251 - DNA, 434 bp, Homo sapiens
  misc_feature (1)...(434): n = A,T,C or G
ggcacgagggggcctccaccggtgactcgggcctggattccacggccatggcctctgccgctgcggcgcagggactgtccggggcgtccgcggacaccctgcccttccacctccagcagcacgtcctggcctctcagggcctggccatgtcccctttcggaagcctgttcccttacccctacacgtacatggacgcagcggcggccgcctcctctgcggcagcctccagctcggtgcaccgccaccccttcctcaatctgaacaccatgcgcccgcggctgcgctacagcccctactccatcccggtgccggtcccggacggcagcagactgctcaccaccgccctgccctccatggcggcggccgcggngcccctggacggcaaagacgccgccctggccgccagcccggcctcggaggcagtggactcggcg

SEQ ID NO: 252 - DNA, 425 bp, Homo sapiens
ggcacgagaaagcactcagcctggggaatgaactctgccacaatgatgatggctgtgaccactccccgcagagagttcttgaagaggagctcggcagggactggcaggccaaggtggcctccttggaggaggtgccctttgccgctgcctcaattgggcaggtgcaccagggcctgctgagggacgggacggaggtggccgtgaagatccaggtgagaggggaggctgggcagggtaggggcgggcaccctgctagcccagagaagtgactcccaccttctctccctcccttctccctttacagtaccccggcatagcccagagcattcagagcgatgtccagaacctgctggcggtactcaagatgagcgcggccctgcccgcgggcctgtttgccgagcagagcctgcaggccttgcagcagg

SEQ ID NO: 253 - DNA, 395 bp, Homo sapiens
  misc_feature (1)...(395): n = A,T,C or G
ggcacgagcagacatctagttggcaggagccaaagatgttgccaaacatgtagtannnnnnnnnnnnnnnnnnnnnnnnnnnnnnnnnnnnnnnnnnnnnnnnnnnnnnnnnnnnnnnnnnnnnngagtagaggatgcctggtatgaggcaatatttgggatagggaagggaagcttgggattttagctacgtagagacacttgaaaattggagggaggaaaggagtgggtggctttggagatgttctggaatatgtgaatgaggggagtggaggggtcctggaggctctggggaaggccaagcccgttttcctgtctttcaacctcttccaggaaaattacgggcagaaggaggctgagaaagtggcccgggtgaatgcgctatatgacgagct

SEQ ID NO: 254 - DNA, 307 bp, Homo sapiens
  misc_feature (1)...(307): n = A,T,C or G
agtcgtcttcttttaatgtaatcattttgaacatgtgtgaaagttgatcatacgaattggatcaatcttgaaatactcaaccaaaagacagtcgagaagccagggggagaaagaactcagggcacaaaatattggtctgagaatggaattctctgtaagcctagttgctgaaatttcctgctgtaaccagaagccagttttatctaacggctactgaaacacccactgtgttttgctcactccctcactcaccgatcaaaacctgctacctccccaagactttactagtgccgataaactttctcan

SEQ ID NO: 255 - DNA, 312 bp, Homo sapiens
agtcgtcttcttttaatgtaatcattttgaacatgtgtgaaagttgatcatacgaattggatcaatcttgaaatactcaaccaaaagacagtcgagaagccagggggagaaagaactcagggcacaaaatattggtctgagaatggaattctctgtaagcctagttgctgaaatttcctgctgtaaccagaagccagttttatctaacggctactgaaacacccactgtgttttgctcactccctcactcaccgatcaaaacctgctacctccccaagactttactagtgccgataaactttctcaaagagc

SEQ ID NO: 256 - DNA, 415 bp, Homo sapiens
  misc_feature (1)...(415): n = A,T,C or G
ggcacgagcaggagcagctggcaagggagaaggacacggtgaagatgctgcaggaacagctggaaaaggcagcgcgtgcctggcgccaaagcagggcgggaggagtcgagctgccgggagccccggggaggcaggaccgggagaggcagagctgggcggagtcgtcaagctgctgggagcgctgggctgggagccccaggggaggcagagctgggcggaggtagtggggacagagacttcctaacgagggcttcagcccacccggcccaccacccacccttctggggttcccttgctgggaagcgagtgtctgatccccctgctggcccaggtcctcactttgcacctgtgtgggccccttagccagtgctccagcccctgccctgcaggatgatggtttcccctcagctcccan

SEQ ID NO: 257 - DNA, 396 bp, Homo sapiens
agaaagggtgagtgaggtgctgtcctggggttctccaagtttgagagcatggatgcatgtggtttgaagctgaagtgggcctgggggaatgggttgaaggcagaagcaaccagtttggagggaaggcatttggatatccagccctttctctgtggccttggccctgggtctgtcctgttacccccacccatacctgtctgctgcgcactctgtgcttctgtagcattctcgcttctggcctttaaagttggcaaggggaggttaataagcacctaggtggctgagtgtctctgtcttctggcttgttcacaggacttcgagtaagaaggtgatttacagccagcctagtgcccgaagtgaaggagaattcaaacagacctcgtcattcctggtgtg

SEQ ID NO: 258 - DNA, 431 bp, Homo sapiens
  misc_feature (1)...(431): n = A,T,C or G
gnnggagggcctgcggcaaagagatgagcttattgagaaacatggcttagttataatccccgatggcactcccaatggtgatgtcagtcatgaaccagtggctggagccatcactgttgtgtctcaggaagctgctcaggtcttggagtcagcaggagaagggccattagatgtaaggctacgaaaacttgctggagagaaggaagaactactgtcacagattagaaaactgaagcttcagttagaggaggaacgacagaaatgctccaggaatgatggcacagtgggtgacctggcaggactgcagaatggctcagacttgcagttcatcgaaatgcagagagatgccaatagacaaattagcgaatacaaatttaagctttcaaaagcagaacaggatataactaccttggagcaaagtattagccggc

SEQ ID NO: 259 - DNA, 404 bp, Homo sapiens
  misc_feature (1)...(404): n = A,T,C or G
ggcacgagcaggagcagctggcaagggagaaggacacggtgaagaagctgcaggaacagctggaaaaggcagcgcgtgcctggcgccaaagcagggcgggaggagtcgagctgccgggagccccggggaggcaggaccgggagaggcagagctgggcggagtcgtcaagctgctgggagcgctgggctgggagccccaggggaggcagagctgggcggaggtagtggggacagagacttcctaacgagggcttcagcccacccggcccaccacccacccttctggggttcccttgctgggaagcgagtgtctgatccccctgctggcccaggtcctcactttgcacctgtgtgggccccttagccagtgctccagcccctgccctgcaggatgatggtttcccn

SEQ ID NO: 260 - DNA, 402 bp, Homo sapiens
ggcacgagatctccctgccttgtgagcagctggccggcggctctgggacaggcggggatgggagggagtctaccgggccactgtagagctggtagctgggagctggagctgtagagttccaggctgggagctggagagccctgggtgagagggaggcctataggggccccgggggacacaccaggcttgagggtagtaggtgctggaggcagagcctggcctgtccagggtgggacctcacgacccaccctgtccggcccccagctcggaggagcttctacgtgtatgcgggcatcctggcactgctcaacctactgcaggggctggggagtgagctgctgtgcttcgacatcatcgaggggctctggtgcgtgggggccgcagggagtctgcctcgtgggg

SEQ ID NO: 261 - DNA, 402 bp, Homo sapiens
ggcacgagatctccctgccttgtgagcagctggccggcggctctgggacaggcggggatgggagggagtctaccgggccactgtagagctggtagctgggagctggagctgtagagttccaggctgggagctggagagccctgggtgagagggaggcctagaggggccccgggggacacaccaggcttgagggtagtaggtgctggaggcagagcctggcctgtccagggtgggacctcacgacccaccctgtccggcccccagctcggaggagcttctacgtgtatgcgggcatcctggcactgctcaacctactgcaggggctggggagtgtgctgctgtgcttcgacatcatcgaggggctctggtgcgtgggggccgcagggtgtctgcctcgtgggg

SEQ ID NO: 262 - DNA, 151 bp, Homo sapiens
gccgaatatgaagctacgtccgggtatccgggttccctgtaattgctttctgatccctggtacttagatttgattacctatggaccacattggtagaactactatatgggggaacctcctgattttgggcggtctcaaaaacaaaaaaaac

SEQ ID NO: 263 - DNA, 404 bp, Homo sapiens
  misc_feature (1)...(404): n = A,T,C or G
ggcacgagggaacgtggaaggactagactgcctgagtcttctgannnnnnnnnnnnnnnnnnnnnnnnnnnnnnnnnnnnnnnnnnnnnnnnnnnnnnnnnnnnnnnnnnnnnnnnnnttggacctaccccccgagtggtttgccaggggctctcaggccttcggctacagactgagggctgcattatcagctttcctacttttgaggttttgggactttactggctttcttgctcctcaacttgcagatggcctgttgtgggacctcaccttgtgatcatgtacatgagggaaatacacacccctcccagggatgatggaaggttaaggtcctaacacctcctgcacatctgagcagctgcacattgaaccagatagtcctggaatgtgggaaaacagaggcn

SEQ ID NO: 264 - DNA, 380 bp, Homo sapiens
ggcacgaggggaacgggaagccgggacccagaactcttgtctttcaggataaagtggccagggtgtacgaagccccgggctttttcctggacctggagcccatcccgggagccttggacgctgtgcgggagatgaacgacctaccggacacgcaggtcttcatctgcaccagccccctgctgaagtaccaccactgtgtgggtgagaagtaccgctgggtggagcagcacctggggccccagttcgtagaacgaattatcctgacaagggacaagacggtggtcttgggggacctgctcattgatgacaaggacacagctcgaggccaggaggagaccccaagctgggagcacatcttgttcacctgctgccacaatcgg

SEQ ID NO: 265 - DNA, 440 bp, Homo sapiens
  misc_feature (1)...(440): n = A,T,C or G
ggcagaggcgtggacaccacctcagcccactgagcaggagtcacagcacgaagaccaagcgcaaagcgacccctgccctccatcctgactgctcctcctaagagagatggcaccggccagagcaggattctgcccccttctgctgcttctgctgctggggctgtgggtggcagagatcccagtcagtgccaagcccaagggcatgacctcatcacagtggtttaaaattcagcacatgcagcccagccctcaagcatgcaactcagccatgaaaaacattaacaagcacacaaaacggtgcaaagacctcaacaccttcctgcacgagcctttctccagtgtggccgccacctgccagacccccaaaatagcctgcaagaatggcgataaaaactgccaccaaagccacgggcccgtgttcctgaccatgtgaagctccn

SEQ ID NO: 266 - DNA, 396 bp, Homo sapiens
  misc_feature (1)...(396): n = A,T,C or G
gcacgaggaggaacgtggaaggactagactgcctgagtcttctgannnnnnnnnnnnnnnnnnnnnnnnnnnnnnnnnnnnnnnnnnnnnnnnnnnnnnnnnnnnnnnnnnnnnnnnnnttggacctaccccccgagtggtttgccaggggctctcaggccttcggctacagactgagggctgcattatcagctttcctacttttgaggttttgggactttactggctttcttgctcctcaacttgcagatggcctgttgtgggacctcaccttgtgatcatgtacatgagggaaatacacacccctcccagggatgatggaagggtaaggtcctaacacctcctgcacatctgagcagctgcacattgaaccagatagtcctggaatgtgggaac

SEQ ID NO: 267 - DNA, 429 bp, Homo sapiens
ggcacgaggatctgacagcctaggagtgcgtggacaccacctcagcccactgagcaggagtcacagcacgaagaccaagcgcaaagcgacccctgccctccatcctgactgctcctcctaagagagatggcaccggccagagcaggattctgcccccttctgctgcttctgctgctggggctgtgggtggcagagatcccagtcagtgccaagcccaagggcatgacctcatcacagtggtttaaaattcagcacatgcagcccagccctcaagcatgcaactcagccatgaaaaacattaacaagcacacaaaacggtgcaaagacctcaacaccttcctgcacgagcctttctccagtgtggccgccacctgccagacccccaaaatagcctgcaagaatggcgataaaaactgccaccagagccac

SEQ ID NO: 268 - DNA, 405 bp, Homo sapiens
ggcacgaggcggcttcctggcccgcgagcagtaccgcgccctgcggcccgacctggcggataaagtggccagtgtgtacgaagccccgggctttttcctggacctggagcccatcccgggagccttggacgctgtgcgggagatgaacgacctaccggacacgcaggtcttcatctgcaccagccccctgctgaagtaccaccactgtgtgggtgagaagtaccgctgggtggagcagcacctggggccccagttcgtagaacgaattatcctgacaagggacaagacggtggtcttgggggacctgctcattgatgacaaggacacagttcgaggccaggaggagaccccaagctgggagcacatcttgttcacctgctgccacaatcggcacctggcctgccc

SEQ ID NO: 269 - DNA, 372 bp, Homo sapiens
ggcacgagaaccctgaggcctggctatggtaccaccgggtggtaggtgcccagcgctgccccatcgtggacaccttctggcaaacagagacaggtggccacatgttgactccccttcctggtgccacacccatgaaacccggttctgctactttcccattctttggtgtagctcctgcaatcctgaatgagtccggggaagagttggaaggcgaagctgaaggttatctggctgccagcgggaccaggatggctattactggatcactggcaggattgatgacatgctcaatgtatctggacacctgctgagtacagcagaggtggagtcagcacttgtggaacatgaggctgttgcataggcacctgtggg

SEQ ID NO: 270 - DNA, 411 bp, Homo sapiens
  misc_feature (1)...(411): n = A,T,C or G
ggcacgagagctctcggcgcacggcccagcttccttcaaaatgtctactgttcacgaaatcctgtgcaagctcagcttggagggtgattgtccaggaagttattccagatgaagacttatacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 271 - DNA, 302 bp, Homo sapiens
  misc_feature (1)...(302): n = A,T,C or G
ctgagtgtgacactcagagagtgtgttatatagacagagagagagcgcgcgcctctgtccccccccttgtgtgtgccccactccagtgcgcccagatccgtgcccccccccggagcgccgtgctccctnnnnnnnnnnagtgtgcacacccccctccccctctcatgagtgcccacatatatattcctgtgtgacccctccccccccctgccagtcagtgtccccgccggagcgcgagtcactgttttattttttctcgcccccaagaagggatagcgatgtgtctctcccctcctcccaca

SEQ ID NO: 272 - DNA, 429 bp, Homo sapiens
  misc_feature (1)...(429): n = A,T,C or G
ggcacgagatgtggtacagacatctagttggcaggagccaaagatgttgccaaacatgtagtannnnnnnnnnnnnnnnnnnnnnnnnnnnnnnnnnnnnnnnnnnnnnnnnnnnnnnnnnnnnnnnnnnnnngagtagaggatgcctggtatgaggcaatatttgggatagggaagggaagcttgggattttagctacgtagagacacttgaaaattggagggaggaaaggagtgggtggctttggagatgttctggaatatgtgaatgaggggaagtggaggggcctggaggctctggggaaggccaagcccgttttcctgtctttcaacctcttccaggaaaattacgggcagaaggaggctgagaaagtggcccgggtgaaggcgctatatgaggagctggaactgtcaacagtggtcttgcaaa

SEQ ID NO: 273 - DNA, 471 bp, Homo sapiens
  misc_feature (1)...(471): n = A,T,C or G
tgttgtgcatttgggcatcccaccgattcgaattcggcacgagaaagcattgaagagacctcaaggctttaagaaatgagtaggccaaaatctaagtcaaaggagaatctgtactggggccccccgtgccctgaggtcattggccaagccaagccgaacctgagctttgatcctgatggtttggggagtgaggaagacagaagtggaagcccagttctcaccccaagaggggacacaaatggatgaccctcccatgatgctgagaccccaaaaggctacacactcaagctaaaagccagaggaaatcccatcctgccacccacaagacttcaaggaaagttgttttggtgctgagcagagcaggggaagaaggaaaacagcccttaaggagctccagccactggccagccttcatgtgactctagcccaaattcattcccatcacctggggtggaagggccagaaatctcn

SEQ ID NO: 274 - DNA, 391 bp, Homo sapiens
  misc_feature (1)...(391): n = A,T,C or G
ggcacgaggtaaactctctataagtgttcagtgttgacatagcctttgtgcatagnnnnnnnnnnnnnnnnnnnnnnnnnnnnnnnnttttttgccccacctggaaaaaaggggatgcnnnnnnnnnnggggggaaaaacaattcttaagggccctttggccataaacttttttccgggccacctttgttacttttggtcctggaaggggtttttttggggggcccacggggaggggccccataggtaaactcggaaaactttttctaacccgggttagtgttttaaattaaaaccaaaaannnnnnnnnnnttggaatccttttctttaaaaaaattaatctctcaaaggaaaacaaagnnnnnnnnnnnggggggcccctttcgtttag

SEQ ID NO: 275 - DNA, 339 bp, Homo sapiens
cactccgggggctctatttgtgtgctctgcacccagttttttatacactccacgctttggatataacatctagcgccacggtgcctatgtgtacacaccctctctctatatatagatacctctgtgcgcacatatagaggggaaaagagagatatatctattatatatacatttctacacaactgtctctggggggtcagagaacgcgcgcacccctctcttttgagagaaggagactctgtcccccctctctggggcgcagggaggccccatggcatgaagaaaaatactcacttatatctctctctctcactctctgtttgcgaaaaaacacacagg

SEQ ID NO: 276 - DNA, 434 bp, Homo sapiens
  misc_feature (1)...(434): n = A,T,C or G
ccctagctacttgctctttgtgcaggatgccatagattcgtgggctcctgccttttctcaaccccgaggtgcctgaccagttctaccgcctgtggctatccctcttcctgcacgccgggatcttgcactgcctggtgtccatctgcttccagatgactgtcctgcgggacctggagaagctggcaggctggcaccgcatagccatcatctacctgctgagtggtgtcaccggcaacctggccagtgccatcttcctgccataccgagcagaggtgggtcctgctggctcccagttcggcatcctggcctgcctcttcgtggagctcttccagagctggcagatcctggcgcggccctggcgtgccttcttcaagctgctggctggggaggcttttctcttcacctttgggctgctgccgtggattgacaacttn

SEQ ID NO: 277 - DNA, 378 bp, Homo sapiens
  misc_feature (1)...(378): n = A,T,C or G
ggcacgagaaaaagtaccgctccagagcaggagcctaggcagccgagagggtgcccgaacctgagtctgagttgcggccacttcaggagctgagaggagcaggatggaactgcaggatccaaagatgaatggagccctcccttcggatgctgtgggctacaggcaagaacgtgagggcttcctgcccagtcgtggtcctgctcctgggagcaagccggtccagttcatggatttcgaggggaagacatcgtttggaatgtcagtgttcaacctcagcaacgccatcatgggcagcggcatcctggggctggcctatgccatgggccacacgggggtcattttctttctgggcctgctgctgngccatgcgcttctgcc

SEQ ID NO: 278 - DNA, 302 bp, Homo sapiens
  misc_feature (1)...(302): n = A,T,C or G
cccccnctctcgccnnnnnnnnnnncgttttcactcccgggagtccccttgtttttggcccggatccgggttctttctttcccgtggtgccgcgggttggagtgttttatcttttcttcacatggggggctggggagttccccagaacccccagggggaaacccccctcctatgaaaatgacacatgagcccctccttccggtggcggggacctgtctctctaagacccttttctgggaaaggggtctttgtttgtatgaccccaccgacgcggggggctttctatgggccgcccccccccg

SEQ ID NO: 279 - DNA, 405 bp, Homo sapiens
ggcacgaggcctcattggagacattgacaatgccatgaggaccttcctcaactactacactgtatggaagcagtttggggggctcccggaattctacaacattcctcagggatacacagcggagaagcgagagggctacccacttcggccagaacttattgaaagcgcaatgtacctctaccgtgccacgggggatcccaccctcctagaactcggaagagatgctgtggaatccattgaaaaaatcaccaaggtggagtgcggatttgcaacaatcaaagatctgcgagaccacacgctggacaaccgcatggagtcgttcttcctggccgagactgtgaaatacctctacctcctgtttgacccaaccaacttcatccacaacaatgggtgcaccttcgacgc

SEQ ID NO: 280 - DNA, 415 bp, Homo sapiens
  misc_feature (1)...(415): n = A,T,C or G
ggcacgagggtcacctgtgctgcccctccttaatctcgtatgatggtcacagtccggtggccgtgggggtgctctgccttccctggtccccactgcccatatctgtggactgccccttccaaagacccctggggggggttggananattcaatcttaccaaactcaacgatccatccatttcatgttactgatattacatgcggacacccctggatcatattattcaaatccagtcatctattctgcattcatgaccttttgataactccatcatgacctacttgacggtcactgaccatgcttactggattccgccttgtaacaataaaatctatttaaactnnnnnnnnnnnnnnnnnnnnaccagcccacataaaatatgattgaatcaatttcttataccttcactagaat

SEQ ID NO: 281 - DNA, 389 bp, Homo sapiens
ggcacgaggtagactgggggctcactgattgcattgacacttttcatcatgggtccccgggggctcacgtggagtctgacacatgaatacatggctatcatgtctgtcaccttcaatggggaaaacaaactttgtaatggtaggaaacacaacaggtacaataatttacaaaaatatgtttgccacatttcagggcaaggcaaaatgcagaggagacatatgttaaaatcttatcattcacatttgttctttttatctttaagatgaagctcttacaccaagtgtcacgagtctggagaacagatgggttgaagagctgttcttataaaataagatctggggaacacaatcctttatatatcaacatcacagtggatttttggattggg

SEQ ID NO: 282 - DNA, 371 bp, Homo sapiens
ggcacgagatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctc

SEQ ID NO: 283 - DNA, 413 bp, Homo sapiens
  misc_feature (1)...(413): n = A,T,C or G
ggcacgaggtgggagacaccacttgtctttatgtgggtctcaaagatgatgtagaatttcctttaatttctcgcagtcttcctggaaaatattttcctttgagcagcaaatcttgtagggatatcagtgaaggtctctccctccctcctctcctgnnnnnnnnnnnnggaaacaaagttttgcttttgttccccagcctgaaggggaagggctcaattttggttaaccaaaaccttggcctccggggttaaagcaattctccggcctaaccctttggagaacctgggttaataggcgcaggcccccaggccgggttaattttgggttttaagaaaaaacagggtttctcaatgtggggcaggcgtggccaaaacccccaccctaaggggatcggccctccttggcctcccaan

SEQ ID NO: 284 - DNA, 409 bp, Homo sapiens
ggcacgaggcctggggatgctccctgctaagtgggcctgctcccacccttgccataaagctctgaggcagcctgagcctgccgtgggggccccactgtgaccctgccgcagtcttcctgggtccctgcgtcctcttaaggggcagtgacacctgcctcgctggccctgtgtgggtggcaggccccactgtttgggatatcacatggccaggcacgtggtgagcctgctcagggcggacgcctgcaggcgcgtgctcggtcacacactgccttgtgtggccctcctgtccggtgcagcctggacctggacgcctggatcaatgagccactctcggacagcgagtcagaggacgagaggcccagggccgtcttccacgaggaggagcagcggcgtcccaagcaccggccgt

SEQ ID NO: 285 - DNA, 404 bp, Homo sapiens
  misc_feature (1)...(404): n = A,T,C or G
ggcacgagccacttcacccccttgggggctgcttattcactctggggattcgccatggacacgtctcaactgcgcaagctgctgcccatgtttccctgcccctccagattgcctggagatctattttgtttccttttgtgtttctttttctgttttgagtgtctttctttgcaggtttctgtagccggaagatctccgttccgctcccagcggctccagtgtaaattccccttccccctggggaaatgcactaccttgttttggggggtttaggggtgtttttgtttttcagnngntttgnttttttggnnnnnnnnnnngntttgacttttttnncttttattttggagggtaatggaaagaataggaaaatcaggcaggggggagaatggttgtttattctt

SEQ ID NO: 286 - DNA, 441 bp, Homo sapiens
  misc_feature (1)...(441): n = A,T,C or G
ggcacgagggaagcgggtggtgtgtgtcccctgtttacttttagctgagctggggttgggtgtacgggttctgttcctctgagccctgcggcccacctgatgtttacgtgtgtgtgtgagggggggcggcgctncncnnncaccccccannggcctctatccttgtgaagctctcctcaatctaatacttattgcccctgactccaaatcttccaccttttgcctcttattatatctatgttcattaccttaggtcagctgttctctattatgacactgattcatacttttgttttttgataagtacttatttcctctctcattgttgctaatatcctcttccttttttcctttgtctactctcacttcatctataaaactcttacatatctctccactaatttctttgaactaacaatttttatatagaatttaagcctg

SEQ ID NO: 287 - DNA, 387 bp, Homo sapiens
ggcacgagcagccctggaattccgcaagcacccggaggccggggggtctccgcgggcgtcccatgcggaggacatggtgcgccgtgtactcttccccacgacctcagggaccggtccccccgccggaactgcttcctacctggtccggtcccggcagctgaatctggccagcccaacctcccggtcgctatggcacccacaggcctaacattcgcgagtccaccttccgccgtccgcgaggaaaacctgattggcgccctcttggcgatcttcgggcacctcgtggtcagcattgcacttaacctccagaagtactgccacatccgcctggcaggctccaaagatccccgggccttatttaaagaccaaaactggtggcttgggcct

SEQ ID NO: 288 - DNA, 439 bp, Homo sapiens
  misc_feature (1)...(439): n = A,T,C or G
ggcacgagggaggctggaagcgggtggtgtgtgtcccctgtttacttttagctgagctggggttgggtgtacgggttctgttcctctgagccctgcggcccacctgatgtttacgtgtgtgtgtgagggggggcggcggnnncannnnnnnnnnnnngantctttttccaataacaatattaattaatccaatcttttttcttcttctcttctttctactctttttcctcctttttttttatttacttttactcatcctcctttcttcatttactctgtcttttgtattactagcttcttctctttcgcaattttcctttattgttgtcactcttttgggaataacgtactcttatgagaagttgtttcctctttatttacatttggttgtcttctcctttcataatttattttacgtatgtttgtggagttttttctt

SEQ ID NO: 289 - DNA, 170 bp, Homo sapiens
atgagtggtcttttaattaggaacaaatctaatggaaaggagagttgactgaagttggcccacaggattgtgagctgggcagagccttcatgaaggcttgccaccttgggacgcccaatttaatgggggggcctgctgtaaggcaaaaggctttttggcaaattgctggg

SEQ ID NO: 290 - DNA, 393 bp, Homo sapiens
ggcacgaggtagactgggggctcactgagtgcagtgacacttttcatcatgggtccccgggttctcacgtggagtctgacacatgaatacatggctatcatgtctgtcaccttcaatggggaaaacaaactttgtaatggtaggaaacacaacaggtacaataatttacaaaaatatgtttgccacatttcagggcaaggcaaaatgcagtggagacatatgttaaattcttatcattcacatttgatctttttatctttaggatgaagctcttacaccaagtgtcacgagtctggagaacagatggggtgagtagttgttcttataaattagtatctgtggaacacaatcctttatatatcaacatcacagtggatttctggcttggtgcat

SEQ ID NO: 291 - DNA, 430 bp, Homo sapiens
ggcacgagggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagaaaagaaaaagatgtggctccttcacgggggcctcttgccacccttcaagtgggtcccttgtgacacccgtcaatcccagatcactgaggcc

SEQ ID NO: 292 - DNA, 423 bp, Homo sapiens
atcccatcgattcgaattcggcacgagggaagcaagggcacccgccttatggatggaattgaggggaaggcacccggggctcctgcatcgagcttccctcctatattcaatgaggaaatgaccctgcagaaggctggctgcagatgcccctgcctcccggctttgcctgcttggagtttgatggacacgtggtcctgtcagggctacagcaggtctatggtctttggtaacggaaagcgctggtgaaacagtgagctttcccgtgggtgcttttccctgacgccaacaaccagggcaagctgcctgtcctgctgcttggccgctcctcagagctgcggccgggagagttcgtggtcgccatcggaagcccgttttcccttcaaaacacagtcaccaccgggatcgtgagcaccacccagcgag

SEQ ID NO: 293 - DNA, 409 bp, Homo sapiens
ggcacgaggctaggagtactggcctagatggttatagaagtccatgccaggaggtcgtctgcagtcagagggtggttctgggctggactccagccccttcctgtcggaggccaatgccgagcggattgtgcagaccttatgtacagttcgaggggccgccctcaaggttggccagatgctcagcatccaggacaacagcttcatcagccctcagctgcagcgcatctttgagcgggtccgccagagcgccgacttcatgccccgctggcagatgctgagagttcttgaagaggagctcggcagggactggcaggccaaggtggcctccttggaggaggtgccctttgccgctgcctcaattgggcaggtgcaccagggcctgctgagggacgggacggatgtgggcgtg

SEQ ID NO: 294 - DNA, 369 bp, Homo sapiens
  misc_feature (1)...(369): n = A,T,C or G
ggcacgaggccagctgctggtggagcggcactggggactggaggctggaagcgggtggtgtgtgtcccctgtttacttttagctgagctggggttgggtgtacgggttctgttcctctgagccctgcggcccacctgatgtttacgtgtgtgtgtgagggggggcggngnnnnnnnnnnnnnnnnnnnnnnnnnnnnnnnnnnnnnnnnnnnnnnnnnnnnnnnnnnnnnnnnnnnnnnannnnnnnnnnntnaatatatttttttgtttaatgggtnnnnnnnnnnnnnnnnnnnnnnnnnnnnnnnnnnnnnnnnnnnnnnnnnnnnnnnnnnnnnnnnnnnnnnnnnnnnataaattattaaattt

SEQ ID NO: 295 - DNA, 403 bp, Homo sapiens
  misc_feature (1)...(403): n = A,T,C or G
ggcacgagtgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcnnnnnnnnnnnnnnnnnccccgccccttaaaaacaatggggggccgtttaccgaaaacccaaactggaaaaaacccttggtggagttggaccaccccccacctaaagggcggggaaaaaaaggctttattggaaaaattggggaggctttggtttaattggaacccataaaagccggcaaaaaacaggtaaccaccaccattggctttctttttaggttcagggg

SEQ ID NO: 296 - DNA, 384 bp, Homo sapiens
ggcacgaggagaacttctggatcgggcccagctcggaggccctcatccacctgggcgccaagttttcgccctgcatgcgccaggacccgcaggtgcacagcttcattcgctcggcgcgcgagcgcgagaagcactccgcctgctgcgtgcgcaacgacaggtcgggctgcgtgcagacctcggaggaggagtgctcgctaacaggaattatgccgtcaaactcctttccacgctggcagtgtgggtgaagtggcccatccatcccagcgccccagagcttgcgggccacaagagacagtttggctctgtctgccaccaggatcccagggtgtgtgatgagccctcctccgaagaccctcatgagtggccagaagacatcaccaa

SEQ ID NO: 297 - DNA, 401 bp, Homo sapiens
ggcacgagattaagtgaattgcgttatatttatgaccttaaggaccagatacaggaggtagaagggagatacatgcaggggcttaaagaactaaaggaatctttgtctgaagtggaagaaaaatacaagaaagccatggtttccaatgcacagttagacaatgagaagaacaatttgatctaccatgtagacacactcaaggatgttattgaagagcaggaggaacagatggcagaattttatagagaaaatgaagaaaaatcaaaggagttagaaaggcagaaacatatgtgtagtgtgctgcagcataagatggaagaacttaaagaaggcctgcggcaaagagatgagcttattgagaaacatggcttaagtataatccccgatggcactcccaatgg

SEQ ID NO: 298 - DNA, 430 bp, Homo sapiens
  misc_feature (1)...(430): n = A,T,C or G
aaatgggagaactctgaggtncccaacgattcgcattcggcacgaggccagctgctgggggagcggctctggggactggaggctggaagcgggtggtgtgtgtcccctgtttacttttagctgagctggggttgggtgtacgggttctgttcctctgagccctgcggcccacctgatgtttacgtgtgtgtgtgagggggggcgggnnacgntatanacccatcttattatcaaattacaaaatcccantaataggtatctccatcaagctgcangaggaggagagagaaatgagagacaattatgttcctgtggtctcagccttggatcaggagattattgaagatgattcttgccctaaggagatgctgaagcttttggactttgggggtctgttcaaccttcatggtacttcaacttcagctgggaa

SEQ ID NO: 299 - DNA, 387 bp, Homo sapiens
ggcacgaggtttcataccttctcagaattggtatatcaagacacatttaaatataagccctctggaaatggatttatatacagtcatcataattacccccttagaaattggtaatattttatagccaggtttaggtttagtgtcaagtatagtgattgctggtctatcactactcatgaagtggaaccccctctactcataaaaaccccaatcagacatatagatgaatagaaccttgataacattagaatgccttgttctctgaaggcttacaagactatacgtcaggatatattaaggagaagctgaggaacgaaagaaacttcgacaagagaatggaaatgtacatgctatagcataactgaagaataaaatacaggtttgagg

SEQ ID NO: 300 - DNA, 373 bp, Homo sapiens
ggcacgagactagtccgactttttatgtgctatgcaaaatagacatctttaacatagtcctgttactatggtaacactttgctttctgaattggaagggaaaaaaatgtaacgacagcattttaaggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagt

SEQ ID NO: 301 - DNA, 369 bp, Homo sapiens
ggcacgagactagtccgactttttatgtgctatgcaaaatagacatctttaacatagtcctgttactatggtaacactttgctttctgaattggaagggaaaaaaatgtagcgacagcattttaaggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaa

SEQ ID NO: 302 - DNA, 399 bp, Homo sapiens
ggcacgaggcagcagacacggctgatgatgatcaaccatgaccttcggcaccgggtcacggtggtggaggcccaggggaaagccctgatcgaacagaaggtggagctggaggcagacctgcagaccaaggagcaggagatgggcagcctgcgagcagagctggggaagttgcgagagaggctgcagggggagcacagccagaatggggaggaggagcctgagacggagccggtgggagaggagagcatctccgacgcagagaaggtggccatggatctcaaggaccccaaccgcccccggttcaccctgcaggagctgcgggacgtgctgcacgagaggaacgagctcaagtccaaggtgttcttgctgcaggaggagctggcttactataagagtgag

SEQ ID NO: 303 - DNA, 391 bp, Homo sapiens
  misc_feature (1)...(391): n = A,T,C or G
ggcacgagcacagcccctgactgccgcagcccccacagagcccgccgcgcaccccacgtcccccacgccagcgcccagccatggaggccatcaagnnnnnnnnnnnnnnnnnnnnnnnggacaaggagaatgccatcgaccgcgcggagcaggcggaggcggataagaaagccgctgaggacaagtgcaagcaggtggaggaggagctgacgcacctccagaagaaactaaaagggacagaggacgagctggataaatattccgaggacctgaaggacgcgcaggagaagctggagctcacggagaagaaggcctccgacgctgaaggtgatgtggccgccctcaaccgacgcatccagctcgttgaggaggagttggacagggctcanga

SEQ ID NO: 304 - DNA, 418 bp, Homo sapiens
  misc_feature (1)...(418): n = A,T,C or G
ggcacgagtgccgcagcccccacagagcccgccgcgcaccccacgtcccccacgccagcgcccagccatggaggccatcaagnnnnnnnnnnnnnnnnnnnnnnnggacaaggagaatgccatcgaccgcgcggagcaggcggaggcggataagaaagccgctgaggacaagtgcaagcaggtggaggaggagctgacgcacctccagaagaaactaaaagggacagaggacgagctggataaatattccgaggacctgaaggacgcgcaggagaagctggagctcacggagaagaaggcctccgacgctgaaggtgatgtggccgccctcaaccgacgcatccagctcgttgaggaggagttggacagggctcangaacgactggccacggccctgcagaagctggaggaggcagaa

SEQ ID NO: 305 - DNA, 420 bp, Homo sapiens
ggcacgaggatttcggcaacaatttacacagctggctggaccagacatggaggtgggtgccactgatctgatgaatattctcaacaaagtcctttctaagcacaaagatcttaagactgacggttttagtcttgacacctgccggagcattgtgtctgtcatggacagtgacacgactggtaagctgggctttgaagaatttaagtatctgtggaacaacatcaagaaatggcagtgtgtttataagcagtatgacagggaccattctgggtctctgggaagttctcagctgcggggagctctgcaggccgcaggcttccagctaaatgaacaactttaccaaatgattgtccgccggtatgctaatgaagatggagatatggattttaacaatttcatcagctgcttggtccgcctgga

SEQ ID NO: 306 - DNA, 399 bp, Homo sapiens
  misc_feature (1)...(399): n = A,T,C or G
ggcacgagccacgtcccccacgccagcgcccagccatggaggccatcaagnnnnnnnnnnnnnnnnnnnnnnnggacaaggagaatgccatcgaccgcgcggagcaggcggaggcggataagaaagccgctgaggacaagtgcaagcaggtggaggaggagctgacgcacctccagaagaaactaaaagggacagaggacgagctggataaatattccgaggacctgaaggacgcgcaggagaagctggagctcacggagaagaaggcctccgacgctgaaggtgatgtggccgccctcaaccgacgcatccagctcgttgaggaggagttggacagggctcaggaacgactggccacggccctgcagaagctggaggaggcagaanaagctgcagatg

SEQ ID NO: 307 - DNA, 438 bp, Homo sapiens
  misc_feature (1)...(438): n = A,T,C or G
atcccatcgattcgaattcggcacgagcccccacagagcccgccgtgcaccccacgtcccccacgccagcgcccagccatggaggccatcaagnnnnnnnnnnnnnnnnnnnnnnnggacaaggagaatgccatcgaccgcgcggagcaggcggaggcggataagaaagccgctgaggacaagtgcaagcaggtggaggaggagctgacgcacctccagaagaaactaaaagggacagaggacgagctggataaatattccgaggacctgaaggacgcgcaggagaagctggagctcacggagaagaaggcctccgacgctgaaggtgatgtggccgccctcaaccgacgcatccagctcgttgaggaggagttggacagggctcatgaacgactgggcacggacctgcagaagctggaggagggcagaaaaagctgc

SEQ ID NO: 308 - DNA, 419 bp, Homo sapiens
ggcacgagctttggcctgcccgctcctctcctttctggcgacccgactctggctacgcaacggggcccgcgtcaatgcctgggcctactgccacgtgctacccactggggacctgctgctggtgggcacccaacagctgggggagttccagtgctggtcactagaggagggcttccagcagctggtagccagctactgcccacaggtggtggaggacggcgtggcagaccaaacagatgagggtggcagtgtacccgtcattatcagcacatcgcgtgtgagtgcaccacctggtggcaaggccagctggggtgcagacaggtcctactggaaggagttcctggtgatgtgcacgctctttgtgctggccgtgctgctcccagttttattcttgctctaccggcaccggaacagcatgg

SEQ ID NO: 309 - DNA, 415 bp, Homo sapiens
ggcacgaggctgagccagagacgccctccattctctcttcgcgcccgctctccggctggcctcccgatgcgctgcccgccctgccaccatgacggaacaggccatctccttcgacaaagacttcttggccggaggcatcgtcgccgtcatcttcaagacggacgtggctcctatcgagcgggtcaagctgctgctgccgtccagcacgccagcaagcagatcgccgccgactagcagtacaagggcatcgtggactgcattgtccgcatccccaaagagcatggagtgctgtccttctggaagggcaaccttgccaacgtcaatcgctacttccccactcaagccctcaacttcgtcttcaaggataatgacatgcagatcttactggggggcgtggacaaacacacgcaggtct

SEQ ID NO: 310 - DNA, 396 bp, Homo sapiens
ggcacgagcgggtcctgccggtgccacatggggtaccagggcccgctgtgcactgactgcatggacggctacttcagctcgctccggaacgagacccacagcatctgcacagcctgtgacgagtcctgcaagacgtgctcgggcctgaccaacagagactgcggcgagtgtgaagtgggctgggtgctggacgagggcgcctgtgtggatgtggacgagtgtgcggccgagccgcctccctgcagcgctgcgcagttctgtaagaacgccaacggctcctacacgtgcgaagagtgtgactccagctgtgtgggctgcacaggggaaggcccaggaaactgtaaagagtgtatctctggctacgcgagggagcacggacagtgtgcagatgtggac

SEQ ID NO: 311 - DNA, 394 bp, Homo sapiens
ggcacgaggcctctgggccctacagctcatcctggtcacgtgcccctcactgctcgtggtcatgcacgtggcctaccgcgaggaacgcgagcgcaagcaccacctgaaacacgggcccaatgccccgtccctgtacgacaacctgagcaagaagcggggcggactgtggtggacgtacttgctgagcctcatcttcaaggccgccgtggatgctggcttcctctatatcttccaccgcctctacaaggattatgacatgccccgcgtggtggcctgctccgtggagccttgcccccacactgtggactgttacatctcccggcccacggagaagaaggtcttcacctacttcatggtgaccacagctgcatggagatcttcggccccaggcacc

SEQ ID NO: 312 - DNA, 384 bp, Homo sapiens
ggcacgaggcgaggaacgcgagcgcaagcaccacctgaaacacgggcccaatgccccgtccctgtacgacaacctgagcaagaagcggggcggactgtggtggacgtacttgctgagcctcatcttcaaggccgccgtggatgctggcttcctctatatcttccaccgcctctacaaggattatgacatgccccgcgtggtggcctgctccgtggagccttgcccccacactgtggactgttacatctcccggcccacggagaagaaggtcttcacctacttcatggtgaccacagctgccatctgcatcctgctcaacctcagtgaagtcttctacctggtgggcaagaggtgcatggagatcttcggccccaggcaccggcg

SEQ ID NO: 313 - DNA, 430 bp, Homo sapiens
  misc_feature (1)...(430): n = A,T,C or G
ggcacgagccggctcgtaagcaacctcttcagtctgcagtgggacccgcgcgtcatgcagcgtgccagcagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggacttagtgtcaccgggaggaan

SEQ ID NO: 314 - DNA, 408 bp, Homo sapiens
ggcacgagagcagaaggactttgtctgcaacaccaagcagcccggctgccccaacgtctgctatgacgagttcttccccgtgtcccacgtgcgcctctgggccctacagctcatcctggtcacgtgcccctcactgctcgtggtcatgcacgtggcctaccgcgaggaacgcgagcgcaagcaccacctgaaacacgggcccaatgccccgtccctgtacgacaacctgagcaagaagcggggcggactgtggtggacgtacttgctgagcctcatcttcaaggccgccgtggatgctggcttcctctatatcttccaccgcctctacaaggattatgacatgccccgcgtggtggcctgctccgtggagccttgcccccacactgtggactgttacatctcccggcc

SEQ ID NO: 315 - DNA, 412 bp, homo sapiens
tcggagcccatgcgcagcggggcgcgttagctcgcgctcttcctgacccccgatcctggggccgaggtacctttgacaggagcgtgaccctgctggaggtgtgcgggagctggcctgagggcttcgggctgcggcacatgtcctccatggagcacacggaggagggcctccgggagcgacttgccgacgccatggccgagtcacctagccgggacgtcgtgggatccggaacagaacttcagcgagagggaagcatcgagactctgagtaacagctcaggctccaccagcggcagcataccaagaaactttgatggctaccgatctccgctgcccaccaatgagagccagcccctcagcctcttcccgactggcttcccgtaggtaccagcaacctgcttctgactggccag

SEQ ID NO: 316 - DNA, 300 bp, homo sapiens
gccagcccctcagcctcttcccgactggcttcccgtaggtaccagcaacctgcttctgactggccagccccctcccctgctggaggaggggagaagccccgctctggtcctacccttcagtctctgctcttccttcatcaaccaccttccccaagcttagtgacagcagccgcccatcctacctggatggagaagagacccttctccaagcacctcagcgcacttgccctctgccacacctgtcggtggaggctgtggccaggagagactgtagaagctcggtccctgtgtatgtttgca

SEQ ID NO: 317 - DNA, 2064 bp, homo sapiens
acctcagccagattcggcacgaggggcgtaggaccctccgagccaggtgtgggatatagtctcgtggtgcgccgttttttaagccggtctgaaaagcgcaatattcgggtgggagtgacccgattttccaggctgctatccatgtccagggccaaacatgaatcctattgctcttggggagccgctggcttgcttatgcagaaaacaagttgattcgatgtcatcagtcccgtggtggagcctgtggagacaacattcagtcttatactgccacagtcattagtgctgctaaaacattgaaaagtggcctgacaatggtagggaaagtggtgactcagctgacaggcacactgccttcaggtgtgacagaagatgatgttgccatccacagtaattcacggcggagtcctttggtcccaggcatcatcacagttattgacaccgaaaccgttggagagggccaggtgcttgtgagtgaggattctgacagtgatggcattgtggcccacttccctgcccatgagaagccagtgtgctgcatggcttttaatacaagtggaatgcttctagtcacaacagacacccttggccatgactttcatgtcttccaaattctgactcatccttggtcctcatcacaatgtgctgtccaccatctgtatactcttcacaggggagaaactgaagccaaagtacaggacatctgcttcagccatgactgtcgctgggttgtggtcagtactctccggggtacttcccacgttttccccatcaacccttatggtggccagccttgtgttcgtacacatatgtcaccacgagtagtgaatcgcatgagccgtttccagaaaagtgctggactggaagagattgaacaagaactgacgtctaagcaaggaggtcgctgtagccctgttccaggtctatcaagcagcccttctgggtcacccttgcatgggaaactgaacagccaagactcctataacaattttaccaacaacaaccctggcaaccctcggctctctcctcttcccagcttgatggtagtgatgcctcttgcacaaatcaagcagccaatgacattggggaccatcaccaaacgaaccggcaaagttaaacctcctccacaaatttcacccagcaaatcgatgggcggagaattttgtgtggctgctatcttcggaacatccaggtcatggtttgcaaataatgcaggtctgaaaagagaaaaagatcagtccaaacaagttgtagttgagtccctgtacattatcagttgctatggcaccttagtggaacacatgatggagccgcgacccctcagcactgcacccaagattagtgacgacacaccactggaaatgatgacatcgcctcgagccagctggactctggttagaacccctcaatggaatgaattgcagccaccgtttaatgcaaaccaccctctgctcctcgctgcagatgcagtacagtattatcagttcctgcttgctggcctggttccccctggaagtcctgggcccattactcgacatgggtcttacgacagtttagcttctgaccatagtggacaggaagatgaagaatggctttcccaggttgaaattgtaacacacactggaccccatagacgtctgtggatgggtccacagttccagttcaaaaccatccatccctcaggccaaaccacagttatctcatccagttcatctgtgttgcagtctcatggtccgagtgacacgccacagcctcttttggattttgatacagatgatcttgatctcaacagtctcaggatccagccagtccgctctgaccccgtcagcatgccagggtcatcccgtccagtctctgatcgaaggggagtttccacagtgattgatgctgcctcaggtacctttgacaggagcgtgaccctgctggaggtgtgcgggagctggcctgagggcttcgggctgcggcacatgtcctccatggagcacacggaggagggcctccgggagcgacttgccgacgccatggccgagtcacctagccgggacgtcgtgggatccggaacag

SEQ ID NO: 318 - DNA, 1365 bp, homo sapiens
cgagaactctgagtaacagctcaggctccaccagcggcagcataccaagaaactttgatggctaccgatctccgctgcccaccaatgagagccagcccctcagcctcttcccgactggcttcccgtaggtaccagcaacctgcttctgactggccagccccctcccctgctggaggaggggagaagccccgctctggtcctacccttcagtctctgctcttccttcatcaaccaccttccccaagcttagtgacagcagccgcccatcctacctggatggagaagagacccttctccaagcacctcagcgcacttgccctctgccacacctgtcggtggaggctgtggccaggagagactgtagaagctcggtccctgtgtatgtttgcatatgacatcctgcattggatccgcttttgtattttttaaccatacccacggtggggcgggtggggggagcctggaacagtgaccagatctgggggcctgagtggggacagagttgatcgtccacctggccattttgaccctgagtggacagtcacagcctcagctcatgtctggctgtgacacacactgcccccagcttcccttggtcagccccactccagcacggggtgaacggaggcccagagtactagggaaggaggaagggaggacatgcctcttcttcctcctttctttccccatctgttcctgggaagagtttgtctttcttatctttaagcccctttaccctggtcctgtactgatcagtgaaggaaaccgtggttactgaggccctgttgaaaagtgcacgtcttgtccaataaatcacgctgcagttggaaaaaaaaaaaaaaaaaaaaaggatctttaattaagcggccgcaagcttattccctttagtgagggttaattttagcttggcactggccgtcgttttacaacgtcgtgactggtaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttaccgcgcagcgtgaccgctacacttgccagcgccctagcgcccgctcctttcgctttcttccccttcctttttcgccacgttcgccggctttcccccgtcaagctctaaatcgggggctcccctttagggttcccgatttagtgctttaccggcacctcgaccccaaaaaacttgattagggtgatggttcacgtagtgggccatcgccctgataagacggtttttcgccctttgacgttggagtccacgttct

SEQ ID NO: 319 - DNA, 22 bp, Artificial Sequence
  synthesized primer
tgggatatagtctcgtggtgcg

SEQ ID NO: 320 - DNA, 22 bp, Artificial Sequence
  synthesized primer
tgattcgatgtcatcagtcccg

SEQ ID NO: 321 - DNA, 22 bp, Artificial Sequence
  synthesized primer
tgtgtcacagccagacatgagc

SEQ ID NO: 322 - DNA, 21 bp, Artificial Sequence
  synthesized primer
tgcaaacatacacagggaccg

SEQ ID NO: 323 - DNA, 24 bp, Artificial Sequence
  synthesized primer
tttagcagcactaatgactgtggc

SEQ ID NO: 324 - DNA, 22 bp, Artificial Sequence
  synthesized primer
cgccgtgaattactgtggatgg